(19) United States
(12) Patent Application Publication
(10) Pub. No.: US 2024/0150358 A1
(43) Pub. Date: May 9, 2024

(54) PURINES AND METHODS OF THEIR USE

(71) Applicant: Kineta, Inc., Seattle, WA (US)

(72) Inventors: Kerem OZBOYA, Cambridge, MA (US); Iwona WRONA, Sharon, MA (US); Bertrand LE BOURDONNEC, Northborough, MA (US); Matthew LUCAS, Lexington, MA (US); Vanessa KURIA, Somerville, MA (US); Madeline MACDONNELL, Brighton, MA (US); Byron DELABARRE, Arlington, MA (US)

(21) Appl. No.: 18/007,983

(22) PCT Filed: Jun. 3, 2021
(86) PCT No.: PCT/US2021/035673

§ 371 Date: Dec. 2, 2022

Related U.S. Application Data
(60) Provisional application No. 63/034,293, filed on Jun. 3, 2020.

(51) Int. Cl. C07D 487/04

(52) U.S. Cl. CPC C07D 487/04

(57) ABSTRACT

Disclosed are compounds useful in the treatment of neurological disorders. The compounds described herein, alone or in combination with other pharmaceutically active agents, can be used for treating or preventing neurological diseases.

### FIELD OF THE INVENTION

The invention relates to bicyclic heteroarenes and their use for therapeutic treatment of neurological disorders in patients, such as human patients.

### BACKGROUND

An incomplete understanding of the molecular perturbations that cause disease, as well as a limited arsenal of robust model systems, has contributed to a failure to generate successful disease-modifying therapies against common and progressive neurological disorders, such as ALS and FTD. Progress is being made on many fronts to find agents that can arrest the progress of these disorders. However, the present therapies for most, if not all, of these diseases provide very little relief. Accordingly, a need exists to develop therapies that can alter the course of neurodegenerative diseases. More generally, a need exists for better methods and compositions for the treatment of neurodegenerative diseases in order to improve the quality of the lives of those afflicted by such diseases.

### SUMMARY

TDP-43 is a nuclear DNA/RNA binding protein involved in RNA splicing. Under pathological cell stress, TDP-43 translocates to the cytoplasm and aggregates into stress granules and related protein inclusions. These phenotypes are hallmarks of degenerating motor neurons and are found in 97% of all ALS cases. The highly penetrant nature of this pathology indicates that TDP-43 is broadly involved in both familial and sporadic ALS. Additionally, TDP-43 mutations that promote aggregation are linked to higher risk of developing ALS, suggesting protein misfolding and aggregation act as drivers of toxicity. TDP-43 toxicity can be recapitulated in yeast models, where the protein induces a viability deficit and localizes to stress granules.

In an aspect, the invention provides a compound of formula I:

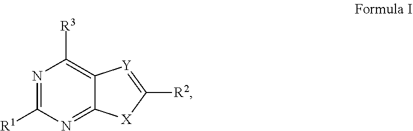

or a pharmaceutically acceptable salt thereof,
where
X is NR^{A}, S, or O;
Y is CR^{A} or N;
R¹ is hydrogen, optionally substituted C₁₋₆ alkyl, optionally substituted C₆₋₁₀ aryl, optionally substituted C₁₋₉ heterocyclyl, or optionally substituted C₁₋₉ heteroaryl;
R² is optionally substituted C₁₋₆ alkyl, optionally substituted C₆₋₁₀ aryl, optionally substituted C₁₋₉ heterocyclyl, or optionally substituted C₁₋₉ heteroaryl, or —NH—N═CHR^{B};
R³ is

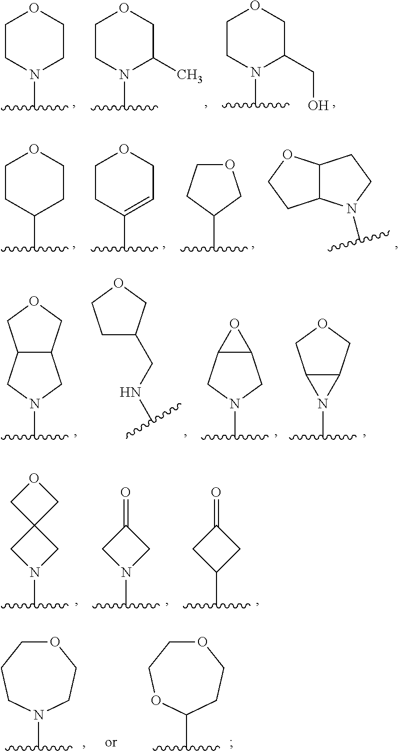

and
each R^{A} is independently H, optionally substituted C₁₋₆ alkyl, optionally substituted C₆₋₁₀ aryl, optionally substituted C₁₋₉ heteroaryl.

In some preferred embodiments, R¹ is optionally substituted C₁₋₉ heteroaryl comprising a 5-membered ring having a nitrogen atom at position 2 relative to the bond to the core, optionally substituted pyrimidin-6-yl, or optionally substituted benzodioxanyl.

In some preferred embodiments, R² is optionally substituted C₆₋₁₀ aryl, optionally substituted C₁₋₉ heterocyclyl, or optionally C₁₋₉ substituted heteroaryl.

In some embodiments, X is NR^{A}. In some embodiments, Y is N.

In some embodiments, R³ is

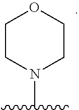 [SMILES: *N1CCOCC1]

In some embodiments, the compound is of formula Ia:

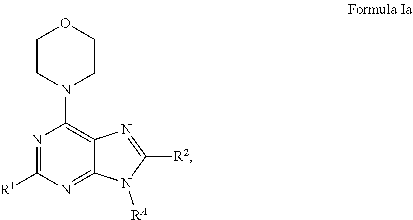

or a pharmaceutically acceptable salt thereof.

In some embodiments, R^{A} is optionally substituted C₁₋₆ alkyl. In some embodiments, R^{A} is H.

In some embodiments, the compound is of formula Ib:

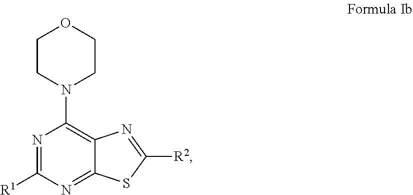

or a pharmaceutically acceptable salt thereof.

In some embodiments, the compound is of formula Ic:

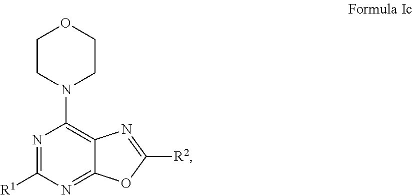

or a pharmaceutically acceptable salt thereof.

In some embodiments, the compound is of formula Id:

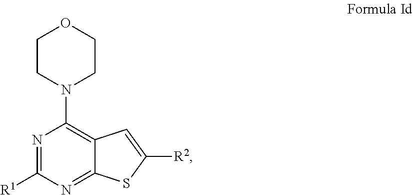

or a pharmaceutically acceptable salt thereof.

In some embodiments, R¹ is optionally substituted C₁₋₉ heteroaryl comprising a 5-membered ring having a nitrogen atom at position 2 relative to the bond to the core.

In some embodiments, R¹ is optionally substituted pyrazol-1-yl. In some embodiments, R¹ is pyrazol-1-yl substituted at position 3. In some embodiments, the pyrazol-1-yl substituted with optionally substituted C₆₋₁₀ aryl, optionally substituted C₁₋₉ heterocyclyl, optionally substituted C₁₋₉ heteroaryl, or optionally substituted C₃₋₈ cycloalkyl. In some embodiments, R¹ is optionally substituted pyrazol-3-yl. In some embodiments, R¹ is pyrazol-3-yl substituted at position 1. In some embodiments, the pyrazol-3-yl substituted with optionally substituted C₆₋₁₀ aryl, optionally substituted C₁₋₉ heterocyclyl, optionally substituted C₁₋₉ heteroaryl, or optionally substituted C₃₋₈ cycloalkyl. In some embodiments, R¹ is optionally substituted pyrimidin-6-yl.

In some embodiments, R² is optionally substituted C₁₋₉ heteroaryl. In some embodiments, R² is optionally substituted pyridyl. In some embodiments, R² is optionally substituted tetrahydropyranyl, optionally substituted dihydropyranyl, optionally substituted piperidinyl, or optionally substituted azetidinyl. In some embodiments, R² is optionally substituted tetrahydropyran-4-yl, optionally substituted 5,6-dihydro-2H-pyran-4-yl, optionally substituted piperidin-4-yl, or optionally substituted piperidin-3-yl.

In some embodiments, the compound is:

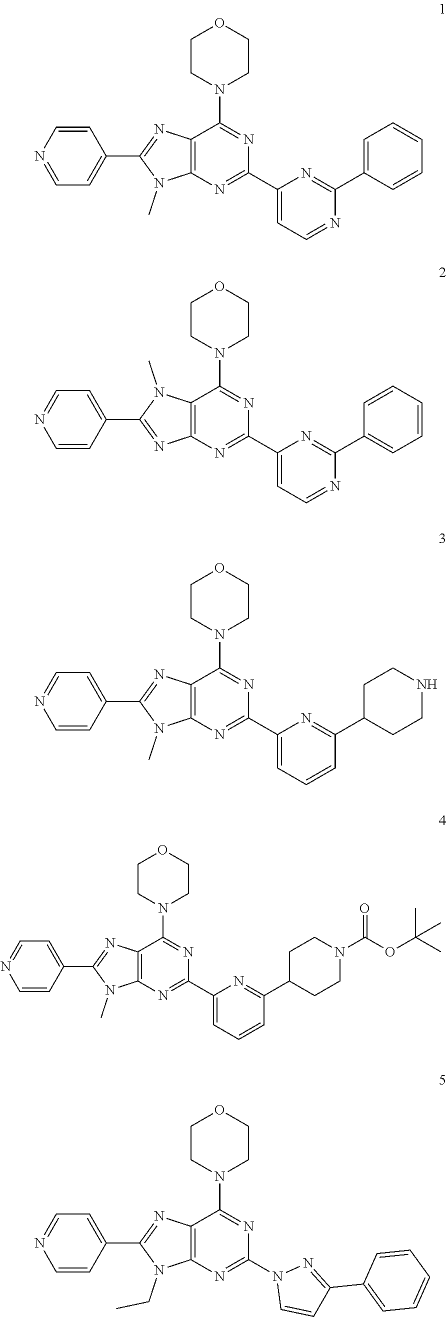 [SMILES: Cn1c(-c2ccncc2)nc2c(N3CCOCC3)nc(-c3ccnc(-c4ccccc4)n3)nc21 ; Cn1c(-c2ccncc2)nc2nc(-c3ccnc(-c4ccccc4)n3)nc(N3CCOCC3)c21 ; Cn1c(-c2ccncc2)nc2c(N3CCOCC3)nc(-c3cccc(C4CCNCC4)n3)nc21 ; Cn1c(-c2ccncc2)nc2c(N3CCOCC3)nc(-c3cccc(C4CCN(C(=O)OC(C)(C)C)CC4)n3)nc21 ; CCn1c(-c2ccncc2)nc2c(N3CCOCC3)nc(-n3ccc(-c4ccccc4)n3)nc21]

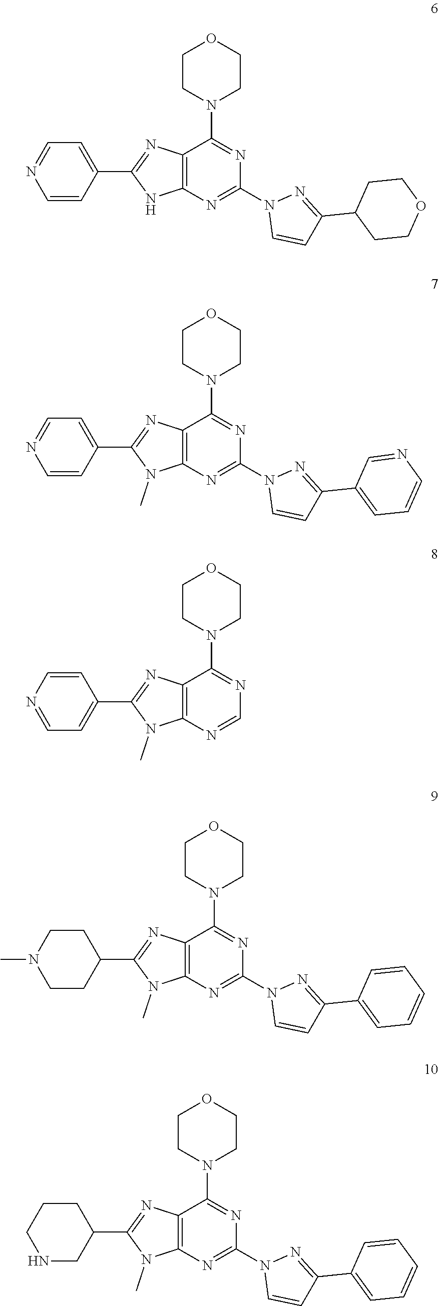 [SMILES: c1cc(-c2nc3c(N4CCOCC4)nc(-n4ccc(C5CCOCC5)n4)nc3[nH]2)ccn1 ; Cn1c(-c2ccncc2)nc2c(N3CCOCC3)nc(-n3ccc(-c4cccnc4)n3)nc21 ; Cn1c(-c2ccncc2)nc2c(N3CCOCC3)ncnc21 ; CN1CCC(c2nc3c(N4CCOCC4)nc(-n4ccc(-c5ccccc5)n4)nc3n2C)CC1 ; Cn1c(C2CCCNC2)nc2c(N3CCOCC3)nc(-n3ccc(-c4ccccc4)n3)nc21]

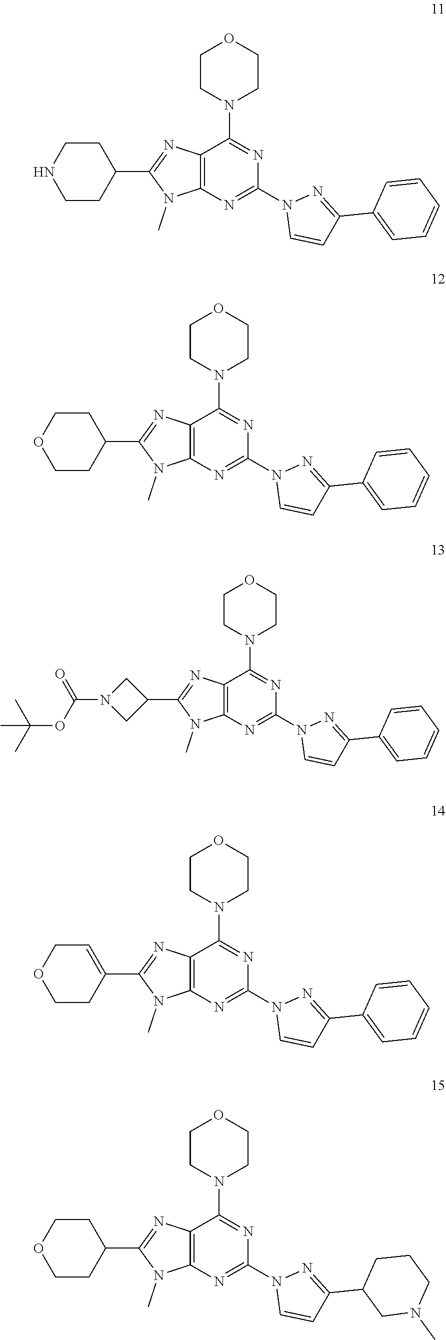 [SMILES: Cn1c(C2CCNCC2)nc2c(N3CCOCC3)nc(-n3ccc(-c4ccccc4)n3)nc21 ; Cn1c(C2CCOCC2)nc2c(N3CCOCC3)nc(-n3ccc(-c4ccccc4)n3)nc21 ; Cn1c(C2CN(C(=O)OC(C)(C)C)C2)nc2c(N3CCOCC3)nc(-n3ccc(-c4ccccc4)n3)nc21 ; Cn1c(C2=CCOCC2)nc2c(N3CCOCC3)nc(-n3ccc(-c4ccccc4)n3)nc21 ; CN1CCCC(c2ccn(-c3nc(N4CCOCC4)c4nc(C5CCOCC5)n(C)c4n3)n2)C1]

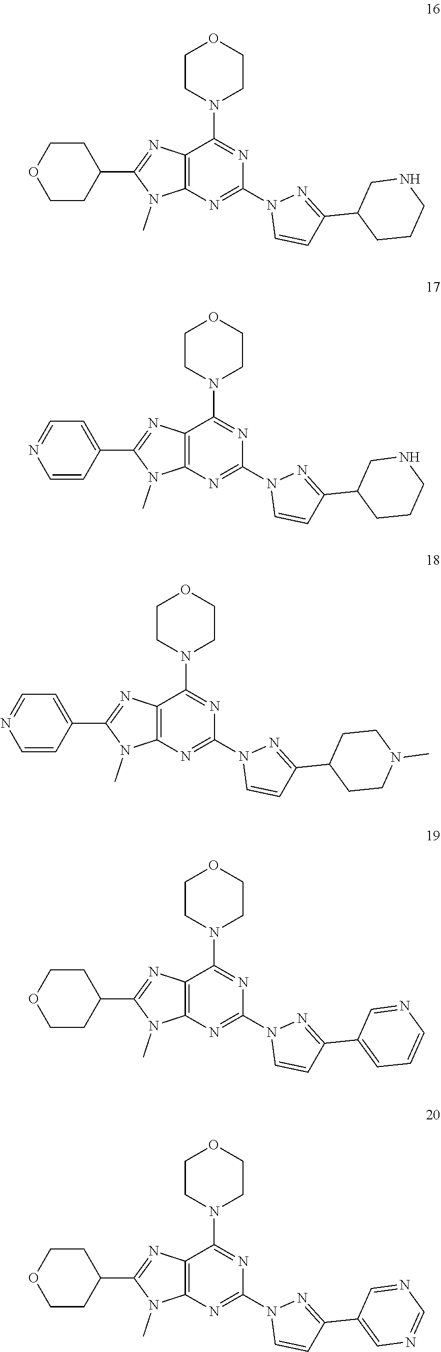 [SMILES: Cn1c(C2CCOCC2)nc2c(N3CCOCC3)nc(-n3ccc(C4CCCNC4)n3)nc21 ; Cn1c(-c2ccncc2)nc2c(N3CCOCC3)nc(-n3ccc(C4CCCNC4)n3)nc21 ; CN1CCC(c2ccn(-c3nc(N4CCOCC4)c4nc(-c5ccncc5)n(C)c4n3)n2)CC1 ; Cn1c(C2CCOCC2)nc2c(N3CCOCC3)nc(-n3ccc(-c4cccnc4)n3)nc21 ; Cn1c(C2CCOCC2)nc2c(N3CCOCC3)nc(-n3ccc(-c4cncnc4)n3)nc21]

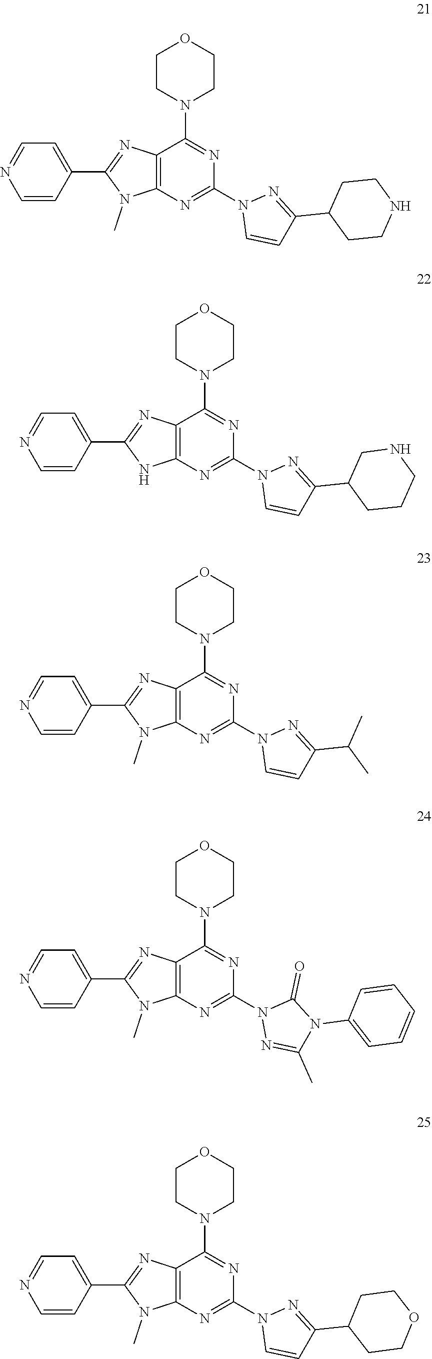 [SMILES: Cn1c(-c2ccncc2)nc2c(N3CCOCC3)nc(-n3ccc(C4CCNCC4)n3)nc21 ; c1cc(-c2nc3c(N4CCOCC4)nc(-n4ccc(C5CCCNC5)n4)nc3[nH]2)ccn1 ; CC(C)c1ccn(-c2nc(N3CCOCC3)c3nc(-c4ccncc4)n(C)c3n2)n1 ; Cc1nn(-c2nc(N3CCOCC3)c3nc(-c4ccncc4)n(C)c3n2)c(=O)n1-c1ccccc1 ; Cn1c(-c2ccncc2)nc2c(N3CCOCC3)nc(-n3ccc(C4CCOCC4)n3)nc21]

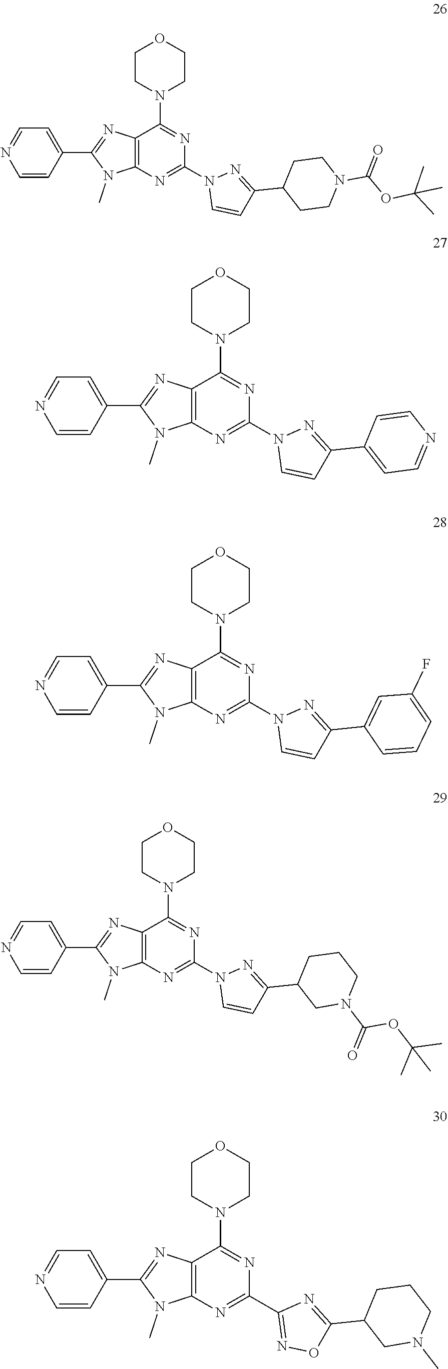 [SMILES: Cn1c(-c2ccncc2)nc2c(N3CCOCC3)nc(-n3ccc(C4CCN(C(=O)OC(C)(C)C)CC4)n3)nc21 ; Cn1c(-c2ccncc2)nc2c(N3CCOCC3)nc(-n3ccc(-c4ccncc4)n3)nc21 ; Cn1c(-c2ccncc2)nc2c(N3CCOCC3)nc(-n3ccc(-c4cccc(F)c4)n3)nc21 ; Cn1c(-c2ccncc2)nc2c(N3CCOCC3)nc(-n3ccc(C4CCCN(C(=O)OC(C)(C)C)C4)n3)nc21 ; CN1CCCC(c2nc(-c3nc(N4CCOCC4)c4nc(-c5ccncc5)n(C)c4n3)no2)C1]

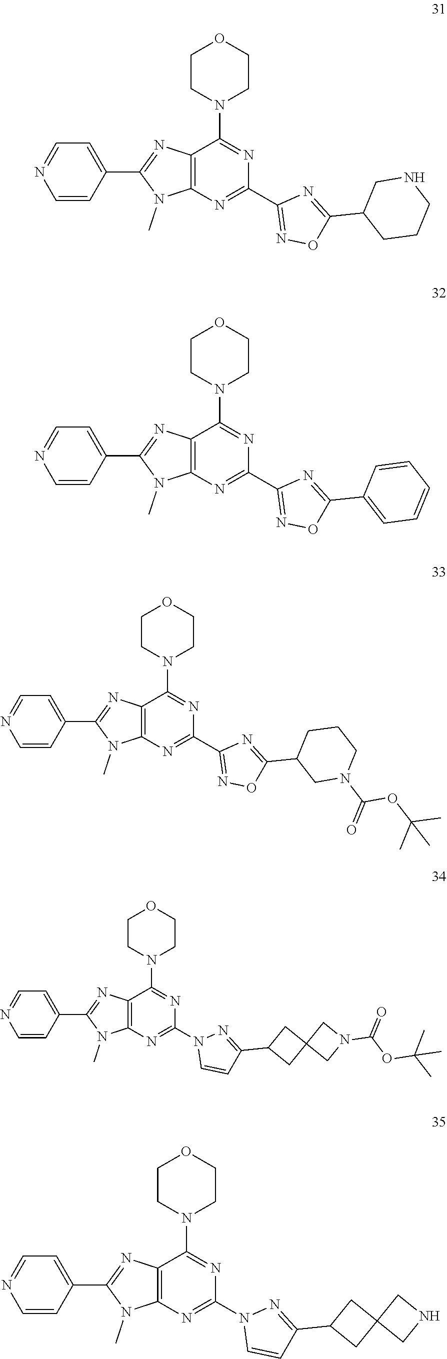 [SMILES: Cn1c(-c2ccncc2)nc2c(N3CCOCC3)nc(-c3noc(C4CCCNC4)n3)nc21 ; Cn1c(-c2ccncc2)nc2c(N3CCOCC3)nc(-c3noc(-c4ccccc4)n3)nc21 ; Cn1c(-c2ccncc2)nc2c(N3CCOCC3)nc(-c3noc(C4CCCN(C(=O)OC(C)(C)C)C4)n3)nc21 ; Cn1c(-c2ccncc2)nc2c(N3CCOCC3)nc(-n3ccc(C4CC5(C4)CN(C(=O)OC(C)(C)C)C5)n3)nc21 ; Cn1c(-c2ccncc2)nc2c(N3CCOCC3)nc(-n3ccc(C4CC5(CNC5)C4)n3)nc21]

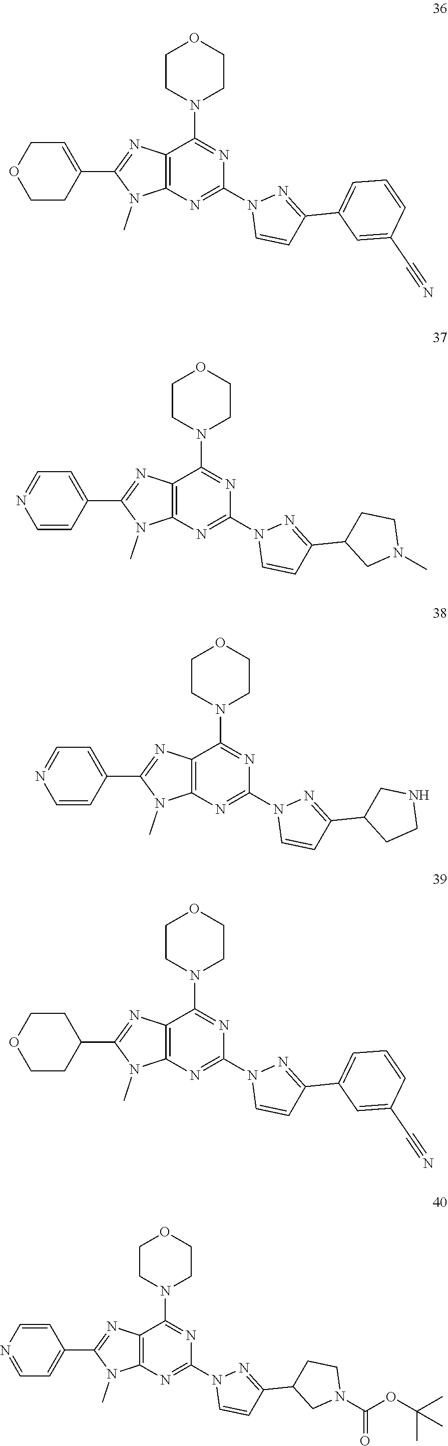 [SMILES: Cn1c(C2=CCOCC2)nc2c(N3CCOCC3)nc(-n3ccc(-c4cccc(C#N)c4)n3)nc21 ; CN1CCC(c2ccn(-c3nc(N4CCOCC4)c4nc(-c5ccncc5)n(C)c4n3)n2)C1 ; Cn1c(-c2ccncc2)nc2c(N3CCOCC3)nc(-n3ccc(C4CCNC4)n3)nc21 ; Cn1c(C2CCOCC2)nc2c(N3CCOCC3)nc(-n3ccc(-c4cccc(C#N)c4)n3)nc21 ; Cn1c(-c2ccncc2)nc2c(N3CCOCC3)nc(-n3ccc(C4CCN(C(=O)OC(C)(C)C)C4)n3)nc21]

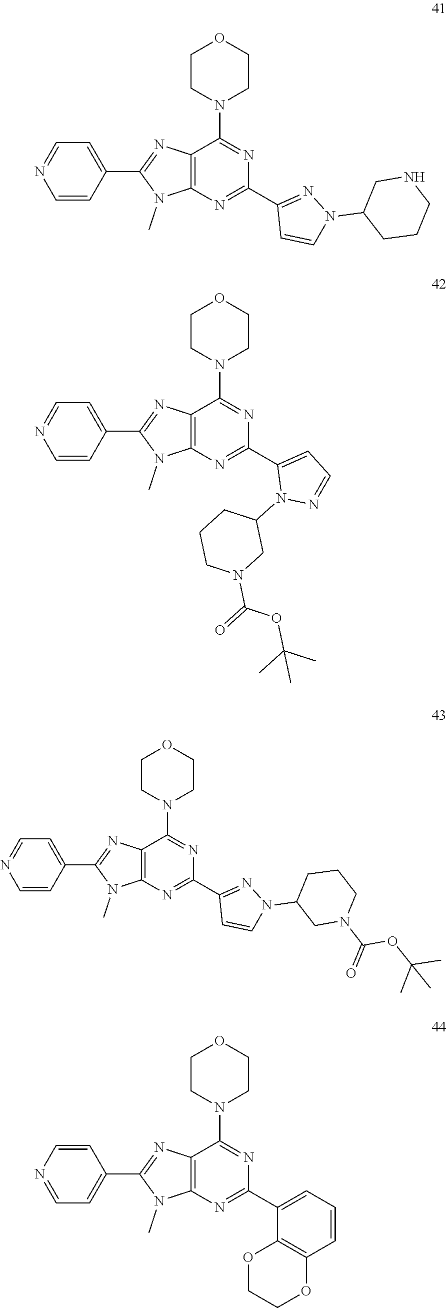 [SMILES: Cn1c(-c2ccncc2)nc2c(N3CCOCC3)nc(-c3ccn(C4CCCNC4)n3)nc21 ; Cn1c(-c2ccncc2)nc2c(N3CCOCC3)nc(-c3ccnn3C3CCCN(C(=O)OC(C)(C)C)C3)nc21 ; Cn1c(-c2ccncc2)nc2c(N3CCOCC3)nc(-c3ccn(C4CCCN(C(=O)OC(C)(C)C)C4)n3)nc21 ; Cn1c(-c2ccncc2)nc2c(N3CCOCC3)nc(-c3cccc4c3OCCO4)nc21]

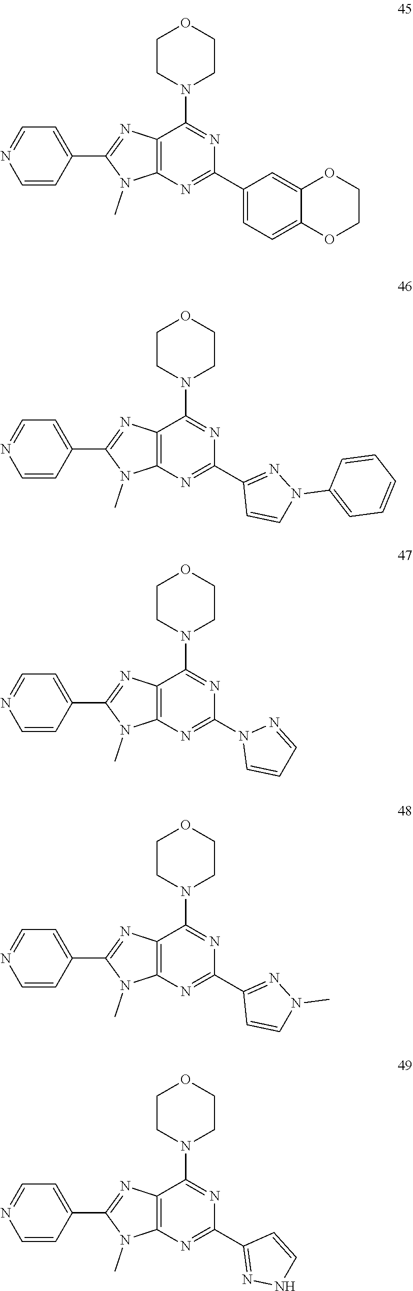 [SMILES: Cn1c(-c2ccncc2)nc2c(N3CCOCC3)nc(-c3ccc4c(c3)OCCO4)nc21 ; Cn1c(-c2ccncc2)nc2c(N3CCOCC3)nc(-c3ccn(-c4ccccc4)n3)nc21 ; Cn1c(-c2ccncc2)nc2c(N3CCOCC3)nc(-n3cccn3)nc21 ; Cn1ccc(-c2nc(N3CCOCC3)c3nc(-c4ccncc4)n(C)c3n2)n1 ; Cn1c(-c2ccncc2)nc2c(N3CCOCC3)nc(-c3cc[nH]n3)nc21]

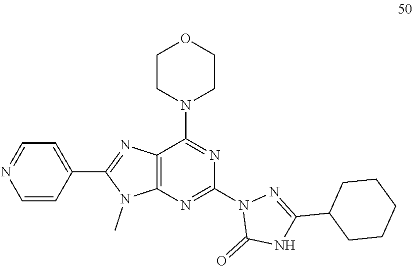 [SMILES: Cn1c(-c2ccncc2)nc2c(N3CCOCC3)nc(-n3nc(C4CCCCC4)[nH]c3=O)nc21]

or a pharmaceutically acceptable salt thereof.

In some embodiments, the compound is:

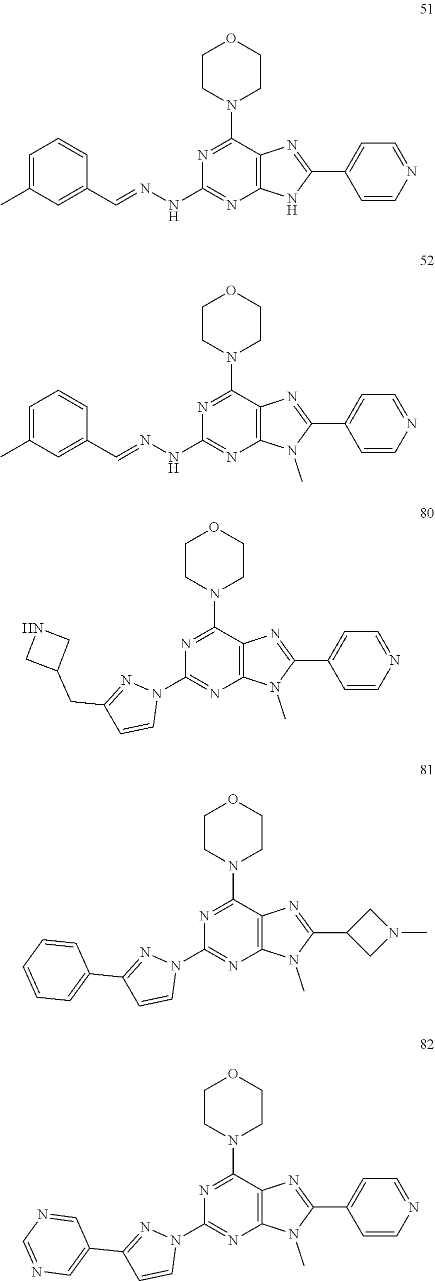 [SMILES: Cc1cccc(/C=N/Nc2nc(N3CCOCC3)c3nc(-c4ccncc4)[nH]c3n2)c1 ; Cc1cccc(/C=N/Nc2nc(N3CCOCC3)c3nc(-c4ccncc4)n(C)c3n2)c1 ; Cn1c(-c2ccncc2)nc2c(N3CCOCC3)nc(-n3ccc(CC4CNC4)n3)nc21 ; CN1CC(c2nc3c(N4CCOCC4)nc(-n4ccc(-c5ccccc5)n4)nc3n2C)C1 ; Cn1c(-c2ccncc2)nc2c(N3CCOCC3)nc(-n3ccc(-c4cncnc4)n3)nc21]

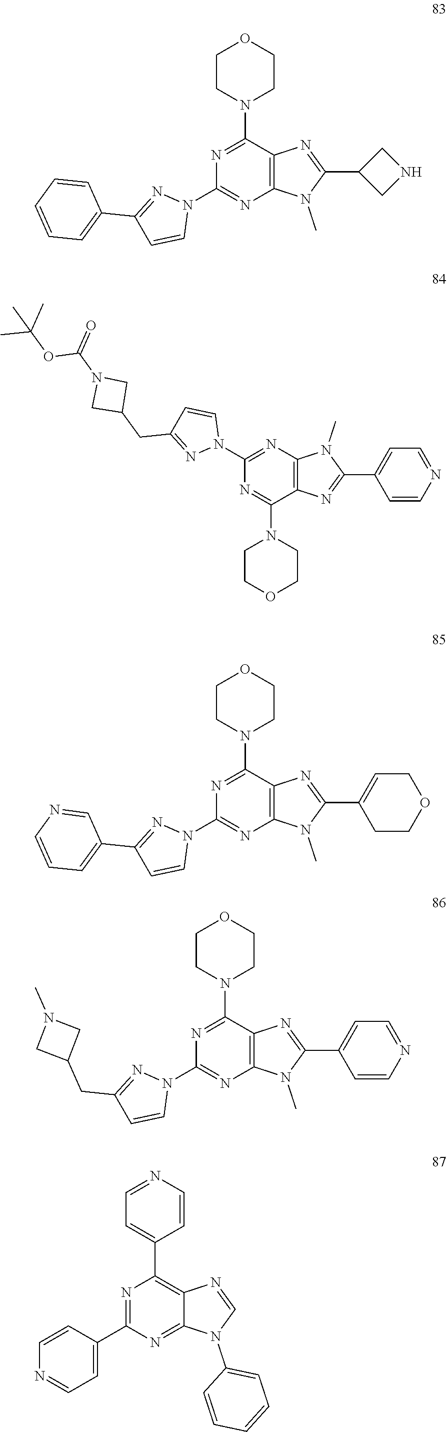 [SMILES: Cn1c(C2CNC2)nc2c(N3CCOCC3)nc(-n3ccc(-c4ccccc4)n3)nc21 ; Cn1c(-c2ccncc2)nc2c(N3CCOCC3)nc(-n3ccc(CC4CN(C(=O)OC(C)(C)C)C4)n3)nc21 ; Cn1c(C2=CCOCC2)nc2c(N3CCOCC3)nc(-n3ccc(-c4cccnc4)n3)nc21 ; CN1CC(Cc2ccn(-c3nc(N4CCOCC4)c4nc(-c5ccncc5)n(C)c4n3)n2)C1 ; c1ccc(-n2cnc3c(-c4ccncc4)nc(-c4ccncc4)nc32)cc1]

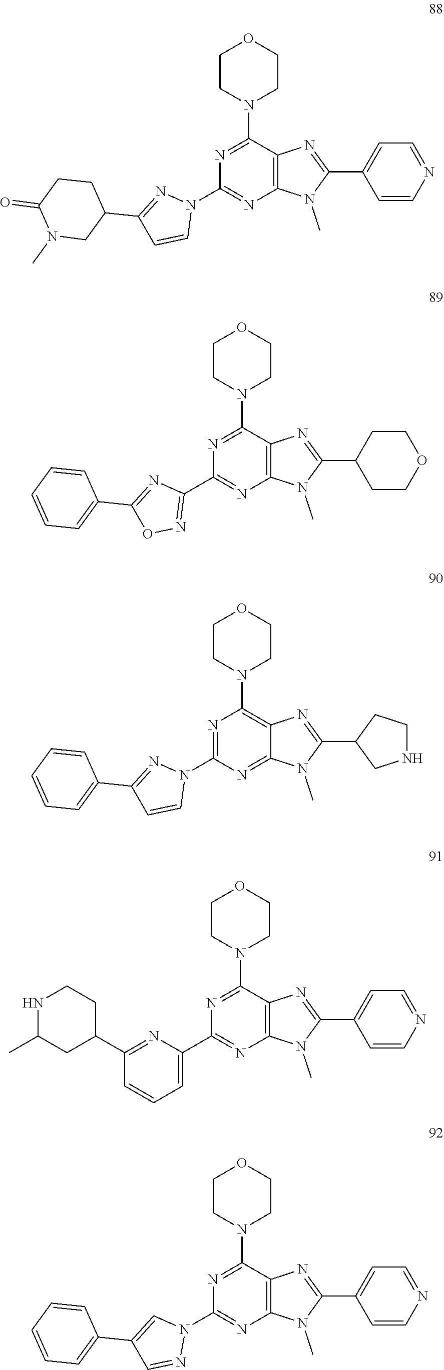 [SMILES: CN1CC(c2ccn(-c3nc(N4CCOCC4)c4nc(-c5ccncc5)n(C)c4n3)n2)CCC1=O ; Cn1c(C2CCOCC2)nc2c(N3CCOCC3)nc(-c3noc(-c4ccccc4)n3)nc21 ; Cn1c(C2CCNC2)nc2c(N3CCOCC3)nc(-n3ccc(-c4ccccc4)n3)nc21 ; CC1CC(c2cccc(-c3nc(N4CCOCC4)c4nc(-c5ccncc5)n(C)c4n3)n2)CCN1 ; Cn1c(-c2ccncc2)nc2c(N3CCOCC3)nc(-n3cc(-c4ccccc4)cn3)nc21]

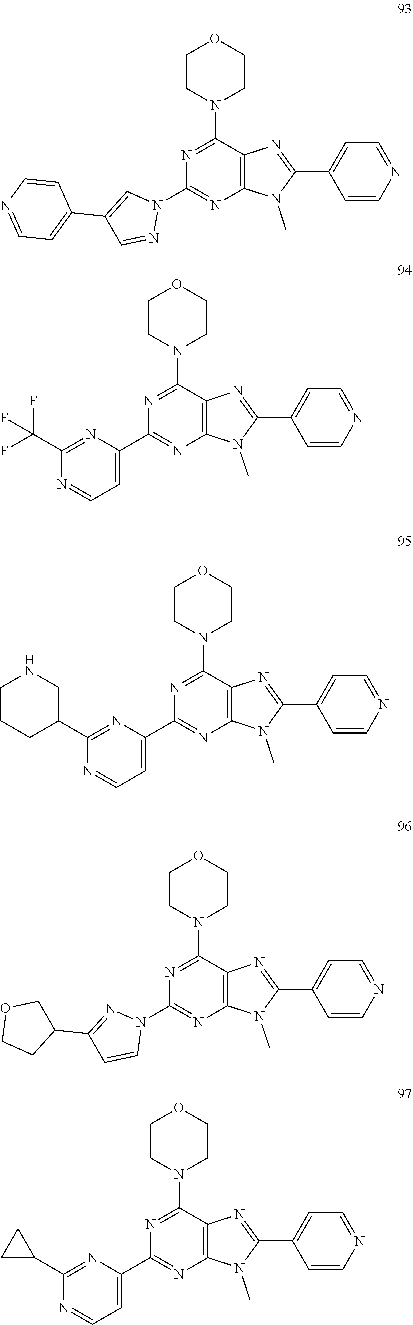 [SMILES: Cn1c(-c2ccncc2)nc2c(N3CCOCC3)nc(-n3cc(-c4ccncc4)cn3)nc21 ; Cn1c(-c2ccncc2)nc2c(N3CCOCC3)nc(-c3ccnc(C(F)(F)F)n3)nc21 ; Cn1c(-c2ccncc2)nc2c(N3CCOCC3)nc(-c3ccnc(C4CCCNC4)n3)nc21 ; Cn1c(-c2ccncc2)nc2c(N3CCOCC3)nc(-n3ccc(C4CCOC4)n3)nc21 ; Cn1c(-c2ccncc2)nc2c(N3CCOCC3)nc(-c3ccnc(C4CC4)n3)nc21]

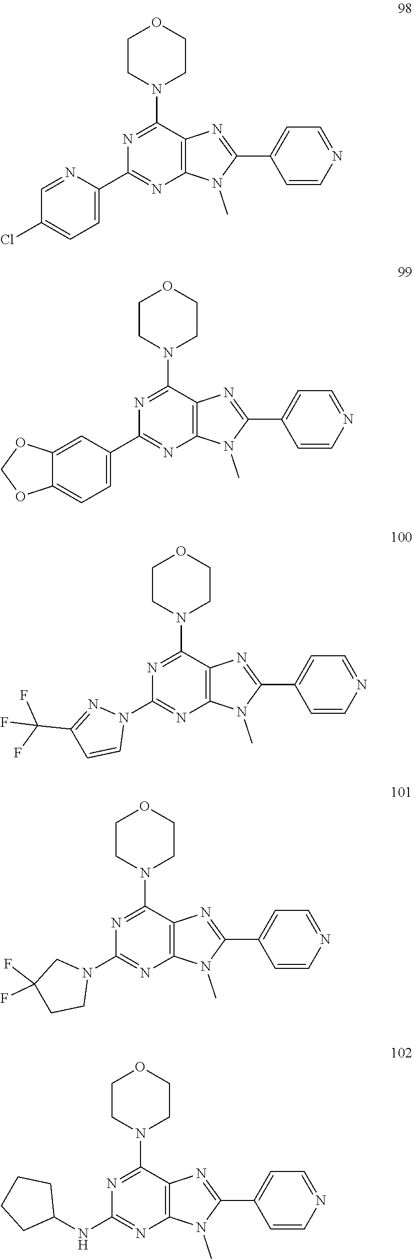 [SMILES: Cn1c(-c2ccncc2)nc2c(N3CCOCC3)nc(-c3ccc(Cl)cn3)nc21 ; Cn1c(-c2ccncc2)nc2c(N3CCOCC3)nc(-c3ccc4c(c3)OCO4)nc21 ; Cn1c(-c2ccncc2)nc2c(N3CCOCC3)nc(-n3ccc(C(F)(F)F)n3)nc21 ; Cn1c(-c2ccncc2)nc2c(N3CCOCC3)nc(N3CCC(F)(F)C3)nc21 ; Cn1c(-c2ccncc2)nc2c(N3CCOCC3)nc(NC3CCCC3)nc21]

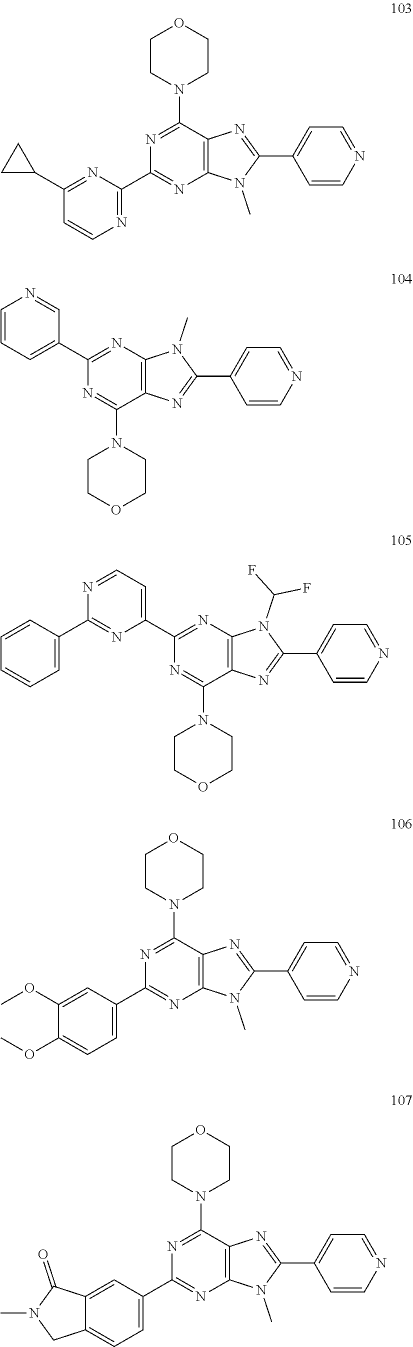 [SMILES: Cn1c(-c2ccncc2)nc2c(N3CCOCC3)nc(-c3nccc(C4CC4)n3)nc21 ; Cn1c(-c2ccncc2)nc2c(N3CCOCC3)nc(-c3cccnc3)nc21 ; FC(F)n1c(-c2ccncc2)nc2c(N3CCOCC3)nc(-c3ccnc(-c4ccccc4)n3)nc21 ; COc1ccc(-c2nc(N3CCOCC3)c3nc(-c4ccncc4)n(C)c3n2)cc1OC ; CN1Cc2ccc(-c3nc(N4CCOCC4)c4nc(-c5ccncc5)n(C)c4n3)cc2C1=O]

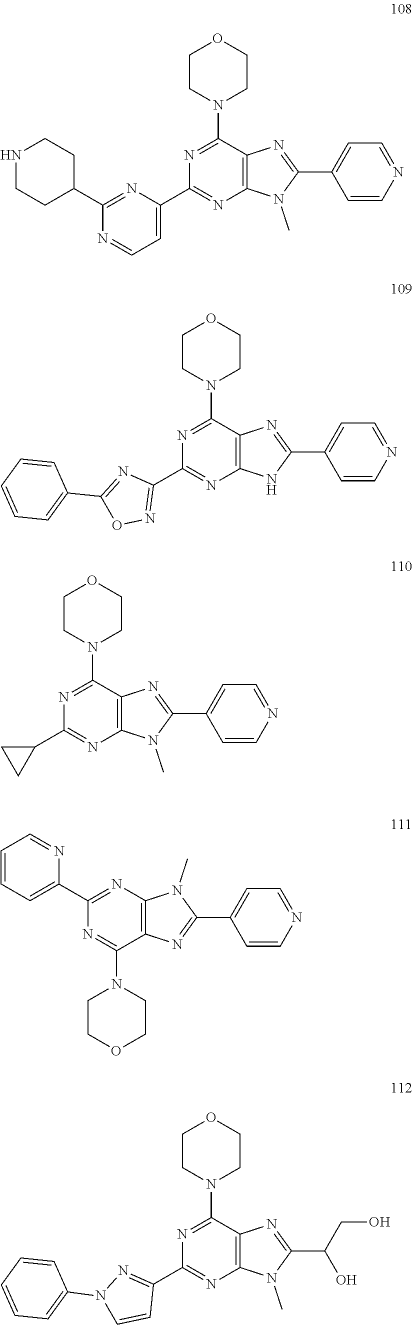 [SMILES: Cn1c(-c2ccncc2)nc2c(N3CCOCC3)nc(-c3ccnc(C4CCNCC4)n3)nc21 ; c1ccc(-c2nc(-c3nc(N4CCOCC4)c4nc(-c5ccncc5)[nH]c4n3)no2)cc1 ; Cn1c(-c2ccncc2)nc2c(N3CCOCC3)nc(C3CC3)nc21 ; Cn1c(-c2ccncc2)nc2c(N3CCOCC3)nc(-c3ccccn3)nc21 ; Cn1c(C(O)CO)nc2c(N3CCOCC3)nc(-c3ccn(-c4ccccc4)n3)nc21]

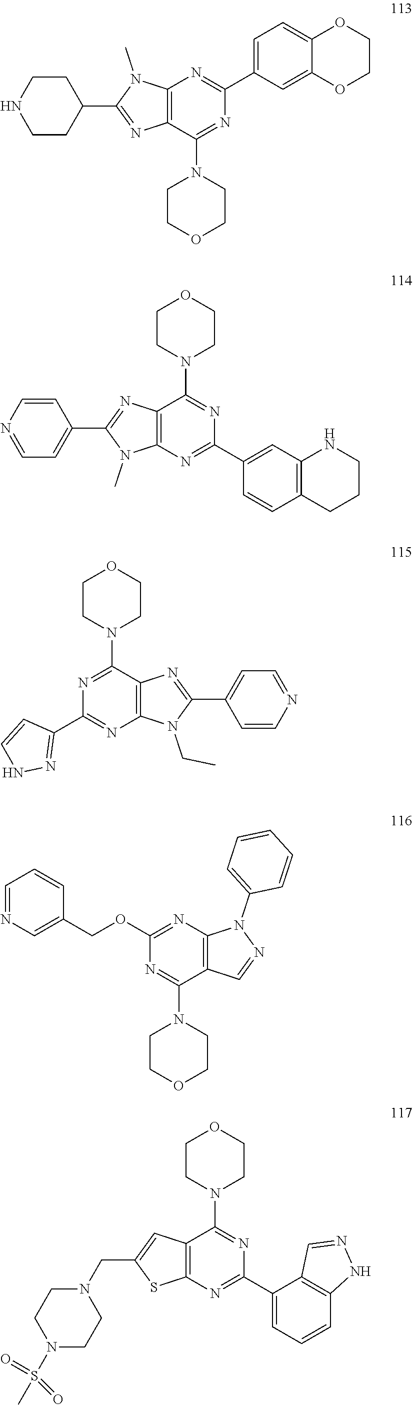 [SMILES: Cn1c(C2CCNCC2)nc2c(N3CCOCC3)nc(-c3ccc4c(c3)OCCO4)nc21 ; Cn1c(-c2ccncc2)nc2c(N3CCOCC3)nc(-c3ccc4c(c3)NCCC4)nc21 ; CCn1c(-c2ccncc2)nc2c(N3CCOCC3)nc(-c3cc[nH]n3)nc21 ; c1ccc(-n2ncc3c(N4CCOCC4)nc(OCc4cccnc4)nc32)cc1 ; CS(=O)(=O)N1CCN(Cc2cc3c(N4CCOCC4)nc(-c4cccc5[nH]ncc45)nc3s2)CC1]

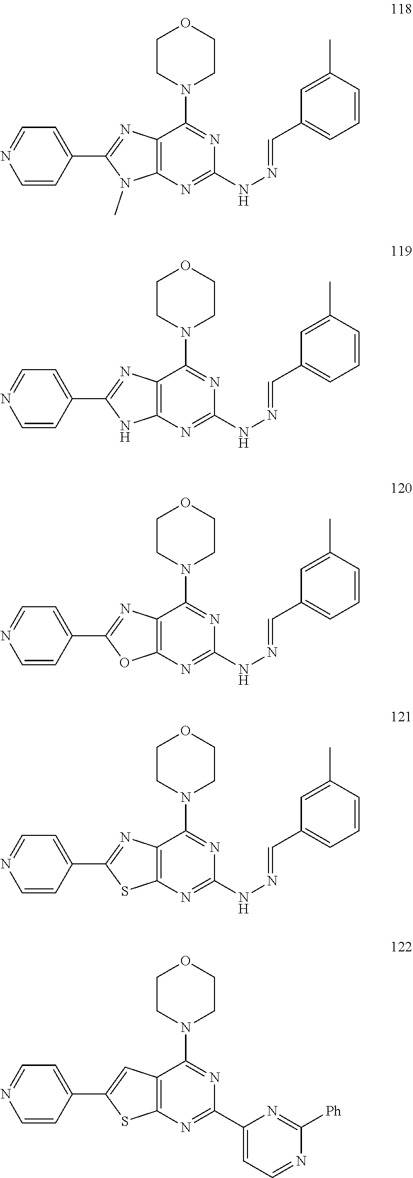 [SMILES: Cc1cccc(/C=N/Nc2nc(N3CCOCC3)c3nc(-c4ccncc4)n(C)c3n2)c1 ; Cc1cccc(/C=N/Nc2nc(N3CCOCC3)c3nc(-c4ccncc4)[nH]c3n2)c1 ; Cc1cccc(/C=N/Nc2nc(N3CCOCC3)c3nc(-c4ccncc4)oc3n2)c1 ; Cc1cccc(/C=N/Nc2nc(N3CCOCC3)c3nc(-c4ccncc4)sc3n2)c1 ; c1ccc(-c2nccc(-c3nc(N4CCOCC4)c4cc(-c5ccncc5)sc4n3)n2)cc1]

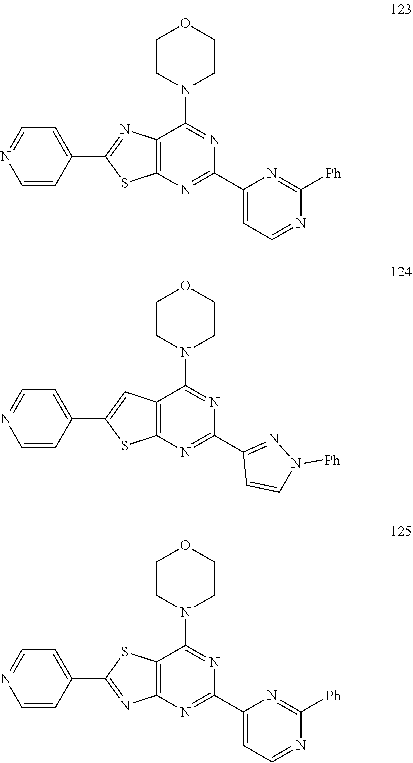 [SMILES: c1ccc(-c2nccc(-c3nc(N4CCOCC4)c4nc(-c5ccncc5)sc4n3)n2)cc1 ; c1ccc(-n2ccc(-c3nc(N4CCOCC4)c4cc(-c5ccncc5)sc4n3)n2)cc1 ; c1ccc(-c2nccc(-c3nc(N4CCOCC4)c4sc(-c5ccncc5)nc4n3)n2)cc1]

or a pharmaceutically acceptable salt thereof.

In an aspect, the invention features a pharmaceutical composition comprising any of the foregoing compounds and a pharmaceutically acceptable excipient.

In an aspect, the invention features a method of treating a neurological disorder (e.g., frontotemporal dementia (FTLD-TDP), chronic traumatic encephalopathy, ALS, Alzheimer's disease, limbic-predominant age-related TDP-43 encephalopathy (LATE), or frontotemporal lobar degeneration) in a subject in need thereof. This method includes administering an effective amount of any of the foregoing compounds or pharmaceutical compositions.

In an aspect, the invention features a method of inhibiting toxicity in a cell (e.g., mammalian neural cell) related to a protein (e.g., TDP-43 or C9orf72). This method includes administering an effective amount of any of the foregoing compounds or pharmaceutical compositions.

In an aspect, the invention features a method of treating a TDP-43-associated disorder or C9orf72-associated disorder (e.g., FTLD-TDP, chronic traumatic encephalopathy, ALS, Alzheimer's disease, LATE, or frontotemporal lobar degeneration) in a subject in need thereof. This method includes administering to the subject an effective amount of a compounds described herein or a pharmaceutical composition containing one or more compounds described herein. In some embodiments, the method includes administering to the subject in need thereof an effective amount of the compound of formula II:

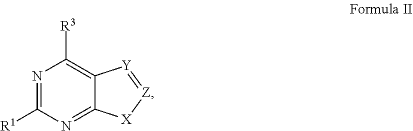

or a pharmaceutically acceptable salt thereof,
where
X is NR^{A}, S, or O;
Y is CR^{A} or N;
Z is CR² or N;
R¹ is hydrogen, optionally substituted C₁₋₆ alkyl, optionally substituted C₆₋₁₀ aryl, optionally substituted C₁₋₉ heterocyclyl, or optionally substituted C₁₋₉ heteroaryl;
R² is optionally substituted C₁₋₆ alkyl, optionally substituted C₆₋₁₀ aryl, optionally substituted C₁₋₉ heterocyclyl, or optionally substituted C₁₋₉ heteroaryl, or —NH—N═CHR^{B};
R³ is

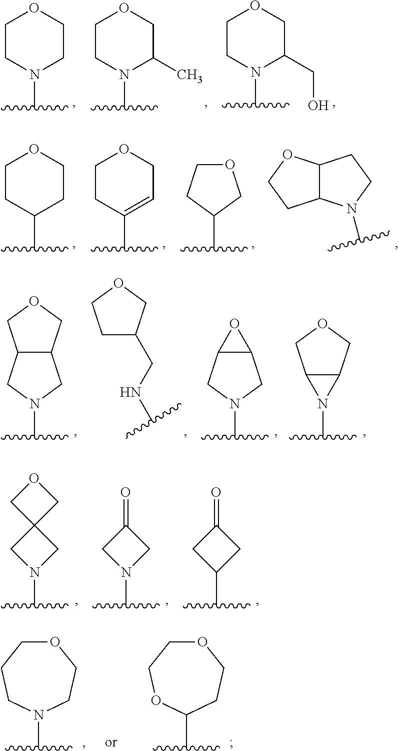

each R^{A} is independently H, optionally substituted C₁₋₆ alkyl, optionally substituted C₆₋₁₀ aryl, optionally substituted C₁₋₉ heteroaryl; and
each R^{B} is independently optionally substituted C₆₋₁₀ aryl or optionally substituted C₁₋₉ heteroaryl.

In some preferred embodiments, R¹ is optionally substituted C₁₋₉ heteroaryl comprising a 5-membered ring having a nitrogen atom at position 2 relative to the bond to the core, optionally substituted pyrimidin-6-yl, or optionally substituted benzodioxanyl.

In some preferred embodiments, R² is optionally substituted C₆₋₁₀ aryl, optionally substituted C₁₋₉ heterocyclyl, or optionally C₁₋₉ substituted heteroaryl.

In some embodiments, X is NR^{A}. In some embodiments, Y is N. In some preferred embodiments, Z is CR².

In some embodiments, R³ is

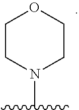 [SMILES: *N1CCOCC1]

In some embodiments, the compound is of formula Ia:

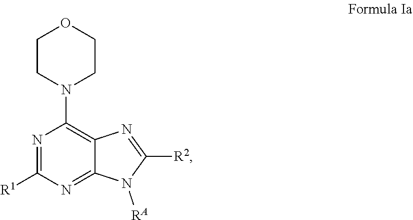

or a pharmaceutically acceptable salt thereof.

In some embodiments, R^{A} is optionally substituted C₁₋₆ alkyl. In some embodiments, R^{A} is H.

In some embodiments, the compound is of formula Ib:

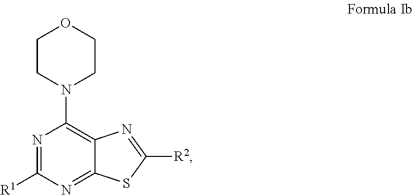

or a pharmaceutically acceptable salt thereof.

In some embodiments, the compound is of formula Ic:

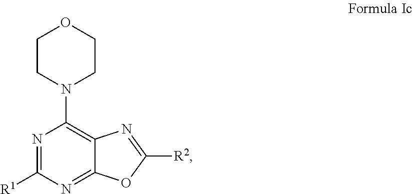

or a pharmaceutically acceptable salt thereof.

In some embodiments, the compound is of formula Id:

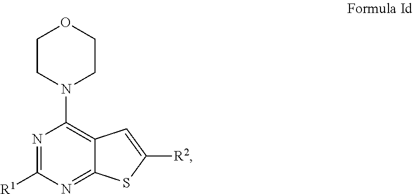

or a pharmaceutically acceptable salt thereof.

In some embodiments, R¹ is optionally substituted C₁₋₉ heteroaryl comprising a 5-membered ring having a nitrogen atom at position 2 relative to the bond to the core.

In some embodiments, R¹ is optionally substituted pyrazol-1-yl. In some embodiments, R¹ is pyrazol-1-yl substituted at position 3. In some embodiments, the pyrazol-1-yl substituted with optionally substituted C₆₋₁₀ aryl, optionally substituted C₁₋₉ heterocyclyl, optionally substituted C₁₋₉ heteroaryl, or optionally substituted C₃₋₈ cycloalkyl. In some embodiments, R¹ is optionally substituted pyrazol-3-yl. In some embodiments, R¹ is pyrazol-3-yl substituted at position 1. In some embodiments, the pyrazol-3-yl substituted with optionally substituted C₆₋₁₀ aryl, optionally substituted C₁₋₉ heterocyclyl, optionally substituted C₁₋₉ heteroaryl, or optionally substituted C₃₋₈ cycloalkyl. In some embodiments, R¹ is optionally substituted pyrimidin-6-yl.

In some embodiments, R² is optionally substituted C₁₋₉ heteroaryl. In some embodiments, R² is optionally substituted pyridyl. In some embodiments, R² is optionally substituted tetrahydropyranyl, optionally substituted dihydropyranyl, optionally substituted piperidinyl, or optionally substituted azetidinyl. In some embodiments, R² is optionally substituted tetrahydropyran-4-yl, optionally substituted 5,6-dihydro-2H-pyran-4-yl, optionally substituted piperidin-4-yl, or optionally substituted piperidin-3-yl.

In an aspect, the invention features a method of inhibiting PIKfyve. This method includes contacting a cell with an effective amount of any of the foregoing compounds or pharmaceutical compositions.

In another aspect, the invention features a method of treating a neurological disorder in a patient, such as a human patient, identified as likely to benefit from treatment with a compound of the invention on the basis of TDP-43 toxicity. In this aspect, the method may include (i) determining that the patient exhibits, or is prone to develop, TDP-43 toxicity, and (ii) providing to the patient a therapeutically effective amount of a compound of the invention. In some embodiments, the patient has previously been determined to exhibit, or to be prone to developing, TDP-43 toxicity, and the method includes providing to the patient a therapeutically effective amount of a compound of the invention. The susceptibility of the patient to developing TDP-43 aggregation may be determined, e.g., by determining whether the patient expresses a mutant isoform of TDP-43 containing a mutation that is associated with TDP-43 aggregation and toxicity, such as a mutation selected from Q331K, M337V, Q343R, N345K, R361S, and N390D. This may be performed, for example, by determining the amino acid sequence of a TDP-43 isoform isolated from a sample obtained from the patient or by determining the nucleic acid sequence of a TDP-43 gene isolated from a sample obtained from the patient. In some embodiments, the method includes the step of obtaining the sample from the patient.

In an additional aspect, the invention features a method of treating a neurological disorder in a patient, such as a human patient, identified as likely to benefit from treatment with a compound of the invention on the basis of TDP-43 expression. In this aspect, the method includes (i) determining that the patient expresses a mutant form of TDP-43 having a mutation associated with TDP-43 aggregation (e.g., a mutation selected from Q331K, M337V, Q343R, N345K, R361S, and N390D), and (ii) providing to the patient a therapeutically effective amount of a compound of the invention. In some embodiments, the patient has previously been determined to express a mutant form of TDP-43 having a mutation associated with TDP-43 aggregation, such as a Q331K, M337V, Q343R, N345K, R361S, or N390D mutation, and the method includes providing to the patient a therapeutically effective amount of a compound of the invention.

In another aspect, the invention features a method of determining whether a patient (e.g., a human patient) having a neurological disorder is likely to benefit from treatment with a compound of the invention by (i) determining whether the patient exhibits, or is prone to develop, TDP-43 aggregation and (ii) identifying the patient as likely to benefit from treatment with a compound of the invention if the patient exhibits, or is prone to develop, TDP-43 aggregation. In some embodiments, the method further includes the step of (iii) informing the patient whether he or she is likely to benefit from treatment with a compound of the invention. The susceptibility of the patient to developing TDP-43 aggregation may be determined, e.g., by determining whether the patient expresses a mutant isoform of TDP-43 containing a mutation that is associated with TDP-43 aggregation and toxicity, such as a mutation selected from Q331K, M337V, Q343R, N345K, R361S, and N390D. This may be performed, for example, by determining the amino acid sequence of a TDP-43 isoform isolated from a sample obtained from the patient or by determining the nucleic acid sequence of a TDP-43 gene isolated from a sample obtained from the patient. In some embodiments, the method includes the step of obtaining the sample from the patient.

In another aspect, the invention features a method of determining whether a patient (e.g., a human patient) having a neurological disorder is likely to benefit from treatment with a compound of the invention by (i) determining whether the patient expresses a TDP-43 mutant having a mutation associated with TDP-43 aggregation (e.g., a mutation selected from Q331K, M337V, Q343R, N345K, R361S, and N390D) and (ii) identifying the patient as likely to benefit from treatment with a compound of the invention if the patient expresses a TDP-43 mutant. In some embodiments, the method further includes the step of (iii) informing the patient whether he or she is likely to benefit from treatment with a compound of the invention. The TDP-43 isoform expressed by the patient may be assessed, for example, by isolated TDP-43 protein from a sample obtained from the patient and sequencing the protein using molecular biology techniques described herein or known in the art. In some embodiments, the TDP-43 isoform expressed by the patient is determined by analyzing the patient's genotype at the TDP-43 locus, for example, by sequencing the TDP-43 gene in a sample obtained from the patient. In some embodiments, the method includes the step of obtaining the sample from the patient.

In some embodiments of any of the above aspects, the compound of the invention is provided to the patient by administration of the compound of the invention to the patient. In some embodiments, the compound of the invention is provided to the patient by administration of a prodrug that is converted in vivo to the compound of the invention.

In some embodiments of any of the above aspects, the neurological disorder is a neuromuscular disorder, such as a neuromuscular disorder selected from amyotrophic lateral sclerosis, congenital myasthenic syndrome, congenital myopathy, cramp fasciculation syndrome, Duchenne muscular dystrophy, glycogen storage disease type II, hereditary spastic paraplegia, inclusion body myositis, Isaac's Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barré syndrome. In some embodiments, the neurological disorder is amyotrophic lateral sclerosis.

In some embodiments of any of the above aspects, the neurological disorder is selected from frontotemporal degeneration (also referred to as frontotemporal lobar degeneration and frontotemporal dementia), Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy.

In some embodiments, the neurological disorder is amyotrophic lateral sclerosis, and following administration of the compound of the invention to the patient, the patient exhibits one or more, or all, of the following responses:
(i) an improvement in condition as assessed using the amyotrophic lateral sclerosis functional rating scale (ALSFRS) or the revised ALSFRS (ALSFRS-R), such as an improvement in the patient's ALSFRS or ALSFRS-R score within one or more days, weeks, or months following administration of the compound of the invention (e.g., an improvement in the patient's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, weeks, 46 weeks, 47 weeks, 48 weeks, or more, following the initial administration of the compound of the invention to the patient);
(ii) an increase in slow vital capacity, such as an increase in the patient's slow vital capacity within one or more days, weeks, or months following administration of the compound of the invention (e.g., an increase in the patient's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(iii) a reduction in decremental responses exhibited by the patient upon repetitive nerve stimulation, such as a reduction that is observed within one or more days, weeks, or months following administration of the compound of the invention (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(iv) an improvement in muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the compound of the invention (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(v) an improvement in quality of life, as assessed, for example, using the amyotrophic lateral sclerosis-specific quality of life (ALS-specific QOL) questionnaire, such as an improvement in the patient's quality of life that is observed within one or more days, weeks, or months following administration of the compound of the invention (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(vi) a decrease in the frequency and/or severity of muscle cramps, such as a decrease in cramp frequency and/or severity within one or more days, weeks, or months following administration of the compound of the invention (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient); and/or
(vii) a decrease in TDP-43 aggregation, such as a decrease in TDP-43 aggregation within one or more days, weeks, or months following administration of the compound of the invention (e.g., a decrease in TDP-43 aggregation within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient.

### Chemical Terms

It is to be understood that the terminology employed herein is for the purpose of describing particular embodiments and is not intended to be limiting.

Those skilled in the art will appreciate that certain compounds described herein can exist in one or more different isomeric (e.g., stereoisomers, geometric isomers, tautomers) and/or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone—enol pairs, amide—imidic acid pairs, lactam—lactim pairs, amide—imidic acid pairs, enamine—imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion, e.g., the interconversion illustrated in the scheme below:

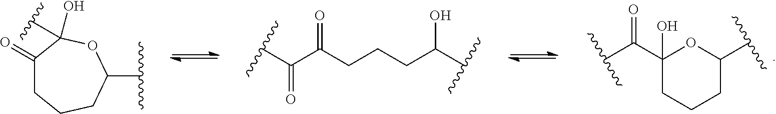 [SMILES: *C1CCCC(=O)C(*)(O)O1 ; *C(=O)C(=O)CCCC(*)O ; *C(=O)C1(O)CCCC(*)O1]

Those skilled in the art will appreciate that, in some embodiments, isotopes of compounds described herein may be prepared and/or utilized in accordance with the present invention. “Isotopes” refers to atoms having the same atomic number but different mass numbers resulting from a different number of neutrons in the nuclei. For example, isotopes of hydrogen include tritium and deuterium. In some embodiments, an isotopic substitution (e.g., substitution of hydrogen with deuterium) may alter the physiciochemical properties of the molecules, such as metabolism and/or the rate of racemization of a chiral center.

As is known in the art, many chemical entities (in particular many organic molecules and/or many small molecules) can adopt a variety of different solid forms such as, for example, amorphous forms and/or crystalline forms (e.g., polymorphs, hydrates, solvates, etc). In some embodiments, such entities may be utilized in any form, including in any solid form. In some embodiments, such entities are utilized in a particular form, e.g., in a particular solid form.

In some embodiments, compounds described and/or depicted herein may be provided and/or utilized in salt form.

In certain embodiments, compounds described and/or depicted herein may be provided and/or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substitutes are disclosed in groups or in ranges, unless otherwise indicated, the present disclosure is intended to cover individual compounds and groups of compounds (e.g., genera and subgenera) containing each and every individual subcombination of members at each position.

Herein a phrase of the form “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g., alkyl) per se is optional.

The term “acyl,” as used herein, represents a hydrogen or an alkyl group, as defined herein that is attached to a parent molecular group through a carbonyl group, as defined herein, and is exemplified by formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, or 1 to 6 carbon atoms). An alkylene is a divalent alkyl group.

The term “alkenyl,” as used herein, alone or in combination with other groups, refers to a straight-chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms).

The term “alkynyl,” as used herein, alone or in combination with other groups, refers to a straight-chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms).

The term “amino,” as used herein, represents —N(RN 1)₂, wherein each RN 1 is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each |R^{N2} is, independently, H, alkyl, or aryl. The amino groups of the invention can be an unsubstituted amino (i.e., —NH₂) or a substituted amino (i.e., —N(R^{N1})₂).

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and/H-indenyl.

The term “arylalkyl,” as used herein, represents an alkyl group substituted with an aryl group. Exemplary unsubstituted arylalkyl groups are from 7 to 30 carbons (e.g., from 7 to 16 or from 7 to 20 carbons, such as C₁-C₈ alkyl C₆₋₁₀ aryl, C₁-C₁₀ alkyl C₆₋₁₀ aryl, or C₁-C₂₀ alkyl C₆₋₁₀ aryl), such as, benzyl and phenethyl. In some embodiments, the alkyl and the aryl each can be further substituted with 1, 2, 3, or 4 substituent groups as defined herein for the respective groups.

The term “azido,” as used herein, represents a —N₃ group.

The term “cyano,” as used herein, represents a CN group.

The term “carbocyclyl,” as used herein, refer to a non-aromatic C3-C12 monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, norbornyl, and adamantyl. A polycyclic cycloalkyl may be fused, bridged, or spiro cycloalkyl.

The term “halo,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group.

The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group.

The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—. A heteroalkynylene is a divalent heteroalkynyl group.

The term “heteroaryl,” as used herein, refers to an aromatic mono- or polycyclic radical of 5 to 12 atoms having at least one aromatic ring containing one, two, or three ring heteroatoms selected from N, O, and S, with the remaining ring atoms being C. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₁-C₆ alkyl C₂-C₉ heteroaryl, C₁-C₁₀ alkyl C₂-C₉ heteroaryl, or C₁-C₂₀ alkyl C₂-C₉ heteroaryl). In some embodiments, the alkyl and the heteroaryl each can be further substituted with 1, 2, 3, or 4 substituent groups as defined herein for the respective groups.

The term “heterocyclyl,” as used herein, denotes a mono- or polycyclic radical having 3 to 12 atoms having at least one ring containing one, two, three, or four ring heteroatoms selected from N, O or S and no aromatic ring containing any N, O, or S atoms. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl. A heterocyclyl group may be aromatic or non-aromatic. An aromatic heterocyclyl is also referred to as heteroaryl. A polycyclic heterocyclyl may be fused, bridged, or spiro heterocyclyl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₁-C₆ alkyl C₂-C₉ heterocyclyl, C₁-C₁₀ alkyl C₂-C₉ heterocyclyl, or C₁-C₂₀ alkyl C₂-C₉ heterocyclyl). In some embodiments, the alkyl and the heterocyclyl each can be further substituted with 1, 2, 3, or 4 substituent groups as defined herein for the respective groups.

The term “hydroxyl,” as used herein, represents an —OH group.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3rd Edition (John Wiley & Sons, New York, 1999). N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an NO₂ group.

The term “oxyheteroaryl,” as used herein, represents a heteroaryl group having at least one endocyclic oxygen atom.

The term “oxyheterocyclyl,” as used herein, represents a heterocyclyl group having at least one endocyclic oxygen atom.

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: aryl (e.g., substituted and unsubstituted phenyl), carbocyclyl (e.g., substituted and unsubstituted cycloalkyl), halo (e.g., fluoro), hydroxyl, oxo, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained, for example, by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system.

Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99% or 99.9%) by weight relative to the other stereoisomers. When a single enantiomer is named or depicted by structure, the depicted or named enantiomer is at least 60%, 70%, 80%, 90%, 99% or 99.9% by weight optically pure. When a single diastereomer is named or depicted by structure, the depicted or named diastereomer is at least 60%, 70%, 80%, 90%, 99% or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99% or 99.9% by mole fraction pure relative to the other stereoisomers. When a single enantiomer is named or depicted by structure, the depicted or named enantiomer is at least 60%, 70%, 80%, 90%, 99% or 99.9% by mole fraction pure. When a single diastereomer is named or depicted by structure, the depicted or named diastereomer is at least 60%, 70%, 80%, 90%, 99% or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

### Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

As used herein, the term “administration” refers to the administration of a composition (e.g., a compound, a complex or a preparation that includes a compound or complex as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and vitreal.

As used herein, the term “animal” refers to any member of the animal kingdom. In some embodiments, “animal” refers to humans, at any stage of development. In some embodiments, “animal” refers to non-human animals, at any stage of development. In some embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, genetically engineered animal, and/or a clone.

As used herein, the terms “approximately” and “about” are each intended to encompass normal statistical variation as would be understood by those of ordinary skill in the art as appropriate to the relevant context. In certain embodiments, the terms “approximately” or “about” each refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility of the disease, disorder, or condition (e.g., across a relevant population).

As used herein, the terms “benefit” and “response” are used interchangeably in the context of a subject, such as a human subject undergoing therapy for the treatment of a neurological disorder, for example, amyotrophic lateral sclerosis, frontotemporal degeneration (also referred to as frontotemporal lobar degeneration and frontotemporal dementia), Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy. The terms “benefit” and “response” refer to any clinical improvement in the subject's condition. Exemplary benefits in the context of a subject undergoing treatment for a neurological disorder using the compositions and methods described herein (e.g., in the context of a human subject undergoing treatment for a neurological disorder described herein, such as amyotrophic lateral sclerosis, with a FYVE-type zinc finger containing phosphoinositide kinase (PIKfyve) inhibitor described herein, such as an inhibitory small molecule, antibody, antigen-binding fragment thereof, or interfering RNA molecule) include the slowing and halting of disease progression, as well as suppression of one or more symptoms associated with the disease. Particularly, in the context of a patient (e.g., a human patient) undergoing treatment for amyotrophic lateral sclerosis with a compound of the invention, examples of clinical “benefits” and “responses” are (i) an improvement in the subject's condition as assessed using the amyotrophic lateral sclerosis functional rating scale (ALSFRS) or the revised ALSFRS (ALSFRS-R) following administration of the compound of the invention, such as an improvement in the subject's ALSFRS or ALSFRS-R score within one or more days, weeks, or months following administration of the compound of the invention (e.g., an improvement in the subject's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (ii) an increase in the subject's slow vital capacity following administration of the compound of the invention, such as an increase in the subject's slow vital capacity within one or more days, weeks, or months following administration of the compound of the invention (e.g., an increase in the subject's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (iii) a reduction in decremental responses exhibited by the subject upon repetitive nerve stimulation, such as a reduction that is observed within one or more days, weeks, or months following administration of the compound of the invention (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (iv) an improvement in the subject's muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the compound of the invention (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (v) an improvement in the subject's quality of life, as assessed, for example, using the amyotrophic lateral sclerosis-specific quality of life (ALS-specific QOL) questionnaire, such as an improvement in the subject's quality of life that is observed within one or more days, weeks, or months following administration of the compound of the invention (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); and (vi) a decrease in the frequency and/or severity of muscle cramps exhibited by the subject, such as a decrease in cramp frequency and/or severity within one or more days, weeks, or months following administration of the compound of the invention (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject).

As used herein, the term “dosage form” refers to a physically discrete unit of an active compound (e.g., a therapeutic or diagnostic agent) for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount In some embodiments, a dosing regimen is correlated with a desired or beneficial outcome when administered across a relevant population (i.e., is a therapeutic dosing regimen).

In the practice of the methods of the present invention, an “effective amount” of any one of the compounds of the invention or a combination of any of the compounds of the invention or a pharmaceutically acceptable salt thereof, is administered via any of the usual and acceptable methods known in the art, either singly or in combination.

The term “pharmaceutical composition,” as used herein, represents a composition containing a compound described herein formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example, antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of formula (I). For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

The terms “PIKfyve” and “FYVE-type zinc finger containing phosphoinositide kinase” are used interchangeably herein and refer to the enzyme that catalyzes phosphorylation of phosphatidylinositol 3-phosphate to produce phosphatidylinositol 3,5-bisphosphate, for example, in human subjects. The terms “PIKfyve” and “FYVE-type zinc finger containing phosphoinositide kinase” refer not only to wild-type forms of PIKfyve, but also to variants of wild-type PIKfyve proteins and nucleic acids encoding the same. The gene encoding PIKfyve can be accessed under NCBI Reference Sequence No. NG_021188.1. Exemplary transcript sequences of wild-type form of human PIKfyve can be accessed under NCBI Reference Sequence Nos. NM_015040.4, NM_152671.3, and NM_001178000.1. Exemplary protein sequences of wild-type form of human PIKfyve can be accessed under NCBI Reference Sequence Nos. NP_055855.2, NP_689884.1, and NP_001171471.1.

As used herein, the term “PIKfyve inhibitor” refers to substances, such as compounds of Formula I. Inhibitors of this type may, for example, competitively inhibit PIKfyve activity by specifically binding the PIKfyve enzyme (e.g., by virtue of the affinity of the inhibitor for the PIKfyve active site), thereby precluding, hindering, or halting the entry of one or more endogenous substrates of PIKfyve into the enzyme's active site. Additional examples of PIKfyve inhibitors that suppress the activity of the PIKfyve enzyme include substances that may bind PIKfyve at a site distal from the active site and attenuate the binding of endogenous substrates to the PIKfyve active site by way of a change in the enzyme's spatial conformation upon binding of the inhibitor. In addition to encompassing substances that modulate PIKfyve activity, the term “PIKfyve inhibitor” refers to substances that reduce the concentration and/or stability of PIKfyve mRNA transcripts in vivo, as well as those that suppress the translation of functional PIKfyve enzyme.

The term “pure” means substantially pure or free of unwanted components (e.g., other compounds and/or other components of a cell lysate), material defilement, admixture or imperfection.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

A variety of clinical indicators can be used to identify a patient as “at risk” of developing a particular neurological disease. Examples of patients (e.g., human patients) that are “at risk” of developing a neurological disease, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy, include (i) subjects exhibiting or prone to exhibit aggregation of TAR-DNA binding protein (TDP)-43, and (ii) subjects expressing a mutant form of TDP-43 containing a mutation associated with TDP-43 aggregation and toxicity, such as a mutation selected from Q331K, M337V, Q343R, N345K, R361S, and N390D. Subjects that are “at risk” of developing amyotrophic lateral sclerosis may exhibit one or both of these characteristics, for example, prior to the first administration of a PIKfyve inhibitor in accordance with the compositions and methods described herein.

As used herein, the terms “TAR-DNA binding protein-43” and “TDP-43” are used interchangeably and refer to the transcription repressor protein involved in modulating HIV-1 transcription and alternative splicing of the cystic fibrosis transmembrane conductance regulator (CFTR) pre-mRNA transcript, for example, in human subjects. The terms “TAR-DNA binding protein-43” and “TDP-43” refer not only to wild-type forms of TDP-43, but also to variants of wild-type TDP-43 proteins and nucleic acids encoding the same. The amino acid sequence and corresponding mRNA sequence of a wild-type form of human TDP-43 are provided under NCBI Reference Sequence Nos. NM_007375.3 and NP_031401.1, respectively.

The terms “TAR-DNA binding protein-43” and “TDP-43” as used herein include, for example, forms of the human TDP-43 protein that have an amino acid sequence that is at least 85% identical to the amino acid sequence of NCBI Reference Sequence No. NP_031401.1 (e.g., 85%, 86%, 87%, 88%, 89%, 90%, 91%, 92%, 93%, 94%, 95%, 96%, 97%, 98%, 99%, 99.9%, or 100% identical to the amino acid sequence of NCBI Reference Sequence No. NP_031401.1) and/or forms of the human TDP-43 protein that contain one or more substitutions, insertions, and/or deletions (e.g., one or more conservative and/or nonconservative amino acid substitutions, such as up to 5, 10, 15, 20, 25, or more, conservative or nonconservative amino acid substitutions) relative to a wild-type TDP-43 protein. For instance, patients that may be treated for a neurological disorder as described herein, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy, include human patients that express a form of TDP-43 having a mutation associated with elevated TDP-43 aggregation and toxicity, such as a mutation selected from Q331K, M337V, Q343R, N345K, R361S, and N390D. Similarly, the terms “TAR-DNA binding protein-43” and “TDP-43” as used herein include, for example, forms of the human TDP-43 gene that encode an mRNA transcript having a nucleic acid sequence that is at least 85% identical to the nucleic acid sequence of NCBI Reference Sequence No. NM_007375.3 (e.g., 85%, 86%, 87%, 88%, 89%, 90%, 91%, 92%, 93%, 94%, 95%, 96%, 97%, 98%, 99%, 99.9%, or 100% identical to the amino acid sequence of NCBI Reference Sequence No. NM_007375.3).

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

A “therapeutic regimen” refers to a dosing regimen whose administration across a relevant population is correlated with a desired or beneficial therapeutic outcome.

The term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” To give but one example, a refractory subject may have a low bioavailability such that clinical efficacy is not obtainable. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

### BRIEF DESCRIPTION OF THE DRAWINGS

FIG. 1 is a scheme showing an approach to generation of a control TDP-43 yeast model (FAB1 TDP-43). A control yeast TDP-43 model was generated by integrating the human TDP-43 gene and the GAL1 promoter into the yeast genome. The yeast ortholog of human PIKFYVE is FAB1.

FIG. 2 is a scheme showing an approach to generation of a humanized PIKFYVE TDP-43 yeast model (PIKFYVE TDP-43). FAB1 gene through homologous recombination with a G418 resistance cassette (fab1::G418^{R}) (FIG. 2). PIKFYVE was cloned downstream of the GPD promoter harbored on a URA3-containing plasmid and introduced into the fab1::G418R ura3 strain. The pGAL/-TDP-43 construct was then introduced into the “humanized” yeast strain and assessed for cytotoxicity.

FIG. 3 is a histogram generated from the flow cytometry-based viability assay of FAB1 TDP-43.

FIG. 4 is a histogram generated from the flow cytometry-based viability assay of PIKFYVE TDP-43. Upon induction of TDP-43, there was a marked increase in inviable cells (rightmost population), with a more pronounced effect in PIKFYVE TDP-43 than in FAB1 TDP-43 strain (see FIG. 3).

FIG. 5 is an overlay of histograms generated from the flow cytometry-based viability assay of FAB1 TDP-43 in the presence of APY0201.

FIG. 6 is an overlay of histograms generated from the flow cytometry-based viability assay of PIKFYVE TDP-43 in the presence of APY0201.

FIG. 7 is a scatter plot comparing cytoprotection efficacy in PIKFYVE TDP-43 to PIKfyve inhibitory activity of test compounds.

### DETAILED DESCRIPTION

The present invention features compositions and methods for treating neurological disorders, such as amyotrophic lateral sclerosis and other neuromuscular disorders, as well as frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy among others. Particularly, the invention provides inhibitors of FYVE-type zinc finger containing phosphoinositide kinase (PIKfyve), that may be administered to a patient (e.g., a human patient) so as to treat or prevent a neurological disorder, such as one or more of the foregoing conditions. In the context of therapeutic treatment, the PIKfyve inhibitor may be administered to the patient to alleviate one or more symptoms of the disorder and/or to remedy an underlying molecular pathology associated with the disease, such as to suppress or prevent aggregation of TAR-DNA binding protein (TDP)-43.

The disclosure herein is based, in part, on the discovery that PIKfyve inhibition modulates TDP-43 aggregation in cells. Suppression of TDP-43 aggregation exerts beneficial effects in patients suffering from a neurological disorder. Many pathological conditions have been correlated with TDP-43-promoted aggregation and toxicity, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, IBMPFD, sporadic inclusion body myositis, myofibrillar myopathy, dementia pugilistica, chronic traumatic encephalopathy, Alexander disease, and hereditary inclusion body myopathy. Without being limited by mechanism, by administering an inhibitor of PIKfyve, patients suffering from diseases associated with TDP-43 aggregation and toxicity may be treated, for example, due to the suppression of TDP-43 aggregation induced by the PIKfyve inhibitor.

Patients that are likely to respond to PIKfyve inhibition as described herein include those that have or are at risk of developing TDP-43 aggregation, such as those that express a mutant form of TDP-43 associated with TDP-43 aggregation and toxicity in vivo. Examples of such mutations in TDP-43 that have been correlated with elevated TDP-43 aggregation and toxicity include Q331K, M337V, Q343R, N345K, R361S, and N390D, among others. The compositions and methods described herein thus provide the additional clinical benefit of enabling the identification of patients that are likely to respond to PIKfyve inhibitor therapy, as well as processes for treating these patients accordingly.

The sections that follow provide a description of exemplary PIKfyve inhibitors that may be used in conjunction with the compositions and methods disclosed herein. The sections below additionally provide a description of various exemplary routes of administration and pharmaceutical compositions that may be used for delivery of these substances for the treatment of a neurological disorder.

### PIKfyve Inhibitors

Exemplary PIKfyve inhibitors described herein include compounds of formula I:

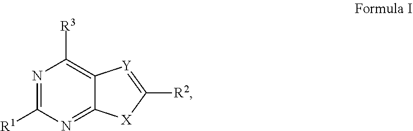

or a pharmaceutically acceptable salt thereof,
where
X is NR^{A}, S, or O;
Y is CR^{A} or N;
R¹ is optionally substituted C₁₋₉ heteroaryl comprising a 5-membered ring having a nitrogen atom at position 2 relative to the bond to the core, optionally substituted pyrimidin-6-yl, or optionally substituted benzodioxanyl;
R² is optionally substituted C₆₋₁₀ aryl, optionally substituted C₁₋₉ heterocyclyl, or optionally C₁₋₉ substituted heteroaryl; and
R³ is

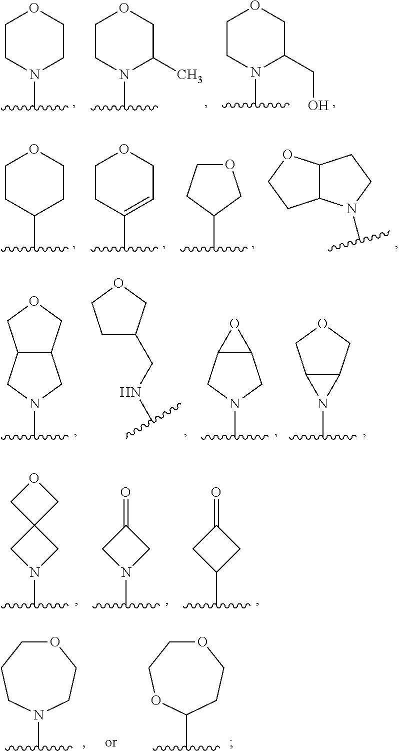

and
each R^{A} is independently H, optionally substituted C₁₋₆ alkyl, optionally substituted C₆₋₁₀ aryl, optionally substituted C₁₋₉ heteroaryl.

Exemplary PIKfyve inhibitors described herein also include compounds of formula I:

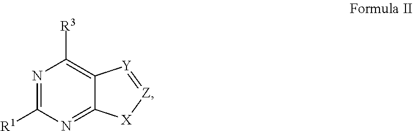

or a pharmaceutically acceptable salt thereof,
where
X is NR^{A}, S, or O;
Y is CR^{A} or N;
Z is CR² or N;
R¹ is hydrogen, optionally substituted C₁₋₆ alkyl, optionally substituted C₆₋₁₀ aryl, optionally substituted C₁₋₉ heterocyclyl, or optionally substituted C₁₋₉ heteroaryl;
R² is optionally substituted C₁₋₆ alkyl, optionally substituted C₆₋₁₀ aryl, optionally substituted C₁₋₉ heterocyclyl, or optionally substituted C₁₋₉ heteroaryl, or —NH—N═CHR^{B};
R³ is

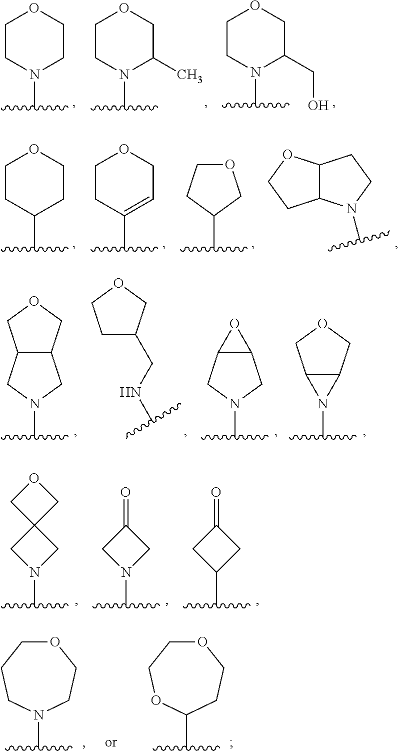

each R^{A} is independently H, optionally substituted C₁₋₆ alkyl, optionally substituted C₆₋₁₀ aryl, optionally substituted C₁₋₉ heteroaryl; and
each R^{B} is independently optionally substituted C₆₋₁₀ aryl or optionally substituted C₁₋₉ heteroaryl.

In some preferred embodiments, R¹ is optionally substituted C₁₋₉ heteroaryl comprising a 5-membered ring having a nitrogen atom at position 2 relative to the bond to the core, optionally substituted pyrimidin-6-yl, or optionally substituted benzodioxanyl. In some preferred embodiments, R² is optionally substituted C₆₋₁₀ aryl, optionally substituted C₁₋₉ heterocyclyl, or optionally C₁₋₉ substituted heteroaryl. In some preferred embodiments, Z is CR².

### Methods of Treatment

Suppression of PIKfyve Activity and TDP-43 Aggregation to Treat Neurological Disorders

Using the compositions and methods described herein, a patient suffering from a neurological disorder may be administered a PIKfyve inhibitor, such as a small molecule described herein, so as to treat the disorder and/or to suppress one or more symptoms associated with the disorder. Exemplary neurological disorders that may be treated using the compositions and methods described herein are, without limitation, amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, IBMPFD, sporadic inclusion body myositis, myofibrillar myopathy, dementia pugilistica, chronic traumatic encephalopathy, Alexander disease, and hereditary inclusion body myopathy, as well as neuromuscular diseases such as congenital myasthenic syndrome, congenital myopathy, cramp fasciculation syndrome, Duchenne muscular dystrophy, glycogen storage disease type II, hereditary spastic paraplegia, inclusion body myositis, Isaac's Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barré syndrome.

The present disclosure is based, in part, on the discovery that PIKfyve inhibitors, such as the agents described herein, are capable of attenuating TDP-43 toxicity. TDP-43-promoted toxicity has been associated with various neurological diseases. The discovery that PIKfyve inhibitors modulate TDP-43 aggregation provides an important therapeutic benefit. Using a PIKfyve inhibitor, such as a PIKfyve inhibitor described herein, a patient suffering from a neurological disorder or at risk of developing such a condition may be treated in a manner that remedies an underlying molecular etiology of the disease. Without being limited by mechanism, the compositions and methods described herein can be used to treat or prevent such neurological conditions, for example, by suppressing the TDP-43 aggregation that promotes pathology.

Additionally, the compositions and methods described herein provide the beneficial feature of enabling the identification and treatment of patients that are likely to respond to PIKfyve inhibitor therapy. For example, in some embodiments, a patient (e.g., a human patient suffering from or at risk of developing a neurological disease described herein, such as amyotrophic lateral sclerosis) is administered a PIKfyve inhibitor if the patient is identified as likely to respond to this form of treatment. Patients may be identified as such on the basis, for example, of susceptibility to TDP-43 aggregation. In some embodiments, the patient is identified is likely to respond to PIKfyve inhibitor treatment based on the isoform of TDP-43 expressed by the patient. For example, patients expressing TDP-43 isoforms having a mutation selected from Q331K, M337V, Q343R, N345K, R361S, and N390D, among others, are more likely to develop TDP-43-promoted aggregation and toxicity relative to patients that do not express such isoforms of TDP-43. Using the compositions and methods described herein, a patient may be identified as likely to respond to PIKfyve inhibitor therapy on the basis of expressing such an isoform of TDP-43, and may subsequently be administered a PIKfyve inhibitor so as to treat or prevent one or more neurological disorders, such as one or more of the neurological disorders described herein.

Assessing Patient Response

A variety of methods known in the art and described herein can be used to determine whether a patient having a neurological disorder (e.g., a patient at risk of developing TDP-43 aggregation, such as a patient expressing a mutant form of TDP-43 having a mutation associated with elevated TDP-43 aggregation and toxicity, for example, a mutation selected from Q331K, M337V, Q343R, N345K, R361S, and N390D) is responding favorably to PIKfyve inhibition. For example, successful treatment of a patient having a neurological disease, such as amyotrophic lateral sclerosis, with a PIKfyve inhibitor described herein may be signaled by:
(i) an improvement in condition as assessed using the amyotrophic lateral sclerosis functional rating scale (ALSFRS) or the revised ALSFRS (ALSFRS-R), such as an improvement in the patient's ALSFRS or ALSFRS-R score within one or more days, weeks, or months following administration of the PIKfyve inhibitor (e.g., an improvement in the patient's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(ii) an increase in slow vital capacity, such as an increase in the patient's slow vital capacity within one or more days, weeks, or months following administration of the PIKfyve inhibitor (e.g., an increase in the patient's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(iii) a reduction in decremental responses exhibited by the patient upon repetitive nerve stimulation, such as a reduction that is observed within one or more days, weeks, or months following administration of the PIKfyve inhibitor (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(iv) an improvement in muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the PIKfyve inhibitor (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(v) an improvement in quality of life, as assessed, for example, using the amyotrophic lateral sclerosis-specific quality of life (ALS-specific QOL) questionnaire, such as an improvement in the patient's quality of life that is observed within one or more days, weeks, or months following administration of the PIKfyve inhibitor (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(vi) a decrease in the frequency and/or severity of muscle cramps, such as a decrease in cramp frequency and/or severity within one or more days, weeks, or months following administration of the PIKfyve inhibitor (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient); and/or
(vii) a decrease in TDP-43 aggregation, such as a decrease in TDP-43 aggregation within one or more days, weeks, or months following administration of the PIKfyve inhibitor (e.g., a decrease in TDP-43 aggregation within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient.

### Combination Formulations and Uses Thereof

The compounds of the invention can be combined with one or more therapeutic agents. In particular, the therapeutic agent can be one that treats or prophylactically treats any neurological disorder described herein.

Combination Therapies

A compound of the invention can be used alone or in combination with other agents that treat neurological disorders or symptoms associated therewith, or in combination with other types of treatment to treat, prevent, and/or reduce the risk of any neurological disorders. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6, 2005). In this case, dosages of the compounds when combined should provide a therapeutic effect.

### Pharmaceutical Compositions

The compounds of the invention are preferably formulated into pharmaceutical compositions for administration to human subjects in a biologically compatible form suitable for administration in vivo. Accordingly, in another aspect, the present invention provides a pharmaceutical composition comprising a compound of the invention in admixture with a suitable diluent, carrier, or excipient.

The compounds of the invention may be used in the form of the free base, in the form of salts, solvates, and as prodrugs. All forms are within the scope of the invention. In accordance with the methods of the invention, the described compounds or salts, solvates, or prodrugs thereof may be administered to a patient in a variety of forms depending on the selected route of administration, as will be understood by those skilled in the art. The compounds of the invention may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound of the invention may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a compound of the invention may be incorporated with an excipient and used in the form of ingestible tablets, buccal tablets, troches, capsules, elixirs, suspensions, syrups, and wafers.

A compound of the invention may also be administered parenterally. Solutions of a compound of the invention can be prepared in water suitably mixed with a surfactant. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2003, 20^{th} ed.) and in The United States Pharmacopeia: The National Formulary (USP 24 NF19), published in 1999.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe.

Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter.

The compounds of the invention may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds of the invention, and/or compositions comprising a compound of the invention, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds of the invention may be administered initially in a suitable dosage that may be adjusted as required, depending on the clinical response. In general, satisfactory results may be obtained when the compounds of the invention are administered to a human at a daily dosage of, for example, between 0.05 mg and 3000 mg (measured as the solid form). Dose ranges include, for example, between 10-1000 mg.

Alternatively, the dosage amount can be calculated using the body weight of the patient. For example, the dose of a compound, or pharmaceutical composition thereof, administered to a patient may range from 0.1-50 mg/kg.

The following examples are meant to illustrate the invention. They are not meant to limit the invention in any way.

### EXAMPLES

### List of Abbreviations

DIPEA=N,N-diisopropylethylamine
EtOH=ethanol
THF=tetrahydrofuran
nBuLi=n-butyl lithium
I₂=iodine
Pd(dppf)Cl₂=[1,1′-Bis(diphenylphosphino)ferrocene]dichloropalladium(II)
Cs₂CO₃=cesium carbonate
H₂O=water
Pd(PPh₃)Cl₂=Bis(triphenylphosphine)palladium(II) dichloride
Pd(PPh₃)₄=tetrakis(triphenylphosphine)palladium(0)
LiCl=lithium chloride
MeOH=methanol
NBS=N-bromosuccinimide
ACN=acetonitrile
K₂CO₃=potassium carbonate
DMA=N,N-dimethylacetamide
Zn(CN)₂=zinc cyanide
HATU=1-[Bis(dimethylamino)methylene]-1H-1,2,3-triazolo[4,5-[b]pyridinium 3-oxid hexafluorophosphate DMF=N,N-dimethylformamide
Pd(t-Bu₃P)₂=Bis(tri-tert-butylphosphine)palladium(0)
DMF-DMA=N,N-dimethylformamide dimethyl acetal
N₂H₄H₂O=hydrazine hydrate
Pd₂(dba)₃=tris(dibenzylideneacetone) dipalladium
X-Phos=2-dicyclohexylphosphino-2′,4′,6′-triisopropylbiphenyl
Pd(PPh₃)Cl₂ DCM=Bis(triphenylphosphine)palladium(II) dichloride dichloromethane complex
DMSO=dimethylsulfoxide

### Example 1. Preparation of Compounds

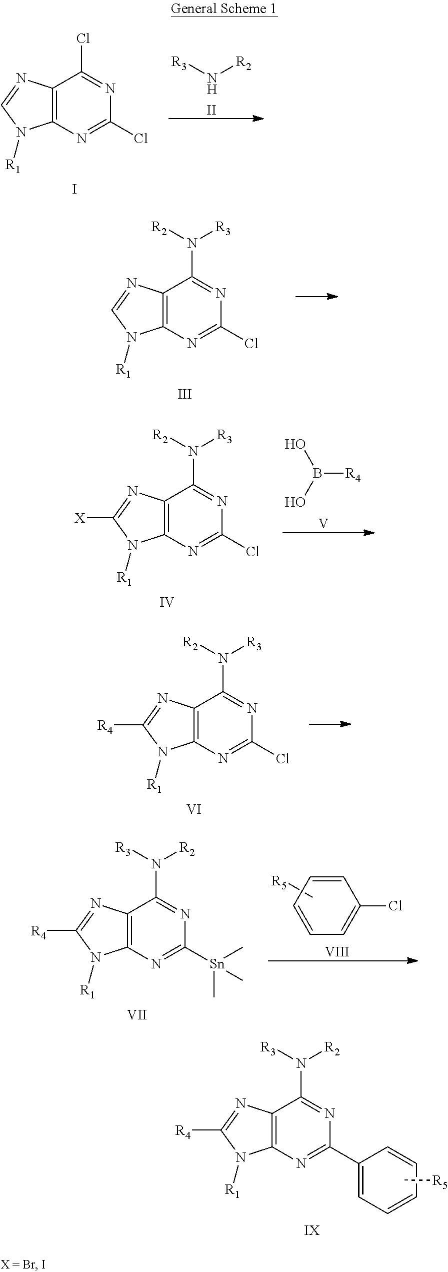

An appropriately substituted aryl chloride I is reacted with an appropriately substituted amine II under basic conditions (e.g., N,N-diisopropylethylamine) to afford appropriately substituted aryl chloride III. Aryl chloride III is halogenated with a bromine or iodide source (e.g., N-bromosuccinimide) to afford appropriately substituted aryl halide IV. Aryl halide IV is reacted with appropriately substituted boronic acid V in the presence of a palladium source (e.g., 1,1′-Bis(diphenylphosphino)ferrocene dichloropalladium(II)) to afford appropriately substituted aryl chloride VI. Aryl chloride VI is coupled with 1,1,1,2,2,2-hexamethyldistannane in the presence of a palladium source (e.g., bis(triphenylphosphine)palladium(II) dichloride) to afford appropriately substituted organostannane VII. Organostannane VII is coupled with appropriately substituted aryl chloride VIII in the presence of a palladium source (e.g., tetrakis(triphenylphosphine)palladium(0)) to afford desired purine IX.

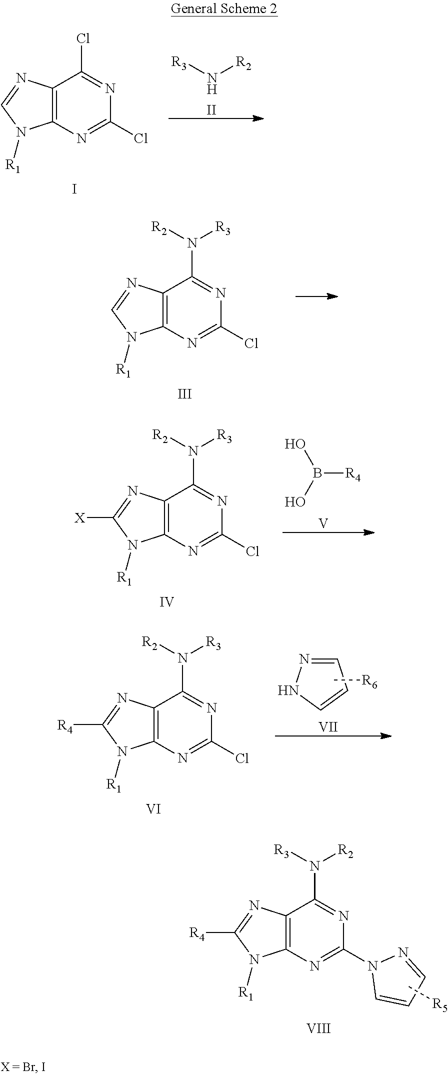

An appropriately substituted aryl chloride I is reacted with an appropriately substituted amine II under basic conditions (e.g., triethylamine) to afford appropriately substituted aryl chloride III. Aryl chloride III is halogenated with a bromine or iodide source (e.g., N-bromosuccinimide) to afford appropriately substituted aryl halide IV. Aryl halide IV is reacted with appropriately substituted boronic acid V in the presence of a palladium source (e.g., 1,1′-bis(diphenylphosphino)ferrocene dichloropalladium(II)) to afford appropriately substituted aryl chloride VI. Aryl chloride VI is coupled with appropriately substituted pyrazole VII under basic conditions (e.g., cesium carbonate) to afford desired purine VIII.

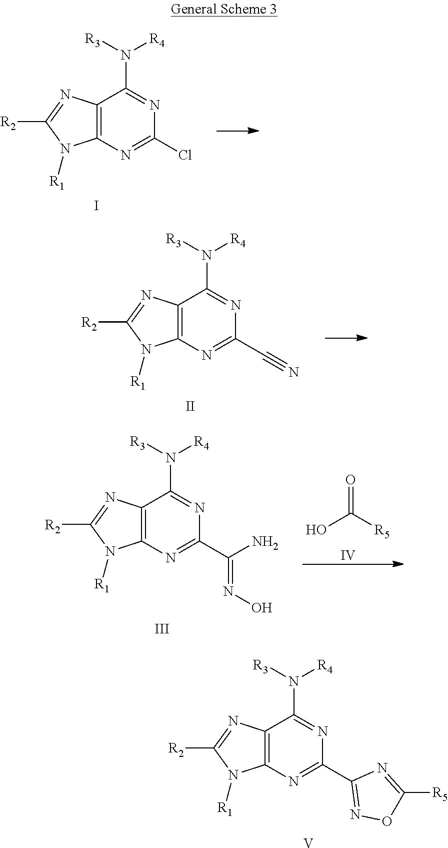

An appropriately substituted aryl chloride I is coupled with zinc cyanide in the presence of a palladium source (e.g., tetrakis(triphenylphosphine)palladium(0)) to afford appropriately substituted aryl nitrile II. Aryl nitrile II is coupled with hydroxylamine to afford appropriately substituted oxime III. Oxime III is reacted with appropriately substituted carboxylic acid IV in the presence of a coupling agent (e.g., HATU) to afford desired purine V.

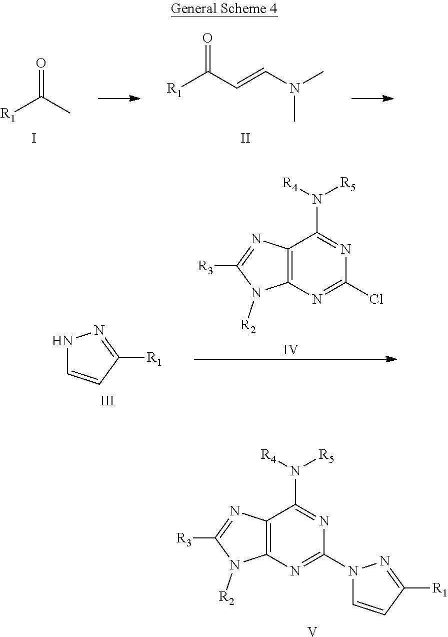

An appropriately substituted methyl ketone I is coupled N,N-dimethylformamide dimethyl acetal with heat to afford appropriately substituted enone II. Enone II is condensed with hydrazine monohydrate to afford appropriately substituted pyrazole III. Pyrazole III is reacted with appropriately substituted aryl chloride IV under basic conditions (e.g., cesium carbonate) and/or in the presence of a palladium source (e.g., tris(dibenzylideneacetone) dipalladium) to afford desired purine V.

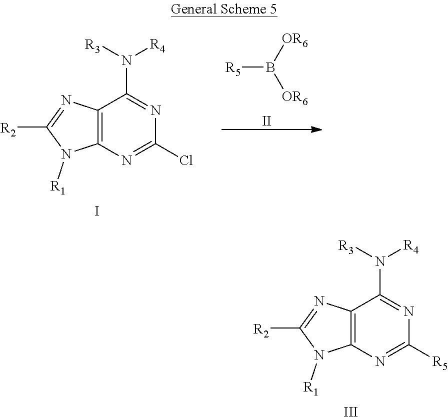

An appropriately substituted aryl chloride I is reacted with appropriately substituted boronic acid or ester II in the presence of a palladium catalyst (e.g., 1,1′-Bis(diphenylphosphino)ferrocene palladium(II)dichloride) to afford desired purine III.

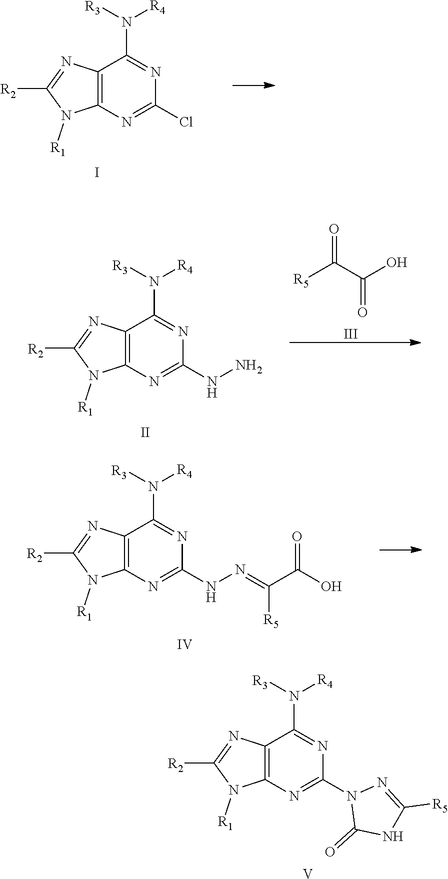

An appropriately substituted aryl chloride I is reacted with hydrazine hydrate with heat to afford appropriately substituted hydrazine II. Hydrazine II is reacted with appropriately substituted alpha-keto acid III under acidic conditions (e.g., hydrochloric acid) to afford appropriately substituted hydrazone IV. Hydrazone IV is condensed with diphenyl phosphorylazide under basic conditions (e.g., triethylamine) to afford desired purine V.

### General Procedure 1 (Compounds 1-4)

### Preparation of 7-methyl-6-(morpholin-4-yl)-2-(2-phenylpyrimidin-4-yl)-8-(pyridin-4-yl)-7H-purine

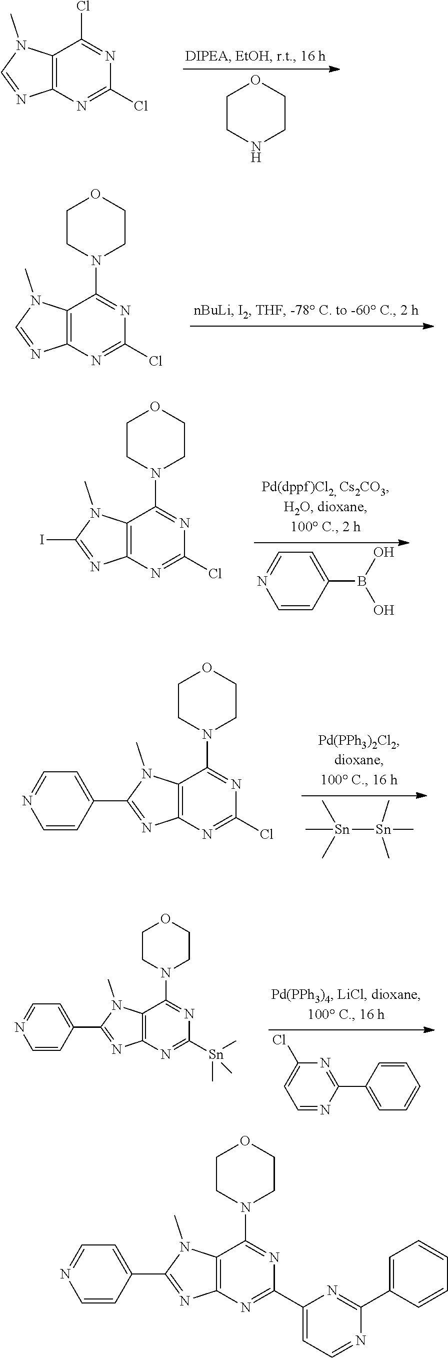

### Step 1: Preparation of 4-(2-chloro-7-methyl-7H-purin-6-yl)morpholine

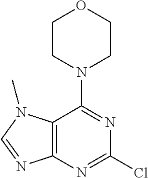 [SMILES: Cn1cnc2nc(Cl)nc(N3CCOCC3)c21]

A solution of 2,6-dichloro-7-methyl-7H-purine (4.80 g, 24 mmol), morpholine (2.27 g, 26 mmol) in ethanol (100 mL) was added N,N-diisopropyethylamine (3.06 g, 24 mmol). The reaction was stirred at room temperature for 16 hours. The precipitate was collected by filtration, washed with ethanol, and dried under vacuum to afford 4-(2-chloro-7-methyl-7H-purin-6-yl)morpholine (5.00 g, 20 mmol, 83%) as a white solid. ¹H NMR (500 MHz, Chloroform-d) δ 7.97 (s, 1H), 4.01 (s, 3H), 3.93-3.83 (m, 4H), 3.58-3.48 (m, 4H); LCMS (ESI) m/z: 254.1 [M+H]⁺.

### Step 2: Preparation of 4-(2-chloro-8-iodo-7-methyl-7H-purin-6-yl)morpholine

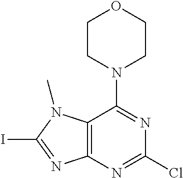 [SMILES: Cn1c(I)nc2nc(Cl)nc(N3CCOCC3)c21]

To a solution of 4-(2-chloro-7-methyl-7H-purin-6-yl)morpholine (4.50 g, 18 mmol) in tetrahydrofuran (270 ml) was added a 2.5 M solution of n-butyllithium in hexanes (8.5 mL, 21 mmol) at −78° C. The reaction was stirred at 78° C. for 30 minutes, then a solution of iodine (6.75 g, 27 mmol) in tetrahydrofuran (30 mL) was added to the reaction mixture. The reaction was allowed to warm to −60° C. over 2 hours with stirring. A solution of saturated sodium thiosulfate (200 mL) was added to the reaction vial at −60° C., then organics were extracted with ethyl acetate (2×500 mL). The organic layers were pooled, washed with brine (100 mL), dried over anhydrous sodium sulfate, filtered and concentrated under reduced pressure. The crude residue was purified via flash column chromatography through silica gel using a gradient of 0-5% methanol in dichloromethane. Product 4-(2-chloro-8-iodo-7-methyl-7H-purin-6-yl)morpholine (2.30 g, 6.1 mmol, 34%) was afforded as a yellow solid. NMR data unavailable; LCMS (ESI) m/z: 216.1 [M+H]⁺.

### Step 3: Preparation of 4-(2-chloro-7-methyl-8-(pyridin-4-yl)-7H-purin-6-yl)morpholine

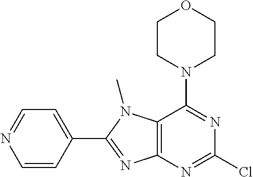 [SMILES: Cn1c(-c2ccncc2)nc2nc(Cl)nc(N3CCOCC3)c21]

To a solution of 4-(2-chloro-8-iodo-7-methyl-7H-purin-6-yl)morpholine (2.30 g, 6.1 mmol) in dioxane (120 mL) and water (30 mL) was added pyridin-4-ylboronic acid (0.372 g, 3.0 mmol), cesium carbonate (0.197 g, 0.61 mmol), [1,1′Bis(diphenylphosphino)ferrocene]dichloropalladium(II) (0.219 g, 0.30 mmol) and stirred at 100° C. under argon for 2 hours. Water (500 mL) was added to the reaction mixture, and the organics were extracted with ethyl acetate (3×500 mL). The organic layers were pooled, dried over anhydrous sodium sulfate, filtered and concentrated under reduced pressure. The crude residue was purified via flash column chromatography through silica gel using a gradient of 0-10% methanol in dichloromethane. Product 4-(2-chloro-7-methyl-8-(pyridin-4-yl)-7H-purin-6-yl)morpholine (0.750 g, 2.3 mmol, 75%) was afforded as a yellow solid. NMR data unavailable; LCMS (ESI) m/z: 331.0 [M+H]⁺.

### Step 4: Preparation of 4-(7-methyl-8-(pyridin-4-yl)-2-(trimethylstannyl)-7H-purin-6-yl)morpholine

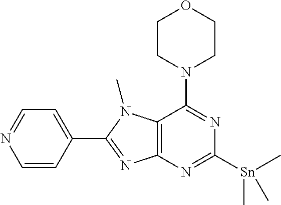 [SMILES: Cn1c(-c2ccncc2)nc2n[c]([Sn]([CH3])([CH3])[CH3])nc(N3CCOCC3)c21]

To a solution of 4-(2-chloro-7-methyl-8-(pyridin-4-yl)-7H-purin-6-yl)morpholine (281 mg, 0.85 mmol) in dioxane (10 mL) was added 1,1,1,2,2,2-hexamethyldistannane (557 mg, 1.7 mmol) and bis(triphenylphosphine)palladium(II) dichloride (91.0 mg, 0.13 mmol). The reaction was stirred at 100° C. for 2 hours. The reaction mixture was allowed to cool to room temperature, then a 4 M solution of aqueous potassium fluoride (50 mL) was added to the reaction mixture and stirred for 30 minutes. The mixture was filtered over celite. The organics were extracted from the filtrate with dichloromethane (2×60 mL), washed with brine (40 mL), dried over sodium sulfate, and concentrated under reduced pressure. Crude product 4-(7-methyl-8-(pyridin-4-yl)-2-(trimethylstannyl)-7H-purin-6-yl)morpholine (390 mg, 0.85 mmol, 100%) was afforded as a brown solid and carried onto next step without further purification. NMR data unavailable; LCMS (ESI) m/z: 459.0 [M+H]⁺.

### Step 5: Preparation of 4-(7-methyl-2-(2-phenylpyrimidin-4-yl)-8-(pyridin-4-yl)-7H-purin-6-yl)morpholine

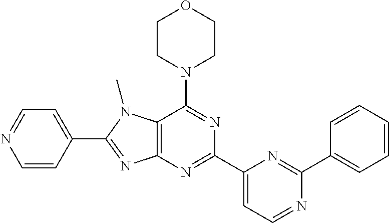 [SMILES: Cn1c(-c2ccncc2)nc2nc(-c3ccnc(-c4ccccc4)n3)nc(N3CCOCC3)c21]

To a solution of 4-(7-methyl-8-(pyridin-4-yl)-2-(trimethylstannyl)-7H-purin-6-yl)morpholine (390 mg, 0.85 mmol), 4-chloro-2-phenylpyrimidine (194 mg, 1.0 mmol), lithium chloride (89.0 mg, 2.13 mmol) in dioxane (10 mL) was added tetrakis(triphenylphosphine)palladium(0) (98.0 mg, 0.085 mmol). The reaction mixture was stirred at 100° C. for 16 hours under argon. The reaction was allowed to cool to room temperature, then filtered over celite and washed with ethyl acetate (2×30 mL). The filtrate was concentrated under reduced pressure. Crude product was purified by pre-HPLC (the crude samples were dissolved in N,N-dimethylformamide unless otherwise noted before purification. Boston pHlex ODS 10 um 21.2×250 mm 120A. The mobile phase was acetonitrile/0.1% Ammonium bicarbonate) to give product 4-(7-methyl-2-(2-phenylpyrimidin-4-yl)-8-(pyridin-4-yl)-7H-purin-6-yl)morpholine (14.1 mg, 0.031 mmol, 3.3%) as a white solid. ¹H NMR (500 MHz, Chloroform-d) δ 8.98 (d, J=5.1 Hz, 1H), 8.88 (d, J=5.1 Hz, 2H), 8.69-8.62 (m, 2H), 8.37 (d, J=5.1 Hz, 1H), 7.85 (d, J=5.2 Hz, 2H), 7.56-7.48 (m, 3H), 4.12 (s, 3H), 4.05-3.98 (m, 4H), 3.75 (t, J=4.6 Hz, 4H). LCMS (ESI) m/z: 451.0 [M+H]⁺.

### Compounds 1˜4

| # | Structure | LCMS Data | ¹H NMR Data |
|---|---|---|---|
| 1 | 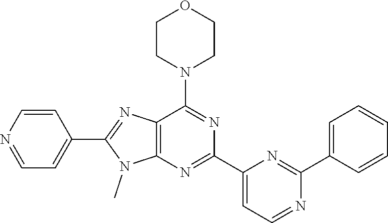 [SMILES: Cn1c(-c2ccncc2)nc2c(N3CCOCC3)nc(-c3ccnc(-c4ccccc4)n3)nc21] | LCMS (ESI) m/z: 451.2 [M + H]⁺. | ¹H NMR (500 MHz, Chloroform-d) δ 8.97 (d, J = 5.1 Hz, 1H), 8.87-8.79 (m, 2H), 8.66-8.58 (m, 2H), 8.31 (d, J = 5.1 Hz, 1H), 7.82-7.74 (m, 2H), 7.57-7.48 (m, 3H), 4.51 (s, 4H), 4.13 (s, 3H), 3.97-3.89 (m, 4H). |
| 2 | 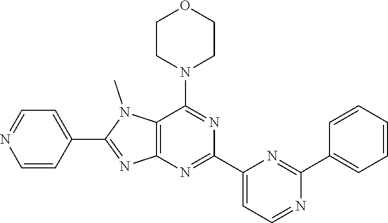 [SMILES: Cn1c(-c2ccncc2)nc2nc(-c3ccnc(-c4ccccc4)n3)nc(N3CCOCC3)c21] | LCMS (ESI) m/z: 451.0 [M + H]⁺. | ¹H NMR (500 MHz, Chloroform-d) δ 8.98 (d, J = 5.1 Hz, 1H), 8.88 (d, J = 5.1 Hz, 2H), 8.69-8.62 (m, 2H), 8.37 (d, J = 5.1 Hz, 1H), 7.85 (d, J = 5.2 Hz, 2H), 7.56-7.48 (m, 3H), 4.12 (s, 3H), 4.05-3.98 (m, 4H), 3.75 (t, J = 4.6 Hz, 4H). |
| 3 | 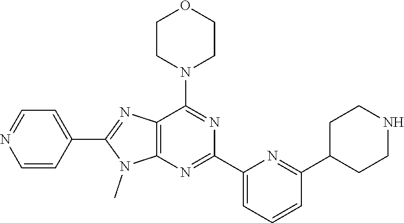 [SMILES: Cn1c(-c2ccncc2)nc2c(N3CCOCC3)nc(-c3cccc(C4CCNCC4)n3)nc21] | LCMS (ESI) m/z: 457.2 [M + H]⁺. | ¹H NMR (400 MHz, Dimethylsulfoxide-d₆) δ 8.80 (dd, J = 4.5, 1.6 Hz, 2H), 8.20 (d, J = 7.4 Hz, 1H), 7.94 (dd, J = 4.5, 1.6 Hz, 2H), 7.85 (t, J = 7.8 Hz, 1H), 7.34 (d, J = 7.6 Hz, 1H), 4.36 (s, 4H), 4.00 (s, 3H), 3.87-3.71 (m, 4H), 3.07 (d, J = 11.6 Hz, 2H), 2.86 (t, J = 12.0 Hz, 1H), 2.64 (t, J = 11.5 Hz, 2H), 1.84 (d, J = 11.7 Hz, 2H), 1.69 (qd, J = 12.3, 3.8 Hz, 2H). |
| 4 | 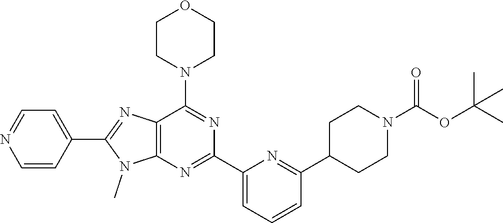 [SMILES: Cn1c(-c2ccncc2)nc2c(N3CCOCC3)nc(-c3cccc(C4CCN(C(=O)OC(C)(C)C)CC4)n3)nc21] | LCMS (ESI) m/z: 557.3 [M + H]⁺. | ¹H NMR (400 MHz, Dimethylsulfoxide-d₆) δ 8.80 (dd, J = 4.5, 1.6 Hz, 2H), 8.22 (d, J = 7.2 Hz, 1H), 7.94 (dd, J = 4.5, 1.6 Hz, 2H), 7.86 (t, J = 7.8 Hz, 1H), 7.38 (d, J = 7.3 Hz, 1H), 4.36 (s, 4H), 4.09 (d, J = 12.5 Hz, 2H), 4.00 (s, 3H), 3.86-3.70 (m, 4H), 2.99 (t, J = 11.7 Hz, 3H), 1.91 (d, J = 11.3 Hz, 2H), 1.68 (dt, J = 12.2, 8.4 Hz, 2H), 1.55-1.21 (m, 9H). |

### General Procedure 2 (Compounds 5-29)

### Preparation of 9-methyl-6-(morpholin-4-yl)-2-[3-(pyridin-3-yl)-1H-pyrazol-1-yl]-8-(pyridin-4-yl)-9H-purine

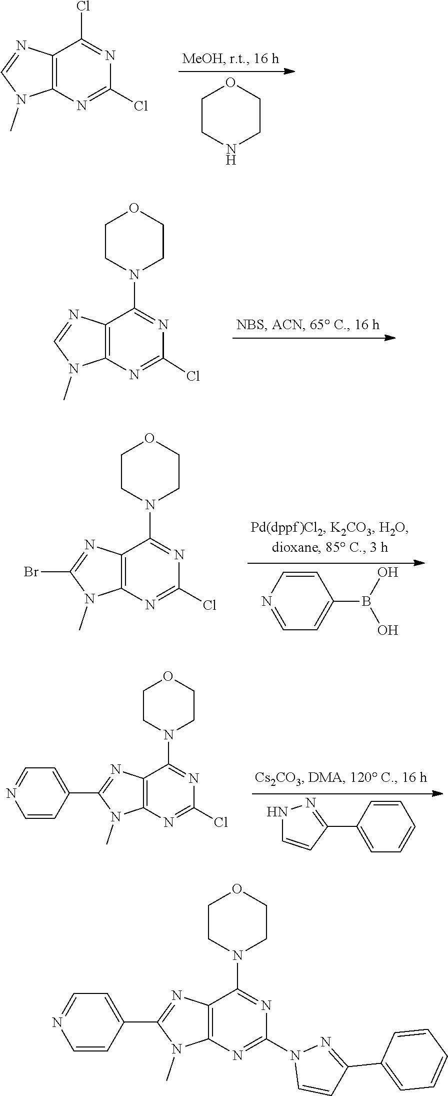

### Step 1: Preparation of 4-(2-Chloro-9-methyl-9H-purin-6-yl)morpholine

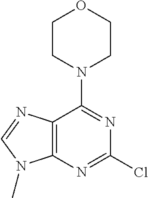 [SMILES: Cn1cnc2c(N3CCOCC3)nc(Cl)nc21]

A mixture of 2,6-dichloro-9-methyl-9H-purine (6.00 g, 30 mmol) and morpholine (6.50 g, 74 mmol) in methanol (300 mL) was stirred at room temperature for 16 hours. The mixture was filtered directly, and the residue was triturated with methanol. Product 4-(2-Chloro-9-methyl-9H-purin-6-yl)morpholine (7.00 g, 28 mmol, 93%) was afforded as a white solid and carried onto next step without further purification. NMR data unavailable; LCMS (ESI) m/z: 254.1 [M+H]⁺.

### Step 2: Preparation of 4-(8-Bromo-2-chloro-9-methyl-9H-purin-6-yl)morpholine

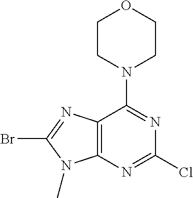 [SMILES: Cn1c(Br)nc2c(N3CCOCC3)nc(Cl)nc21]

A mixture of 4-(2-chloro-9-methyl-9H-purin-6-yl)morpholine (7.00 g, 28 mmol) and N-bromosuccinimide (8.80 g, 50 mmol) in acetonitrile (500 mL) was stirred at 65° C. for 16 hours. The mixture was filtered directly, and the residue was triturated with acetonitrile. Product 4-(8-Bromo-2-chloro-9-methyl-9H-purin-6-yl)morpholine (8.00 g, 24 mmol, 87%) was afforded as light yellow solid and carried onto next step without further purification. NMR data unavailable; LCMS (ESI) m/z: 332.3 [M+H]⁺.

### Step 3: Preparation of 4-(2-Chloro-9-methyl-8-(pyridin-4-yl)-9H-purin-6-yl)morpholine

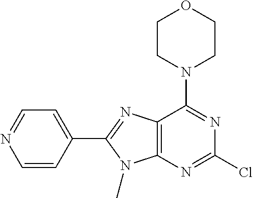 [SMILES: Cn1c(-c2ccncc2)nc2c(N3CCOCC3)nc(Cl)nc21]

In a reaction vial, 4-(8-bromo-2-chloro-9-methyl-9H-purin-6-yl)morpholine (5.00 g, 15 mmol), pyridin-4-ylboronic acid (2.20 g, 18 mmol), 1,1′-bis(diphenylphosphino)ferrocene-palladium(II)dichloride (1.10 g, 1.5 mmol) and potassium carbonate (5.20 g, 38 mmol) were suspended in dioxane (50 mL) and water (5 mL) under nitrogen. The reaction mixture was stirred at 85° C. for 3 hours. The reaction was filtered over celite and washed with ethyl acetate (3×25 mL). The filtrate was concentrated under reduced pressure. Crude product was purified via flash column chromatography through silica gel using a gradient of 0-5% methanol in dichloromethane. Product 4-(2-Chloro-9-methyl-8-(pyridin-4-yl)-9H-purin-6-yl)morpholine (3.00 g, 9.1 mmol, 60%) was afforded as a light yellow solid. NMR data unavailable; LCMS (ESI) m/z: 331.1 [M+H]⁺.

### Step 4: Preparation of 4-(9-methyl-2-(3-phenyl-1H-pyrazol-1-yl)-8-(pyridin-4-yl)-9H-purin-6-yl)morpholine

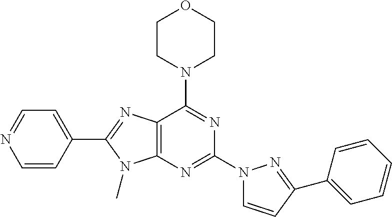 [SMILES: Cn1c(-c2ccncc2)nc2c(N3CCOCC3)nc(-n3ccc(-c4ccccc4)n3)nc21]

A mixture of 4-(2-chloro-9-methyl-8-(pyridin-4-yl)-9H-purin-6-yl)morpholine (100 mg, 0.30 mmol), 3-phenyl-1H-pyrazole (58.0 mg, 0.40 mmol) and cesium carbonate (196 mg, 0.60 mmol) in N,N-dimethylacetamide (5 mL) was stirred at 120° C. for 16 hours. The product was indicated present via UPLC analysis. The mixture was allowed to cool to room temperature, quenched with water (10 mL) and the organics were extracted with ethyl acetate (3×10 mL). The organic layers were pooled, washed with brine (10 mL), dried over sodium sulfate, filtered, and concentrated under reduced pressure. Crude product was purified by prep-HPLC (the crude samples were dissolved in methanol unless otherwise noted before purification. Boston C18 21*250 mm 10 μm column. The mobile phase was acetonitrile/0.01% aqueous ammonium bicarbonate). Product 4-(9-methyl-2-(3-phenyl-1H-pyrazol-1-yl)-8-(pyridin-4-yl)-9H-purin-6-yl)morpholine (25.6 mg, 0.058 mmol, 19%) was afforded as a white solid. ¹H NMR (400 MHz, Dimethylsulfoxide-d₆) δ 9.17 (d, J=2.3 Hz, 1H), 8.82 (d, J=2.7 Hz, 1H), 8.80-8.76 (m, 2H), 8.59 (dd, J=4.8, 1.6 Hz, 1H), 8.33 (dt, J=7.9, 1.9 Hz, 1H), 7.94-7.87 (m, 2H), 7.51 (dd, J=7.9, 4.8 Hz, 1H), 7.17 (d, J=2.5 Hz, 1H), 4.23 (s, 4H), 3.97 (s, 3H), 3.80 (t, J=4.8 Hz, 4H); LCMS (ESI) m/z: 439 [M+H]⁺.

### Compounds 5-29

| # | Structure | LCMS Data | ¹H NMR Data |
|---|---|---|---|
| 5 | 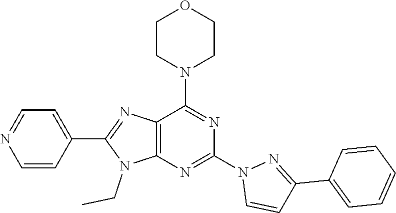 [SMILES: CCn1c(-c2ccncc2)nc2c(N3CCOCC3)nc(-n3ccc(-c4ccccc4)n3)nc21] | LCMS (ESI) m/z: 453.2 [M + H]⁺. | ¹H NMR (400 MHz, Dimethylsulfoxide-d₆) δ 8.79 (dd, J = 11.1, 4.3 Hz, 3H), 8.08-7.95 (m, 2H), 7.86 (dd, J = 4.5, 1.6 Hz, 2H), 7.48 (t, J = 7.5 Hz, 2H), 7.40 (d, J = 7.3 Hz, 1H), 7.06 (d, J = 2.7 Hz, 1H), 4.46 (m, 6H), 3.86-3.59 (m, 4H), 1.36 (t, J = 7.2 Hz, 3H). |
| 6 | 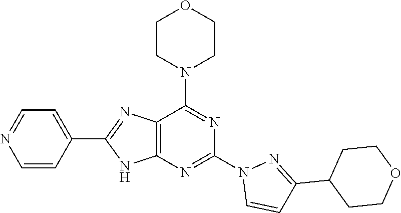 [SMILES: c1cc(-c2nc3c(N4CCOCC4)nc(-n4ccc(C5CCOCC5)n4)nc3[nH]2)ccn1] | LCMS (ESI) m/z: 433 [M + H]⁺. | ¹H NMR (400 MHz, Dimethylsulfoxide-d₆) δ 8.76-8.70 (m, 2H), 8.54 (d, J = 2.6 Hz, 1H), 8.04-7.99 (m, 2H), 6.42 (d, J = 2.6 Hz, 1H), 4.35 (s, 4H), 3.97-3.89 (m, 2H), 3.80 (t, J = 4.7 Hz, 4H), 3.46 (td, J = 11.6, 2.2 Hz, 2H), 2.96-2.88 (m, 1H), 1.93-1.85 (m, 2H), 1.77-1.64 (m, 2H). |
| 7 | 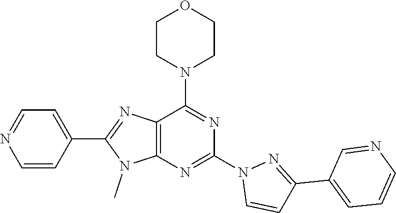 [SMILES: Cn1c(-c2ccncc2)nc2c(N3CCOCC3)nc(-n3ccc(-c4cccnc4)n3)nc21] | LCMS (ESI) m/z: 439 [M + H]⁺. | ¹H NMR (400 MHz, Dimethylsulfoxide-d₆) δ 9.17 (d, J = 2.3 Hz, 1H), 8.82 (d, J = 2.7 Hz, 1H), 8.80-8.76 (m, 2H), 8.59 (dd, J = 4.8, 1.6 Hz, 1H), 8.33 (dt, J = 7.9, 1.9 Hz, 1H), 7.94-7.87 (m, 2H), 7.51 (dd, J = 7.9, 4.8 Hz, 1H), 7.17 (d, J = 2.5 Hz, 1H), 4.23 (s, 4H), 3.97 (s, 3H), 3.80 (t, J = 4.8 Hz, 4H). |
| 8 | 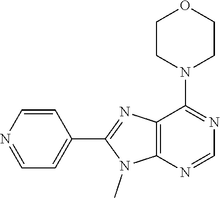 [SMILES: Cn1c(-c2ccncc2)nc2c(N3CCOCC3)ncnc21] | LCMS (ESI) m/z: 297.1 [M + H]⁺. | ¹H NMR (400 MHz, Dimethylsulfoxide-d₆) δ 8.71 (d, J = 5.9 Hz, 2H), 8.32-8.23 (m, 3H), 4.32 (s, 4H), 3.83 (s, 3H), 3.81-3.74 (m, 4H). |
| 9 | 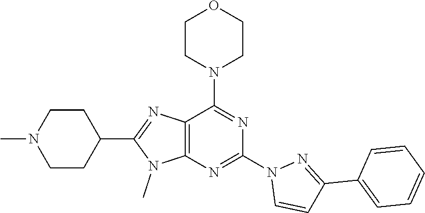 [SMILES: CN1CCC(c2nc3c(N4CCOCC4)nc(-n4ccc(-c5ccccc5)n4)nc3n2C)CC1] | LCMS (ESI) m/z: 459 [M + H]⁺. | ¹H NMR (400 MHz, Dimethylsulfoxide-d₆) δ 8.718-8.723 (d, J = 2.0 Hz, 1H), 7.949-7.966 (m, 2H), 7.437-7.490 (t, 2H), 7.024-7.029 (d, J = 2.0 Hz, 1H), 4.256-4.333 (m, 4H), 7.765 (s, 7H), 2.867-2.935 (m, 3H), 2.215 (s, 3H), 2.012-2.065 (m, 2H), 1.804-1.897 (m, 4H). |
| 10 | 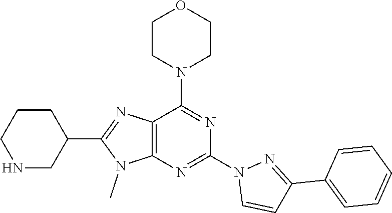 [SMILES: Cn1c(C2CCCNC2)nc2c(N3CCOCC3)nc(-n3ccc(-c4ccccc4)n3)nc21] | LCMS (ESI) m/z: 445.2 [M + H]⁺. | ¹H NMR (400 MHz, Dimethylsulfoxide-d₆) δ 8.72 (d, J = 2.6 Hz, 1H), 7.96 (d, J = 7.2 Hz, 2H), 7.47 (t, J = 7.5 Hz, 2H), 7.38 (t, J = 7.3 Hz, 1H), 7.02 (d, J = 2.6 Hz, 1H), 4.29 (s, 4H), 3.77 (s, 7H), 3.24 (d, J = 13.8 Hz, 1H), 3.12 (s, 1H), 3.02 (d, J = 11.5 Hz, 1H), 2.78 (t, J = 11.2 Hz, 1H), 2.65 (d, J = 16.7 Hz, 1H), 2.06 (d, J = 11.0 Hz, 1H), 1.75 (t, J = 12.2 Hz, 2H), 1.57 (d, J = 12.5 Hz, 1H). |
| 11 | 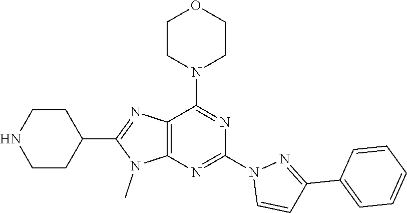 [SMILES: Cn1c(C2CCNCC2)nc2c(N3CCOCC3)nc(-n3ccc(-c4ccccc4)n3)nc21] | LCMS (ESI) m/z: 445 [M + H]⁺. | ¹H NMR (400 MHz, Dimethylsulfoxide-d₆) δ 8.716-8.721 (d, J = 2.0 Hz, 1H), 7.948-7.962 (d, J = 5.6 Hz, 2H), 7.456-7.486 (m, 2H), 7.364-7.392 (m, 1H), 7.022-7.027 (d, J = 2.0 Hz, 1H), 4.277-4.311 (m, 4H), 3.769 (s, 7H), 3.325 (s, 2H), 3.072-3.124 (m, 2H), 2.751-2.770 (m, 1H), 1.899-1.970 (m, 2H), 1.750-1.772 (m, 2H). |
| 12 | 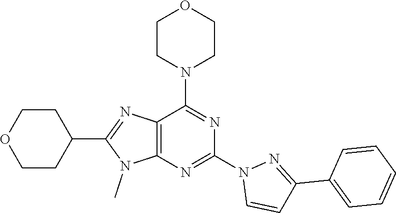 [SMILES: Cn1c(C2CCOCC2)nc2c(N3CCOCC3)nc(-n3ccc(-c4ccccc4)n3)nc21] | LCMS (ESI) m/z: 446 [M + H]⁺. | ¹H NMR (400 MHz, Dimethylsulfoxide-d₆) δ 8.72 (d, J = 2.7 Hz, 1H), 7.99-7.93 (m, 2H), 7.47 (dd, J = 8.3, 7.0 Hz, 2H), 7.41-7.35 (m, 1H), 7.02 (d, J = 2.7 Hz, 1H), 4.30 (s, 4H), 3.99-3.94 (m, 2H), 3.80-3.75 (m, 7H), 3.51 (td, J = 11.3, 3.2 Hz, 2H), 3.31-3.25 (m, 1H), 1.92-1.77 (m, 4H). |
| 13 | 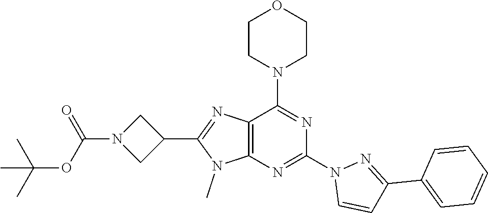 [SMILES: Cn1c(C2CN(C(=O)OC(C)(C)C)C2)nc2c(N3CCOCC3)nc(-n3ccc(-c4ccccc4)n3)nc21] | LCMS (ESI) m/z: 517.2 [M + H]⁺. | ¹H NMR (400 MHz, Dimethylsulfoxide-d₆) δ 8.73 (d, J = 2.6 Hz, 1H), 7.96 (d, J = 7.2 Hz, 2H), 7.47 (t, J = 7.5 Hz, 2H), 7.38 (t, J = 7.3 Hz, 1H), 7.04 (d, J = 2.7 Hz, 1H), 4.84-4.05 (m, 9H), 3.92-3.72 (m, 4H), 3.64 (s, 3H), 1.40 (s, 9H). |
| 14 | 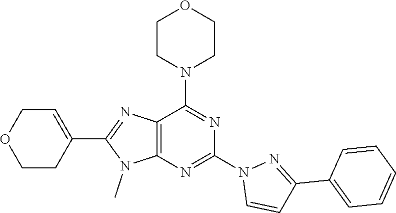 [SMILES: Cn1c(C2=CCOCC2)nc2c(N3CCOCC3)nc(-n3ccc(-c4ccccc4)n3)nc21] | LCMS (ESI) m/z: 444 [M + H]⁺. | ¹H NMR (400 MHz, Dimethylsulfoxide-d₆) δ 8.72 (d, J = 2.7 Hz, 1H), 7.99-7.90 (m, 2H), 7.47 (t, J = 7.6 Hz, 2H), 7.38 (t, J = 7.4 Hz, 1H), 7.02 (d, J = 2.6 Hz, 1H), 6.57 (dd, J = 6.2, 2.1 Hz, 1H), 4.88 (dd, J = 6.3, 3.3 Hz, 1H), 4.43-4.17 (m, 4H), 4.09-4.04 (m, 1H), 3.96-3.91 (m, 1H), 3.82-3.72 (m, 7H), 3.30 (s, 1H), 2.24-2.11 (m, 2H). |
| 15 | 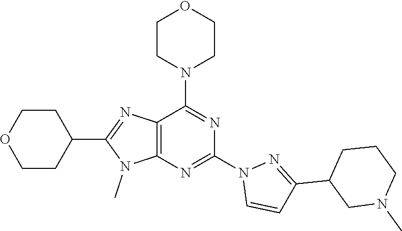 [SMILES: CN1CCCC(c2ccn(-c3nc(N4CCOCC4)c4nc(C5CCOCC5)n(C)c4n3)n2)C1] | LCMS (ESI) m/z: 467 [M + H]⁺. | ¹H NMR (400 MHz, Dimethylsulfoxide-d₆) δ 8.54 (d, J = 2.6 Hz, 1H), 6.39 (d, J = 2.7 Hz, 1H), 4.26 (s, 4H), 4.00-3.92 (m, 2H), 3.74 (d, J = 4.3 Hz, 7H), 3.50 (td, J = 11.1, 3.5 Hz, 3H), 3.09-3.01 (m, 1H), 3.00-2.82 (m, 2H), 2.29 (s, 3H), 2.14 (t, J = 11.0 Hz, 1H), 2.04 (t, J = 11.6 Hz, 1H), 2.00-1.91 (m, 1H), 1.90-1.78 (m, 4H), 1.77-1.59 (m, 2H), 1.46-1.36 (m, 1H). |
| 16 | 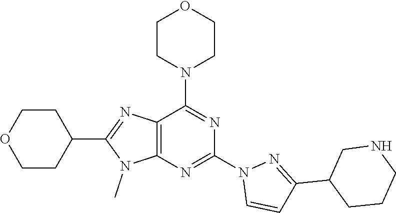 [SMILES: Cn1c(C2CCOCC2)nc2c(N3CCOCC3)nc(-n3ccc(C4CCCNC4)n3)nc21] | LCMS (ESI) m/z: 453 [M + H]⁺. | ¹H NMR (400 MHz, Dimethylsulfoxide-d₆) δ 8.58-8.49 (m, 1H), 6.47-6.31 (m, 1H), 4.26 (s, 4H), 4.00-3.91 (m, 2H), 3.80-3.70 (m, 7H), 3.50 (td, J = 11.2, 3.5 Hz, 2H), 3.26 (dd, J = 10.2, 4.8 Hz, 2H), 3.10 (d, J = 12.5 Hz, 1H), 2.93 (d, J = 12.2 Hz, 1H), 2.77 (s, 1H), 2.62-2.53 (m, 2H), 1.99 (d, J = 11.5 Hz, 1H), 1.90-1.75 (m, 4H), 1.69-1.42 (m, 3H). |
| 17 | 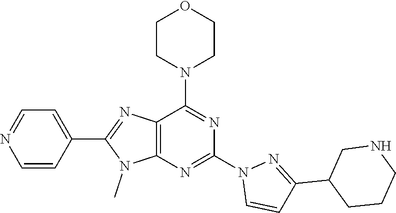 [SMILES: Cn1c(-c2ccncc2)nc2c(N3CCOCC3)nc(-n3ccc(C4CCCNC4)n3)nc21] | LCMS (ESI) m/z: 446 [M + H]⁺. | ¹H NMR (400 MHz, Dimethylsulfoxide-d₆) δ 8.83-8.74 (m, 2H), 8.58 (d, J = 2.6 Hz, 1H), 7.95-7.87 (m, 2H), 6.40 (d, J = 2.6 Hz, 1H), 3.93 (s, 8H), 3.78 (t, J = 4.8 Hz, 4H), 3.11 (d, J = 12.0 Hz, 1H), 2.93 (d, J = 12.3 Hz, 1H), 2.85-2.55 (m, 3H), 2.01 (d, J = 12.0 Hz, 1H), 1.77-1.35 (m, 3H). |
| 18 | 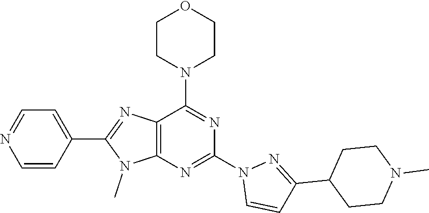 [SMILES: CN1CCC(c2ccn(-c3nc(N4CCOCC4)c4nc(-c5ccncc5)n(C)c4n3)n2)CC1] | LCMS (ESI) m/z: 460 [M + H]⁺. | ¹H NMR (400 MHz, Dimethylsulfoxide-d₆) δ 8.79-8.77 (m, 2H), 8.59 (d, J = 2.4 Hz, 1H), 7.91-7.90 (m, 2H), 6.42 (d, J = 2.4 Hz, 1H), 5.50-4.00 (m, 4H), 3.93 (s, 3H), 3.79-3.77 (m, 4H), 2.91-2.82 (m, 2H), 2.70-2.59 (m, 1H), 2.21 (s, 3H), 2.08-1.97 (m, 2H), 1.93-1.84 (m, 2H), 1.76-1.62 (m, 2H). |
| 19 | 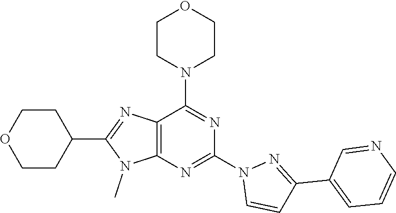 [SMILES: Cn1c(C2CCOCC2)nc2c(N3CCOCC3)nc(-n3ccc(-c4cccnc4)n3)nc21] | LCMS (ESI) m/z: 447 [M + H]⁺. | ¹H NMR (400 MHz, Dimethylsulfoxide-d₆) δ 9.16 (d, J = 2.3 Hz, 1H), 8.78 (d, J = 2.7 Hz, 1H), 8.58 (dd, J = 4.8, 1.7 Hz, 1H), 8.32 (dt, J = 8.0, 2.0 Hz, 1H), 7.51 (dd, J = 7.9, 4.8 Hz, 1H), 7.15 (d, J = 2.7 Hz, 1H), 4.30 (s, 4H), 3.97 (dt, J = 11.4, 3.2 Hz, 2H), 3.82-3.72 (m, 7H), 3.51 (td, J = 11.2, 3.4 Hz, 2H), 3.29-3.25 (m, 1H), 1.93-1.77 (m, 4H). |
| 20 | 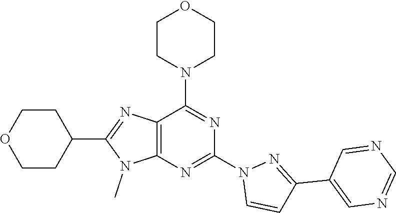 [SMILES: Cn1c(C2CCOCC2)nc2c(N3CCOCC3)nc(-n3ccc(-c4cncnc4)n3)nc21] | LCMS (ESI) m/z: 448 [M + H]⁺. | ¹H NMR (400 MHz, Dimethylsulfoxide-d₆) δ 9.34 (s, 2H), 9.20 (s, 1H), 8.83 (d, J = 2.7 Hz, 1H), 7.24 (d, J = 2.7 Hz, 1H), 4.31 (s, 4H), 4.03-3.92 (m, 2H), 3.78 (d, J = 4.7 Hz, 7H), 3.51 (td, J = 11.2, 3.4 Hz, 2H), 3.29-3.25 (m, 1H), 1.96-1.74 (m, 4H). |
| 21 | 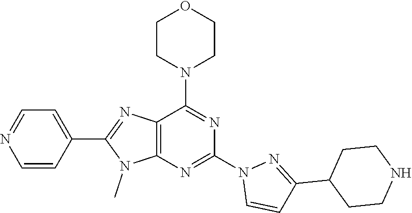 [SMILES: Cn1c(-c2ccncc2)nc2c(N3CCOCC3)nc(-n3ccc(C4CCNCC4)n3)nc21] | LCMS (ESI) m/z: 446 [M + H]⁺. | 1H NMR (400 MHz, Dimethylsulfoxide-d6) δ 8.79-8.77 (m, 2H), 8.59 (d, J = 2.4 Hz, 1H), 7.91-7.90 (m, 2H), 6.40 (d, J = 2.4 Hz, 1H), 4.37-4.28 (m, 4H), 3.93 (s, 3H), 3.79-3.77 (m, 4 H),3.04-3.01 (m, 2H), 2.81-2.58 (m, 3H), 1.85-1.82 (m, 2H), 1.61-1.51 (m, 2H). |
| 22 | 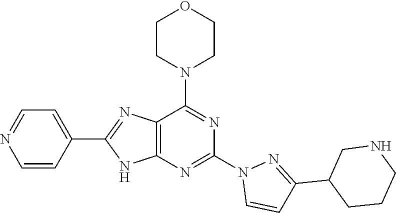 [SMILES: c1cc(-c2nc3c(N4CCOCC4)nc(-n4ccc(C5CCCNC5)n4)nc3[nH]2)ccn1] | LCMS (ESI) m/z: 432 [M + H]⁺. | ¹H NMR (400 MHz, Dimethylsulfoxide-d₆) δ 8.66 (d, J = 5.2 Hz, 2H), 8.52 (d, J = 2.6 Hz, 1H), 8.05-7.99 (m, 2H), 6.39 (d, J = 2.6 Hz, 1H), 4.34 (s, 4H), 3.11 (d, J = 12.1 Hz, 2H), 2.97-2.62 (m, 4H), 2.06 (s, 1H), 1.75 (s, 1H), 1.67-1.61 (m, 2H). |
| 23 | 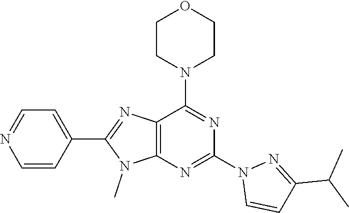 [SMILES: CC(C)c1ccn(-c2nc(N3CCOCC3)c3nc(-c4ccncc4)n(C)c3n2)n1] | LCMS (ESI) m/z: 405 [M + H]⁺. | ¹H NMR (400 MHz, Dimethylsulfoxide-d₆) δ 8.79-8.77 (m, 2H), 8.59 (d, J = 2.8 Hz, 1H), 7.91-7.90 (m, 2H), 6.41 (d, J = 2.8 Hz, 1H), 4.39-4.22 (m, 4H), 3.93 (s, 1H), 3.79-3.77 (m, 4H), 3.06-3.00 (m, 1H), 1.27-1.26 (m, 6H). |
| 24 | 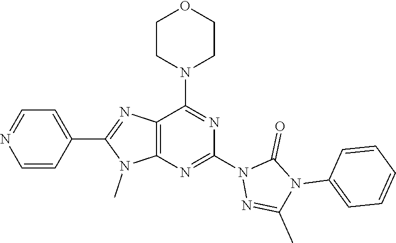 [SMILES: Cc1nn(-c2nc(N3CCOCC3)c3nc(-c4ccncc4)n(C)c3n2)c(=O)n1-c1ccccc1] | LCMS (ESI) m/z: 470.1 [M + H]⁺. | ¹H NMR (400 MHz, Dimethylsulfoxide-d₆) δ 8.78 (dd, J = 4.5, 1.6 Hz, 2H), 7.91 (dd, J = 4.5, 1.6 Hz, 2H), 7.67-7.43 (m, 5H), 4.34 (s, 4H), 3.91 (s, 3H), 3.76 (d, J = 4.3 Hz, 4H), 2.17 (s, 3H). |
| 25 | 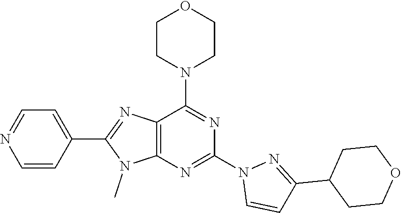 [SMILES: Cn1c(-c2ccncc2)nc2c(N3CCOCC3)nc(-n3ccc(C4CCOCC4)n3)nc21] | LCMS (ESI) m/z: 447 [M + H]⁺. | ¹H NMR (400 MHz, Dimethylsulfoxide-d₆) δ 8.773-8.788 (m, 2H), 8.604-8.610 (d, J = 2.4 Hz, 1H), 7.898-7.913 (m, 2H), 6.442-6.449 (d, J = 2.8 Hz, 1H), 3.996-4.620 (m, 4H), 3.933-3.955 (m, 5H), 3.767-3.790 (t, 4H), 3.460-3.490 (t, 2H), 2.966 (m, 1H), 1.833-1.863 (m, 2H), 1.694-1.754 (m, 2H). |
| 26 | 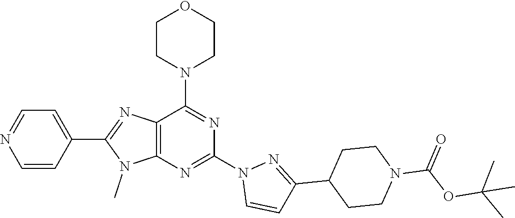 | LCMS (ESI) m/z: 460 [M + H]⁺. | ¹H NMR (400 MHz, Dimethylsulfoxide-d₆) δ 8.79-8.77 (m, 2H), 8.59 (d, J = 2.4 Hz, 1H), 7.91-7.90 (m, 2H), 6.42 (d, J = 2.4 Hz, 1H), 5.50-4.00 (m, 4H), 3.93 (s, 3H), 3.79-3.77 (m, 4H), 2.91-2.82 (m, 2H), 2.70-2.59 (m, 1H), 2.21 (s, 3H), 2.08-1.97 (m, 2H), 1.93-1.84 (m, 2H), 1.76-1.62 (m, 2H). |
| 27 | 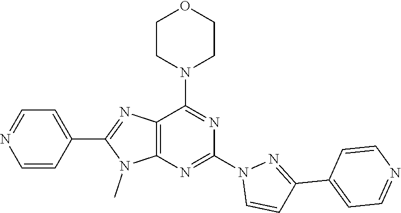 [SMILES: Cn1c(-c2ccncc2)nc2c(N3CCOCC3)nc(-n3ccc(-c4ccncc4)n3)nc21] | LCMS (ESI) m/z: 440.2 [M + H]⁺. | ¹H NMR (400 MHz, Chloroform- d) δ 8.81 (dd, J = 4.8 Hz, 2H), 8.68-8.69 (m, 3H), 7.78 (dd, J = 4.4 Hz, 2H), 7.76 (dd, J = 4.8 Hz, 2H), 6.88 (d, J = 2.4 Hz, 1H), 4.43 (bs, 4H), 4.06 (s, 3H), 3.91-3.93 (m, 4H). |
| 28 | 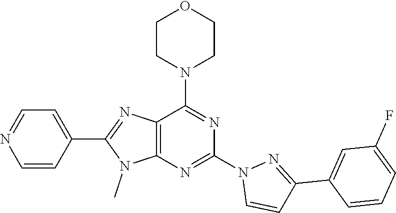 [SMILES: Cn1c(-c2ccncc2)nc2c(N3CCOCC3)nc(-n3ccc(-c4cccc(F)c4)n3)nc21] | LCMS (ESI) m/z: 457.1 [M + H]⁺. | ¹H NMR (400 MHz, Chloroform- d) δ 8.81 (dd, J = 4.4 Hz, 2H), 8.63 (d, J = 2.8 Hz, 1H), 7.72-7.76 (m, 4H), 7.39-7.41 (m, 1H), 7.04-7.05 (m, 1H), 6.78 (d, J = 2.4 Hz, 1H), 4.43 (bs, 4H), 4.05 (s, 3H), 3.90-3.93 (m, 4H). |
| 29 | 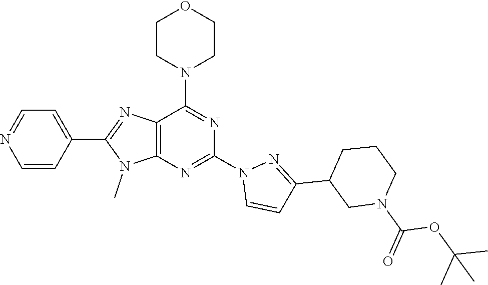 [SMILES: Cn1c(-c2ccncc2)nc2c(N3CCOCC3)nc(-n3ccc(C4CCCN(C(=O)OC(C)(C)C)C4)n3)nc21] | LCMS (ESI) m/z: 546 [M + H]⁺. | ¹H NMR (400 MHz, Dimethylsulfoxide-d₆) δ 8.83-8.74 (m, 2H), 8.62 (d, J = 2.5 Hz, 1H), 7.95-7.86 (m, 2H), 6.45 (d, J = 2.5 Hz, 1H), 3.94 (s, 9H), 3.78 (t, J = 4.8 Hz, 4H), 3.00-2.70 (m, 3H), 2.04 (d, J = 12.2 Hz, 1H), 1.75-1.62 (m, 2H), 1.50-1.45 (m, 1H), 1.42 (s, 9H). |

### General Procedure 3 (Compounds 30-33)

### Preparation of tert-butyl 3-{3-[9-methyl-6-(morpholin-4-yl)-8-(pyridin-4-yl)-9H-purin-2-yl]-1,2,4-oxadiazol-5-yl}piperidine-1-carboxylate

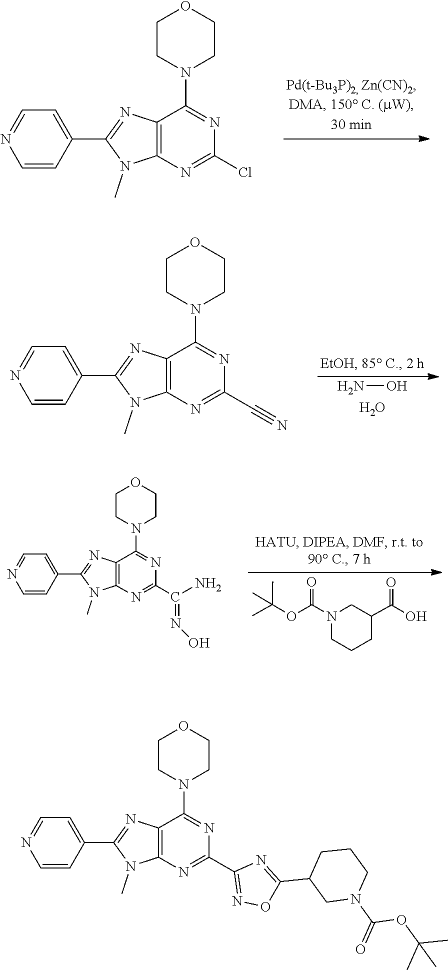

### Step 1: Preparation of 9-methyl-6-morpholino-8-(pyridin-4-yl)-9H-purine-2-carbonitrile

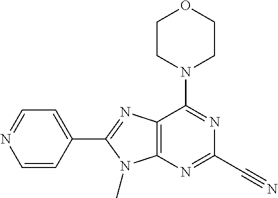 [SMILES: Cn1c(-c2ccncc2)nc2c(N3CCOCC3)nc(C#N)nc21]

A solution of 4-(2-chloro-9-methyl-8-(pyridin-4-yl)-9H-purin-6-yl)morpholine (500 mg, 1.5 mmol), bis(tri-tert-butylphosphine) palladium(0) (77.0 mg, 0.15 mmol), zinc cyanide (351 mg, 3.0 mmol) in N,N-dimethylacetamide (6 mL) was microwaved with stirring at 150° C. for 30 minutes. The product was indicated present via UPLC analysis. The reaction mixture was filtered over celite and washed with ethyl acetate (2×20 mL). The filtrate was concentrated under reduced pressure. Crude product was purified by HPLC (SunFire C18, 4.6*50 mm, 3.5 um column Xbridge C18 3.5 μm 4.6×50 mm column. The elution system used was a gradient of 5%-95% over 1.5 min at 2 mL/min and the solvent was acetonitrile/0.01% aqueous ammonium bicarbonate). Product 9-methyl-6-morpholino-8-(pyridin-4-yl)-9H-purine-2-carbonitrile (393 mg, 1.2 mmol, 82%) was afforded as a light yellow solid. NMR data unavailable; LCMS (ESI) m/z: 322.1 [M+H]⁺.

### Step 2: Preparation of (Z)-N′-hydroxy-9-methyl-6-morpholino-8-(pyridin-4-yl)-9H-purine-2-carboximidamide

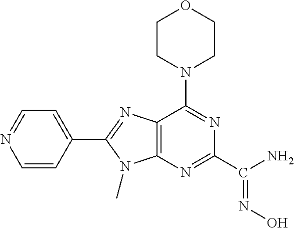 [SMILES: Cn1c(-c2ccncc2)nc2c(N3CCOCC3)nc(/C(N)=N/O)nc21]

A solution of 9-methyl-6-morpholino-8-(pyridin-4-yl)-9H-purine-2-carbonitrile (390 mg, 1.2 mmol) and 50% w/v hydroxylamine aqueous solution (241 mg, 3.6 mmol) in ethanol (15 mL) was stirred at 85° C. for 2 hours. The product was indicated present via UPLC analysis. Water (20 mL) and ethyl acetate (40 mL) were added to the reaction mixture and the layers were separated. The organic layer was separated, dried over sodium sulfate, filtered and concentrated under reduced pressure. Crude product (Z)-N′-hydroxy-9-methyl-6-morpholino-8-(pyridin-4-yl)-9H-purine-2-carboximidamide (353 mg, 1.0 mmol, 81%) was afforded as a light yellow solid and carried onto the next step without further purification. NMR data unavailable; LCMS (ESI) m/z: 355.1 [M+H]⁺.

### Step 3: Preparation of tert-butyl 3-{3-[9-methyl-6-(morpholin-4-yl)-8-(pyridin-4-yl)-9H-purin-2-yl]-1,2,4-oxadiazol-5-yl}piperidine-1-carboxylate

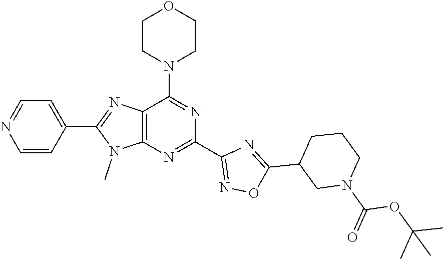 [SMILES: Cn1c(-c2ccncc2)nc2c(N3CCOCC3)nc(-c3noc(C4CCCN(C(=O)OC(C)(C)C)C4)n3)nc21]

To a solution of (Z)-N′-hydroxy-9-methyl-6-morpholino-8-(pyridin-4-yl)-9H-purine-2-carboximidamide (180 mg, 1.0 mmol), 1-(tert-butoxycarbonyl)piperidine-3-carboxylic acid (228 mg, 1.0 mmol), N,N-diisopropylethylamine (387 mg, 3.0 mmol) in N,N-dimethylformamide (5 mL) was added 1-[Bis(dimethylamino)methylene]-1H-1,2,3-triazolo[4,5-b]pyridinium 3-oxid hexafluorophosphate (569 mg, 1.5 mmol). The reaction mixture was stirred at room temperature for 1 hour, then heated to 90° C. and stirred for an additional 6 hours. The product was indicated present via UPLC analysis. Water (20 mL) and ethyl acetate (100 mL) were added to the reaction mixture. The organic layer was separated, washed with brine (100 mL), dried over sodium sulfate, filtered, and concentrated under reduced pressure. Crude product was purified by HPLC (SunFire C18, 4.6*50 mm, 3.5 um column Xbridge C18 3.5 μm 4.6×50 mm column. The elution system used was a gradient of 5%-95% over 1.5 min at 2 mL/min and the solvent was acetonitrile/0.01% aqueous ammonium bicarbonate) to offer tert-butyl 3-{3-[9-methyl-6-(morpholin-4-yl)-8-(pyridin-4-yl)-9H-purin-2-yl]-1,2,4-oxadiazol-5-yl}piperidine-1-carboxylate (200 mg, 0.37 mmol, 37%) as a white solid. ¹H NMR (400 MHz, Dimethylsulfoxide-d₆) δ 8.80 (d, J=6.0 Hz, 2H), 7.93 (dd, J=4.5, 1.6 Hz, 2H), 4.23 (m, 5H), 3.96 (s, 3H), 3.84-3.74 (m, 4H), 3.60 (s, 2H), 3.16 (s, 2H), 2.16 (s, 1H), 1.92 (s, 1H), 1.75 (s, 1H), 1.52 (dd, J=9.7, 3.6 Hz, 1H), 1.39 (s, 9H); LCMS (ESI) m/z: 355.1 [M+H]⁺.

### Compounds 30-33

| # | Structure | LCMS Data | ¹H NMR Data |
|---|---|---|---|
| 30 | 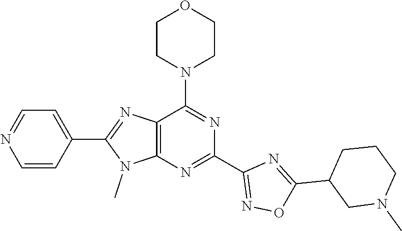 [SMILES: CN1CCCC(c2nc(-c3nc(N4CCOCC4)c4nc(-c5ccncc5)n(C)c4n3)no2)C1] | LCMS (ESI) m/z: 462.1 [M + H]⁺. | ¹H NMR (400 MHz, Dimethylsulfoxide-d₆) δ 8.81 (dd, J = 4.5, 1.6 Hz, 2H), 7.93 (dd, J = 4.5, 1.6 Hz, 2H), 4.33 (s, 4H), 3.96 (s, 3H), 3.88-3.68 (m, 4H), 3.02 (d, J = 9.5 Hz, 1H), 2.62 (s, 1H), 2.37 (s, 1H), 2.23 (s, 3H), 2.06 (d, J = 8.3 Hz, 2H), 1.82-1.71 (m, 1H), 1.64 (d, J = 8.1 Hz, 2H). |
| 31 | 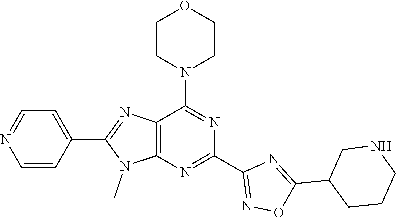 [SMILES: Cn1c(-c2ccncc2)nc2c(N3CCOCC3)nc(-c3noc(C4CCCNC4)n3)nc21] | LCMS (ESI) m/z: 448.2 [M + H]⁺. | ¹H NMR (400 MHz, Dimethylsulfoxide-d₆) δ 8.80 (dd, J = 4.5, 1.6 Hz, 2H), 7.93 (dd, J = 4.5, 1.6 Hz, 2H), 4.33 (s, 4H), 3.96 (s, 3H), 3.88-3.63 (m, 4H), 3.19 (m, 2H), 2.94-2.76 (m, 2H), 2.61-2.53 (m, 1H), 2.15 (d, J = 9.1 Hz, 1H), 1.81 (td, J = 14.4, 3.9 Hz, 1H), 1.73- 1.63 (m, 1H), 1.57-1.38 (m, 1H). |
| 32 | 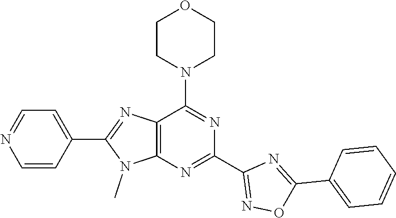 [SMILES: Cn1c(-c2ccncc2)nc2c(N3CCOCC3)nc(-c3noc(-c4ccccc4)n3)nc21] | LCMS (ESI) m/z: 441.1 [M + H]⁺. | ¹H NMR (400 MHz, Chloroform-d) δ 8.85-8.80 (m, 2H), 8.34-8.26 (m, 2H), 7.81-7.73 (m, 2H), 7.66-7.52 (m, 3H), 4.38 (s, 4H), 4.10 (s, 3H), 3.94-3.86 (m, 4H). |
| 33 | 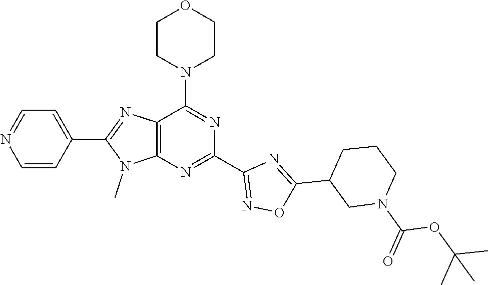 | LCMS (ESI) m/z: 355.1 [M + H]⁺. | ¹H NMR (400 MHz, Dimethylsulfoxide-d₆) δ 8.80 (d, J = 6.0 Hz, 2H), 7.93 (dd, J = 4.5, 1.6 Hz, 2H), 4.23 (m, 5H), 3.96 (s, 3H), 3.84-3.74 (m, 4H), 3.60 (s, 2H), 3.16 (s, 2H), 2.16 (s, 1H), 1.92 (s, 1H), 1.75 (s, 1H), 1.52 (dd, J = 9.7, 3.6 Hz, 1H), 1.39 (s, 9H). |

### General Procedure 4 (Compounds 34-40)

### Preparation of 3-{1-[8-(3,6-dihydro-2H-pyran-4-yl)-9-methyl-6-(morpholin-4-yl)-9H-purin-2-yl]-1H-pyrazol-3-yl}benzonitrile

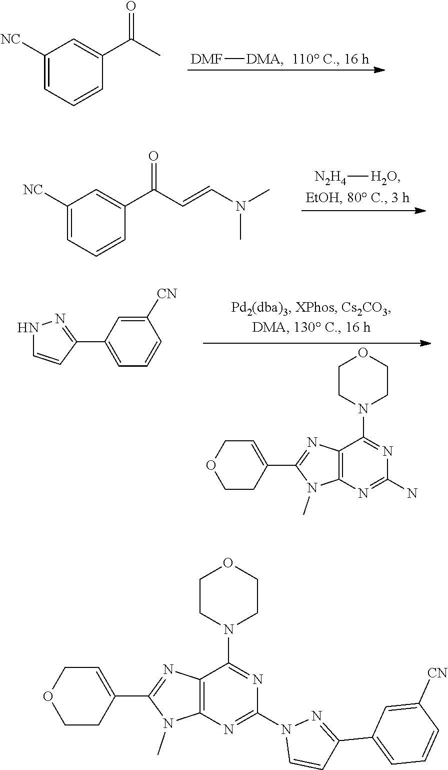

### Step 1: Preparation of (E)-3-(3-(Dimethylamino)acryloyl)benzonitrile

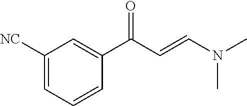

A mixture of 3-acetylbenzonitrile (1.50 g, 10 mmol) in N,N-dimethylformamide dimethyl acetal (10 mL) was stirred at 110° C. for 16 hours. The product was indicated present via UPLC analysis. The mixture concentrated under reduced pressure. Crude product (E)-3-(3-(Dimethylamino)acryloyl)benzonitrile (2.00 g, 10 mmol, 100%) was afforded as light yellow oil and was used directly in the next step without further purification. NMR data unavailable; LCMS (ESI) m/z: 201 [M+H]⁺.

### Step 2: Preparation of 3-(1H-pyrazol-3-yl)benzonitrile

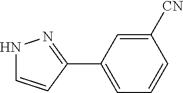 [SMILES: N#Cc1cccc(-c2cc[nH]n2)c1]

A mixture of (E)-3-(3-(Dimethylamino)acryloyl)benzonitrile (2.00 g, 10 mmol) and hydrazine monohydrate (1.50 g, 30 mmol) in ethanol (20 mL) was stirred at 80° C. for 3 hours. The product was indicated present via UPLC analysis. The mixture concentrated under reduced pressure, and the residue was purified by flash column chromatography through silica gel using a gradient of 0-30% ethyl acetate in petroleum ether. Product 3-(1H-pyrazol-3-yl)benzonitrile (1.40 g, 8.3 mmol, 83%) was afforded as a light yellow solid. NMR data unavailable; LCMS (ESI) m/z: 170 [M+H]⁺.

### Step 3: Preparation of 3-(1-(8-(3,6-Dihydro-2H-pyran-4-yl)-9-methyl-6-morpholino-9H-purin-2-yl)-1H-pyrazol-3-yl)benzonitrile

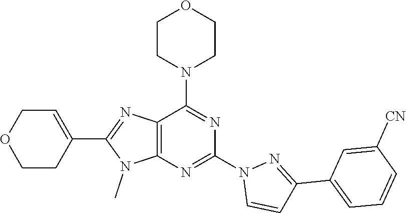 [SMILES: Cn1c(C2=CCOCC2)nc2c(N3CCOCC3)nc(-n3ccc(-c4cccc(C#N)c4)n3)nc21]

A mixture of 4-(2-chloro-8-(3,6-dihydro-2H-pyran-4-yl)-9-methyl-9H-purin-6-yl)morpholine (200 mg, 0.60 mmol), 3-(1H-pyrazol-3-yl)benzonitrile (128 mg, 0.76 mmol), tris(dibenzylideneacetone) dipalladium (56.0 mg, 0.060 mmol), 2-dicyclohexylphosphino-2′,4′,6′-triisopropylbiphenyl (56.0 mg, 0.12 mmol) and cesium carbonate (392 mg, 1.2 mmol) in N,N-dimethylacetamide (8 mL). The reaction was stirred at 130° C. under nitrogen for 16 hours. The product was indicated present via UPLC analysis. The mixture was cooled to room temperature, quenched with water (10 mL) and the organics were extracted with ethyl acetate (3×10 mL). The organic layers were pooled, washed with water and brine (20 mL), dried over sodium sulfate, filtered, and concentrated under reduced pressure. Crude product was purified by prep-HPLC (the crude samples were dissolved in methanol unless otherwise noted before purification. Boston C18 21*250 mm 10 μm column. The mobile phase was acetonitrile/0.01% aqueous ammonium bicarbonate). Product 3-(1-(8-(3,6-Dihydro-2H-pyran-4-yl)-9-methyl-6-morpholino-9H-purin-2-yl)-1H-pyrazol-3-yl)benzonitrile (80.0 mg, 0.17 mmol, 29%) was afforded as white solid. ¹H NMR (400 MHz, Dimethylsulfoxide-d₆) δ 8.78 (d, J=2.7 Hz, 1H), 8.38 (t, J=1.7 Hz, 1H), 8.32 (dt, J=8.0, 1.4 Hz, 1H), 7.85 (dt, J=7.7, 1.4 Hz, 1H), 7.70 (t, J=7.8 Hz, 1H), 7.19 (d, J=2.7 Hz, 1H), 6.57 (dd, J=6.3, 2.1 Hz, 1H), 4.88 (q, J=3.2 Hz, 1H), 4.47-4.15 (m, 4H), 4.10-4.03 (m, 1H), 3.98-3.91 (m, 1H), 3.83-3.71 (m, 7H), 3.31 (s, 1H), 2.22-2.12 (m, 2H); LCMS (ESI) m/z: 469 [M+H]⁺.

### Compounds 34-40

| # | Structure | LCMS Data | ¹H NMR Data |
|---|---|---|---|
| 34 | 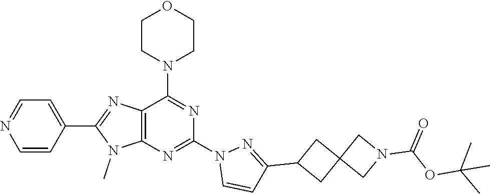 [SMILES: Cn1c(-c2ccncc2)nc2c(N3CCOCC3)nc(-n3ccc(C4CC5(C4)CN(C(=O)OC(C)(C)C)C5)n3)nc21] | LCMS (ESI) m/z: 558 [M + H]⁺. | ¹H NMR (400 MHz, Dimethylsulfoxide-d₆) δ 8.79-8.77 (m, 2H), 8.60 (d, J = 1.6 Hz, 1H), 7.91-7.90 (m, 2H), 6.44 (d, J = 1.6 Hz, 1H), 4.53-4.00 (m, 4H), 4.01- 3.91 (m, 5H), 3.85-3.75 (m, 6H), 3.49-3.41 (m, 1H), 2.59-2.54 (m, 2H), 2.39-2.34 (m, 2H). |
| 35 | 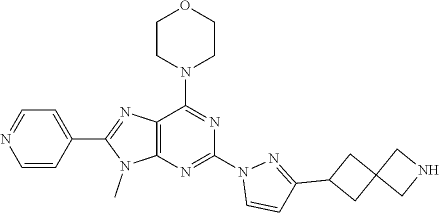 [SMILES: Cn1c(-c2ccncc2)nc2c(N3CCOCC3)nc(-n3ccc(C4CC5(CNC5)C4)n3)nc21] | LCMS (ESI) m/z: 458 [M + H]⁺. | ¹H NMR (400 MHz, Dimethylsulfoxide-d₆) δ 8.78-8.77 (m, 2H), 8.59 (d, J = 2.0 Hz, 1H), 7.91-7.90 (m, 2H), 6.44 (d, J = 2.0 Hz, 1H), 4.88-3.99 (m, 4H), 3.98- 3.88 (m, 4H), 3.83-3.72 (m, 2H), 3.54-3.44 (m, 3H), 2.38-2.24 (m, 2H). |
| 36 | 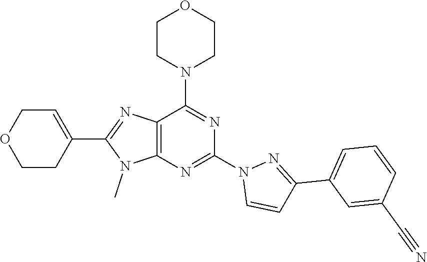 [SMILES: Cn1c(C2=CCOCC2)nc2c(N3CCOCC3)nc(-n3ccc(-c4cccc(C#N)c4)n3)nc21] | LCMS (ESI) m/z: 469 [M + H]⁺. | ¹H NMR (400 MHz, Dimethylsulfoxide-d₆) δ 8.78 (d, J = 2.7 Hz, 1H), 8.38 (t, J = 1.7 Hz, 1H), 8.32 (dt, J = 8.0, 1.4 Hz, 1H), 7.85 (dt, J = 7.7, 1.4 Hz, 1H), 7.70 (t, J = 7.8 Hz, 1H), 7.19 (d, J = 2.7 Hz, 1H), 6.57 (dd, J = 6.3, 2.1 Hz, 1H), 4.88 (q, J = 3.2 Hz, 1H), 4.47-4.15 (m, 4H), 4.10-4.03 (m, 1H), 3.98-3.91 (m, 1H), 3.83-3.71 (m, 7H), 3.31 (s, 1H), 2.22-2.12 (m, 2H). |
| 37 | 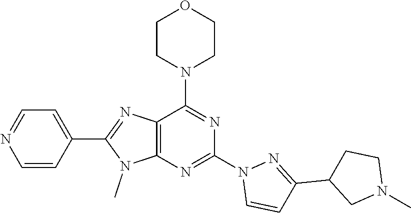 [SMILES: CN1CCC(c2ccn(-c3nc(N4CCOCC4)c4nc(-c5ccncc5)n(C)c4n3)n2)C1] | LCMS (ESI) m/z: 446.2 [M + H]⁺. | ¹H NMR (500 MHz, Dimethylsulfoxide-d₆) δ 8.78 (d, J = 6.0 Hz, 2H), 8.61 (d, J = 2.6 Hz, 1H), 8.25 (s, 1H), 7.90 (dd, J = 4.5, 1.6 Hz, 2H), 6.45 (d, J = 2.6 Hz, 1H), 4.26 (s, 4H), 3.93 (s, 3H), 3.80- 3.75 (m, 4H), 3.52 (dt, J = 15.2, 7.5 Hz, 1H), 3.06 (t, J = 8.7 Hz, 1H), 2.79 (dd, J = 15.1, 8.3 Hz, 1H), 2.76- 2.65 (m, 2H), 2.42 (s, 3H), 2.32- 2.22 (m, 1H), 2.03-1.89 (m, 1H). |
| 38 | 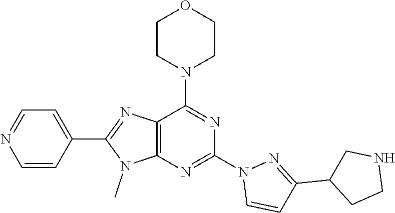 [SMILES: Cn1c(-c2ccncc2)nc2c(N3CCOCC3)nc(-n3ccc(C4CCNC4)n3)nc21] | LCMS (ESI) m/z: 432.3 [M + H]⁺. | ¹H NMR (400 MHz, Chloroform-d) δ 8.79 (dd, J = 4.5, 1.6 Hz, 2H), 8.50 (d, J = 2.6 Hz, 1H), 7.74 (dd, J = 4.5, 1.6 Hz, 2H), 6.31 (d, J = 2.6 Hz, 1H), 4.42 (s, 4H), 4.02 (s, 3H), 3.95- 3.86 (m, 4H), 3.52 (dd, J = 15.8, 7.6 Hz, 1H), 3.38 (dd, J = 11.0, 7.8 Hz, 1H), 3.17 (m, 1H), 3.06 (m, 2H), 2.38- 2.19 (m, 1H), 2.00 (m, 1H). |
| 39 | 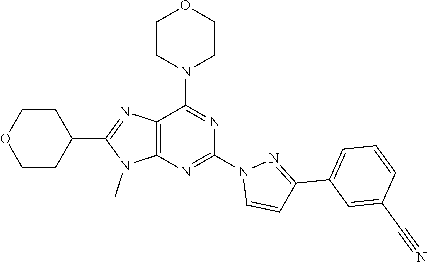 [SMILES: Cn1c(C2CCOCC2)nc2c(N3CCOCC3)nc(-n3ccc(-c4cccc(C#N)c4)n3)nc21] | LCMS (ESI) m/z: 471 [M + H]⁺. | ¹H NMR (400 MHz, Dimethylsulfoxide-d₆) δ 8.78 (d, J = 2.6 Hz, 1H), 8.38 (t, J = 1.7 Hz, 1H), 8.31 (dt, J = 8.0, 1.5 Hz, 1H), 7.85 (dt, J = 7.7, 1.4 Hz, 1H), 7.69 (t, J = 7.8 Hz, 1H), 7.19 (d, J = 2.7 Hz, 1H), 4.30 (s, 3H), 3.99-3.94 (m, 2H), 3.83-3.74 (m, 7H), 3.51 (td, J = 11.1, 3.4 Hz, 2H), 3.30 (s, 1H), 1.92- 1.78 (m, 4H). |
| 40 | 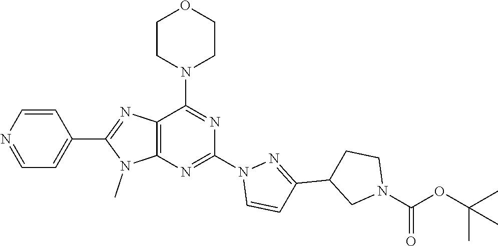 [SMILES: Cn1c(-c2ccncc2)nc2c(N3CCOCC3)nc(-n3ccc(C4CCN(C(=O)OC(C)(C)C)C4)n3)nc21] | LCMS (ESI) m/z: 532.3 [M + H]⁺. | ¹H NMR (400 MHz, Chloroform-d) δ 8.80 (dd, J = 4.5, 1.6 Hz, 2H), 8.52 (d, J = 2.5 Hz, 1H), 7.74 (dd, J = 4.5, 1.6 Hz, 2H), 6.32 (d, J = 2.7 Hz, 1H), 4.41 (s, 4H), 4.02 (s, 3H), 3.96- 3.71 (m, 5H), 3.65 (m, 2H), 3.52- 3.26 (m, 2H), 2.34 (m, 1H), 2.11 (m, 1H), 1.48 (s, 8H). |

### General Procedure 5 (Compounds 41-49)

### Preparation of 9-methyl-6-(morpholin-4-yl)-2-(1-phenyl-1H-pyrazol-3-yl)-8-(pyridin-4-yl)-9H-purine

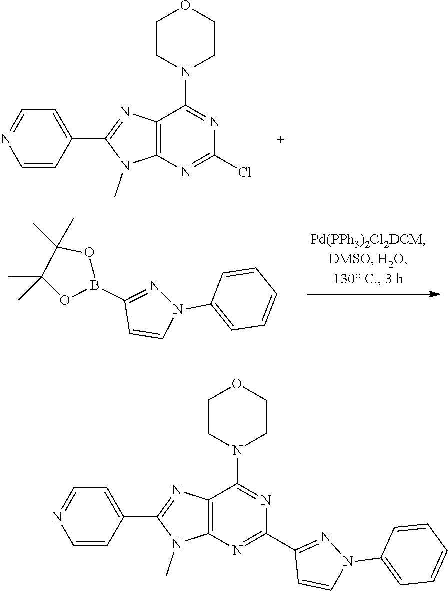

### Step 1: Preparation of 9-methyl-6-(morpholin-4-yl)-2-(1-phenyl-1H-pyrazol-3-yl)-8-(pyridin-4-yl)-9H-purine

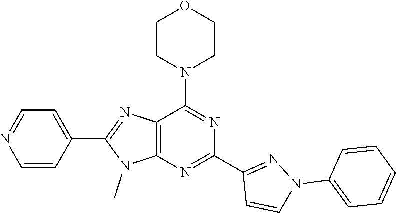 [SMILES: Cn1c(-c2ccncc2)nc2c(N3CCOCC3)nc(-c3ccn(-c4ccccc4)n3)nc21]

A mixture of 4-(2-chloro-9-methyl-8-(pyridin-4-yl)-9H-purin-6-yl)morpholine (100 mg, 0.30 mmol), (4,5,5-trimethyl-2-(1-phenyl-1H-pyrazol-3-yl)-1,3,2-dioxaborolan-4-yl)methylium (105 mg, 0.39 mmol), 1,1′-bis(diphenylphosphino)ferrocene palladium(II)dichloride dichloromethane complex (49.0 mg, 0.060 mmol) and cesium carbonate (293 mg, 0.90 mmol) in water (2 mL) and dimethylsulfoxide (8 mL) was stirred at 130° C. for 3 hours under argon. The product was indicated present via UPLC analysis. The mixture was filtered over celite and washed with ethyl acetate (50 mL). The filtrate was further diluted with water (50 mL) and the layers were separated. The organic layer was washed with brine (20 mL), dried over sodium sulfate, filtered and concentrated under reduced pressure. Crude product was purified by prep-HPLC (the crude samples were dissolved in N,N-dimethylformamide unless otherwise noted before purified. Boston pHlex ODS 10 um 21.2×250 mm 120A. The mobile phase was acetonitrile/0.1% ammonium bicarbonate). Product 9-methyl-6-(morpholin-4-yl)-2-(1-phenyl-1H-pyrazol-3-yl)-8-(pyridin-4-yl)-9H-purine (64.5 mg, 0.15 mmol, 30%) was afforded as a yellow solid. ¹H NMR (400 MHz, Chloroform-d) δ 8.83-8.76 (m, 2H), 8.01 (d, J=2.5 Hz, 1H), 7.89-7.82 (m, 2H), 7.79-7.74 (m, 2H), 7.53-7.44 (m, 2H), 7.35-7.29 (m, 1H), 7.23 (d, J=2.5 Hz, 1H), 4.47 (s, 4H), 4.07 (s, 3H), 3.96-3.84 (m, 4H); LCMS (ESI) m/z: 439.2 [M+H]⁺.

### Compounds 41-49

| # | Structure | LCMS Data | ¹H NMR Data |
|---|---|---|---|
| 41 | 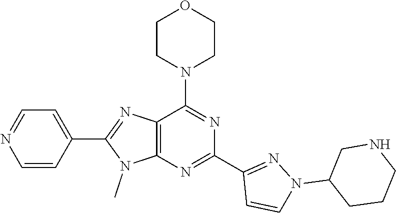 [SMILES: Cn1c(-c2ccncc2)nc2c(N3CCOCC3)nc(-c3ccn(C4CCCNC4)n3)nc21] | LCMS (ESI) m/z: 446.3 [M + H]⁺. | ¹H NMR (400 MHz, Dimethylsulfoxide-d₆) δ 8.80 (dd, J = 4.5, 1.6 Hz, 2H), 7.93 (dd, J = 4.5, 1.6 Hz, 2H), 7.53 (d, J = 1.8 Hz, 1H), 6.93 (d, J = 1.8 Hz, 1H), 5.58 (s, 1H), 4.32 (s, 4H), 3.96 (s, 3H), 3.86-3.60 (m, 4H), 3.22 (m, 1H), 3.09-2.89 (m, 2H), 2.56 (m, 1H), 2.12 (m, 1H), 2.00 (m, 1H), 1.78 (m, 1H), 1.56 (m, 1H). |
| 42 | 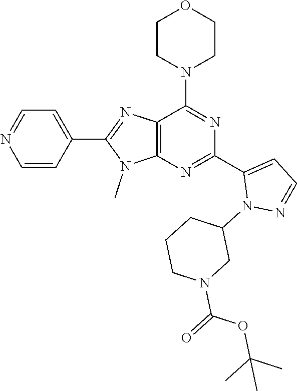 [SMILES: Cn1c(-c2ccncc2)nc2c(N3CCOCC3)nc(-c3ccnn3C3CCCN(C(=O)OC(C)(C)C)C3)nc21] | LCMS (ESI) m/z: 546.3 [M + H]⁺. | ¹H NMR (400 MHz, Dimethylsulfoxide-d₆) δ 8.78 (d, J = 6.0 Hz, 2H), 7.92 (d, J = 6.1 Hz, 2H), 7.88 (d, J = 2.3 Hz, 1H), 6.93 (d, J = 2.3 Hz, 1H), 4.24 (m, 7H), 3.96 (s, 3H), 3.86 (d, J = 13.1 Hz, 1H), 3.78 (d, J = 4.5 Hz, 4H), 2.92 (m, 1H), 2.14 (m, 1H), 2.06 (m, 1H), 1.81-1.74 (m, 1H), 1.53 (m, 1H), 1.42 (s, 9H). |
| 43 | 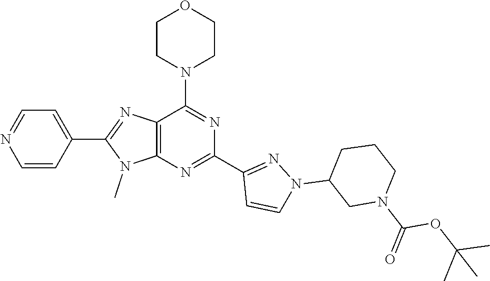 [SMILES: Cn1c(-c2ccncc2)nc2c(N3CCOCC3)nc(-c3ccn(C4CCCN(C(=O)OC(C)(C)C)C4)n3)nc21] | LCMS (ESI) m/z: 546.3 [M + H]⁺. | ¹H NMR (400 MHz, Dimethylsulfoxide-d₆) δ 8.79 (d, J = 5.8 Hz, 2H), 7.91 (d, J = 6.1 Hz, 2H), 7.56 (d, J = 1.8 Hz, 1H), 6.95 (d, J = 1.8 Hz, 1H), 5.62 (s, 1H), 4.28 (s, 4H), 3.97 (d, J = 2.8 Hz, 1H), 3.93 (s, 3H), 3.77 (t, J = 4.6 Hz, 4H), 2.86-2.77 (m, 1H), 2.12 (s, 2H), 1.85 (m, 1H), 1.40 (m, 11H). |
| 44 | 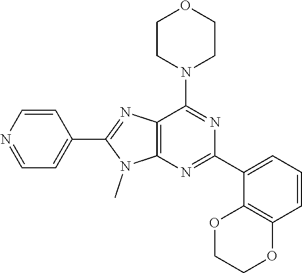 [SMILES: Cn1c(-c2ccncc2)nc2c(N3CCOCC3)nc(-c3cccc4c3OCCO4)nc21] | LCMS (ESI) m/z: 431.3 [M + H]⁺. | ¹H NMR (400 MHz, Chloroform-d) δ 8.86-8.75 (m, 2H), 7.79-7.72 (m, 2H), 7.41-7.35 (m, 1H), 7.00- 6.86 (m, 2H), 4.65-4.10 (m, 8H), 3.99 (s, 3H), 3.93-3.78 (m, 4H). |
| 45 | 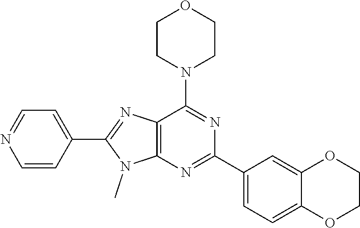 [SMILES: Cn1c(-c2ccncc2)nc2c(N3CCOCC3)nc(-c3ccc4c(c3)OCCO4)nc21] | LCMS (ESI) m/z: 431.1 [M + H]⁺. | ¹H NMR (500 MHz, Chloroform-d) δ 8.81-8.76 (m, 2H), 8.06-7.98 (m, 2H), 7.79-7.72 (m, 2H), 6.94 (d, J = 8.4 Hz, 1H), 4.57-4.21 (m, 8H), 4.00 (s, 3H), 3.93-3.84 (m, 4H). |
| 46 | 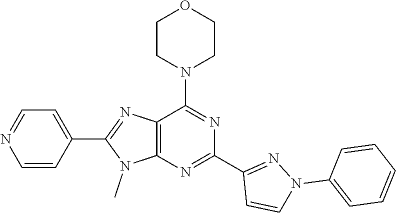 [SMILES: Cn1c(-c2ccncc2)nc2c(N3CCOCC3)nc(-c3ccn(-c4ccccc4)n3)nc21] | LCMS (ESI) m/z: 439.2 [M + H]⁺. | ¹H NMR (400 MHz, Chloroform-d) δ 8.83-8.76 (m, 2H), 8.01 (d, J = 2.5 Hz, 1H), 7.89-7.82 (m, 2H), 7.79-7.74 (m, 2H), 7.53-7.44 (m, 2H), 7.35-7.29 (m, 1H), 7.23 (d, J = 2.5 Hz, 1H), 4.47 (s, 4H), 4.07 (s, 3H), 3.96-3.84 (m, 4H) |
| 47 | 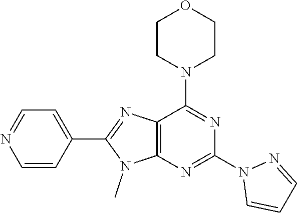 [SMILES: Cn1c(-c2ccncc2)nc2c(N3CCOCC3)nc(-n3cccn3)nc21] | LCMS (ESI) m/z: 363.1 [M + H]⁺. | 1H NMR (400 MHz, Dimethylsulfoxide-d6) 0 8.78 (dd, J = 4.5, 1.5 Hz, 2H), 8.71 (d, J = 2.2 Hz, 1H), 7.91 (dd, J = 4.5, 1.6 Hz, 2H), 7.78 (d, J = 0.8 Hz, 1H), 6.54 (dd, J = 2.5, 1.6 Hz, 1H), 4.30 (s, 4H), 3.94 (s, 3H), 3.84-3.71 (m, 4H). |
| 48 | 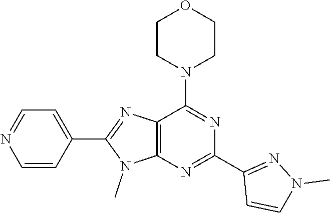 [SMILES: Cn1ccc(-c2nc(N3CCOCC3)c3nc(-c4ccncc4)n(C)c3n2)n1] | LCMS (ESI) m/z: 377.1 [M + H]⁺. | 1H NMR (400 MHz, Dimethylsulfoxide-d6) 0 8.79 (s, 2H), 7.91 (d, J = 4.8 Hz, 2H), 7.75 (s, 1H), 6.89 (s, 1H), 4.32 (s, 4H), 3.94 (d, J = 3.8 Hz, 6H), 3.77 (s, 4H). |
| 49 | 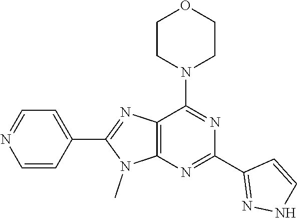 [SMILES: Cn1c(-c2ccncc2)nc2c(N3CCOCC3)nc(-c3cc[nH]n3)nc21] | LCMS (ESI) m/z: 363.1 [M + H]⁺. | 1H NMR (400 MHz, Dimethylsulfoxide-d6) o 13.49 (s, 1H), 8.78 (d, J = 6.0 Hz, 2H), 7.92 (dd, J = 4.6, 1.5 Hz, 2H), 7.59 (s, 1H), 6.90 (d, J = 1.7 Hz, 1H), 4.37 (s, 4H), 3.97 (s, 3H), 3.82-3.69 (m, 4H). |

### General Procedure 6 (Compound 50)

### Preparation of 3-cyclohexyl-1-[9-methyl-6-(morpholin-4-yl)-8-(pyridin-4-yl)-9H-purin-2-yl]-4,5-dihydro-1H-1,2,4-triazol-5-one

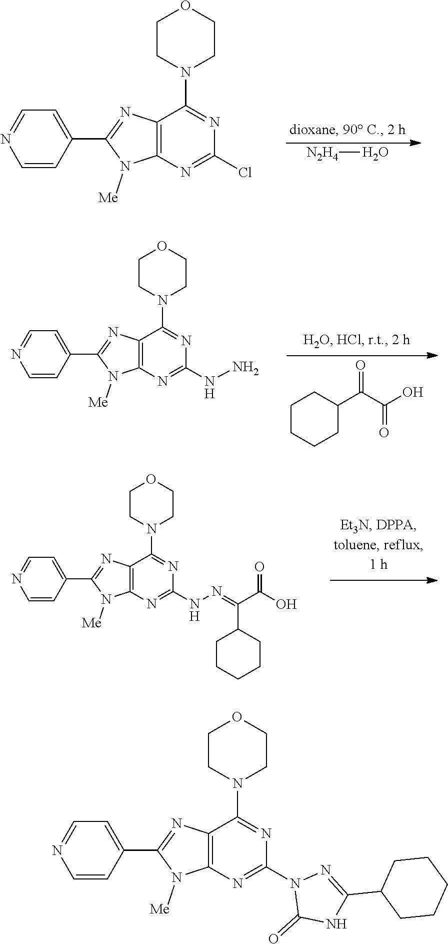

### Step 1: Preparation of 4-(2-hydrazinyl-9-methyl-8-(pyridin-4-yl)-9H-purin-6-yl)morpholine

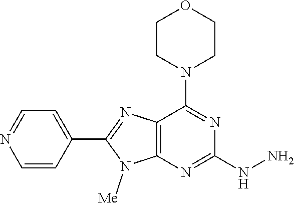 [SMILES: Cn1c(-c2ccncc2)nc2c(N3CCOCC3)nc(NN)nc21]

A solution of 4-(2-chloro-9-methyl-8-(pyridin-4-yl)-9H-purin-6-yl)morpholine (300 mg, 0.91 mmol) in dioxane (12 mL) was added hydrazine hydrate (3 mL). The reaction was heated to 90° C. and stirred for 2 hours. The product was indicated present via UPLC analysis. The reaction was concentrated under reduced pressure. Crude product was purified via flash column chromatography through silica gel using a gradient of 0-5% methanol in dichloromethane. Product 4-(2-hydrazinyl-9-methyl-8-(pyridin-4-yl)-9H-purin-6-yl)morpholine (283 mg, 0.87 mmol, 95%) was afforded as a white solid. NMR data unavailable; LCMS (ESI) m/z: 327.1 [M+H]⁺.

### Step 2: Preparation of (E)-2-cyclohexyl-2-(2-(9-methyl-6-morpholino-8-(pyridin-4-yl)-9H-purin-2-yl)hydrazono)acetic acid

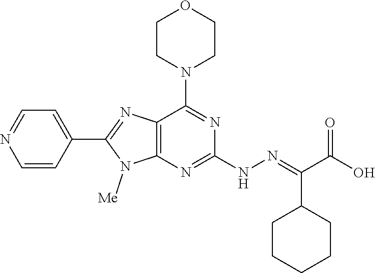 [SMILES: Cn1c(-c2ccncc2)nc2c(N3CCOCC3)nc(N/N=C(/C(=O)O)C3CCCCC3)nc21]

To a mixture of 4-(2-hydrazinyl-9-methyl-8-(pyridin-4-yl)-9H-purin-6-yl)morpholine (283 mg, 0.87 mmol) and 2-cyclohexyl-2-oxoacetic acid (271 mg, 1.7 mmol) in water (10 mL) was added concentrated hydrochloric acid (0.5 mL). The mixture was stirred at room temperature for 2 hours. The product was indicated present via UPLC analysis. The reaction was filtered and the precipitate was washed by water (20 mL) and collected. Crude product (E)-2-cyclohexyl-2-(2-(9-methyl-6-morpholino-8-(pyridin-4-yl)-9H-purin-2-yl)hydrazono)acetic acid (379 mg, 0.82 mmol, 94%) was afforded as a white solid and carried onto next step without further purification. NMR data unavailable; LCMS (ESI) m/z: 465.2 [M+H]⁺.

### Step 3: Preparation of 3-cyclohexyl-1-[9-methyl-6-(morpholin-4-yl)-8-(pyridin-4-yl)-9H-purin-2-yl]-4,5-dihydro-1H-1,2,4-triazol-5-one

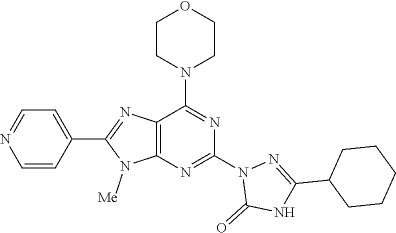 [SMILES: Cn1c(-c2ccncc2)nc2c(N3CCOCC3)nc(-n3nc(C4CCCCC4)[nH]c3=O)nc21]

A solution of (E)-2-cyclohexyl-2-(2-(9-methyl-6-morpholino-8-(pyridin-4-yl)-9H-purin-2-yl)hydrazono)acetic acid (379 mg, 0.82 mmol), triethylamine (165 mg, 1.6 mmol) in toluene (5 mL) was added diphenyl phosphorylazide (449 mg, 1.6 mmol). The reaction was refluxed with stirring for 2 hours. The product was indicate present via UPLC analysis. The reaction was concentrated under reduced pressure. Crude product was purified by prep-HPLC (SunFire C18, 4.6*50 mm, 3.5 um column Xbridge C18 3.5 μm 4.6×50 mm column. The elution system used was a gradient of 5%-95% over 1.5 min at 2 mL/min and the solvent was acetonitrile/0.01% aqueous ammonium bicarbonate). Product 3-cyclohexyl-1-[9-methyl-6-(morpholin-4-yl)-8-(pyridin-4-yl)-9H-purin-2-yl]-4,5-dihydro-1H-1,2,4-triazol-5-one (92.5 mg, 0.20 mmol, 25%) was afforded as a white solid. ¹H NMR (500 MHz, Chloroform-d) δ 11.97 (s, 1H), 8.80 (dd, J=4.5, 1.6 Hz, 2H), 7.75 (dd, J=4.5, 1.6 Hz, 2H), 4.58 (s, 4H), 4.00 (s, 3H), 3.88 (dd, J=11.7, 7.0 Hz, 4H), 2.75 (tt, J=11.9, 3.4 Hz, 1H), 2.13 (d, J=11.9 Hz, 2H), 1.91-1.82 (m, 2H), 1.78 (d, J=12.7 Hz, 1H), 1.59 (qd, J=12.6, 3.2 Hz, 2H), 1.39 (dt, J=12.8, 7.9 Hz, 2H), 1.28 (m, 1H); LCMS (ESI) m/z: 462.3 [M+H]⁺.

### Compound 50

| # | Structure | LCMS Data | ¹H NMR Data |
|---|---|---|---|
| 50 | 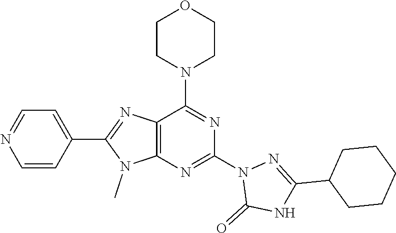 [SMILES: Cn1c(-c2ccncc2)nc2c(N3CCOCC3)nc(-n3nc(C4CCCCC4)[nH]c3=O)nc21] | LCMS (ESI) m/z: 462.3 [M + H]⁺. | ¹H NMR (500 MHz, Chloroform-d) δ 11.97 (s, 1H), 8.80 (dd, J = 4.5, 1.6 Hz, 2H), 7.75 (dd, J = 4.5, 1.6 Hz, 2H), 4.58 (s, 4H), 4.00 (s, 3H), 3.88 (dd, J = 11.7, 7.0 Hz, 4H), 2.75 (tt, J = 11.9, 3.4 Hz, 1H), 2.13 (d, J = 11.9 Hz, 2H), 1.91-1.82 (m, 2H), 1.78 (d, J = 12.7 Hz, 1H), 1.59 (qd, J = 12.6, 3.2 Hz, 2H), 1.39 (dt, J = 12.8, 7.9 Hz, 2H), 1.28 (m, 1H). |

### Compound 51: Synthesis of (E)-4-(2-(2-(3-methylbenzylidene)hydrazinyl)-8-(pyridin-4-yl)-9H-purin-6-yl)morpholine

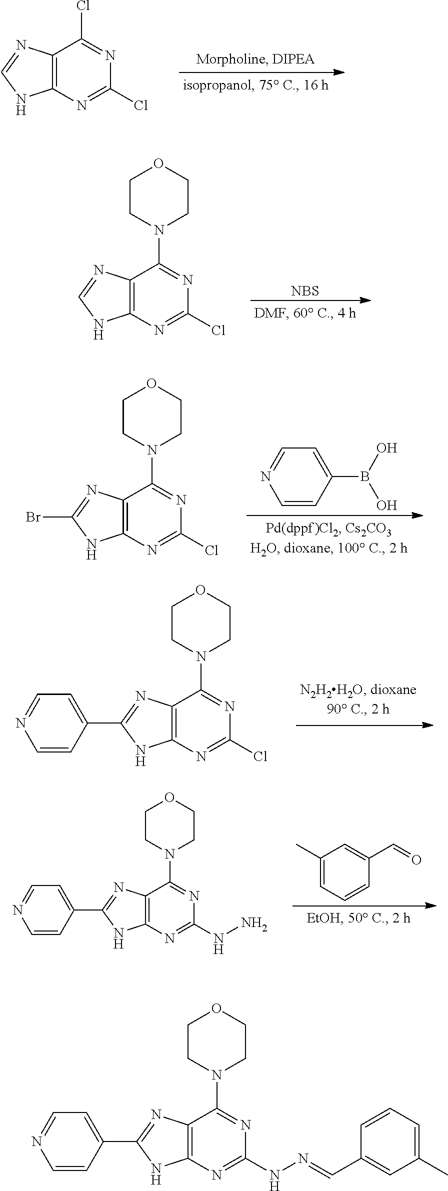

### Step 1: Preparation of 4-(2-chloro-9H-purin-6-yl)morpholine

A solution of 2,6-dichloro-9H-purine (5 g, 26 mmol), morpholine (2 g, 26 mmol) and N,N-diisopropylethylamine (6.7 g, 52 mmol) in isopropanol (200 mL) was stirred at 75° C. for 16 h. The mixture was filtered to obtain 4-(2-chloro-9H-purin-6-yl)morpholine (5 g, 80%) as white solid. ¹H NMR (400 MHz, DMSO-d₆) δ 13.24 (s, 1H), 8.16 (s, 1H), 4.19 (s, 4H), 3.83-3.59 (m, 4H).

### Step 2: Preparation of 4-(8-bromo-2-chloro-9H-purin-6-yl)morpholine

A solution of 4-(2-chloro-9H-purin-6-yl)morpholine (4.8 g, 20 mmol) and N-bromosuccinimide (5.24 g, 30 mmol) in DMF (25 mL) was stirred at 60° C. for 4 h. The mixture was cooled to 20° C. and filtered. The solid was washed with ethyl acetate to obtain 4-(8-bromo-2-chloro-9H-purin-6-yl)morpholine (0.8 g, 13%) as white solid. LCMS (ESI) m/z: 318.0 [M+H]⁺.

### Step 3: Preparation of 4-(2-chloro-8-(pyridin-4-yl)-9H-purin-6-yl)morpholine

A solution of 4-(8-bromo-2-chloro-9H-purin-6-yl)morpholine (0.1 g, 0.31 mmol), pyridin-4-ylboronic acid (0.19 g, 1.57 mmol), [1,I-Bis(diphenylphosphino)ferrocene]dichloropalladium(11) (0.02 g, 0.03 mmol) and cesium carbonate (0.2 g, 0.62 mmol) in water (0.5 mL) and dioxane (2 mL) was stirred at 100° C. for 2 h under argon. The mixture was diluted with ethyl acetate (10 mL) and washed with water (10 mL). The organic layer was concentrated and purified by Prep-HPLC (Boston C18 21*250 mm 10 μm column. The mobile phase was acetonitrile/0.01% aqueous trifluoroacetic acid.) to give the desired product 4-(2-chloro-8-(pyridin-4-yl)-9H-purin-6-yl)morpholine (0.03 g, 31%) as white solid. LCMS (ESI) m/z: 317.1 [M+H]⁺.

### Step 4: Preparation of 4-(2-hydrazinyl-8-(pyridin-4-yl)-9H-purin-6-yl)morpholine

A solution of 4-(2-chloro-8-(pyridin-4-yl)-9H-purin-6-yl)morpholine (150 mg, 0.47 mmol) and hydrazine hydrate (118 mg, 2.4 mmol) in dioxane (5.0 mL) was stirred at 90° C. under nitrogen for 2 h. The reaction was concentrated and filtered to give the desired product 4-(2-hydrazinyl-8-(pyridin-4-yl)-9H-purin-6-yl)morpholine (100 mg, 68%) as brown solid. LCMS (ESI) m/z: 313.2 [M+H]⁺.

### Step 5: Preparation of (E)-4-(2-(2-(3-methylbenzylidene)hydrazinyl)-8-(pyridin-4-yl)-9H-purin-6-yl)morpholine

To a solution of 4-(2-hydrazinyl-8-(pyridin-4-yl)-9H-purin-6-yl)morpholine (100 mg, 0.32 mmol) and 3-methylbenzaldehyde (77 mg, 0.64 mmol) in ethanol (5.0 mL) was added acetic acid (19 mg, 0.32 mmol) and the resultant mixture was stirred at 20° C. under nitrogen for 2 h. The mixture was then concentrated and the residue was purified by Prep-HPLC (Boston C18 21*250 mm 10 μm column. The mobile phase was acetonitrile/0.01% aqueous trifluoroacetic acid.) to obtain (E)-4-(2-(2-(3-methylbenzylidene)hydrazinyl)-8-(pyridin-4-yl)-9H-purin-6-yl)morpholine (46.6 mg, 35%) as a yellow solid.

¹H NMR (400 MHz, DMSO) δ 13.63 (s, 1H), 10.76 (s, 1H), 8.68 (d, J=5.4 Hz, 2H), 8.07 (s, 1H), 7.97 (d, J=5.7 Hz, 2H), 7.56-7.39 (m, 2H), 7.32-7.28 (m, 1H), 7.15 (d, J=7.3 Hz, 1H), 4.29-4.24 (m, 4H), 3.78-3.75 (m, 4H), 2.35 (s, 3H); LCMS (ESI) m/z: 415.2 [M+H]⁺.

### Compound 51: Synthesis of (E)-4-(9-methyl-2-(2-(3-methylbenzylidene)hydrazineyl)-8-(pyridin-4-yl)-9H-purin-6-yl)morpholine

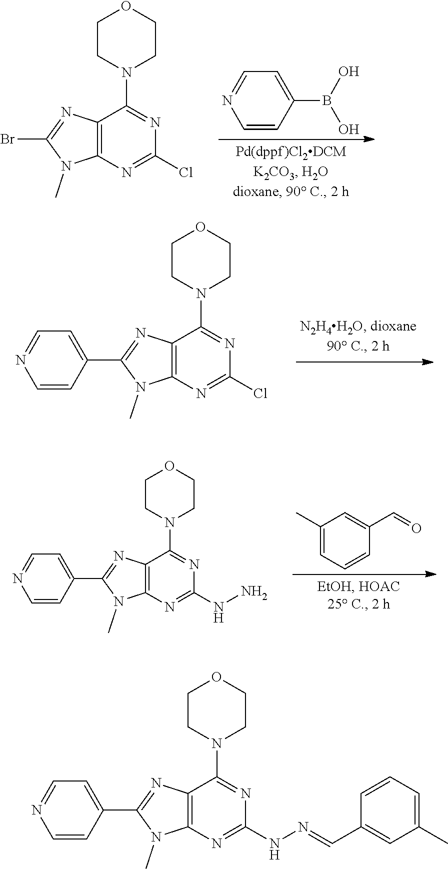

### Step 1: Synthesis of 4-(2-chloro-9-methyl-8-(pyridin-4-yl)-9H-purin-6-yl)morpholine

A mixture of 4-(8-bromo-2-chloro-9-methyl-9H-purin-6-yl)morpholine (4 g, 12 mmol), pyridin-4-ylboronic acid (1.7 g, 14.4 mmol), 1,1′-bis(diphenylphosphino) ferrocene-palladium(II) dichloride (0.45 g, 0.61 mmol) and potassium carbonate (5 g, 36 mmol) in 1,4-dioxane (60 mL) with H₂O (6 mL) was stirred at 90° C. under argon atmosphere for 2 h. The mixture was then concentrated and the residue was purified by silica gel column (petroleum ether:acetic ester=4:1) to afford 4-(2-chloro-9-methyl-8-(pyridin-4-yl)-9H-purin-6-yl)morpholine as white solid. (2.7 g, 66%). LCMS (ESI) m/z: 330.9 [M]+.

### Step 2: Synthesis of 4-(2-hydrazineyl-9-methyl-8-(pyridin-4-yl)-9H-purin-6-yl)morpholine

A mixture of 4-(2-chloro-9-methyl-8-(pyridin-4-yl)-9H-purin-6-yl)morpholine (693 mg, 2.1 mmol) and hydrazine hydrate (2.5 mL) in dioxane (12 mL) was stirred at 100° C. for 2 h. The reaction mixture was concentrated to give 4-(2-hydrazineyl-9-methyl-8-(pyridin-4-yl)-9H-purin-6-yl)morpholine (519.2 mg, 75%). LCMS (ESI) m/z: 327.1 [M+H]+.

### Step 3: Synthesis of (E)-4-(9-methyl-2-(2-(3-methylbenzylidene)hydrazineyl)-8-(pyridin-4-yl)-9H-purin-6-yl)morpholine

A mixture of 4-(2-hydrazineyl-9-methyl-8-(pyridin-4-yl)-9H-purin-6-yl)morpholine (456 mg, 1.4 mmol), 3-methylbenzaldehyde (335 mg, 2.8 mmol) and acetic acid (840 mg, 1.4 mmol) in ethanol (10 mL) was stirred at room temperature under nitrogen atmosphere 2 h. The mixture was filtered and the crude product thus obtained was purified by prep-HPLC (Column Xbridge 21.2*250 mm C18, 10 um, Mobile Phase A: water (10 mmol/L ammonium bicarbonate) B: acetonitrile) to afford 1 (E)-4-(9-methyl-2-(2-(3-methylbenzylidene)hydrazineyl)-8-(pyridin-4-yl)-9H-purin-6-yl)morpholine as yellow solid (387.8 mg, 65%). 1H NMR (400 MHz, DMSO-d₆) δ 10.84 (s, 1H), 8.74 (dd, J=4.5, 1.6 Hz, 2H), 8.07 (s, 1H), 7.87 (dd, J=4.5, 1.6 Hz, 2H), 7.47 (d, J=9.1 Hz, 2H), 7.30 (t, J=7.5 Hz, 1H), 7.15 (d, J=7.5 Hz, 1H), 4.27 (bs, 4H), 3.87 (s, 3H), 3.79-3.73 (m, 4H), 2.35 (s, 3H); LCMS (ESI) m/z: 429.0 [M+H]+.

### Compounds 80, 84, and 86: Synthesis of tert-butyl 3-((1-(9-methyl-6-morpholino-8-(pyridin-4-yl)-9H-purin-2-yl)-1H-pyrazol-3-yl)methyl)azetidine-1-carboxylate (Compound 84), 4-(2-(3-(azetidin-3-ylmethyl)-1H-pyrazol-1-yl)-9-methyl-8-(pyridin-4-yl)-9H-purin-6-yl)morpholine (Compound 80) and 4-(9-methyl-2-(34(1-methylazetidin-3-yl)methyl)-1H-pyrazol-1-yl)-8-(pyridin-4-yl)-9H-purin-6-yl)morpholine (Compound 86)

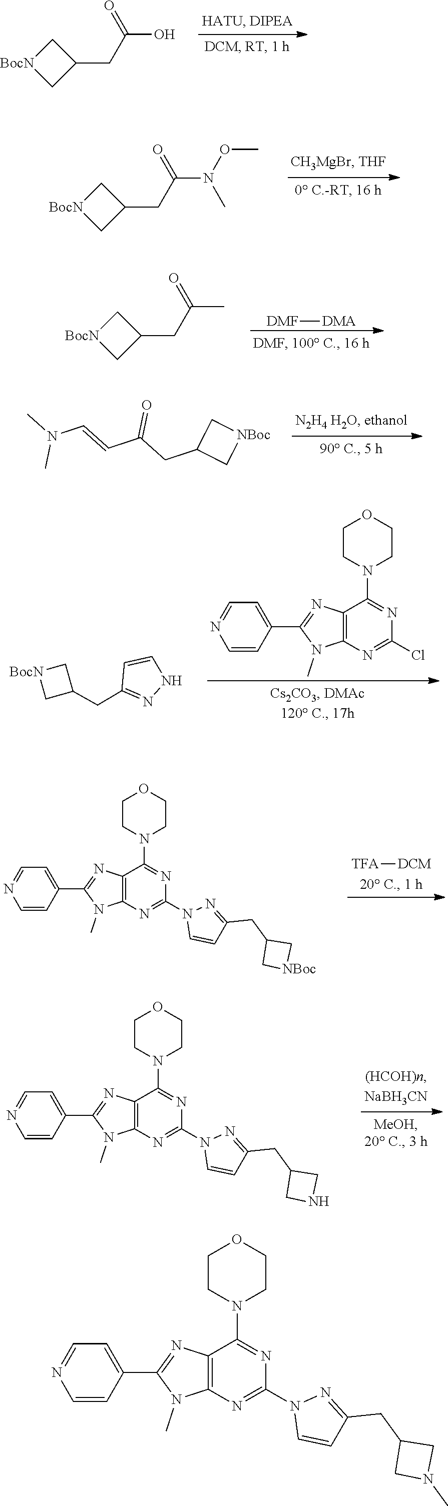

### Step 1: Preparation of tert-butyl 3-(2-(methoxy(methyl)amino)-2-oxoethyl)azetidine-1-carboxylate

A solution of 2-(1-(tert-butoxycarbonyl)azetidin-3-yl)acetic acid (2.15 g, 10 mmol), N,O-dimethylhydroxylamine hydrochloride (1.95 g, 20 mmol), N,N-diisopropylethylamine (5.7 g, 50 mmol) and HATU (5.7 g, 15 mmol) in dichloromethane (50 mL) was stirred at room temperature for 1 h. The mixture was purified by flash (methanol/dichloromethane=1:100) to get tert-butyl 3-(2-(methoxy(methyl)amino)-2-oxoethyl)azetidine-1-carboxylate (1.6 g, 62%) as colorless oil. LCMS (ESI) m/z: 259.2 [M+H]⁺.

### Step 2: Preparation of tert-butyl 3-(2-oxopropyl)azetidine-1-carboxylate

To a solution of tert-butyl 3-(2-(methoxy(methyl)amino)-2-oxoethyl)azetidine-1-carboxylate (1.34 g, 5.1 mmol) in tetrahydrofuran (50 ml) was added methylmagnesium bromide (3M, 2.55 ml, 7.65 mmol) at 0° C. and the mixture was warmed up to 20° C. and stirred for another 16 h. The resultant mixture was quenched with water (2 ml) and dried anhydrous sodium sulfate, filtered and concentrated under reduced pressure. The crude product was purified combi-flash (methanol/dichloromethane:7:100) to obtain tert-butyl 3-(2-oxopropyl)azetidine-1-carboxylate (1.0 g, 92%) as a yellow oil. LCMS (ESI) m/z: 158.1 [M-55]⁺.

### Step 3: Preparation of (E)-tert-butyl 3-(4-(dimethylamino)-2-oxobut-3-enyl)azetidine-1-carboxylate

A solution of tert-butyl 3-(2-oxopropyl)azetidine-1-carboxylate (1.34 g, 6.28 mmol) and DMAc (10 mL) was stirred at 110° C. for 16 h under argon protection. The resultant mixture was concentrated to obtain (E)-tert-butyl 3-(4-(dimethylamino)-2-oxobut-3-enyl)azetidine-1-carboxylate (1.2 g, 71%). as a yellow solid. LCMS (ESI) m/z: 269.3 [M+H]⁺.

### Step 4: Preparation of tert-butyl 3-((1H-pyrazol-3-yl)methyl)azetidine-1-carboxylate

To a mixture of (E)-tert-butyl 3-(4-(dimethylamino)-2-oxobut-3-enyl)azetidine-1-carboxylate (1.2 g, 4.47 mmol) and hydrazine solution (1.2 mL in water, 87%) was added ethanol (100 mL) and the resultant mixture was stirred at reflux for 5 h. The reaction mixture was concentrated under reduced pressure and the crude product was purified combi-flash (dichloromethane/methanol:100:7) to obtain tert-butyl 3-((1H-pyrazol-3-yl)methyl)azetidine-1-carboxylate (470 mg, 44%) as a white solid. LCMS (ESI) m/z: 238.2 [M+H]⁺.

### Step 5: Preparation of tert-butyl 34(1-(9-methyl-6-morpholino-8-(pyridin-4-yl)-9H-purin-2-yl)-1H-pyrazol-3-yl)methyl)azetidine-1-carboxylate

To a solution of 4-(2-chloro-9-methyl-8-(pyridin-4-yl)-9H-purin-6-yl)morpholine (200 mg, 0.60 mmol) in DMA (10 mL) were added tert-butyl 3-((1H-pyrazol-3-yl)methyl)azetidine-1-carboxylate (171 mg, 0.72 mmol) and cesium carbonate (391 mg, 1.2 mmol) and the resultant mixture was stirred at 120° C. under nitrogen for 17 h. It was then filtered, the filtrate was purified by prep-HPLC (BOSTON pHlex ODS 10 um 21.2×250 mm 120A. The mobile phase was acetonitrile/0.1% Ammonium bicarbonate) to obtain tert-butyl 34(1-(9-methyl-6-morpholino-8-(pyridin-4-yl)-9H-purin-2-yl)-1H-pyrazol-3-yl)methyl)azetidine-1-carboxylate (58 mg, 18%) as white solid.

¹H NMR (500 MHz, Chloroform-d) δ 8.82-8.78 (m, 2H), 8.48 (d, J=2.6 Hz, 1H), 7.76-7.72 (m, 2H), 6.23 (d, J=2.6 Hz, 1H), 4.59-4.16 (m, 4H), 4.08 (t, J=8.3 Hz, 2H), 4.02 (s, 3H), 3.94-3.85 (m, 4H), 3.78-3.70 (m, 2H), 3.11-3.04 (m, 2H), 3.01-2.90 (m, 1H), 1.44 (s, 9H). LCMS (ESI) m/z: 532.2 [M+H]⁺.

### Step 6: Preparation of 4-(2-(3-(azetidin-3-ylmethyl)-1H-pyrazol-1-yl)-9-methyl-8-(pyridin-4-yl)-9H-purin-6-yl)morpholine

To a solution of tert-butyl 34(1-(9-methyl-6-morpholino-8-(pyridin-4-yl)-9H-purin-2-yl)-1H-pyrazol-3-yl)methyl)azetidine-1-carboxylate (49.7 mg, 0.093 mmol) in dichloromethane (3 mL) was added TFA (1 mL) and the mixture was stirred at 20° C. for 1 h. It was concentrated and purified by PREP-HPLC (BOSTON pHlex ODS 10 um 21.2×250 mm 120A. The mobile phase was acetonitrile/0.1% Ammonium bicarbonate) to obtain 4-(2-(3-(azetidin-3-ylmethyl)-1H-pyrazol-1-yl)-9-methyl-8-(pyridin-4-yl)-9H-purin-6-yl)morpholine (29.1 mg, 72%) as a white solid.

¹H NMR (400 MHz, Chloroform-d) δ 8.81 (dd, J=4.4, 1.2 Hz, 2H), 8.47 (d, J=2.6 Hz, 1H), 7.76 (dd, J=4.4, 1.6 Hz, 2H), 6.22 (d, J=2.6 Hz, 1H), 4.73-4.10 (m, 4H), 4.02 (s, 3H), 3.92-3.85 (m, 4H), 3.78 (t, J=7.8 Hz, 2H), 3.56 (t, J=7.3 Hz, 2H), 3.25-3.13 (m, 1H), 3.10-3.05 (m, 2H). LCMS (ESI) m/z: 432.2 [M+H]⁺.

### Step 7: Preparation of 4-(9-methyl-2-(34(1-methylazetidin-3-yl)methyl)-1H-pyrazol-1-yl)-8-(pyridin-4-yl)-9H-purin-6-yl)morpholine

A mixture of 4-(2-(3-(azetidin-3-ylmethyl)-1H-pyrazol-1-yl)-9-methyl-8-(pyridin-4-yl)-9H-purin-6-yl)morpholine (20 mg, 0.046 mmol) and paraformaldehyde (4 mg, 0.138 mmol) in methanol (5 mL) was stirred at 20° C. for 1 h under nitrogen protection and then sodium cyanoborohydride (14 mg, 0.23 mmol) was added. The resultant mixture was stirred at room temperature for 2 h and added into water (20 mL) slowly and stirred further at room temperature for 5 min. It was extracted with dichloromethane/methanol (10:1) (3×30 mL), the organic phases was dried over sodium sulfate, filtered and concentrated. The crude product thus obtained was purified by by PREP-HPLC (BOSTON pHlex ODS 10 um 21.2×250 mm 120A. The mobile phase was acetonitrile/0.1% Ammonium bicarbonate) to obtain 4-(9-methyl-2-(3-((1-methylazetidin-3-yl)methyl)-1H-pyrazol-1-yl)-8-(pyridin-4-yl)-9H-purin-6-yl)morpholine (9.6 mg, 43%) as white solid.

¹H NMR (400 MHz, Chloroform-d) δ 8.81 (dd, J=4.8, 1.6 Hz, 2H), 8.47 (d, J=2.6 Hz, 1H), 7.75 (dd, J=4.8, 1.6 Hz, 2H), 6.22 (d, J=2.6 Hz, 1H), 4.80-4.09 (m, 4H), 4.02 (s, 3H), 3.94-3.84 (m, 4H), 3.59 (t, J=7.2 Hz, 2H), 3.07-2.99 (m, 4H), 2.98-2.86 (m, 1H), 2.37 (s, 3H). LCMS (ESI) m/z: 446.2 [M+H]⁺.

### Compounds 13, 81, and 83: Synthesis of tert-butyl 3-(9-methyl-6-morpholino-2-(3-phenyl-1H-pyrazol-1-yl)-9H-purin-8-yl)azetidine-1-carboxylate (Compound 13), 4-(8-(azetidin-3-yl)-9-methyl-2-(3-phenyl-1H-pyrazol-1-yl)-9H-purin-6-yl)morpholine (Compound 83) and 4-(9-methyl-8-(1-methylazetidin-3-yl)-2-(3-phenyl-1H-pyrazol-1-yl)-9H-purin-6-yl)morpholine (Compound 81)

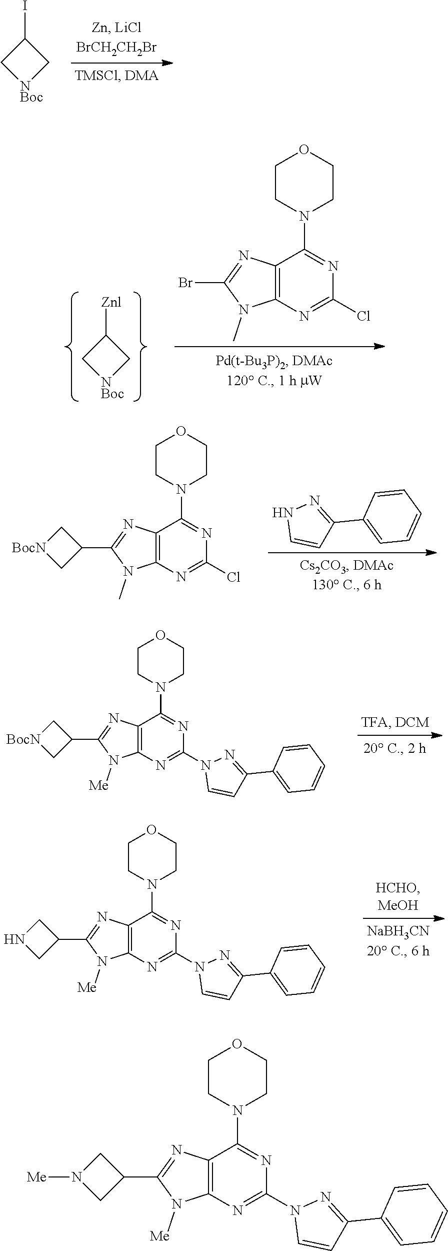

### Step 1: Preparation of tert-butyl 3-(2-chloro-9-methyl-6-morpholino-9H-purin-8-yl)azetidine-1-carboxylate

To a dry 2-necked flask lithium chloride (549 mg, 13.08 mmol) and activated zinc dust (854 mg, 13.06 mmol) were added followed by N,N-dimethylacetamide (8 mL). A solution of 1,2-dibromethane (0.2 mL) in N,N-dimethylacetamide (0.5 mL) was then added dropwise with stirring. A solution of trimethylsilyl chloride (0.1 mL) in N,N-dimethylacetamide (0.5 mL) was also added dropwise and the mixture was stirred for 30 min at 40° C. A solution of tert-butyl 3-iodoazetidine-1-carboxylate (1847 mg, 6.526 mmol) in N,N-dimethylacetamide (1 mL) was then added dropwise. The resulting mixture was stirred at 40° C. for 1 h and then cooled to 20° C. To the above solution was added dropwise a solution of 4-(8-bromo-2-chloro-9-methyl-9H-purin-6-yl)morpholine (720 mg, 2.175 mmol) and bis(tri-tert-butylphosphine)palladium(0) (111 mg, 0.218 mmol) in N,N-dimethylacetamide (4 mL). Then, the mixture was stirred at 120° C. under microwave for 1 h. The resultant crude product was purified by HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous NH₄HCO₃) to obtain tert-butyl 3-(2-chloro-9-methyl-6-morpholino-9H-purin-8-yl)azetidine-1-carboxylate (318 mg, 36%) as white solid. LCMS (ESI) m/z: 409.2 [M+H]⁺.

### Step 2: Preparation of tert-butyl 3-(9-methyl-6-morpholino-2-(3-phenyl-1H-pyrazol-1-yl)-9H-purin-8-yl)azetidine-1-carboxylate

To a solution of tert-butyl 3-(2-chloro-9-methyl-6-morpholino-9H-purin-8-yl)azetidine-1-carboxylate (318 mg, 0.779 mmol) and 3-phenyl-1H-pyrazole (135 mg, 0.935 mmol) in N,N-dimethylacetamide (6 mL) was added cesium carbonate (568 mg, 1.743 mmol). Then the mixture was heated to 130° C. and stirred for 6 h. The resultant crude product was purified by HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous NH₄HCO₃) to obtain tert-butyl 3-(9-methyl-6-morpholino-2-(3-phenyl-1H-pyrazol-1-yl)-9H-purin-8-yl)azetidine-1-carboxylate (198.2 mg, 49%) as light yellow solid.

¹H NMR (400 MHz, DMSO) δ 8.73 (d, J=2.6 Hz, 1H), 7.96 (d, J=7.2 Hz, 2H), 7.47 (t, J=7.5 Hz, 2H), 7.38 (t, J=7.3 Hz, 1H), 7.04 (d, J=2.7 Hz, 1H), 4.84-4.05 (m, 9H), 3.92-3.72 (m, 4H), 3.64 (s, 3H), 1.40 (s, 9H); LCMS (ESI) m/z: 517.2 [M+H]⁺.

### Step 3: Preparation of 4-(8-(azetidin-3-yl)-9-methyl-2-(3-phenyl-1H-pyrazol-1-yl)-9H-purin-6-yl)morpholine

To a solution of tert-butyl 3-(9-methyl-6-morpholino-2-(3-phenyl-1H-pyrazol-1-yl)-9H-purin-8-yl)azetidine-1-carboxylate (187 mg, 0.362 mmol) in dichloromethane (2 mL) was added trifluoroacetic acid (2 mL). The resultant mixture was stirred at 20° C. for 2 h and concentrated. The resultant crude product was purified by HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous NH₄HCO₃) to obtain 4-(8-(azetidin-3-yl)-9-methyl-2-(3-phenyl-1H-pyrazol-1-yl)-9H-purin-6-yl)morpholine (120 mg, 80%) as white solid.

¹H NMR (400 MHz, DMSO) δ 8.72 (d, J=2.6 Hz, 1H), 7.96 (d, J=7.1 Hz, 2H), 7.47 (t, J=7.5 Hz, 2H), 7.38 (t, J=7.3 Hz, 1H), 7.03 (d, J=2.7 Hz, 1H), 4.62-4.06 (m, 6H), 3.94 (t, J=7.3 Hz, 2H), 3.77 (m, 6H), 3.63 (s, 3H); LCMS (ESI) m/z: 417.1 [M+H]⁺.

### Step 4: Preparation of 4-(9-methyl-8-(1-methylazetidin-3-yl)-2-(3-phenyl-1H-pyrazol-1-yl)-9H-purin-6-yl)morpholine

To a solution of 4-(8-(azetidin-3-yl)-9-methyl-2-(3-phenyl-1H-pyrazol-1-yl)-9H-purin-6-yl)morpholine (80 mg, 0.192 mmol) and 30% formalin (192 mg, 1.922 mmol) in methanol (6 mL) was added one drop of acetic acid. The resulting mixture was stirred at 20° C. for 1 h and sodium cyanoborohydride (42 mg, 0.672 mmol) was added. The mixture was stirred for another 1 h and concentrated. The crude product was then purified by HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous NH₄HCO₃) to obtain 4-(9-methyl-8-(1-methylazetidin-3-yl)-2-(3-phenyl-1H-pyrazol-1-yl)-9H-purin-6-yl)morpholine (29.5 mg, 36%) as white solid.

¹H NMR (400 MHz, DMSO) δ 8.72 (d, J=2.7 Hz, 1H), 8.04-7.91 (m, 2H), 7.47 (t, J=7.5 Hz, 2H), 7.38 (t, J=7.3 Hz, 1H), 7.03 (d, J=2.7 Hz, 1H), 4.31 (s, 4H), 3.95 (pent, J=8 Hz, 1H), 3.86-3.75 (m, 4H), 3.71 (t, J=7.3 Hz, 2H), 3.64 (s, 3H), 3.38 (t, J=7.1 Hz, 2H), 2.28 (s, 3H); LCMS (ESI) m/z: 431.2 [M+H]⁺.

### Synthesis of tert-butyl 34(1-(9-methyl-6-morpholino-8-(pyridin-4-yl)-9H-purin-2-yl)-1H-pyrazol-3-yl)methyl)azetidine-1-carboxylate (Compound 96)

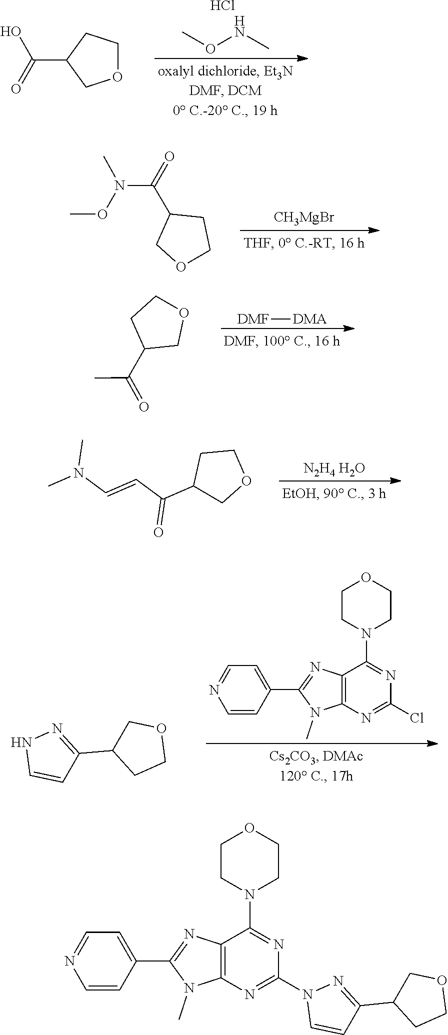

### Step 1: Preparation of N-methoxy-N-methyltetrahydrofuran-3-carboxamide

To a solution of tetrahydrofuran-3-carboxylic acid (986 mg, 8.5 mmol) in dichloromethane (20 ml) was added oxalyl dichloride (1.2 g, 9.35 mmol) at 0° C. followed by 3 drops of N,N-dimethylformamide. After 2 h of stirring at 20° C., the reaction mixture was concentrated under reduced pressure. To the obtained residue 20 ml of chloroform and N,O-dimethylhydroxylamine hydrochloride (1.24 g, 12.75 mmol) were added and the mixture was cooled to 3° C. Triethylamine (2.6 g, 25.5 mmol) was then added dropwise and the mixture was warmed up to room temperature and stirred further for 17 h. The mixture was then acidified with 1 N hydrochloric acid and extracted with chloroform. The organic layer was dried over anhydrous sodium sulfate and concentrated under reduced pressure. The crude residue was purified combi-flash (methanol/dichloromethane: 7:100) to give the crude desired product N-methoxy-N-methyltetrahydrofuran-3-carboxamide (900 mg, 66%) as a yellow oil. ¹H NMR (400 MHz, Chloroform-d) δ 4.06 (t, J=8.4 Hz, 1H), 3.92-3.77 (m, 3H), 3.71 (s, 3H), 3.48-3.36 (m, 1H), 3.21 (s, 3H), 2.28-2.18 (m, 1H), 2.13-2.02 (m, 1H). LCMS (ESI) m/z: 160.2 [M+H]⁺.

### Step 2: Preparation of 1-(tetrahydrofuran-3-yl)ethenone

To a solution of N-methoxy-N-methyltetrahydrofuran-3-carboxamide (900 mg, 5.65 mmol) in THF (50 ml) was added methylmagnesium bromide (2.8 ml, 3M, 8.5 mmol) at 0° C. and the resultant mixture was stirred at 20° C. for 16 h. The resultant mixture was quenched with water (2 ml) and dried anhydrous sodium sulfate, filtered and concentrated. The crude residue was purified combi-flash (methanol/dichloromethane: 10:100) to obtain 1-(tetrahydrofuran-3-yl)ethanone (400 mg, 62%) as yellow oil. ¹H NMR (400 MHz, Chloroform-d) δ 3.98-3.83 (m, 3H), 3.83-3.72 (m, 1H), 3.27-3.13 (m, 1H), 2.21 (s, 3H), 2.16-2.07 (m, 2H). LCMS (ESI) m/z: 115.2 [M+H]⁺.

### Step 3: Preparation of (E)-3-(dimethylamino)-1-(tetrahydrofuran-3-yl)prop-2-en-1-one

A solution of 1-(tetrahydrofuran-3-yl)ethanone (400 mg, 3.5 mmol) and DMAc (4 mL) was stirred at 110° C. for 16 h under argon protection. The mixture was concentrated to obtain (E)-3-(dimethylamino)-1-(tetrahydrofuran-3-yl)prop-2-en-1-one (560 mg, 95%) as yellow oil. LCMS (ESI) m/z: 170.2 [M+H]⁺.

### Step 4: Preparation of 3-(tetrahydrofuran-3-yl)-1H-pyrazole

To a mixture of (E)-3-(dimethylamino)-1-(tetrahydrofuran-3-yl)prop-2-en-1-one (560 mg, 3.3 mmol) and hydrazine solution (0.5 mL) was added ethanol (20 mL) and the resultant mixture was stirred at reflux for 3 h. The reaction mixture was concentrated under reduced pressure and the crude product was purified combi-flash (dichloromethane/methanol: 100:7) to obtain 3-(tetrahydrofuran-3-yl)-1H-pyrazole (220 mg, 48%) as yellow oil. LCMS (ESI) m/z: 139.2 [M+H]⁺.

### Step 5: Preparation of 4-(9-methyl-8-(pyridin-4-yl)-2-(3-(tetrahydrofuran-3-yl)-1H-pyrazol-1-yl)-9H-purin-6-yl)morpholine

To a solution of 4-(2-chloro-9-methyl-8-(pyridin-4-yl)-9H-purin-6-yl)morpholine (198 mg, 0.60 mmol) in N-N-dimethylacetamide (5 mL) were added 3-(tetrahydrofuran-3-yl)-1H-pyrazole (100 mg, 0.72 mmol) and cesium carbonate (586 mg, 1.8 mmol) and the resultant mixture was stirred at 120° C. under nitrogen for 17 h. The resultant mixture was filtered and the filtrate was purified by prep-HPLC (BOSTON pHlex ODS 10 um 21.2×250 mm 120A. The mobile phase was acetonitrile/0.1% Ammonium bicarbonate) to obtain 4-(9-methyl-8-(pyridin-4-yl)-2-(3-(tetrahydrofuran-3-yl)-1H-pyrazol-1-yl)-9H-purin-6-yl)morpholine (98.6 mg, 38%) as white solid.

¹H NMR (400 MHz, Chloroform-d) δ 8.81 (d, J=6 Hz, 2H), 8.52 (d, J=2.7 Hz, 1H), 7.76 (d, J=4 Hz, 2H), 6.35 (d, J=2.7 Hz, 1H), 4.93-3.99 (m, 9H), 3.97-3.83 (m, 6H), 3.76 (pent, J=8 Hz, 1H), 2.50-2.35 (m, 1H), 2.21-2.06 (m, 1H). LCMS (ESI) m/z: 433.2 [M+H]⁺.

Compound 82 was synthesized according to the protocols described above:

| Name | Structure | 1H NMR Data | # |
|---|---|---|---|
| 4-(9-methyl-8- (pyridin-4-yl)-2- (3-(pyrimidin-5- yl)-1H-pyrazol-1- yl)-9H-purin-6- yl)morpholine | 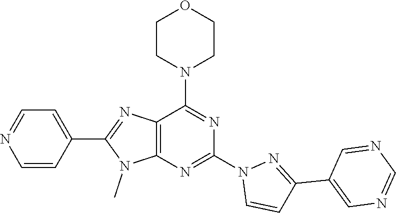 [SMILES: Cn1c(-c2ccncc2)nc2c(N3CCOCC3)nc(-n3ccc(-c4cncnc4)n3)nc21] | ¹H NMR (400 MHz, DMSO) δ 9.36 (s, 2H), 9.21 (s, 1H), 8.89 (d, J = 2.7 Hz, 1H), 8.79 (d, J = 6.0 Hz, 2H), 7.93 (dd, J = 4.5, 1.6 Hz, 2H), 7.27 (d, J = 2.7 Hz, 1H), 4.37 (s, 4H), 3.98 (s, 3H), 3.85-3.76 (m, 4H); LCMS (ESI) m/z: 441.2 [M + H]⁺. | 178 |

### Compounds 19, 85, 92, and 93: Synthesis of 4-(8-(3,6-dihydro-2H-pyran-4-yl)-9-methyl-2-(3-(pyridin-3-yl)-1H-pyrazol-1-yl)-9H-purin-6-yl)morpholine (Compound 85) and 4-(9-methyl-2-(3-(pyridin-3-yl)-1H-pyrazol-1-yl)-8-(tetrahydro-2H-pyran-4-yl)-9H-purin-6-yl)morpholine (Compound 19)

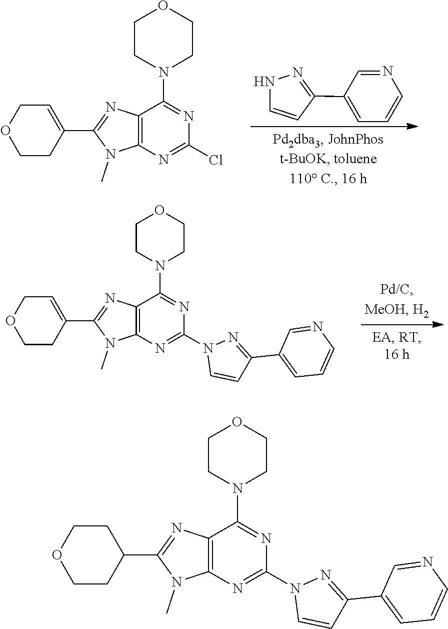

### Step 1: Synthesis of 4-(8-(3,6-Dihydro-2H-pyran-4-yl)-9-methyl-2-(3-(pyridin-3-yl)-1H-pyrazol-1-yl)-9H-purin-6-yl)morpholine

A mixture of 4-(2-chloro-8-(3,6-dihydro-2H-pyran-4-yl)-9-methyl-9H-purin-6-yl)morpholine (200 mg, 0.60 mmol), 3-(1H-pyrazol-3-yl)pyridine (110 mg, 0.76 mmol), tris(dibenzylideneacetone) dipalladium (56 mg, 0.06 mmol), [1,1′-biphenyl]-2-yldi-tert-butylphosphane (36 mg, 0.12 mmol) and potassium tert-butoxide (134 mg, 1.2 mmol) in dry toluene (8 mL) under nitrogen protection was stirred at 110° C. for 16 h. The mixture was cooled to room temperature, quenched with water (10 mL) and extracted with ethyl acetate (10 mL*3). The combined organic phases were washed with water and brine, dried over sodium sulphate, filtered and concentrated. The resultant crude product was purified by prep-HPLC (the crude samples were dissolved in methanol otherwise noted before purified. Boston C18 21*250 mm pm column. The mobile phase was acetonitrile/0.01% aqueous ammonium bicarbonate) to obtain target compound (70 mg, 20.7%) as white solid.

¹H NMR (400 MHz, DMSO-d₆) δ 9.16 (d, J=2.3 Hz, 1H), 8.80 (d, J=2.7 Hz, 1H), 8.58 (dd, J=4.8, 1.7 Hz, 1H), 8.32 (dt, J=8.0, 2.0 Hz, 1H), 7.51 (dd, J=8.0, 4.7 Hz, 1H), 7.16 (d, J=2.7 Hz, 1H), 6.58 (t, J=2.1 Hz, 1H), 4.50-4.30 (m, 6H), 3.90-3.82 (m, 5H), 3.77 (t, J=4.8 Hz, 4H), 2.64-2.58 (m, 2H); LCMS (ESI) m/z: 445.1 [M+H]⁺.

### Step 2: Synthesis of 4-(9-Methyl-2-(3-(pyridin-3-yl)-1H-pyrazol-1-yl)-8-(tetrahydro-2H-pyran-4-yl)-9H-purin-6-yl)morpholine

A mixture of 4-(8-(3,6-dihydro-2H-pyran-4-yl)-9-methyl-2-(3-(pyridin-3-yl)-1H-pyrazol-1-yl)-9H-purin-6-yl)morpholine (30 mg, 0.067 mmol) and Pd/C (10 mg) in methanol (5 mL) and ethyl acetate (2 mL) under hydrogen balloon was stirred at room temperature for 16 h. The mixture was filtered, and the filtrate was concentrated under reduced pressure. The crude product obtained was purified by prep-HPLC (the crude samples were dissolved in methanol otherwise noted before purified. Boston C18 21*250 mm 10 μm column. The mobile phase was acetonitrile/0.01% aqueous ammonium bicarbonate) to obtain the target compound (11.7 mg, 39.2%) as white solid.

¹H NMR (400 MHz, DMSO-d₆) δ 9.16 (d, J=2.3 Hz, 1H), 8.78 (d, J=2.7 Hz, 1H), 8.58 (dd, J=4.8, 1.7 Hz, 1H), 8.32 (dt, J=8.0, 2.0 Hz, 1H), 7.51 (dd, J=7.9, 4.8 Hz, 1H), 7.15 (d, J=2.7 Hz, 1H), 4.30 (bs, 4H), 3.97 (dt, J=11.4, 3.2 Hz, 2H), 3.82-3.72 (m, 7H), 3.51 (td, J=11.2, 3.4 Hz, 2H), 3.29-3.25 (m, 1H), 1.93-1.77 (m, 4H); LCMS (ESI) m/z: 447 [M+H]⁺.

The following compounds were synthesized according to the protocol described above:

| Name | Structure | NMR, MS | # |
|---|---|---|---|
| 4-(9-methyl-2- (4-phenyl-1H- pyrazol-1-yl)-8- (pyridin-4-yl)- 9H-purin-6- yl)morpholine | 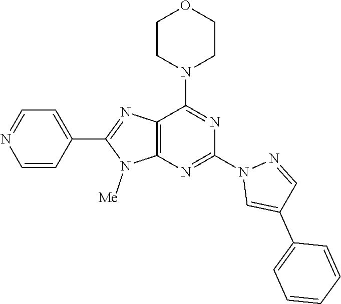 [SMILES: Cn1c(-c2ccncc2)nc2c(N3CCOCC3)nc(-n3cc(-c4ccccc4)cn3)nc21] | ¹H NMR (400 MHz, DMSO-d₆) δ 9.13 (d, J = 0.7 Hz, 1H), 8.79 (dd, J = 4.5, 1.6 Hz, 2H), 8.27 (d, J = 0.7 Hz, 1H), 7.93 (dd, J = 4.5, 1.6 Hz, 2H), 7.86-7.73 (m, 2H), 7.42 (t, J = 7.7 Hz, 2H), 7.28 (t, J = 7.4 Hz, 1H), 4.44-4.39 (m, 4H), 3.97 (s, 3H), 3.86-3.69 (m, 4H); LCMS (ESI) m/z: 439.2 [M + H]⁺. | |
| 4-(9-methyl-8- (pyridin-4-yl)-2- (4-(pyridin-4- yl)-1H-pyrazol- 1-yl)-9H-purin- 6-yl)morpholine | 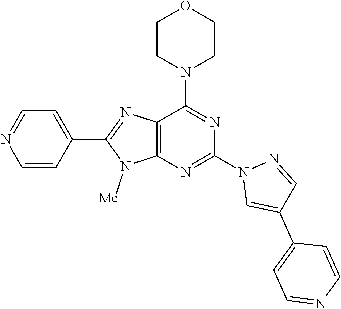 [SMILES: Cn1c(-c2ccncc2)nc2c(N3CCOCC3)nc(-n3cc(-c4ccncc4)cn3)nc21] | ¹H NMR (400 MHz, DMSO-d₆) δ 9.36 (s, 1H), 8.80 (d, J = 5.6 Hz, 2H), 8.58 (s, 2H), 8.44 (s, 1H), 7.93 (dd, J = 4.5, 1.6 Hz, 2H), 7.84 (d, J = 5.8 Hz, 2H), 4.44 (s, 4H), 3.98 (s, 3H), 3.81 (s, 4H); LCMS (ESI) m/z: 440.1 [M + H]⁺. | |

### Compound 87: Synthesis of 9-phenyl-2,6-di(pyridin-4-yl)-9H-purine (Compound 93)

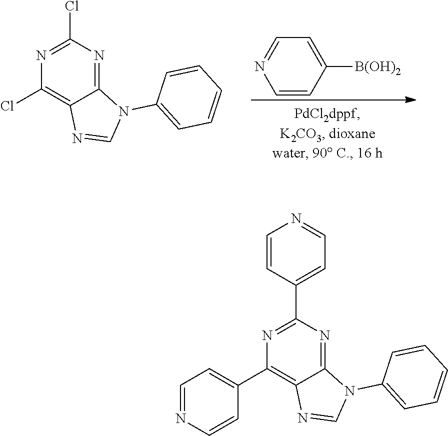

To a solution of 2,6-dichloro-9-phenyl-9H-purine (264 mg, 1 mmol) in dioxane (10 mL) and water (2 mL) were added pyridin-4-ylboronic acid (123 mg, 1 mmol) and [1,1′-bis(diphenylphosphino)ferrocene]dichloropalladium(II) (81 mg, 0.1 mmol) and potassium carbonate (414 mg, 3 mmol) at 25° C. and the resultant mixture was stirred at 90° C. for 16 h under argon protection. It was then extracted with ethyl acetate (20 mL*3) and washed with water (20 mL). The organic layer was dried over sodium sulfate, concentrated and purified by prep-HPLC (BOSTON pHlex ODS 10 um 21.2×250 mm 120A. The mobile phase was acetonitrile/0.1% Ammonium bicarbonate) to give 9-phenyl-2,6-di(pyridin-4-yl)-9H-purine (13 mg, 4%) as a yellow solid. (2-chloro-9-phenyl-6-(pyridin-4-yl)-9H-purine was also isolated as the major product).

¹H NMR (400 MHz, DMSO-d6) δ 9.29 (s, 1H), 8.88-8.94 (m, 4H), 8.81 (d, J=6.0 Hz, 2H), 8.46 (d, J=6.0 Hz, 2H), 8.06 (d, J=7.6 Hz, 2H), 7.73 (t, J=7.6 Hz, 2H), 7.60 (t, J=7.6 Hz, 1H); LCMS (ESI) m/z: 351.1 [M+H]⁺.

### Compound 88: Synthesis of 1-methyl-5-(1-(9-methyl-6-morpholino-8-(pyridin-4-yl)-9H-purin-2-yl)-H-pyrazol-3-yl)piperidin-2-one

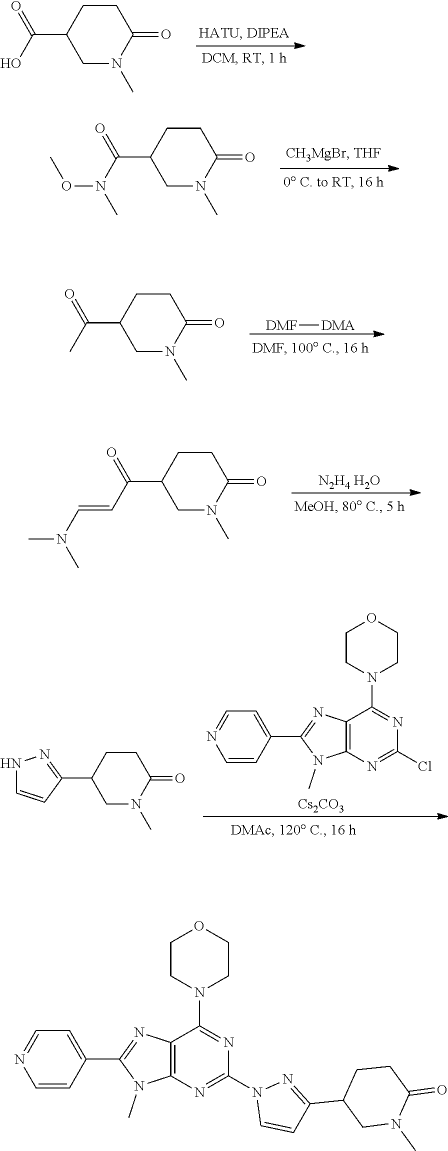

### Step 1: Preparation of N-methoxy-N,1-dimethyl-6-oxopiperidine-3-carboxamide

A mixture of 1-methyl-6-oxopiperidine-3-carboxylic acid (300 mg, 1.91 mmol), DIPEA (1.26 mL, 7.64 mmol) and HATU (1.1 g, 2.86 mmol) in THF (10 mL) was stirred at room temperature for 30 min, then N,O-dimethylhydroxylamine hydrochloride (279 mg, 2.86 mmol) was added and the resultant mixture was stirred at room temperature for 1 h. The mixture was concentrated and purified by column (5% MeOH in DCM) to give N-methoxy-N,1-dimethyl-6-oxopiperidine-3-carboxamide as white solid (350 mg, 92%). LCMS (ESI) m/z: 201 [M+H]⁺.

### Step 2: Preparation of 5-acetyl-1-methylpiperidin-2-one

To a solution of N-methoxy-N,1-dimethyl-6-oxopiperidine-3-carboxamide (300 mg, 1.5 mmol) in THF (8 mL) was added methylmagnesium bromide (0.65 mL, 1.95 mmol) slowly at 0° C. under nitrogen atmosphere and the mixture was warmed up and stirred at room temperature for 16 h. Saturated NH₄Cl (3 mL) solution was added into the mixture and concentrated. The crude product was purified by column chromatography (5% MeOH in DCM) to obtain 5-acetyl-1-methylpiperidin-2-one as colorless oil (150 mg, 65%). LCMS (ESI) m/z: 156 [M+H]⁺.

### Step 3: Preparation of (E)-5-(3-(dimethylamino)acryloyl)-1-methylpiperidin-2-one

A mixture of 5-acetyl-1-methylpiperidin-2-one (80 mg, 0.52 mmol) in DMF-DMA (5 mL) was stirred at 110° C. for 16 h and then concentrated. The crude product thus obtained was purified by column chromatography (8% MeOH in DCM) to obtain (E)-5-(3-(dimethylamino)acryloyl)-1-methylpiperidin-2-one as white solid (80 mg, 65%). LCMS (ESI) m/z: 311 [M+H]⁺.

### Step 4: Preparation of 1-methyl-5-(1H-pyrazol-3-yl)piperidin-2-one

A mixture of (E)-5-(3-(dimethylamino)acryloyl)-1-methylpiperidin-2-one (80 mg, 0.38 mmol) and NH₂NH₂OH (5 mL) in EtOH (5 mL) was stirred at 80° C. for 6 h under nitrogen atmosphere. The mixture was concentrated to give 1-methyl-5-(1H-pyrazol-3-yl)piperidin-2-one as white solid (60 mg, 88%). LCMS (ESI) m/z: 180 [M+H]⁺.

### Step 5: Preparation of 1-methyl-5-(1-(9-methyl-6-morpholino-8-(pyridin-4-yl)-9H-purin-2-yl)-1H-pyrazol-3-yl)piperidin-2-one

A mixture of 1-methyl-5-(1H-pyrazol-3-yl)piperidin-2-one (50 mg, 0.15 mmol), 4-(2-chloro-9-methyl-8-(pyridin-4-yl)-9H-purin-6-yl)morpholine (33 mg, 0.18 mmol) and Cs₂CO₃ (148 mg, 0.45 mmol) in DMAc (5 mL) was stirred at 120° C. for 16 h. The resultant mixture purified by prep-HPLC to give 1-methyl-5-(1-(9-methyl-6-morpholino-8-(pyridin-4-yl)-9H-purin-2-yl)-1H-pyrazol-3-yl)piperidin-2-one as white solid (21.7 mg, 40%).

¹H NMR (400 MHz, DMSO-d6) δ 8.79-8.78 (m, 2H), 8.65 (d, J=2.0 Hz, 1H), 7.91-7.90 (m, 2H), 6.50 (d, J=2.0 Hz, 1H), 4.41-4.24 (m, 4H), 3.93 (s, 3H), 3.79-3.77 (m, 4H), 3.60-3.47 (m, 2H), 3.29-3.25 (m, 1H), 2.87 (s, 3H), 2.45-2.29 (m, 2H), 2.12-1.88 (m, 2H); LCMS (ESI) m/z: 474.3 [M+H]⁺.

### Compound 89: Synthesis of 4-(9-methyl-2-(5-phenyl-1,2,4-oxadiazol-3-yl)-8-(tetrahydro-2H-pyran-4-yl)-9H-purin-6-yl)morpholine

| Name | Structure | 1H NMR Data | # |
|---|---|---|---|
| 4-(9-methyl-2- (5-phenyl-1,2,4- oxadiazol-3-yl)- 8-(tetrahydro- 2H-pyran-4-yl)- 9H-purin-6- yl)morpholine | 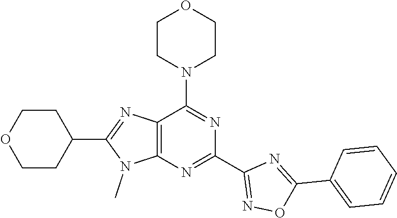 [SMILES: Cn1c(C2CCOCC2)nc2c(N3CCOCC3)nc(-c3noc(-c4ccccc4)n3)nc21] | ¹H NMR (400 MHz, DMSO-d₆) δ 8.20 (d, J = 7.2 Hz, 2H), 7.78-7.72 (m, 1H), 7.71-7.65 (m, 2H), 4.30 (bs, 4H), 3.98-3.95 (m, 2H), 3.81 (s, 3H), 3.77 (t, J = 4.8 Hz, 4H), 3.52 (dt, J = 11.1, 4.0 Hz, 2H), 3.32 (s, 1H), 1.92-1.82 (m, 4H); LCMS (ESI) m/z: 448.2 [M + H]⁺. | 89 |

### Compound 90: Synthesis of 4-(9-methyl-2-(3-phenyl-M-pyrazol-1-yl)-8-(pyrrolidin-3-yl)-9H-purin-6-yl)morpholine

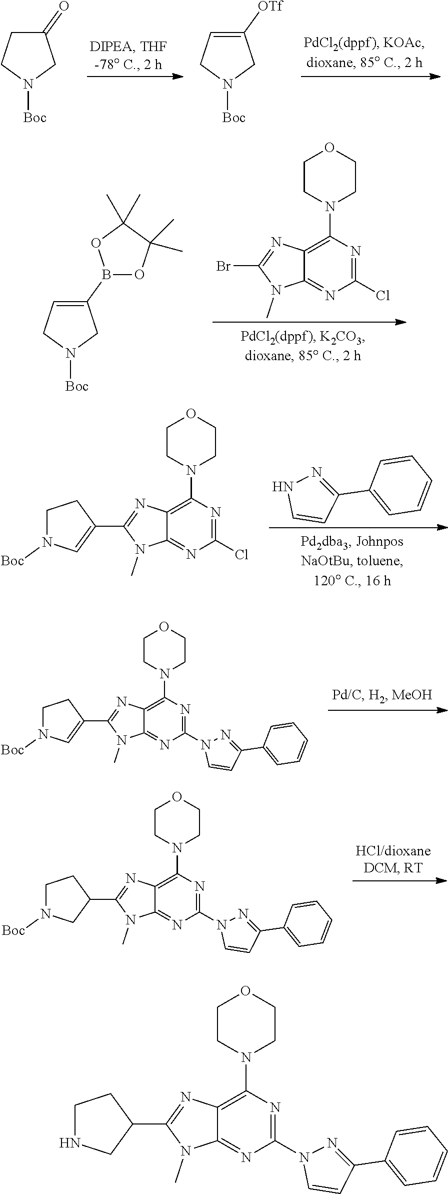

### Step 1: Synthesis of tert-butyl 3-(((trifluoromethyl)sulfonyl)oxy)-2,5-dihydro-1H-pyrrole-1-carboxylate

A solution of tert-butyl 3-oxopyrrolidine-1-carboxylate (1400 mg, 7.567 mmol) and N,N-diisopropylethylamine (2928 mg, 22.701 mmol) in dichloromethane (50 mL) was cooled to −78° C. for 10 mins. Then trifluoromethanesulfonic anhydride (2560 mg, 9.081 mmol) was added. The mixture was warmed up and stirred at 25° C. for 16 h. Then, ammonium chloride (aq) was added and the mixture was extracted with dichloromethane (50 mL×3). The organic layer was dried and concentrated to give tert-butyl 3-(((trifluoromethyl)sulfonyl)oxy)-2,5-dihydro-1H-pyrrole-1-carboxylate (800 mg, 33%) as a yellow oil. LC-MS: m/z=262 (M-56+H)+.

### Step 2: Synthesis of tert-Butyl 3-(4,4,5,5-tetramethyl-1,3,2-dioxaborolan-2-yl)-2,5-dihydro-1H-pyrrole-1-carboxylate

A mixture of tert-butyl 3-(((trifluoromethyl)sulfonyl)oxy)-2,5-dihydro-1H-pyrrole-1-carboxylate (2800 mg, 8.832 mmol), 4,4,4′,4′,5,5,5′,5′-octamethyl-2,2′-bi(1,3,2-dioxaborolane) (4487 mg, 17.665 mmol), [1,1′-bis(diphenylphosphino)ferrocene]dichloropalladium(11) (325 mg, 0.441 mmol) and potassium acetate (2600 mg, 26.532 mmol) in dioxane (80 mL) was stirred at 75° C. for 4 h. Then water was added and the mixture was extracted with ethyl acetate (50 mL×3). The organic layer was dried and concentrated. The crude product was purified by silica gel column (petroleum ether:ethyl acetate from 50:1 to 10:1) to give tert-butyl 3-(4,4,5,5-tetramethyl-1,3,2-dioxaborolan-2-yl)-2,5-dihydro-1H-pyrrole-1-carboxylate (2050 mg, 78%) as a yellow solid. LC-MS: m/z=240 (M-56+H)⁺.

### Step 3: Synthesis of tert-Butyl 4-(2-chloro-9-methyl-6-morpholino-9H-purin-8-yl)-2,3-dihydro-1H-pyrrole-1-carboxylate

A solution of tert-butyl 3-(4,4,5,5-tetramethyl-1,3,2-dioxaborolan-2-yl)-2,5-dihydro-1H-pyrrole-1-carboxylate (307 mg, 1.042 mmol), 4-(8-bromo-2-chloro-9-methyl-9H-purin-6-yl)morpholine (230 mg, 0.695 mmol), bis(diphenylphosphino)ferrocene]dichloropalladium(II) (25 mg, 0.0347 mmol) and potassium carbonate (287 mg, 2.085 mmol) in dioxane/water (8 mL) was stirred at 85° C. for 4 h and concentrated. The crude product thus obtained was purified by silica gel column (petroleum ether:ethyl acetate from 50:1 to 10:1) to obtain tert-butyl 4-(2-chloro-9-methyl-6-morpholino-9H-purin-8-yl)-2,3-dihydro-1H-pyrrole-1-carboxylate (150 mg, 51%) as a yellow solid. LC-MS: m/z=421 (M+H)⁺.

### Step 4: Synthesis of tert-Butyl 4-(9-methyl-6-morpholino-2-(3-phenyl-1H-pyrazol-1-yl)-9H-purin-8-yl)-2,3-dihydro-1H-pyrrole-1-carboxylate

A mixture of tert-butyl 4-(2-chloro-9-methyl-6-morpholino-9H-purin-8-yl)-2,3-dihydro-1H-pyrrole-1-carboxylate (110 mg, 0.262 mmol), 3-phenyl-1H-pyrazole (41 mg, 0.288 mmol), tris(dibenzylideneacetone)dipalladium (23 mg, 0.052 mmol), Johnphos (16 mg, 0.052 mmol) and sodium tert-butoxide (75 mg, 0.786 mmol) in toluene (3 mL) was stirred at 120° C. for 16 h. Water was added and the mixture was extracted ethyl acetate. The organic layer was dried and concentrated. The crude product was purified by silica gel column (petroleum ether:ethyl acetate from 50:1 to 10:1) to give ted-butyl 4-(9-methyl-6-morpholino-2-(3-phenyl-1H-pyrazol-1-yl)-9H-purin-8-yl)-2,3-dihydro-1H-pyrrole-1-carboxylate (70 mg, 51%) as a yellow solid. LC-MS: m/z=529 (M+H)+.

### Step 5: Synthesis of tert-Butyl 3-(9-methyl-6-morpholino-2-(3-phenyl-1H-pyrazol-1-yl)-9H-purin-8-yl)pyrrolidine-1-carboxylate

A suspension of tert-butyl 4-(9-methyl-6-morpholino-2-(3-phenyl-1H-pyrazol-1-yl)-9H-purin-8-yl)-2,3-dihydro-1H-pyrrole-1-carboxylate (70 mg, 0.132 mmol) and Pd/C (10%, 40 mg) in methanol (4 mL) was stirred at 25° C. for 16 h under hydrogen atmosphere. The mixture was filtered and the filtrate was concentrated to obtain tert-butyl 3-(9-methyl-6-morpholino-2-(3-phenyl-1H-pyrazol-1-yl)-9H-purin-8-yl)pyrrolidine-1-carboxylate (50 mg, 71%) as a yellow solid. LC-MS: m/z=531 (M+H)+.

### Step 6: Synthesis of 4-(9-Methyl-2-(3-phenyl-1H-pyrazol-1-yl)-8-(pyrrolidin-3-yl)-9H-purin-6-yl)morpholine

A solution of tert-butyl 3-(9-methyl-6-morpholino-2-(3-phenyl-1H-pyrazol-1-yl)-9H-purin-8-yl)pyrrolidine-1-carboxylate (50 mg, 0.094 mmol), and hydrogen chloride/dioxane (2 mL) in dichloromethane (4 mL) was stirred at 25° C. for 2 h. The mixture was concentrated and the crude was purified by prep-HPLC(Column Xbridge 21.2*250 mm C18, 10 um, mobile phase A: water (10 mmol/L ammonium bicarbonate) B: acetonitrile) to obtain 4-(9-Methyl-2-(3-phenyl-1H-pyrazol-1-yl)-8-(pyrrolidin-3-yl)-9H-purin-6-yl)morpholine (8.5 mg, 21%) as white solid.

¹H NMR (400 MHz, DMSO-d6) δ 8.72 (d, J=2.4 Hz, 1H), 7.96 (d, J=7.2 Hz, 2H), 7.47 (t, J=7.6 Hz, 2H), 7.39-7.36 (m, 1H), 7.03 (d, J=2.0 Hz, 1H), 4.28-4.12 (m, 4H), 3.77-3.75 (m, 7H), 3.56-3.54 (m, 1H), 3.27-3.24 (m, 1H), 3.08 (s, 1H), 2.98-2.93 (m, 2H), 2.16-2.06 (m, 3H); LC-MS: m/z=431 (M-FH)⁺.

### Compound 91: Synthesis of 4-(9-methyl-2-(6-(2-methylpiperidin-4-yl)pyridin-2-yl)-8-(pyridin-4-yl)-9H-purin-6-yl)morpholine

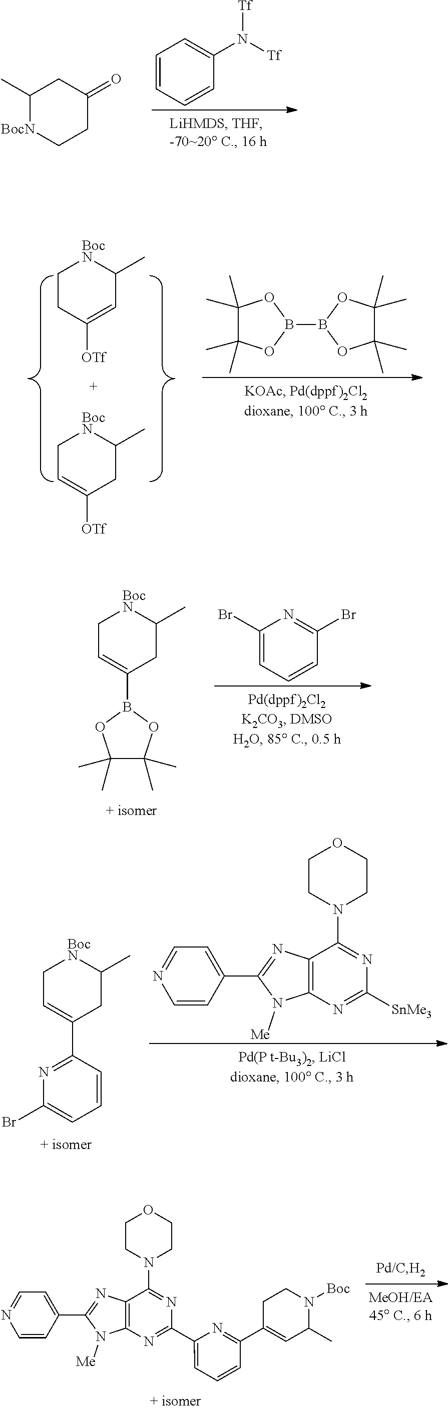

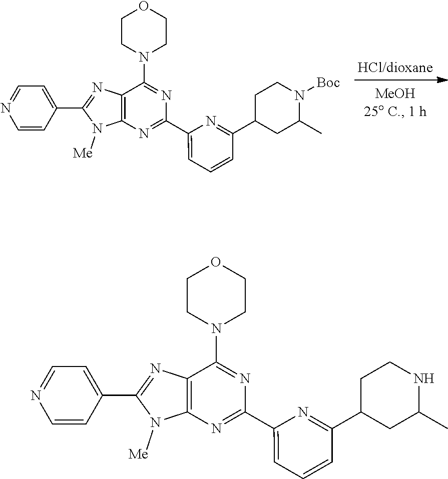 [SMILES: CC1CC(c2cccc(-c3nc(N4CCOCC4)c4nc(-c5ccncc5)n(C)c4n3)n2)CCN1C(=O)OC(C)(C)C ; CC1CC(c2cccc(-c3nc(N4CCOCC4)c4nc(-c5ccncc5)n(C)c4n3)n2)CCN1]

### Step 1: Preparation of tert-butyl 6-methyl-4-(((trifluoromethyl)sulfonyl)oxy)-3,6-dihydropyridine-1(2H)-carboxylate and tert-butyl 2-methyl-4-(((trifluoromethyl)sulfonyl)oxy)-3,6-dihydropyridine-1(2H)-carboxylate

To a solution of tert-butyl 2-methyl-4-oxopiperidine-1-carboxylate (1.5 g, 7 mmol) in tetrahydrofuran (20 mL) was added lithium bis(trimethylsilyl)amide (7.7 mL, 7.7 mmol) at −70° C. slowly. The mixture was stirred at −70° C. for 0.5 h followed by the addition of a solution of 1,1,1-trifluoro-N-phenyl-N-((trifluoromethyl)sulfonyl)methanesulfonamide (2.76 g, 7.7 mmol) in tetrahydrofuran (12 mL) at −70° C., slowly. The mixture was warmed up and stirred at 20° C. for 16 h. Ethyl acetate (50 mL) was added to the reaction mixture and it was washed with aqueous ammonium chloride (20 mL), brine (10 mL), dried over anhydrous sodium sulfate and concentrated. The crude product was purified by flash chromatography (petroleum ether/acetic ester=20:1) to obtain tert-butyl 6-methyl-4-(((trifluoromethyl)sulfonyl)oxy)-3,6-dihydropyridine-1(2H)-carboxylate and tert-butyl 2-methyl-4-(((trifluoromethyl)sulfonyl)oxy)-3,6-dihydropyridine-1(2H)-carboxylate (1.4 g, 58%) as a light yellow oil. LCMS (ESI) m/z: 290.1 [M+H-56]⁺.

### Step 2: Preparation of tert-butyl 2-methyl-4-(4,4,5,5-tetramethyl-1,3,2-dioxaborolan-2-yl)-3,6-dihydropyridine-1(2H)-carboxylate and tert-butyl 6-methyl-4-(4,4,5,5-tetramethyl-1,3,2-dioxaborolan-2-yl)-3,6-dihydropyridine-1(2H)-carboxylate

To a solution of tert-butyl 6-methyl-4-(((trifluoromethyl)sulfonyl)wry)-3,6-dihydropyridine-1(2H)-carboxylate and tert-butyl 2-methyl-4-(((trifluoromethyl)sulfonyl)oxy)-3,6-dihydropyridine-1(2H)-carboxylate (1.45 g, 4.2 mmol) and 4,4,4′,4′,5,5,5′,5′-octamethyl-2,2′-bi(1,3,2-dioxaborolane) (1.17 g, 4.62 mmol) in dioxane (25 mL) were added potassium acetate (0.82 g, 8.4 mmol) and [1,1′-bis(diphenylphosphino)ferrocene]dichloropalladium(II) (0.31 g, 0.42 mmol) and the resultant mixture was stirred at 100° C. under nitrogen for 3 h. The mixture was then concentrated and purified by (petroleum ether:ethyl acetate=10:1) to give tert-butyl 2-methyl-4-(4,4,5,5-tetramethyl-1,3,2-dioxaborolan-2-yl)-3,6-dihydropyridine-1(2H)-carboxylate and tert-butyl 6-methyl-4-(4,4,5,5-tetramethyl-1,3,2-dioxaborolan-2-yl)-3,6-dihydropyridine-1(2H)-carboxylate (650 mg, 48%) as a white solid. LCMS (ESI) m/z: 268.2 [M+H-56]⁺.

### Step 3: Preparation of tert-butyl 6-bromo-2′-methyl-3′,6′-dihydro-[2,4′-bipyridine]-1′(2′H)-carboxylate and tert-butyl 6-bromo-6′-methyl-3′,6′-dihydro-[2,4′-bipyridine]-1′(2′H)-carboxylate

To a solution of tert-butyl 2-methyl-4-(4,4,5,5-tetramethyl-1,3,2-dioxaborolan-2-yl)-3,6-dihydropyridine-1(2H)-carboxylate and tert-butyl 6-methyl-4-(4,4,5,5-tetramethyl-1,3,2-dioxaborolan-2-yl)-3,6-dihydropyridine-1(2H)-carboxylate (0.5 g, 1.55 mmol) and 2,6-dibromopyridine (0.5 g, 2.1 mmol) in DMSO/water (17 mL/1.8 mL) were added potassium carbonate (0.64 g, 4.64 mmol) and [1,1′-bis(diphenylphosphino)ferrocene]dichloropalladium(II) (0.117 g, 0.16 mmol) and the reaction mixture was stirred at 85° C. under nitrogen for 0.5 h. The reaction was quenched with water (50 mL) and extracted with ethyl acetate (30 mL*2) and the organics was concentrated. The crude product was purified by SGC (petroleum ether:ethyl acetate=10:1) to give tert-butyl 6-bromo-2′-methyl-3′,6′-dihydro-[2,4′-bipyridine]-1′(2′H)-carboxylate and tert-butyl 6-bromo-6′-methyl-3′,6′-dihydro-[2,4′-bipyridine]-1′(2′H)-carboxylate (410 mg, 75%) as a light yellow oil. LCMS (ESI) m/z: 297.1 [M+H-56]⁺.

### Step 4: Preparation of tert-butyl 6′-methyl-6-(9-methyl-6-morpholino-8-(pyridin-4-yl)-9H-purin-2-yl)-3′,6′-dihydro-[2,4′-bipyridine]-1′(2′H)-carboxylate and tert-butyl 2′-methyl-6-(9-methyl-6-morpholino-8-(pyridin-4-yl)-9H-purin-2-yl)-3′,6′-dihydro-[2,4′-bipyridine]-1′(2′H)-carboxylate

To a solution of tert-butyl 6-bromo-6′-methyl-3′,6′-dihydro-[2,4′-bipyridine]-1′(2′H)-carboxylate and tert-butyl 6-bromo-2′-methyl-3′,6′-dihydro-[2,4′-bipyridine]-1′(2′H)-carboxylate (0.33 g, 0.72 mmol) in dioxane (8 mL) were added lithium chloride (0.06 g, 1.4 mmol), 4-(9-methyl-8-(pyridin-4-yl)-2-(trimethylstannyl)-9H-purin-6-yl)morpholine (0.3 g, 0.86 mmol) and bis(tri-tert-butylphosphine)palladium(0) (0.08 g, 0.072 mmol) and the reaction was stirred at 100° C. under nitrogen for 4 h. The reaction was quenched with aqueous potassium fluoride (15 mL), filtered and extracted with dichloromethane (20 mL*3). The pooled organic layer was concentrated and the resultant crude product was purified by SGC (petroleum ether:ethyl acetate=2:1) to give mixture of tert-butyl 2′-methyl-6-(9-methyl-6-morpholino-8-(pyridin-4-yl)-9H-purin-2-yl)-3′,6′-dihydro-[2,4′-bipyridine]-1′(2′H)-carboxylate and tert-butyl 6′-methyl-6-(9-methyl-6-morpholino-8-(pyridin-4-yl)-9H-purin-2-yl)-3′,6′-dihydro-[2,4′-bipyridine]-1′(2′H)-carboxylate (100 mg, 24%) as a yellow solid. LCMS (ESI) m/z: 569.3 [M+H]⁺.

### Step 5: Preparation of tert-butyl 2-methyl-4-(6-(9-methyl-6-morpholino-8-(pyridin-4-yl)-9H-purin-2-yl)pyridin-2-yl)piperidine-1-carboxylate

A mixture of tert-butyl 2-methyl-4-(4,4,5,5-tetramethyl-1,3,2-dioxaborolan-2-yl)-3,6-dihydropyridine-1(2H)-carboxylate and tert-butyl 6-methyl-4-(4,4,5,5-tetramethyl-1,3,2-dioxaborolan-2-yl)-3,6-dihydropyridine-1(2H)-carboxylate (0.085 g, 0.15 mmol) and palladium on activated carbon (10% Pd, 0.07 g) in Methanol/ethyl acetate (4 mL/4 mL) was stirred at 45° C. under hydrogen for 6 h. The reaction was filtered and concentrated to give tert-butyl 2-methyl-4-(6-(9-methyl-6-morpholino-8-(pyridin-4-yl)-9H-purin-2-yl)pyridin-2-yl)piperidine-1-carboxylate (40 mg, 47%) as a light yellow solid. LCMS (ESI) m/z: 571.3 [M+H]⁺.

### Step 6: Preparation of 4-(9-methyl-2-(6-(2-methylpiperidin-4-yl)pyridin-2-yl)-8-(pyridin-4-yl)-9H-purin-6-yl)morpholine

A mixture of tert-butyl 2-methyl-4-(6-(9-methyl-6-morpholino-8-(pyridin-4-yl)-9H-purin-2-yl)pyridin-2-yl)piperidine-1-carboxylate (40 mg, 0.07 mmol), hydrochloric acid/dioxane (4 mL) and methanol (1 mL) was stirred at 25° C. for 1 hour. The mixture was filtered and purified by Prep-HPLC (SunFire C18, 4.6*50 mm, 3.5 um column Xbridge C18 3.5 μm 4.6×50 mm column. The mobile phase was acetonitrile/10 mM ammonium bicarbonate aqueous solution.) to obtain 4-(9-methyl-2-(6-(2-methylpiperidin-4-yl)pyridin-2-yl)-8-(pyridin-4-yl)-9H-purin-6-yl)morpholine as white solid. (5.2 mg, 16%).

¹H NMR (400 MHz, DMSO) δ 8.80 (d, J=5.6 Hz, 2H), 8.20 (d, J=8.0 Hz, 1H), 7.95 (d, J=5.4 Hz, 2H), 7.86 (t, J=7.7 Hz, 1H), 7.34 (d, J=7.6 Hz, 1H), 4.36 (s, 4H), 4.01 (s, 3H), 3.80 (s, 4H), 3.14 (d, J=10.1 Hz, 1H), 2.93 (s, 1H), 2.79 (d, J=11.7 Hz, 1H), 2.15-2.05 (m, 1H), 1.95-1.85 (m, 2H), 1.75-1.60 (m, 1H), 1.40-1.30 (m, 1H), 1.10-1.05 (m, 3H); LCMS (ESI) m/z: 471.3 [M+H]⁺.

### Compounds 2, 94, 103, and 108: Preparation of 7-methyl-6-(morpholin-4-yl)-2-(2-phenylpyrimidin-4-yl)-8-(pyridin-4-yl)-7H-purine (Compound 2)

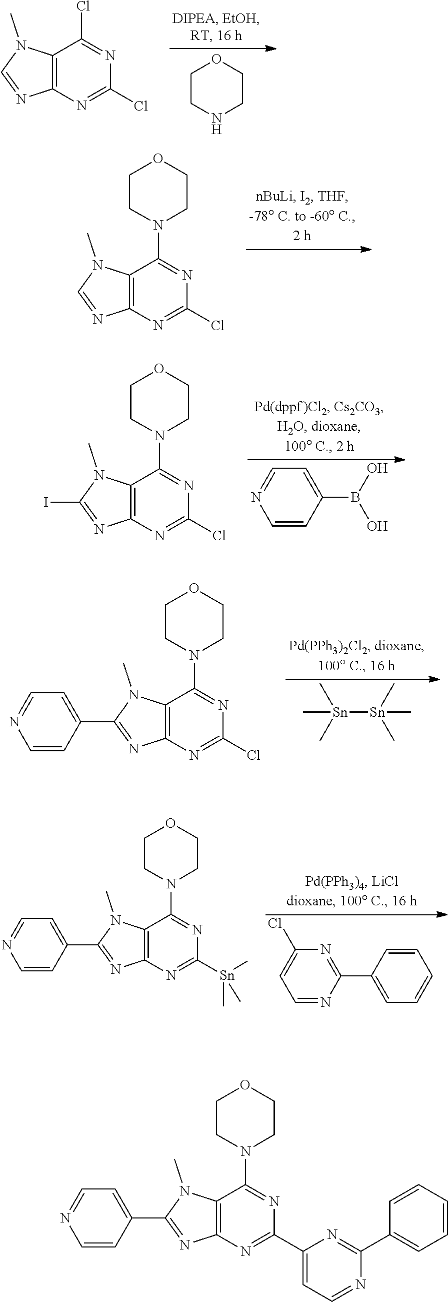

### Step 1: Preparation of 4-(2-chloro-7-methyl-7H-purin-6-yl)morpholine

To a solution of 2,6-dichloro-7-methyl-7H-purine (4.80 g, 24 mmol), morpholine (2.27 g, 26 mmol) in ethanol (100 mL) was added DIPEA (3.06 g, 24 mmol) and the reaction mixture was stirred at room temperature for 16 h. The precipitate formed was collected by filtration, washed with ethanol, and dried under vacuum to afford 4-(2-chloro-7-methyl-7H-purin-6-yl)morpholine (5.00 g, 20 mmol, 83%) as a white solid. ¹H NMR (500 MHz, Chloroform-d) δ 7.97 (s, 1H), 4.01 (s, 3H), 3.93-3.83 (m, 4H), 3.58-3.48 (m, 4H); LCMS (ESI) m/z: 254.1 [M+H]⁺.

### Step 2: Preparation of 4-(2-chloro-8-iodo-7-methyl-7H-purin-6-yl)morpholine

To a solution of 4-(2-chloro-7-methyl-7H-purin-6-yl)morpholine (4.50 g, 18 mmol) in tetrahydrofuran (270 ml) was added a 2.5 M solution of n-butyllithium in hexanes (8.5 mL, 21 mmol) at −78° C. and the resultant mixture was stirred at −78° C. for 30 minutes. A solution of iodine (6.75 g, 27 mmol) in tetrahydrofuran (30 mL) was then added to the reaction mixture and it was allowed to warm to −60° C. over 2 h with stirring. A solution of saturated sodium thiosulfate (200 mL) was added to the reaction vial at −60° C. and then the mixture was extracted with ethyl acetate (2×500 mL). The organic layers were pooled, washed with brine (100 mL), dried over anhydrous sodium sulfate, filtered and concentrated under reduced pressure. The crude residue was purified via flash column chromatography through silica gel using a gradient of 0-5% methanol in dichloromethane to obtain 4-(2-chloro-8-iodo-7-methyl-7H-purin-6-yl)morpholine (2.30 g, 6.1 mmol, 34%) as a yellow solid. LCMS (ESI) m/z: 216.1 [M+H]⁺.

### Step 3: Preparation of 4-(2-chloro-7-methyl-8-(pyridin-4-yl)-7H-purin-6-yl)morpholine

To a solution of 4-(2-chloro-8-iodo-7-methyl-7H-purin-6-yl)morpholine (2.30 g, 6.1 mmol) in dioxane (120 mL) and water (30 mL) was added pyridin-4-ylboronic acid (0.372 g, 3.0 mmol), cesium carbonate (0.197 g, 0.61 mmol) and [1,1′Bis(diphenylphosphino)ferrocene]dichloropalladium(II) (0.219 g, 0.30 mmol) and the mixture was stirred at 100° C. under argon for 2 h. Water (500 mL) was added to the reaction mixture and the mixture was extracted with ethyl acetate (3×500 mL). The organic layers were pooled, dried over anhydrous sodium sulfate, filtered and concentrated under reduced pressure. The crude residue was purified via flash column chromatography through silica gel using a gradient of 0-10% methanol in dichloromethane. The product 4-(2-chloro-7-methyl-8-(pyridin-4-yl)-7H-purin-6-yl)morpholine (0.750 g, 75%) was obtained as a yellow solid. LCMS (ESI) m/z: 331.0 [M+H]⁺.

### Step 4: Preparation of 4-(7-methyl-8-(pyridin-4-yl)-2-(trimethylstannyl)-7H-purin-6-yl)morpholine

To a solution of 4-(2-chloro-7-methyl-8-(pyridin-4-yl)-7H-purin-6-yl)morpholine (281 mg, 0.85 mmol) in dioxane (10 mL) were added 1,1,1,2,2,2-hexamethyldistannane (557 mg, 1.7 mmol) and bis(triphenylphosphine)palladium(II) dichloride (91.0 mg, 0.13 mmol). The reaction mixture was stirred at 100° C. for 2 h, allowed to cool to room temperature and then a 4 M solution of aqueous potassium fluoride (50 mL) was added. The resultant reaction mixture was stirred for 30 minutes and filtered over celite. The filtrate was extracted with dichloromethane (2×60 mL), washed with brine (40 mL), dried over sodium sulfate, and concentrated under reduced pressure. The crude product 4-(7-methyl-8-(pyridin-4-yl)-2-(trimethylstannyl)-7H-purin-6-yl)morpholine (390 mg, 0.85 mmol, 100%) was obtained as a brown solid and carried onto next step without further purification. LCMS (ESI) m/z: 459.0 [M+H]⁺.

### Step 5: Preparation of 4-(7-methyl-2-(2-phenylpyrimidin-4-yl)-8-(pyridin-4-yl)-7H-purin-6-yl)morpholine

To a mixture of 4-(7-methyl-8-(pyridin-4-yl)-2-(trimethylstannyl)-7H-purin-6-yl)morpholine (390 mg, 0.85 mmol), 4-chloro-2-phenylpyrimidine (194 mg, 1.0 mmol), and lithium chloride (89.0 mg, 2.13 mmol) in dioxane (10 mL) was added tetrakis(triphenylphosphine)palladium(0) (98.0 mg, 0.085 mmol). The reaction mixture was stirred at 100° C. for 16 h under argon. The reaction mixture was allowed to cool to room temperature, then filtered over celite and washed with ethyl acetate (2×30 mL). The filtrate was concentrated under reduced pressure. The crude product was purified by prep-HPLC (the crude samples were dissolved in N,N-dimethylformamide unless otherwise noted before purification. Boston pHlex ODS um 21.2×250 mm 120A. The mobile phase was acetonitrile/0.1% Ammonium bicarbonate) to give product 4-(7-methyl-2-(2-phenylpyrimidin-4-yl)-8-(pyridin-4-yl)-7H-purin-6-yl)morpholine (14.1 mg, 0.031 mmol, 3.3%) as a white solid.

¹H NMR (500 MHz, Chloroform-d) δ 8.98 (d, J=5.1 Hz, 1H), 8.88 (d, J=5.1 Hz, 2H), 8.69-8.62 (m, 2H), 8.37 (d, J=5.1 Hz, 1H), 7.85 (d, J=5.2 Hz, 2H), 7.56-7.48 (m, 3H), 4.12 (s, 3H), 4.05-3.98 (m, 4H), 3.75 (t, J=4.6 Hz, 4H). LCMS (ESI) m/z: 451.0 [M+H]⁺.

The following compounds were synthesized according to the above-described protocol.

| Name | Structure | 1H NMR Data | # |
|---|---|---|---|
| 4-(9-methyl-8- (pyridin-4-yl)-2- (2- (trifluoromethyl) pyrimidin-4-yl)- 9H-purin-6- yl)morpholine | 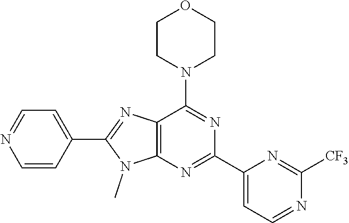 [SMILES: Cn1c(-c2ccncc2)nc2c(N3CCOCC3)nc(-c3ccnc(C(F)(F)F)n3)nc21] | ¹H NMR (400 MHz, DMSO) δ 9.21 (d, J = 5.2 Hz, 1H), 8.81 (d, J = 5.6 Hz, 2H), 8.73 (d, J = 5.2 Hz, 1H), 7.95 (d, J = 5.7 Hz, 2H), 4.38 (bs, 4H), 4.03 (s, 3H), 3.87-3.73 (m, 4H). LCMS (ESI) m/z: 443.1[M + H]+. | 94 |
| 4-(2-(4- cyclopropylpyri midin-2-yl)-9- methyl-8- (pyridin-4-yl)- 9H-purin-6- yl)morpholine | 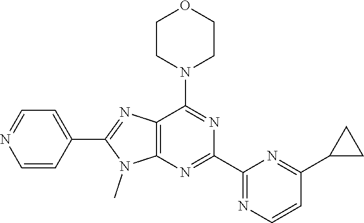 [SMILES: Cn1c(-c2ccncc2)nc2c(N3CCOCC3)nc(-c3nccc(C4CC4)n3)nc21] | 1H NMR (400 MHz, CD₃OD) δ 8.75 (d, J = 4 Hz, 2H), 8.70 (d, J = 5.3 Hz, 1H), 7.99 (dd, J = 4.6, 1.6 Hz, 2H), 7.36 (d, J = 5.3 Hz, 1H), 4.46 (s, 4H), 4.08 (s, 3H), 3.96-3.76 (m, 4H), 2.26 (pent, J = 4 Hz, 1H), 1.30-1.20 (m, 4H); LCMS (ESI) m/z: 415.1 [M + H]⁺ | 103 |
| 4-(9-methyl-2- (2-(piperidin-4- yl)pyrimidin-4- yl)-8-(pyridin-4- yl)-9H-purin-6- yl)morpholine | 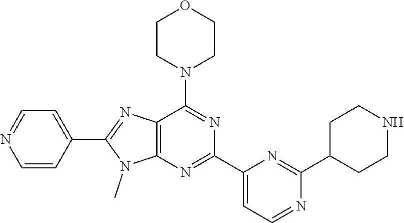 [SMILES: Cn1c(-c2ccncc2)nc2c(N3CCOCC3)nc(-c3ccnc(C4CCNCC4)n3)nc21] | ¹H NMR (400 MHz, DMSO-d₆) δ 8.90 (d, J = 4.8 Hz, 1H), 8.82-8.80 (m, 2H), 8.20 (d, J = 5.2 Hz, 1H), 7.95-7.94 (m, 2H), 4.40-4.33 (m, 4H), 4.01 (s, 3H), 3.81-3.79 (m, 4H), 3.10-3.02 (m, 3H), 2.71- 2.65 (m, 2H), 1.95-1.92 (m, 2H), 1.83-1.77 (m, 2H), LCMS (ESI) m/z: 458 [M + H]⁺ | 108 |

### Compound 95: Synthesis of 4-(9-methyl-2-(2-(piperidin-3-yl)pyrimidin-4-yl)-8-(pyridin-4-yl)-9H-purin-6-yl)morpholine

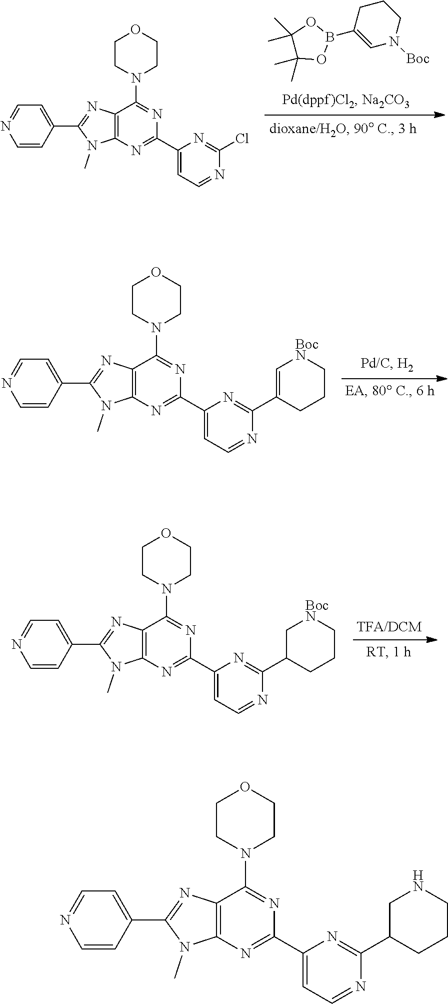 [SMILES: Cn1c(-c2ccncc2)nc2c(N3CCOCC3)nc(-c3ccnc(Cl)n3)nc21 ; CC(C)(C)OC(=O)N1C=C(B2OC(C)(C)C(C)(C)O2)CCC1 ; Cn1c(-c2ccncc2)nc2c(N3CCOCC3)nc(-c3ccnc(C4=CN(C(=O)OC(C)(C)C)CCC4)n3)nc21 ; Cn1c(-c2ccncc2)nc2c(N3CCOCC3)nc(-c3ccnc(C4CCCN(C(=O)OC(C)(C)C)C4)n3)nc21 ; Cn1c(-c2ccncc2)nc2c(N3CCOCC3)nc(-c3ccnc(C4CCCNC4)n3)nc21]

### Step 1: Preparation of tert-butyl 5-(4-(9-methyl-6-morpholino-8-(pyridin-4-yl)-9H-purin-2-yl)pyrimidin-2-yl)-3,4-dihydropyridine-1(2H)-carboxylate

A mixture of 4-(2-(2-chloropyrimidin-4-yl)-9-methyl-8-(pyridin-4-yl)-9H-purin-6-yl)morpholine (75 mg, 0.18 mmol), tert-butyl 5-(4,4,5,5-tetramethyl-1,3,2-dioxaborolan-2-yl)-3,4-dihydropyridine-1(2H)-carboxylate (62 mg, 0.20 mmol), Na₂CO₃ (58 mg, 0.55 mmol) and Pd(dppf)Cl₂ (15 mg, 0.2 mmol) in DMF (8 mL) and H₂O (1 mL) was stirred at 80° C. for 2 h under nitrogen protection. The mixture was concentrated and purified by column chromatography (20% EA in PE) to obtain tert-butyl 5-(4-(9-methyl-6-morpholino-8-(pyridin-4-yl)-9H-purin-2-yl)pyrimidin-2-yl)-3,4-dihydropyridine-1(2H)-carboxylate as white solid (60 mg, 59%). LCMS (ESI) m/z: 556 [M+H]⁺.

### Step 2: Preparation of tert-butyl 3-(4-(9-methyl-6-morpholino-8-(pyridin-4-yl)-9H-purin-2-yl)pyrimidin-2-yl)piperidine-1-carboxylate

A mixture of tert-butyl 5-(4-(9-methyl-6-morpholino-8-(pyridin-4-yl)-9H-purin-2-yl)pyrimidin-2-yl)-3,4-dihydropyridine-1(2H)-carboxylate (70 mg, 0.14 mmol) and 10% Pd/C (70 mg) in MeOH (5 mL) and ethyl acetate (5 mL) was stirred at 80° C. for 16 h under H₂ atmosphere. The mixture was filtered and concentrated to obtain the desired product as white solid (60 mg, 85%). LCMS (ESI) m/z: 558 [M+H]⁺.

### Step 3: Preparation of 4-(9-methyl-2-(2-(piperidin-3-yl)pyrimidin-4-yl)-8-(pyridin-4-yl)-9H-purin-6-yl)morpholine

To a solution of tert-butyl 3-(4-(9-methyl-6-morpholino-8-(pyridin-4-yl)-9H-purin-2-yl)pyrimidin-2-yl)piperidine-1-carboxylate (50 mg, 0.11 mmol) in DCM (5 mL) was added TFA (2 mL) and the mixture was stirred at room temperature for 1 h. The resultant mixture was concentrated and purified by Prep-HPLC to obtain 4-(9-methyl-2-(2-(piperidin-3-yl)pyrimidin-4-yl)-8-(pyridin-4-yl)-9H-purin-6-yl)morpholine. (2.3 mg, 4%) as white solid.

¹H NMR (400 MHz, DMSO-d6) δ 8.91 (d, J=5.2 Hz, 1H), 8.81 (d, J=5.6 Hz, 2H), 8.22 (d, J=5.6 Hz, 1H), 7.95 (d, J=6.0 Hz, 2H), 4.43-4.31 (m, 4H), 4.02 (s, 3H), 3.81-3.77 (m, 4H), 3.40-3.35 (m, 1H), 3.11-2.92 (m, 3H), 2.89-2.60 (m, 1H), 2.44-2.15 (m, 1H), 2.11-1.54 (m, 3H); LCMS (ESI) m/z: 458.2 [M+H]⁺.

### Compound 97: Synthesis of 4-(2-(2-cyclopropylpyrimidin-4-yl)-9-methyl-8-(pyridin-4-yl)-9H-purin-6-yl)morpholine

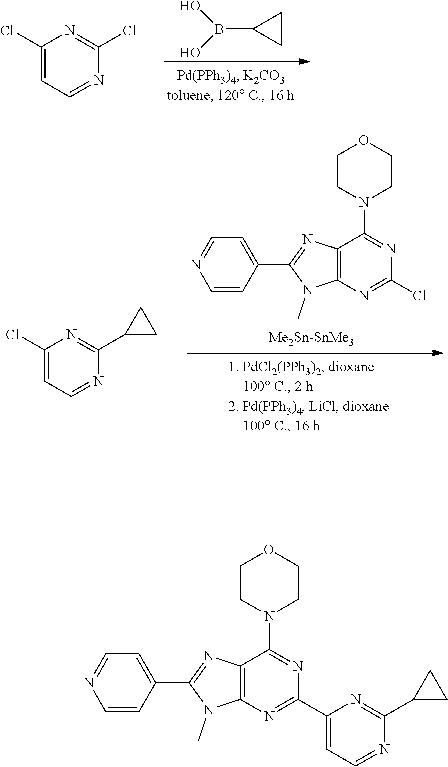 [SMILES: Clc1ccnc(Cl)n1 ; OB(O)C1CC1 ; Clc1ccnc(C2CC2)n1 ; Cn1c(-c2ccncc2)nc2c(N3CCOCC3)nc(Cl)nc21 ; Cn1c(-c2ccncc2)nc2c(N3CCOCC3)nc(-c3ccnc(C4CC4)n3)nc21]

### Step 1: Synthesis of 4-Chloro-2-cyclopropylpyrimidine

A solution of 2,4-dichloropyrimidine (500 mg, 3.355 mmol), cyclopropylboronic acid (288 mg, 3.355 mmol), tetrakis(triphenyl phosphine)palladium (352 mg, 0.3355 mmol) and potassium carbonate (1389 mg, 10.065 mmol) in dioxane (30 mL) was stirred at 100° C. for 16 h. Then water was added and the mixture was extracted with ethyl acetate (50 mL×3). The organic layer was dried and concentrated and the crude product was purified by Pre-TLC (petroleum ether:ethyl acetate from 50:1 to 10:1) to give 4-chloro-2-cyclopropylpyrimidine (310 mg, 60%) as a yellow solid. LC-MS: m/z=155 (M-FH)+.

### Step 2: Synthesis of 4-(9-Methyl-8-(pyridin-4-yl)-2-(trimethylstannyl)-9H-purin-6-yl)morpholine

A solution of 4-(2-chloro-9-methyl-8-(pyridin-4-yl)-9H-purin-6-yl)morpholine (600 mg, 1.812 mmol), 1,1,1,2,2,2-hexamethyl distannane (1185 mg, 3.625 mmol), bis(triphenylphosphine)palladium(II) dichloride (127 mg, 0.181 mmol) in dioxane (25 mL) was stirred at 100° C. for 1 h. To the resultant mixture were added, 4-chloro-2-cyclopropylpyrimidine (250 mg, 1.623 mmol), tetrakis(triphenylphosphine)palladium (170 mg, 0.162 mmol) and lithium chloride (136 mg, 3.246 mmol) in dioxane (30 mL) and the resultant mixture was stirred at 100° C. for 16 h. It was concentrated and the crude product was purified by silica gel column (dichloromethane:methanol from 100:1 to 10:1) to afford 4-(2-(2-cyclopropylpyrimidin-4-yl)-9-methyl-8-(pyridin-4-yl)-9H-purin-6-yl)morpholine (6.3 mg, 1%) as white solid.

¹H NMR (400 MHz, CD₃OD) δ 8.79-8.76 (m, 3H), 8.24 (d, J=5.2 Hz, 1H), 8.02 (d, J=4.8, 1.4 Hz, 2H), 4.48 (bs, 4H), 4.14 (s, 3H), 3.91-3.88 (m, 4H), 2.50-2.42 (m, 1H), 1.26-1.24 (m, 2H), 1.17-1.15 (m, 2H); LC-MS: m/z=415.2 (M+H)+.

### Compounds 98-102, 104, 106, 110, 111, and 115

| Name | Structure | 1H NMR Data | # |
|---|---|---|---|
| 4-(2-(5- chloropyridin-2- yl)-9-methyl-8- (pyridin-4-yl)-9H- purin-6- yl) morpholine | 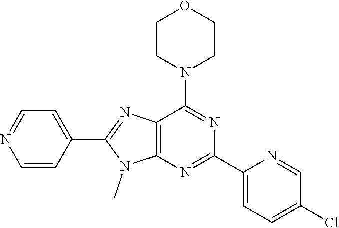 [SMILES: Cn1c(-c2ccncc2)nc2c(N3CCOCC3)nc(-c3ccc(Cl)cn3)nc21] | ¹H NMR (400 MHz, DMSO-d₆) δ 8.81-8.77 (m, 3H), 8.49 (d, J = 8.8 Hz, 1H), 8.08-8.06 (m, 1H), 7.93(d, J = 6.0 Hz, 1H), 4.35 (bs, 4H), 3.99 (s, 3H), 3.81-3.78 (m, 4H); LCMS (ESI) m/z: 408.1 [M + H]⁺. | 98 |
| 4-(2- (benzo[d][1,3]diox ol-5-yl)-9-methyl- 8-(pyridin-4-yl)- 9H-purin-6- yl)morpholine | 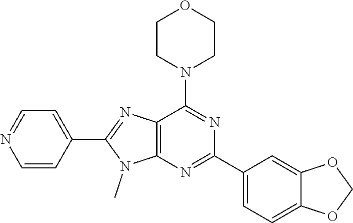 [SMILES: Cn1c(-c2ccncc2)nc2c(N3CCOCC3)nc(-c3ccc4c(c3)OCO4)nc21] | ¹H NMR (400 MHz, DMSO-d₆) δ 8.78 (d, J = 5.5 Hz, 2H), 8.04 (d, J = 8.2 Hz, 1H), 7.91 (d, J = 5.7 Hz, 3H), 7.01 (d, J = 8.2 Hz, 1H), 6.10 (s, 2H), 4.33 (s, 4H), 3.97 (s, 3H), 3.79 (s, 4H); LCMS (ESI) m/z: 417.1 [M + H]⁺. | 99 |
| 4-(9-methyl-8- (pyridin-4-yl)-2-(3- (trifluoromethyl)- 1H-pyrazol-1-yl)- 9H-purin-6- yl)morpholine | 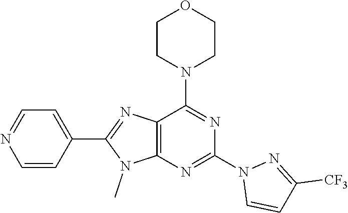 [SMILES: Cn1c(-c2ccncc2)nc2c(N3CCOCC3)nc(-n3ccc(C(F)(F)F)n3)nc21] | ¹H NMR (400 MHz, DMSO-d₆) δ 8.94 (s, 1H), 8.80- 8.79 (m, 2H), 7.92 (d, J = 4.8 Hz, 2H), 7.03 (d, J = 2.0 Hz, 1H), 4.63-4.55 (m, 4H), 4.00 (s, 3H), 3.96-3.79 (m, 4H); LCMS (ESI) m/z: 431.1/433.1 [M + H]⁺. | 100 |
| 4-(2-(3,3- difluoropyrrolidin- 1-yl)-9-methyl-8- (pyridin-4-yl)-9H- purin-6- yl)morpholine | 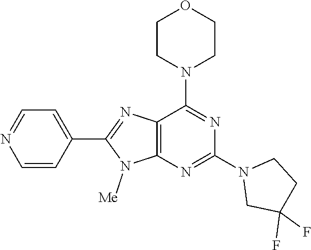 [SMILES: Cn1c(-c2ccncc2)nc2c(N3CCOCC3)nc(N3CCC(F)(F)C3)nc21] | ¹H NMR (400 MHz, CDCl₃) δ 8.73 (d, J = 6.0 Hz, 2H), 7.71 (d, J = 6.0 Hz, 2H), 4.29 (bs, 4H), 3.94-4.01 (m, 2H), 3.82-3.86 (m, 9H), 2.40-2.50 (m, 2H); LCMS (ESI) m/z: 402.1 [M + H]⁺. | 101 |
| N-cyclopentyl-9- methyl-6- morpholino-8- (pyridin-4-yl)-9H- purin-2-amine | 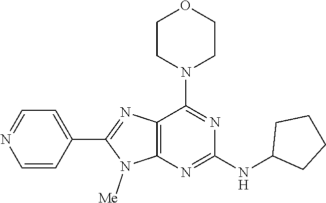 [SMILES: Cn1c(-c2ccncc2)nc2c(N3CCOCC3)nc(NC3CCCC3)nc21] | ¹H NMR (400 MHz, CDCl₃) δ 8.72 (d, J = 5.6 Hz, 2H), 7.69 (d, J = 6.0 Hz, 2H), 4.81-4.79 (m, 1H), 4.32-4.28 (m, 5H), 3.84-3.81 (m, 7H), 2.09-2.05 (m, 2H), 1.75- 1.47 (m, 6H); LCMS (ESI) m/z: 380.3 [M + H]⁺. | 102 |
| 4-(9-methyl-2- (pyridin-3-yl)-8- (pyridin-4-yl)-9H- purin-6- yl)morpholine | 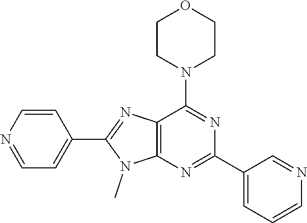 [SMILES: Cn1c(-c2ccncc2)nc2c(N3CCOCC3)nc(-c3cccnc3)nc21] | ¹H NMR (400 MHz, DMSO-d₆) δ 31H NMR (400 MHz, DMSO-d6) o 9.58(d, J = 2.0 Hz, 1H), 8.79(d, J = 6.0 Hz, 2H), 8.72(d, J = 8.0 Hz, 1H), 8.66-8.67(m, 1H), 7.93(d, J = 6.0 Hz, 2H), 7.53(dd, J = 8.0, 4.8 Hz, 1H), 4.00(s, 3H), 4.37(s, 4H), 3.80(t, J = 4.4 Hz, 4H); LCMS (ESI) m/z: 374.3[M + H]⁺. | 104 |
| 4-(2-(3,4- dimethoxyphenyl) -9-methyl-8- (pyridin-4-yl)-9H- purin-6- yl)morpholine | 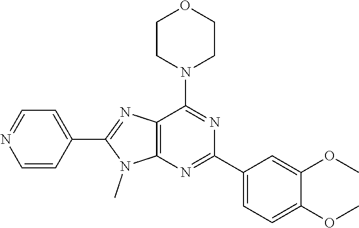 [SMILES: COc1ccc(-c2nc(N3CCOCC3)c3nc(-c4ccncc4)n(C)c3n2)cc1OC] | ¹H NMR (400 MHz, DMSO-d₆) δ 8.78 (d, J = 6.0 Hz, 2H), 8.06-8.03 (m, 2H), 7.92 (d, J = 8.4 Hz, 1H), 4.34- 4.33 (bs, 4H), 4.00(s, 3H), 3.87 (s, 3H), 3.83 (s, 3H), 3.81-3.79 (m, 4H); LCMS (ESI) m/z: 433.1 [M + H]⁺. | 106 |
| 4-(2-cyclopropyl- 9-methyl-8- (pyridin-4-yl)-9H- purin-6- yl)morpholine | 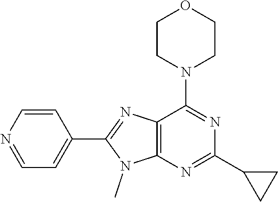 [SMILES: Cn1c(-c2ccncc2)nc2c(N3CCOCC3)nc(C3CC3)nc21] | ¹H NMR (400 MHz, DMSO-d₆) δ 8.76 (d, J = 5.1 Hz, 2H), 7.87 (d, J = 5.3 Hz, 2H), 4.21 (bs, 4H), 3.86 (s, 3H), 3.72 (t, J = 4.8 Hz, 4H), 2.05 (s, 1H), 1.07-0.81 (m, 4H); LCMS (ESI) m/z: 337.2 [M + H]⁺. | 110 |
| 4-(9-methyl-2- (pyridin-2-yl)-8- (pyridin-4-yl)-9H- purin-6- yl)morpholine | 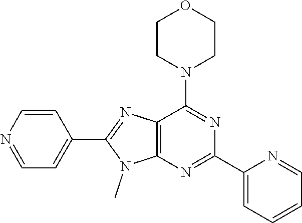 [SMILES: Cn1c(-c2ccncc2)nc2c(N3CCOCC3)nc(-c3ccccn3)nc21] | ¹HNMR (400 MHz, DMSO-d₆) δ 1HNMR (400 MHz, DMSO-d6) 0 8.79 (dd, J = 4.4, 1.6 Hz, 2H), 8.73(d, J = 3.6 Hz, 1H), 8.44(d, J = 8.0 Hz, 1H), 7.91-7.96(m, 3H), 7.46-7.49(m, 1H), 4.36(s, 4H), 4.00(s, 3H), 3.79(t, J = 4.8 Hz, 4H), LCMS (ESI) m/z: 374.3 [M + H]⁺. | 111 |
| 4-(9-ethyl-2-(1H- pyrazol-3-yl)-8- (pyridin-4-yl)-9H- purin-6- yl)morpholine | 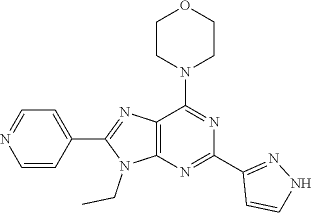 [SMILES: CCn1c(-c2ccncc2)nc2c(N3CCOCC3)nc(-c3cc[nH]n3)nc21] | ¹H NMR (400 MHz, DMSO) δ 13.48 (s, 1H), 8.79 (dd, J = 4.5, 1.6 Hz, 2H), 7.84 (dd, J = 4.5, 1.6 Hz, 2H), 7.61 (s, 1H), 6.89 (d, J = 1.8 Hz, 1H), 4.45 (q, J = 7.2 Hz, 2H), 4.36 (bs, 4H), 3.86-3.70 (m, 4H), 1.34 (t, J = 7.2 Hz, 3H). LCMS (ESI) m/z: 377.2 [M + H]+. | 115 |

### Compound 105: Synthesis of 4-(9-(difluoromethyl)-2-(2-phenylpyrimidin-4-yl)-8-(pyridin-4-yl)-9H-purin-6-yl)morpholine (Batch 1)

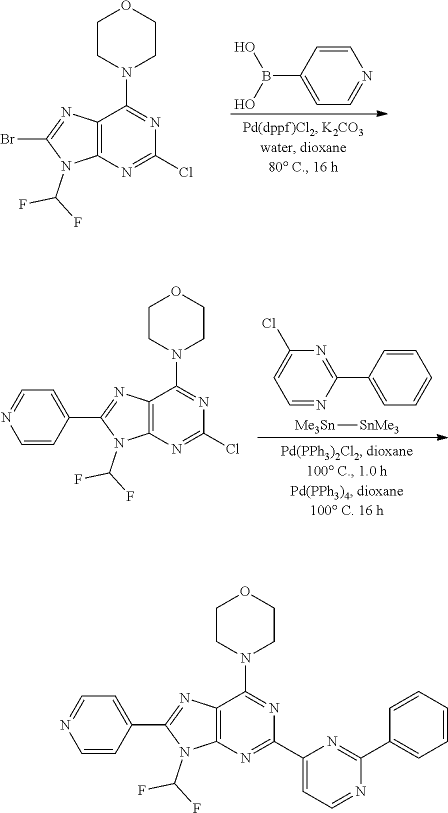

### Step 1: Synthesis of 4-(2-chloro-9-(difluoromethyl)-8-(pyridin-4-yl)-9H-purin-6-yl)morpholine

A solution of 4-(8-bromo-2-chloro-9-(difluoromethyl)-9H-purin-6-yl)morpholine (300 mg, 0.8 mmol), pyridin-4-ylboronic acid (108 mg, 0.88 mmol), 1,1′-bis(diphenylphosphino) ferrocene-palladium(II) dichloride dichloromethane complex (65 mg, 0.08 mmol) and potassium carbonate (330 mg, 2.4 mmol) in water (1.5 mL) and dioxane (15 mL) was stirred at 90° C. for 16 h under argon. The reaction mixture was cooled and concentrated. The crude product was purified by flash chromatography (Biotage, 80 g silica gel, methanol/dichloromethane=3%-4%) to give the desired product 4-(2-chloro-9-(difluoromethyl)-8-(pyridin-4-yl)-9H-purin-6-yl)morpholine (240 mg, 73%) as yellow solid. LCMS: (ESI) m/z 366.8 [M+H]+.

### Step 2: Synthesis of 4-(9-(difluoromethyl)-2-(2-phenylpyrimidin-4-yl)-8-(pyridin-4-yl)-9H-purin-6-yl)morpholine

To a solution of 4-chloro-2-phenylpyrimidine (92 mg, 0.5 mmol) in dioxane (10 mL) were added hexamethyldistannane (196 mg, 0.6 mmol) and bis(triphenylphosphine)palladium(II) chloride (35 mg, 0.05 mmol). The mixture was stirred at 100° C. for 1 h. The reaction mixture was cooled and 4-(2-chloro-9-(difluoromethyl)-8-(pyridin-4-yl)-9H-purin-6-yl)morpholine (92 mg, 0.25 mmol) and tetrakis(triphenylphosphine)palladium (58 mg, 0.05 mmol) were added to the reaction mixture and stirring was continued at 100° C. for 16 h. The reaction mixture was concentrated, the crude product was purified by Prep-HPLC (BOSTON pHlex ODS 10 um 21.2×250 mm 120A. The mobile phase was acetonitrile/0.1% Ammonium bicarbonate) to afford 4-(9-(difluoromethyl)-2-(2-phenylpyrimidin-4-yl)-8-(pyridin-4-yl)-9H-purin-6-yl)morpholine (23.3 mg, 13%) as white solid.

¹H NMR (400 MHz, DMSO-d6) δ 9.10 (d, J=5.1 Hz, 1H), 8.85 (d, J=6.0 Hz, 2H), 8.57 (dd, J=6.7, 3.0 Hz, 2H), 8.33 (d, J=5.2 Hz, 1H), 8.26 (t, J=58 Hz, 1H), 7.87 (d, J=6.0 Hz, 2H), 7.66-7.52 (m, 3H), 4.41 (s, 4H), 3.89-3.74 (m, 4H); LCMS: (ESI) m/z 486.8 [M+H]+.

### Compound 105: Synthesis of 4-(9-(difluoromethyl)-2-(2-phenylpyrimidin-4-yl)-8-(pyridin-4-yl)-9H-purin-6-yl)morpholine (Batch 2)

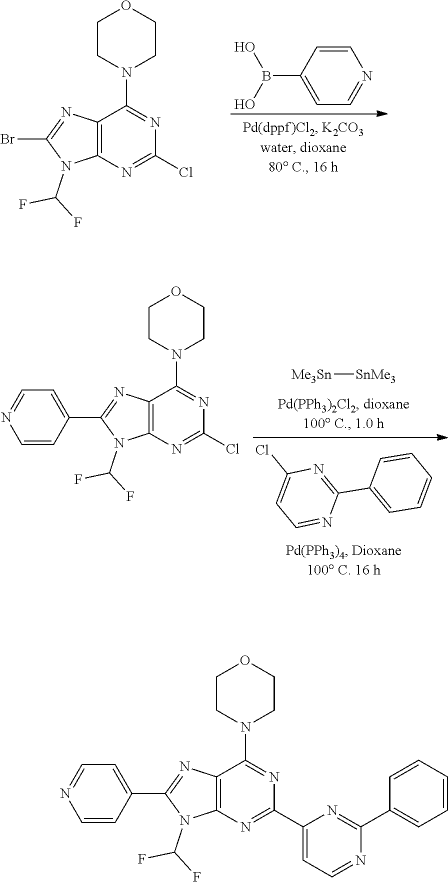

### Step 1: 4-(2-chloro-9-(difluoromethyl)-8-(pyridin-4-yl)-9H-purin-6-yl)morpholine

A solution of 4-(8-bromo-2-chloro-9-(difluoromethyl)-9H-purin-6-yl)morpholine (300 mg, 0.8 mmol), pyridin-4-ylboronic acid (108 mg, 0.88 mmol), 1,1′-bis(diphenylphosphino) ferrocene-palladium(II) dichloride dichloromethane complex (65 mg, 0.08 mmol) and potassium carbonate (330 mg, 2.4 mmol) in water (1.5 mL) and dioxane (15 mL) was stirred at 90° C. for 16 h under argon. It was concentrated and purified by Flash chromatography (Biotage, 80 g silica gel, methanol/dichloromethane=3%-4%) to give the desired product 4-(2-chloro-9-(difluoromethyl)-8-(pyridin-4-yl)-9H-purin-6-yl)morpholine (240 mg, 73%) as yellow solid. LCMS: (ESI) m/z 366.8 [M+H]+.

### Step 2: Synthesis of 4-(9-(difluoromethyl)-2-(2-phenylpyrimidin-4-yl)-8-(pyridin-4-yl)-9H-purin-6-yl)morpholine

To a solution of 4-chloro-2-phenylpyrimidine (92 mg, 0.5 mmol) in dioxane (10 mL) were added hexamethyldistannane (196 mg, 0.6 mmol) and bis(triphenylphosphine)palladiuM(II) chloride (35 mg, 0.05 mmol). The mixture was stirred at 100° C. for 1.0 h. The reaction mixture was cooled, 4-(2-chloro-9-(difluoromethyl)-8-(pyridin-4-yl)-9H-purin-6-yl)morpholine (92 mg, 0.25 mmol) and tetrakis(triphenylphosphine)palladium (58 mg, 0.05 mmol) was added to the reaction mixture and stirring was continued at 100° C. for 16 h. The reaction mixture was concentrated, the crude residue was purified by Prep-HPLC (BOSTON pHlex ODS 10 um 21.2×250 mm 120A. The mobile phase was acetonitrile/0.1% Ammonium bicarbonate) to afford 4-(9-(difluoromethyl)-2-(2-phenylpyrimidin-4-yl)-8-(pyridin-4-yl)-9H-purin-6-yl)morpholine (23.3 mg, 13%) as white solid.

1H NMR (400 MHz, DMSO-d6) δ 9.10 (d, J=5.1 Hz, 1H), 8.85 (d, J=6.0 Hz, 2H), 8.57 (dd, J=6.7, 3.0 Hz, 2H), 8.49-8.12 (m, 2H), 7.87 (d, J=6.0 Hz, 2H), 7.66-7.52 (m, 3H), 4.41 (s, 4H), 3.89-3.74 (m, 4H); LCMS: (ESI) m/z 486.8 [M+H]⁺.

### Compound 107: Synthesis of 2-methyl-6-(9-methyl-6-morpholino-8-(pyridin-4-yl)-9H-purin-2-yl)isoindolin-1-one

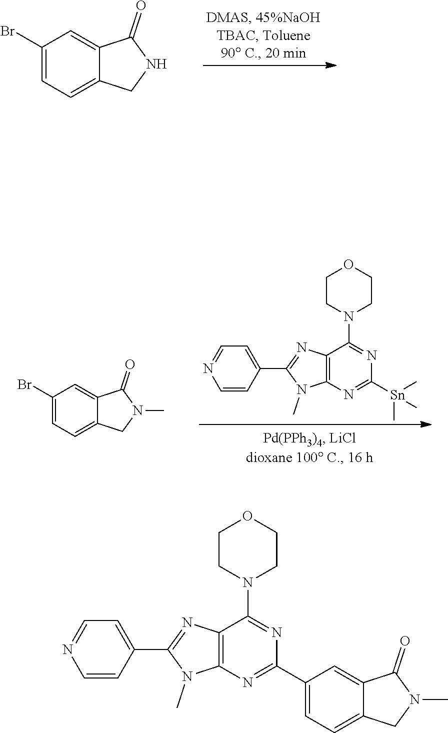 [SMILES: O=C1NCc2ccc(Br)cc21 ; CN1Cc2ccc(Br)cc2C1=O ; Cn1c(-c2ccncc2)nc2c(N3CCOCC3)n[c]([Sn]([CH3])([CH3])[CH3])nc21 ; CN1Cc2ccc(-c3nc(N4CCOCC4)c4nc(-c5ccncc5)n(C)c4n3)cc2C1=O]

### Step 1: Synthesis of 6-bromo-2-methylisoindolin-1-one

A mixture of 6-bromoisoindolin-1-one (100 mg, 0.47 mmol), Me₂SO₄ (0.1 mL, 0.71 mmol), NaOH(aq.45%) (419 mg, 4.72 mmol) and Bu₄NCl (26 mg, 0.09 mmol) in toluene (5 mL) was stirred at 80° C. for 12 min. The mixture was concentrated and purified by column chromatography (50% EA in PE) to give the desired compound as white solid (30 mg, 60%). LCMS (ESI) m/z: 226 [M+H]⁺.

### Step 2: Synthesis of 2-methyl-6-(9-methyl-6-morpholino-8-(pyridin-4-yl)-9H-purin-2-yl)isoindolin-1-one

To a solution of 4-(9-methyl-8-(pyridin-4-yl)-2-(trimethylstannyl)-9H-purin-6-yl)morpholine (80 mg, 0.17 mmol), 6-bromo-2-methylisoindolin-1-one (47 mg, 0.21 mmol) and LiCl (26 mg, 0.51 mmol) in dioxane (10 mL) was added Pd(PPh₃)₄ (25 mg, 0.02 mmol) and the resultant mixture was stirred at 100° C. for 16 h under nitrogen atmosphere. The mixture was then concentrated and purified by Prep-HPLC to obtain the desired product (6 mg, 10%) as yellow solid.

¹H NMR (400 MHz, DMSO-d6) δ 8.79 (d, J=5.2 Hz, 1H), 8.70-8.66 (m, 2H), 7.93 (d, J=5.6 Hz, 2H), 7.69 (d, J=8.0 Hz, 1H), 4.53 (s, 2H), 4.40-4.33 (m, 4H), 4.03 (s, 3H), 4.02-3.82 (m, 4H), 3.11 (s, 3H); LCMS (ESI) m/z: 442.2 [M+H]⁺.

### Compound 109: Synthesis of 4-(2-(5-phenyl-1,2,4-oxadiazol-3-yl)-8-(pyridin-4-yl)-9H-purin-6-yl)morpholine

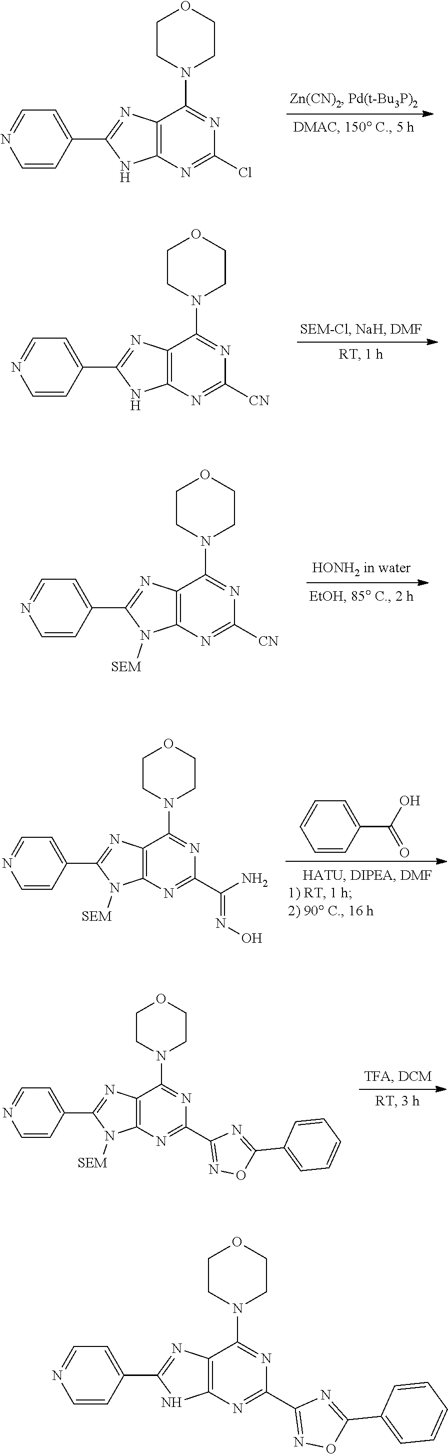

### Step 1: Synthesis of 6-morpholino-8-(pyridin-4-yl)-9H-purine-2-carbonitrile

A mixture of 4-(2-chloro-8-(pyridin-4-yl)-9H-purin-6-yl)morpholine (150 mg, 0.45 mmol), zinc cyanide (63 mg, 0.54 mmol) and bis(tri-tert-butylphosphine)palladium (24 mg, 0.045 mmol) in dry dimethylacetamide (6 mL) under nitrogen protection was stirred at 150° C. for 4 h. The mixture was cooled to room temperature, quenched with water (10 mL) and filtered, and the residue was washed with water and dried to give the crude product as brown solid (150 mg, crude). LCMS (ESI) m/z: 308 [M+H]⁺.

### Step 2: Synthesis of 6-Morpholino-8-(pyridin-4-yl)-94(2-(trimethylsilyl)ethoxy)methyl)-9H-purine-2-carbonitrile

To a solution of 6-morpholino-8-(pyridin-4-yl)-9H-purine-2-carbonitrile (150 mg, 0.49 mmol) in tetrahydrofuran (10 mL) was added sodium hydride (60% in oil, 29 mg, 0.74 mmol), after stirring at room temperature for 10 minutes, 2-(trimethylsilyl)ethoxymethyl chloride (122 mg, 0.73 mmol) was added. The resulting mixture was stirred at room temperature for 2 h, then quenched with methanol (5 mL) and concentrated under reduced pressure. The crude product was then purified by column chromatography (20% ethyl acetate in petroleum ether) to obtain the product as white solid (150 mg, 70.2%). LCMS (ESI) m/z: 438 [M+H]⁺.

### Step 3: Synthesis of (Z)-N′-hydroxy-6-morpholino-8-(pyridin-4-yl)-9-((2-(trimethylsilyl)ethoxy)methyl)-9H-purine-2-carboximidamide

A mixture of 6-morpholino-8-(pyridin-4-yl)-94(2-(trimethylsilyl)ethoxy)methyl)-9H-purine-2-carbonitrile (150 mg, 0.34 mmol) and hydroxylamine (50% in water, 0.034 mL, 34 mg) in ethanol (5 mL) was stirred at 80° C. for 2 h. The mixture was concentrated under reduced pressure to give the crude product as yellow oil (150 mg, crude), which used for next step directly without further purification. LCMS (ESI) m/z: 471 [M+H]⁺.

### Step 4: Synthesis of 4-(2-(5-Phenyl-1,2,4-oxadiazol-3-yl)-8-(pyridin-4-yl)-94(2-(trimethylsilyl)ethoxy)methyl)-9H-purin-6-yl)morpholine

A mixture of (Z)-N′-hydroxy-6-morpholino-8-(pyridin-4-yl)-9-((2-(trimethylsilyl)ethoxy)methyl)-9H-purine-2-carboximidamide (150 mg, 0.32 mmol), benzoic acid (39 mg, 0.32 mmol), 1-[bis(dimethylamino)methylene]-1H-1,2,3-triazolo[4,5-b]pyridinium 3-oxid hexafluorophosphate (182 mg, 0.48 mmol) and N,N-diisopropylethylamine (83 mg, 0.64 mmol) in N,N-dimethylformamide (10 mL) was stirred at room temperature for 1 h at 90° C. for 16 h. The mixture was cooled to room temperature, quenched with water (10 mL) and extracted with ethyl acetate (10 mL*3). The combined organic phases were washed with water and brine, dried over sodium sulfate, filtered and concentrated under reduced pressure to give the crude product as yellow oil (100 mg, crude). LCMS (ESI) m/z: 557 [M+H]⁺.

### Step 5: Synthesis of 4-(2-(5-Phenyl-1,2,4-oxadiazol-3-yl)-8-(pyridin-4-yl)-9H-purin-6-yl)morpholine

A mixture of 4-(2-(5-phenyl-1,2,4-oxadiazol-3-yl)-8-(pyridin-4-yl)-94(2-(trimethylsilyl)ethoxy)methyl)-9H-purin-6-yl)morpholine (100 mg, 0.18 mmol) and trifluoroacetic acid (2 mL) in dichloromethane (5 mL) was stirred at room temperature for 3 h. The mixture was concentrated and the residue was quenched by saturated sodium bicarbonate solution till pH>7 and extracted by ethyl acetate (10 mL*3). The combined organic phases were concentrated and the crude product was purified by prep-HPLC (Boston C18 21*250 mm 10 μm column. The mobile phase was acetonitrile/0.01% aqueous ammonium bicarbonate) to obtain the desired product as white solid (9.2 mg, 12.0%).

¹H NMR (400 MHz, DMSO-d₆) δ 14.39 (s, 1H), 8.78 (d, J=6.0 Hz, 2H), 8.21 (d, J=6.8 Hz, 2H), 8.07 (d, J=6.0 Hz, 2H), 7.79-7.73 (m, 1H), 7.71-7.65 (m, 2H), 4.38 (s, 4H), 3.82 (t, J=4.8 Hz, 4H); LCMS (ESI) m/z: 427.8 [M+H]⁺.

### Compound 112: Synthesis of 1-(9-methyl-6-morpholino-2-(1-phenyl-1H-pyrazol-3-yl)-9H-purin-8-yl)ethane-1,2-diol

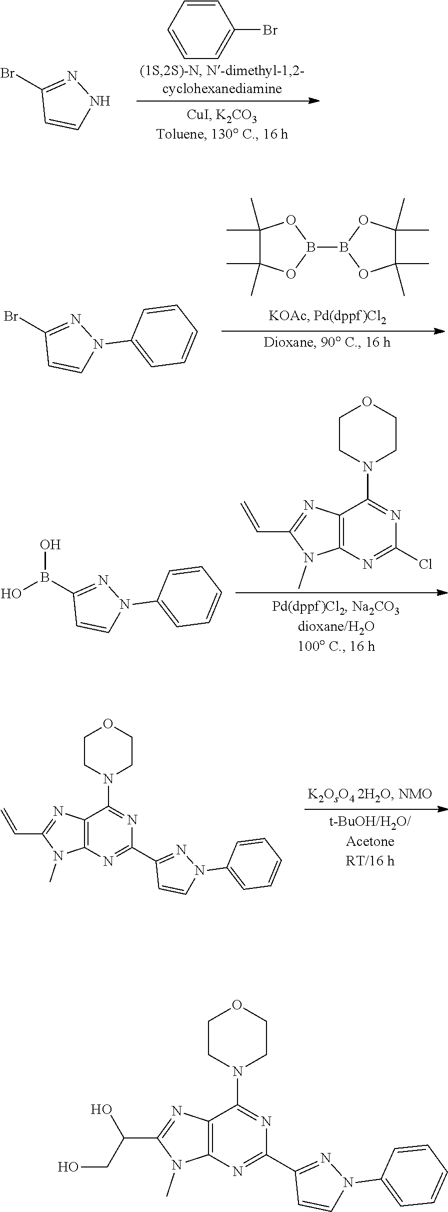

### Step 1: Synthesis of 3-bromo-1-phenyl-1H-pyrazole

A mixture of 3-bromo-1H-pyrazole (500 mg, 3.40 mmol), bromobenzene (1.6 g, 10.21 mmol), (1S,2S)—N1,N2-dimethylcyclohexane-1,2-diamine (97 mg, 0.68 mmol), KOAc (1.41 g, 10.21 mmol) and CuI (32 mg, 0.17 mmol) in toluene (20 mL) was stirred at 130° C. for 16 h under nitrogen protection. The resultant mixture was concentrated and purified by column chromatography (30% EA in PE) to give the target compound as white solid (600 mg, 79%). LCMS (ESI) m/z: 223 [M+H]⁺.

### Step 2: Synthesis of (1-phenyl-1H-pyrazol-3-yl)boronic acid

A mixture of 3-bromo-1-phenyl-1H-pyrazole (150 mg, 0.67 mmol), bis(pinacolato)diboron (512 mg, 2.02 mmol), KOAc (330 mg, 3.36 mmol) and Pd(dppf)Cl₂ (49 mg, 0.07 mmol) in dioxane (8 mL) was stirred at 80° C. for 16 h under nitrogen atmosphere. The resultant mixture was concentrated and purified by column chromatography (5% EA in PE) to obtain the desired compound as white solid (150 mg, 82%). LCMS (ESI) m/z: 189 [M+H]⁺.

### Step 3: Synthesis of 4-(9-methyl-2-(1-phenyl-1H-pyrazol-3-yl)-8-vinyl-9H-purin-6-yl)morpholine

A mixture of 4-(2-chloro-9-methyl-8-vinyl-9H-purin-6-yl)morpholine (100 mg, 0.36 mmol), (1-phenyl-1H-pyrazol-3-yl)boronic acid (101 mg, 0.54 mmol), Cs₂CO₃ (349 mg, 1.07 mmol) and Pd(dppf)C12 (26 mg, 0.04 mmol) in dioxane (8 mL) and H₂O (1 mL) was stirred at 80° C. for 2 h under nitrogen atmosphere. The mixture was concentrated and purified by column chromatography (30% EA in PE) to obtain the desired product as white solid (80 mg, 58%). LCMS (ESI) m/z: 388 [M+H]⁺.

### Step 4: Synthesis of 1-(9-methyl-6-morpholino-2-(1-phenyl-1H-pyrazol-3-yl)-9H-purin-8-yl)ethane-1,2-diol

To a solution of 4-(9-methyl-2-(1-phenyl-1H-pyrazol-3-yl)-8-vinyl-9H-purin-6-yl)morpholine (40 mg, 0.10 mmol) in acetone (4 mL), water (1 mL) and 2-methylpropan-2-ol (1 ml) were added potassium osmate(VI) dehydrate (7 mg, 0.02 mmol) and 4-methylmorpholine N-oxide (36 mg, 0.15 mmol) and the resultant mixture was stirred at 25 0 0 for 4 h. It was then was filtered and the filtrate was concentrated. The crude product was purified by pre-HPLC to obtain 1-(9-methyl-6-morpholino-2-(1-phenyl-1H-pyrazol-3-yl)-9H-purin-8-yl)ethane-1,2-diol as white solid (3.1 mg, 8%).

¹H NMR (400 MHz, DMSO-d6) δ 8.57 (d, J=2.0 Hz, 1H), 7.94 (d, J=7.6 Hz, 2H), 7.57-7.53 (m, 2H), 7.37-7.35 (m, 1H), 7.15 (d, J=2.0 Hz, 1H), 5.84 (d, J=5.2 Hz, 1H), 4.92-4.86 (m, 2H), 4.47-4.25 (m, 4H), 3.86-3.77 (m, 9H); LCMS (ESI) m/z: 422.2 [M+H]⁺.

### Compound 113: Synthesis of 4-(2-(2,3-dihydrobenzo[b][1,4]dioxin-6-yl)-9-methyl-8-(piperidin-4-yl)-9H-purin-6-yl)morpholine

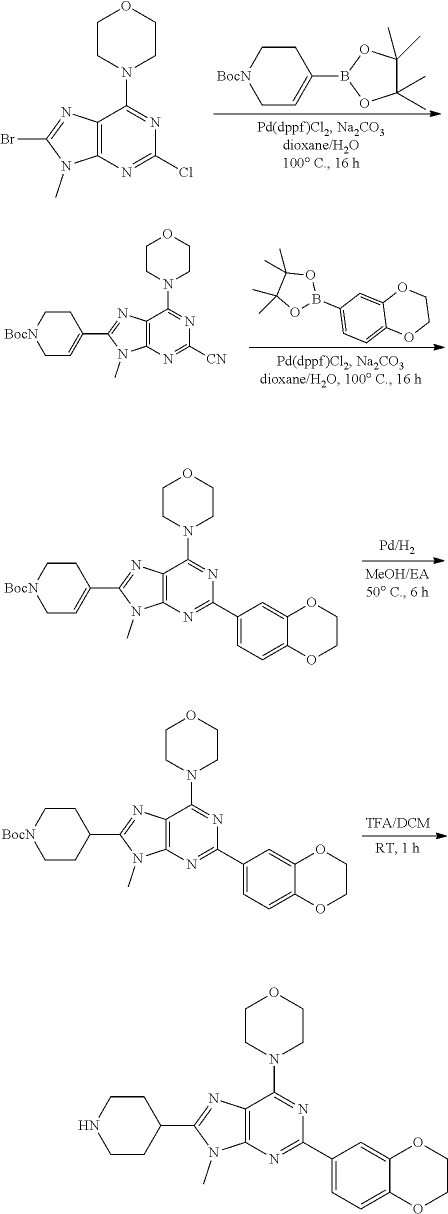

### Step 1: Synthesis of tert-butyl 4-(2-chloro-9-methyl-6-morpholino-9H-purin-8-yl)-3,6-dihydropyridine-1(2H)-carboxylate

A mixture of 4-(8-bromo-2-chloro-9-methyl-9H-purin-6-yl)morpholine (120 mg, 0.36 mmol), tert-butyl 4-(4,4,5,5-tetramethyl-1,3,2-dioxaborolan-2-yl)-3,6-dihydropyridine-1(2H)-carboxylate (112 mg, 0.36 mmol), Na₂CO₃ (115 mg, 1.08 mmol) and Pd(dppf)Cl₂ (26 mg, 0.04 mmol) in dioxane (8 mL) and H₂O (1 mL) was stirred at 80° C. for 2 h under nitrogen atmosphere. The mixture was then concentrated and the crude product was purified by column chromatography (30% EA in PE) to obtain the desired compound as white solid (100 mg, 64%). LCMS (ESI) m/z: 435 [M+H]⁺.

### Step 2: Synthesis of tert-butyl 4-(2-(2,3-dihydrobenzo[b][1,4]dioxin-6-yl)-9-methyl-6-morpholino-9H-purin-8-yl)-3,6-dihydropyridine-1(2H)-carboxylate

A mixture of tert-butyl 4-(2-chloro-9-methyl-6-morpholino-9H-purin-8-yl)-3,6-dihydropyridine-1(2H)-carboxylate (100 mg, 0.23 mmol), 2-(2,3-dihydrobenzo[b][1,4]dioxin-6-yl)-4,4,5,5-tetramethyl-1,3,2-dioxaborolane (121 mg, 0.46 mmol), Na₂CO₃ (73 mg, 0.69 mmol) and Pd(dppf)C12 (17 mg, 0.02 mmol) in dioxane (8 mL) and H₂O (1 mL) was stirred at 80° C. for 2 h under nitrogen atmosphere. The resultant mixture was concentrated and the crude product was purified by column chromatography (30% EA in PE) to obtain the desired product as white solid (80 mg, 65%). LCMS (ESI) m/z: 535 [M+H]⁺.

### Step 3: Synthesis of tert-butyl 4-(2-(2,3-dihydrobenzo[b][1,4]dioxin-6-yl)-9-methyl-6-morpholino-9H-purin-8-yl)piperidine-1-carboxylate

A suspension of tert-butyl 4-(2-(2,3-dihydrobenzo[b][1,4]dioxin-6-yl)-9-methyl-6-morpholino-9H-purin-8-yl)-3,6-dihydropyridine-1(2H)-carboxylate (50 mg, 0.10 mmol) and 10% Pd/C (25 mg) in MeOH (5 mL) and EA (5 mL) was stirred at 80° C. for 16 h under hydrogen atmosphere. The mixture was then filtered and concentrated to obtain the desired product as white solid (30 mg, 60%). LCMS (ESI) m/z: 537 [M+H]⁺.

### Step 4: Synthesis of 4-(2-(2,3-dihydrobenzo[b][1,4]dioxin-6-yl)-9-methyl-8-(piperidin-4-yl)-9H-purin-6-yl)morpholine

To a solution of tert-butyl 4-(2-(2,3-dihydrobenzo[b][1,4]dioxin-6-yl)-9-methyl-6-morpholino-9H-purin-8-yl)piperidine-1-carboxylate (30 mg, 0.10 mmol) in DCM (5 mL) was added TFA (2 mL), the mixture was stirred at room temperature for 1 h. It was concentrated and the crude product was purified by prep-HPLC to obtain the desired product as white solid (3.6 mg, 9%).

¹H NMR (400 MHz, DMSO-d6) δ 7.91-7.86 (m, 2H), 6.92 (d, J=8.4 Hz, 1H), 4.30-4.25 (m, 8H), 3.77-3.75 (m, 7H), 3.33-3.30 (m, 3H), 2.97-3.00 (m, 2H), 2.04-1.93 (m, 4H); LCMS (ESI) m/z: 437.3 [M+H]⁺.

### Compound 114: Synthesis of 4-(9-methyl-8-(pyridin-4-yl)-2-(1,2,3,4-tetrahydroquinolin-7-yl)-9H-purin-6-yl)morpholine

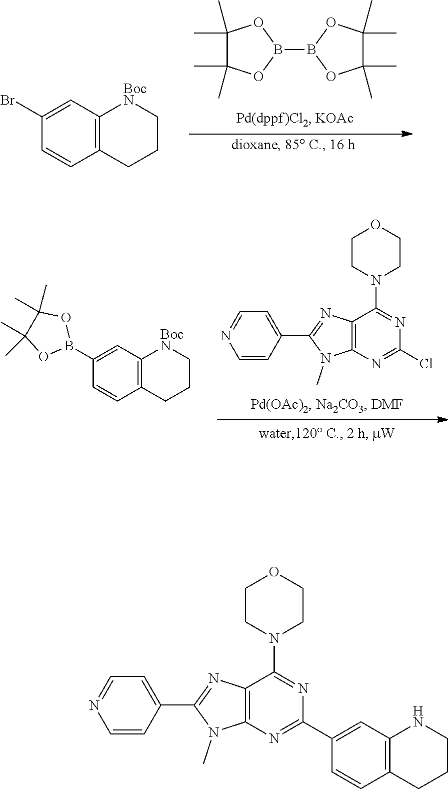

### Step 1: Preparation of tert-butyl 7-(4,4,5,5-tetramethyl-1,3,2-dioxaborolan-2-yl)-3,4-dihydroquinoline-1(2H)-carboxylate

To a solution of tert-butyl 7-bromo-3,4-dihydroquinoline-1(2H)-carboxylate (622 mg, 2 mmol) in dioxane (10 mL) were added 4,4,4′,4′,5,5,5′,5′-octamethyl-2,2′-bi(1,3,2-dioxaborolane) (765 mg, 3 mmol), [1,1′-bis(diphenylphosphino)ferrocene]dichloro palladium (II) (146 mg, 0.2 mmol) and potassium acetate (588 mg, 6 mmol) at 25° C. and the reaction mixture was stirred at 85° C. for 16 h under nitrogen protection. The mixture was then extracted with ethyl acetate (20 mL*2) and washed with water (10 mL*2). The organic layer was dried over sodium sulfate, and concentrated. The crude product was purified by flash chromatography on silica gel (petroleum ether/ester acetic=10:1-3:1) to give crude product tert-butyl 7-(4,4,5,5-tetramethyl-1,3,2-dioxaborolan-2-yl)-3,4-dihydroquinoline-1(2H)-carboxylate as white solid. (610 mg, 84.9%). LCMS (ESI) m/z: 304.2 [M-55]⁺.

### Step 2: Preparation of 4-(9-methyl-8-(pyridin-4-yl)-2-(1,2,3,4-tetrahydroquinolin-7-yl)-9H-purin-6-yl)morpholine

To a solution of 4-(2-chloro-9-methyl-8-(pyridin-4-yl)-9H-purin-6-yl)morpholine (300 mg, 0.83 mmol) in N,N-dimethylformamide (5 mL) was added tert-butyl 7-(4,4,5,5-tetramethyl-1,3,2-dioxaborolan-2-yl)-3,4-dihydroquinoline-1(2H)-carboxylate (132 mg, 0.4 mmol), palladium (II) acetate (20 mg, 0.08 mmol) and sodium carbonate (124 mg, 1.2 mmol) at 25° C. The sealed vial was stirred at 120° C. under microwave for 2 h and the resultant mixture was extracted with ethyl acetate (20 mL*2) and washed with water (10 mL*2). The organic layer was dried over sodium sulfate, and concentrated. The residue (50 mg, 0.1 mmol) was mixed with dichloromethane (5 mL) and trifluoroacetic acid (2 mL), the mixture was stirred at room temperature for 1 h and concentrated. The residue was purified with prep-HPLC (BOSTON pHlex ODS 10 um 21.2×250 mm 120A. The mobile phase was dimethyl sulfoxide/0.1% Ammonium bicarbonate) to obtain 4-(9-methyl-8-(pyridin-4-yl)-2-(1,2,3,4-tetrahydroquinolin-7-yl)-9H-purin-6-yl)morpholine as white solid (17.3 mg, 13.3%).

¹H NMR (400 MHz, DMSO-d6) δ 8.78 (d, J=5.6 Hz, 2H), 7.91 (d, J=5.6 Hz, 2H), 7.62-7.49 (m, 2H), 6.92 (d, J=7.8 Hz, 1H), 5.81 (s, 1H), 4.34 (s, 4H), 3.96 (s, 3H), 3.79 (s, 4H), 3.21 (s, 2H), 2.71 (t, J=5.9 Hz, 2H), 1.82 (s, 2H); LCMS (ESI) m/z: 428.0 [M+H]⁺.

### Example 2. PIKfyve Inhibitory Activity

PIKfyve Biochemical Assay. The biochemical PIKFyve inhibition assays were run by Carna Biosciences according to proprietary methodology based on the Promega ADP-Glo™ Kinase assay. A full-length human PIKFYVE [1-2098(end) amino acids and S696N, L9325, Q995L, T998S, 51033A and Q1183K of the protein having the sequence set forth in NCBI Reference Sequence No. NP_055855.2] was expressed as N-terminal GST-fusion protein (265 kDa) using baculovirus expression system. GST-PIKFYVE was purified by using glutathione sepharose chromatography and used in an ADP-Glo™ Kinase assay (Promega). Reactions were set up by adding the test compound solution, substrate solution, ATP solution and kinase solution, each at 4× final concentrations. Reactions were prepared with assay buffer (50 mM MOPS, 1 mM DTT, pH7.2), mixed, and incubated in black 384 well polystyrene plates for 1 hour at room temperature. ADPGlo™ reagent was then added for 40 minutes, followed by kinase detection reagent for an additional 40 minutes. The kinase activity was evaluated by detecting relative light units on a luminescence plate reader. Samples were run in duplicate from 10 μM to 3 nM. Data was analyzed by setting the control wells (+PIKfyve, no compound) to 0% inhibition and the readout value of background (no PIKfyve) set to 100% inhibition, then the % inhibition of each test solution calculated. IC50 values were calculated from concentration vs % inhibition curves by fitting to a four-parameter logistic curve.

NanoBRET™ TE Intracellular Kinase Assay, K-8 (Promega) Cell-Based Assay. Intracellular inhibition of PIKfyve was assayed using Promega's NanoBRET™ TE Intracellular Kinase Assay, K-8 according to manufacturer's instructions. A dilution series of test compounds was added for 2 hours to HEK293 cells transfected for a minimum of 20 hours with PIKFYVE-NanoLuc® Fusion Vector (Promega) containing a full-length PIKfyve according to manufacturer's specifications in a 96-well plate. Kinase activity was detected by addition of a NanoBRET™ tracer reagent, which was a proprietary PIKfyve inhibitor appended to a fluorescent probe (BRET, bioluminescence resonance energy transfer). Test compounds were tested at concentrations of 10, 3, 1, 0.3, 0.1, 0.03, 0.01, 0.003 μM. BRET signals were measured by a GloMax®Discover Multimode Microplate Reader (Promega) using 0.3 sec/well integration time, 450BP donor filter and 600LP acceptor filters. Active test compounds that bound PIKfyve and displaced the tracer reduced BRET signal. IC50 values were then calculated by fitting the data to the normalized BRET ratio.

The results of the PIKfyve inhibition assays are summarized in the table below.

| | | hPIKfyve |
|---|---|---|
| | hPIKfyve | BRET |
| Compound # | IC₅₀ (μM)^{a} | IC₅₀ (μM)^{a} |
| 1 | +++ | +++ |
| 2 | + | |
| 3 | +++ | |
| 4 | ++ | |
| 5 | +++ | |
| 6 | +++ | +++ |
| 7 | +++ | |
| 8 | ++ | |
| 9 | ++ | |
| 10 | ++ | |
| 11 | ++ | |
| 12 | ++ | |
| 13 | +++ | |
| 14 | +++ | |
| 15 | + | |
| 16 | + | |
| 17 | ++ | |
| 18 | ++ | |
| 19 | + | |
| 20 | + | |
| 21 | +++ | |
| 22 | +++ | |
| 23 | ++ | |
| 24 | ++ | |
| 25 | ++ | |
| 26 | ++ | |
| 27 | +++ | |
| 28 | +++ | +++ |
| 29 | ++ | |
| 30 | ++ | |
| 31 | ++ | |
| 32 | +++ | ++ |
| 33 | ++ | |
| 34 | ++ | |
| 35 | +++ | |
| 36 | + | |
| 37 | ++ | + |
| 38 | ++ | |
| 39 | + | |
| 40 | ++ | |
| 41 | ++ | |
| 42 | ++ | |
| 43 | ++ | |
| 44 | ++ | |
| 45 | +++ | |
| 46 | +++ | |
| 47 | +++ | ++ |
| 48 | ++ | |
| 49 | +++ | |
| 50 | ++ | |
| 51 | ++++ | ++++ |
| 52 | ++++ | ++++ |
| 80 | ++ | |
| 81 | ++ | |
| 82 | +++ | |
| 83 | +++ | |
| 84 | +++ | |
| 85 | +++ | |
| 86 | ++++ | |
| 88 | ++++ | |
| 89 | ++ | |
| 90 | +++ | |
| 91 | ++ | |
| 92 | +++ | |
| 93 | +++ | |
| 94 | +++ | |
| 95 | ++ | |
| 96 | + | |
| 97 | +++ | |
| 98 | +++ | |
| 99 | +++ | |
| 100 | ++++ | |
| 101 | +++ | |
| 102 | +++ | |
| 103 | +++ | |
| 104 | +++ | |
| 105 | + | |
| 106 | ++++ | |
| 107 | ++++ | |
| 108 | ++++ | |
| 109 | +++ | |
| 110 | ++++ | |
| 113 | ++++ | |
| 114 | ++++ | |
| 117 | ++ | |
| 118 | +++ | +++ |
| 119 | ++++ | ++++ |
| 120 | ++++ | |
| 121 | ++++ | |
| 122 | +++ | |
| 123 | +++ | |
| 124 | +++ | |
| 125 | ++ | |

^{a}++++ stands for <10 nM, +++ stands for 10-100 nM, ++ stands for 100-1000 nM, + stands for 1-10 μM, and − stands for >10 μM.

### Example 3. Viability Assay to Assess TDP-43 Toxicity in FAB1 TDP-43 and PIKfyve TDP-43 Yeast Cells

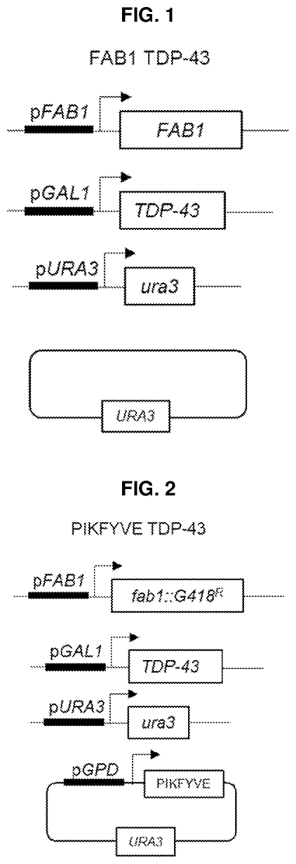

Generation of TDP-43 yeast model expressing human PIKfyve. Human PIKFYVE (“entry clone”) was cloned into pAG416GPDccdB (“destination vector”) according to standard Gateway cloning protocols (Invitrogen, Life Technologies). The resulting pAG416GPD-PIKFYVE plasmids were amplified in E. coli and plasmid identity confirmed by restriction digest and Sanger sequencing. Lithium acetate/polyethylene glycol-based transformation was used to introduce the above PIKFYVE plasmid into a BY4741 yeast strain auxotrophic for the ura3 gene and deleted for two transcription factors that regulate the xenobiotic efflux pumps, a major efflux pump, and FAB1, the yeast ortholog of PIKFYVE (MATa, snq2::KILeu2; pdr3::Klura3; pdr1::NATMX; fab1::G418R, his3; leu2; ura3; met15; LYS2+) (FIG. 2). Transformed yeast were plated on solid agar plates with complete synthetic media lacking uracil (CSM-ura) and containing 2% glucose. Individual colonies harboring the control or PIKFYVE TDP-43 plasmids were recovered. A plasmid containing wild-type TDP-43 under the transcriptional control of the GAL1 promoter and containing the hygromycin-resistance gene as a selectable marker was transformed into the fab/::G418R pAG416GPD-PIKFYVE yeast strain (FIG. 1). Transformed yeast were plated on CSM-ura containing 2% glucose and 200 μg/mL G418 after overnight recovery in media lacking antibiotic. Multiple independent isolates were further evaluated for cytotoxicity and TDP-43 expression levels.

Viability Assay. A control yeast strain with the wild-type yeast FAB1 gene and TDP-43 (“FAB1 TDP-43”, carries empty pAG416 plasmid), and the “PIKFYVE TDP-43” yeast strain, were assessed for toxicity using a propidium iodide viability assay. Both yeast strains were transferred from solid CSM-ura/2% glucose agar plates into 3 mL of liquid CSM-ura/2% glucose media for 6-8 hours at 30° C. with aeration. Yeast cultures were then diluted to an optical density at 600 nm wavelength (OD₆₀₀) of 0.005 in 3 mL of CSM-ura/2% raffinose and grown overnight at 30° C. with aeration to an OD₆₀₀ of 0.3-0.8. Log-phase overnight cultures were diluted to OD₆₀₀ of 0.005 in CSM-ura containing either 2% raffinose or galactose and 150 μL dispensed into each well of a flat bottom 96-well plates. Compounds formulated in 100% dimethyl sulfoxide (DMSO) were serially diluted in DMSO and 1.5 μL diluted compound transferred to the 96-well plates using a multichannel pipet. Wells containing DMSO alone were also evaluated as controls for compound effects. Tested concentrations ranged from 15 μM to 0.11 μM. Cultures were immediately mixed to ensure compound distribution and covered plates incubated at 30° C. for 24 hours in a stationary, humified incubator.

Upon the completion of incubation, cultures were assayed for viability using propidium iodide (PI) to stain for dead/dying cells. A working solution of PI was made where, for each plate, 1 μL of 10 mM PI was added to 10 mL of CSM-ura (raffinose or galactose). The final PI solution (50 4/well) was dispensed into each well of a new round bottom 96-well plate. The overnight 96-well assay plate was then mixed with a multichannel pipet and 50 μL transferred to the PI-containing plate. This plate was then incubated for 30 minutes at 30° C. in the dark. A benchtop flow cytometer (Miltenyi MACSquant) was then used to assess red fluorescence (B2 channel), forward scatter, and side scatter (with following settings: gentle mix, high flow rate, fast measurement, 10,000 events). Intensity histograms were then gated for “PI-positive” or “PI-negative” using the raffinose and galactose cultures treated with DMSO as controls. The DMSO controls for raffinose or galactose-containing cultures were used to determine the window of increased cell death and this difference set to 100. All compounds were similarly gated and then compared to this maximal window to establish the percent reduction in PI-positive cells. IC50 values were then calculated for compounds that demonstrated a concentration-dependent enhancement of viability by fitting a logistic regression curve.

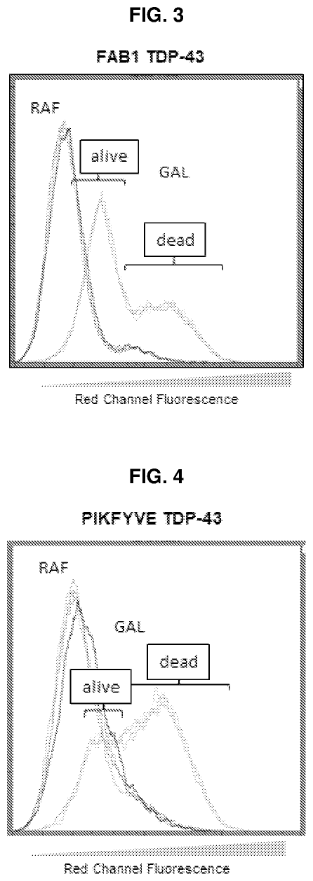

Upon induction of TDP-43 in both strains, there was a marked increase in inviable cells (rightmost population) with both FAB1 TDP-43 and PIKFYVE TDP-43, with a more pronounced effect in PIKFYVE TDP-43 (FIGS. 3 and 4).

PIKfyve Inhibition Suppresses Toxicity in PIKfyve TDP-43 Model. The biochemical PIKFyve inhibition assays were run by Carna Biosciences according to proprietary methodology based on the Promega ADP-Glo™ Kinase assay. A full-length human PIKFYVE [1-2098(end) amino acids and S696N, L9325, Q995L,T998S, 51033A and Q1183K of accession number NP_055855.2] was expressed as N-terminal GST-fusion protein (265 kDa) using baculovirus expression system. GST-PIKFYVE was purified by using glutathione sepharose chromatography and used in an ADP-Glo™ Kinase assay (Promega). Reactions were set up by adding the test compound solution, substrate solution, ATP solution and kinase solution, each at 4× final concentrations. Reactions were prepared with assay buffer (50 mM MOPS, 1 mM DTT, pH7.2), mixed, and incubated in black 384 well polystyrene plates for 1 hour at room temperature. ADP-Glo™ reagent was then added for 40 minutes, followed by kinase detection reagent for an additional 40 minutes. The kinase activity was evaluated by detecting relative light units on a luminescence plate reader. Samples were run in duplicate from 10 uM to 3 nM. Data was analyzed by setting the control wells (+PIKfyve, no compound) to 0% inhibition and the readout value of background (no PIKfyve) set to 100% inhibition, then the % inhibition of each test solution calculated. IC50 values were calculated from concentration vs % inhibition curves by fitting to a four-parameter logistic curve.

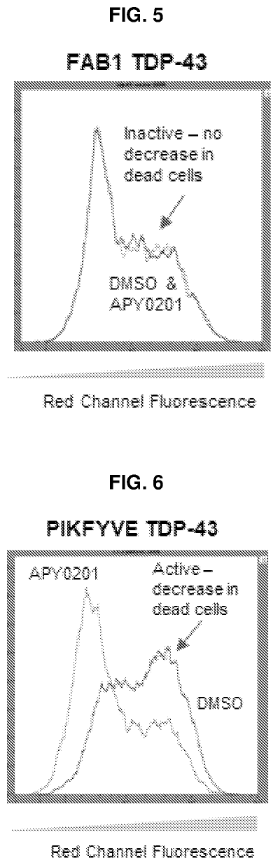

Activity of APY0201, a known PIKFYVE inhibitor, in FAB1 TDP-43 (FIG. 5) and PIKFYVE TDP-43 (FIG. 6). There was no increase in viable cells in FAB1 TDP-43 across a range of compound concentrations as evidenced by a lack in reduction of the right most population of propidium iodide-positive cells (only 0.23 μM is shown). In the PIKFYVE TDP-43 model, 0.23 μM reduced the population of propidium iodide-positive dead cells, indicating PIKFYVE inhibition ameliorated TDP-43 toxicity. Concentrations ranging from 0.5 mM to less than 100 nM afforded increased viability.

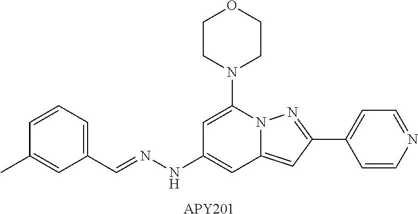 [SMILES: "APY201" ; Cc1cccc(/C=N/Nc2cc(N3CCOCC3)n3nc(-c4ccncc4)cc3c2)c1]

A panel of compounds was tested in a biochemical PIKFYVE assay (ADP-Glo™ with full-length PIKfyve) and IC50's determined (nM) (see the Table below). The same compounds were also tested in both FAB1 and PIKFYVE TDP-43 yeast models. Their activity is reported here as “active” or “inactive.” Compounds with low nanomolar potency in the biochemical assay were active in the PIKFYVE TDP-43 yeast model. Compounds that were less potent or inactive in the biochemical assay were inactive in the PIKFYVE TDP-43 model. Compounds that were inactive in the biochemical or PIKFYVE TDP-43 assays were plotted with the highest concentrations tested in that assay.

| | PIKfyve | FAB1 | PIKfyve |
|---|---|---|---|
| | IC₅₀ | TDP-43 | TDP-43 |
| Structure | (nM) | (active/inactive) | (active/inactive) |
| 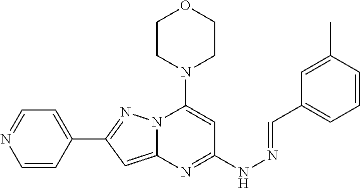 [SMILES: Cc1cccc(/C=N/Nc2cc(N3CCOCC3)n3nc(-c4ccncc4)cc3n2)c1] | 7.5 | Inactive | Active |
| 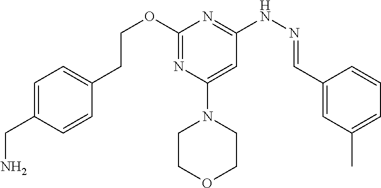 [SMILES: Cc1cccc(/C=N/Nc2cc(N3CCOCC3)nc(OCCc3ccc(CN)cc3)n2)c1] | 12 | Inactive | Active |
| 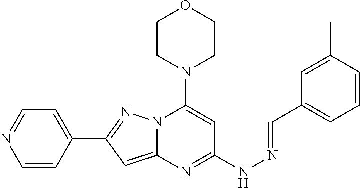 [SMILES: Cc1cccc(/C=N/Nc2cc(N3CCOCC3)n3nc(-c4ccncc4)cc3n2)c1] | 4.9 | Inactive | Active |
| 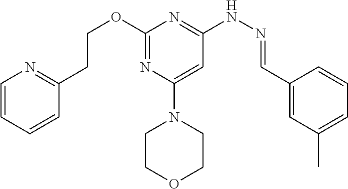 [SMILES: Cc1cccc(/C=N/Nc2cc(N3CCOCC3)nc(OCCc3ccccn3)n2)c1] | 640 | Inactive | Inactive |
| 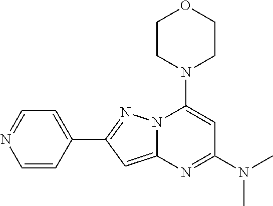 [SMILES: CN(C)c1cc(N2CCOCC2)n2nc(-c3ccncc3)cc2n1] | 2007 | Inactive | Inactive |
| 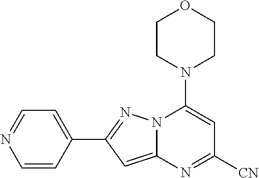 [SMILES: N#Cc1cc(N2CCOCC2)n2nc(-c3ccncc3)cc2n1] | >10000 | Inactive | Inactive |

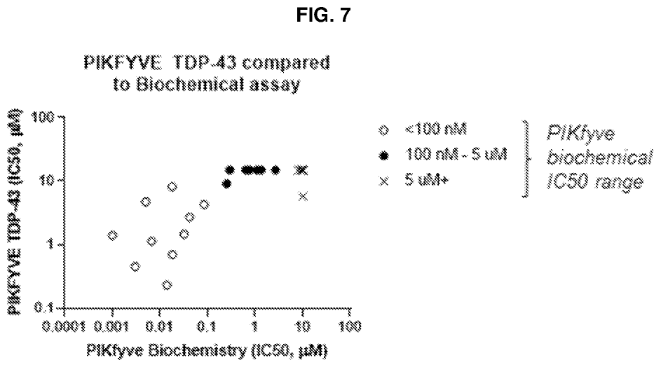

Biochemical and Efficacy Assays. A larger set of PIKfyve inhibitors were evaluated in both a PIKfyve kinase domain binding assay (nanobret) and in the PIKFYVE TDP-43 yeast strain. IC50 values (μM) were plotted. Data points are formatted based on binned potency from the nanobret assay as indicated in the legend (FIG. 7). Below is a table of compounds and their biochemical and PIKFYVE TDP-43 IC50 values plotted in FIG. 7.

| | PIKFYVE | PIKFYVE |
|---|---|---|
| | Biochemistry | TDP-43 |
| Structure | (IC50, μM) | (IC50, μM) |
| 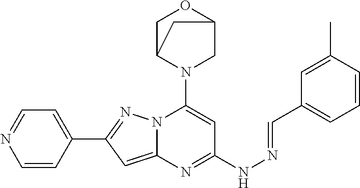 [SMILES: Cc1cccc(/C=N/Nc2cc(N3CC4CC3CO4)n3nc(-c4ccncc4)cc3n2)c1] | 0.003 | 0.450 |
| 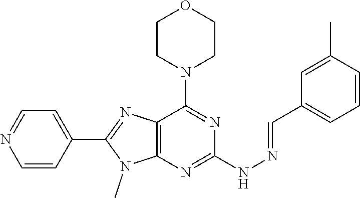 [SMILES: Cc1cccc(/C=N/Nc2nc(N3CCOCC3)c3nc(-c4ccncc4)n(C)c3n2)c1] | 0.001 | 1.390 |
| 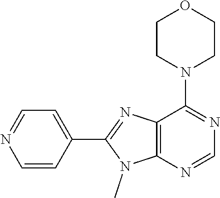 [SMILES: Cn1c(-c2ccncc2)nc2c(N3CCOCC3)ncnc21] | 0.007 | 1.120 |
| 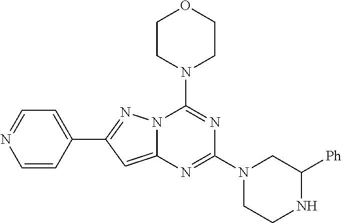 [SMILES: c1ccc(C2CN(c3nc(N4CCOCC4)n4nc(-c5ccncc5)cc4n3)CCN2)cc1] | 2.660 | >15 |
| 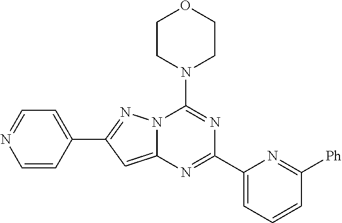 [SMILES: c1ccc(-c2cccc(-c3nc(N4CCOCC4)n4nc(-c5ccncc5)cc4n3)n2)cc1] | 0.014 | 0.230 |
| 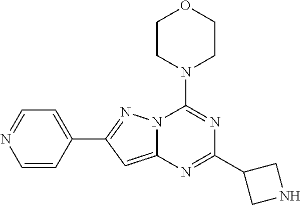 [SMILES: c1cc(-c2cc3nc(C4CNC4)nc(N4CCOCC4)n3n2)ccn1] | 8.020 | >15 |
| 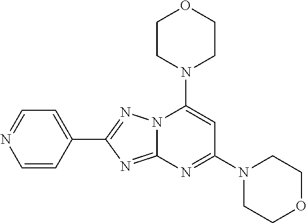 [SMILES: c1cc(-c2nc3nc(N4CCOCC4)cc(N4CCOCC4)n3n2)ccn1] | 9.200 | >15 |
| 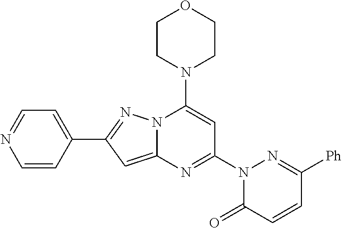 [SMILES: O=c1ccc(-c2ccccc2)nn1-c1cc(N2CCOCC2)n2nc(-c3ccncc3)cc2n1] | 0.295 | >15 |
| 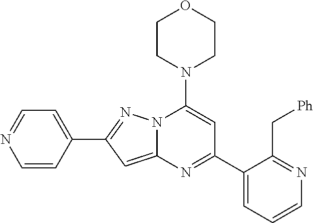 [SMILES: c1ccc(Cc2ncccc2-c2cc(N3CCOCC3)n3nc(-c4ccncc4)cc3n2)cc1] | 1.090 | >15 |
| 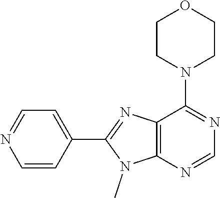 [SMILES: Cn1c(-c2ccncc2)nc2c(N3CCOCC3)ncnc21] | 0.640 | >15 |
| 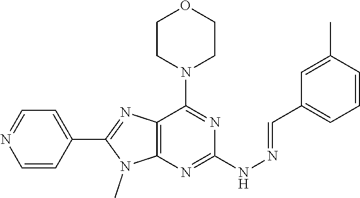 [SMILES: Cc1cccc(/C=N/Nc2nc(N3CCOCC3)c3nc(-c4ccncc4)n(C)c3n2)c1] | 0.005 | 4.720 |
| 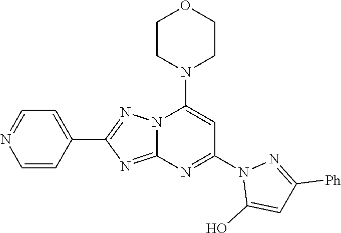 [SMILES: Oc1cc(-c2ccccc2)nn1-c1cc(N2CCOCC2)n2nc(-c3ccncc3)nc2n1] | 0.018 | 0.693 |
| 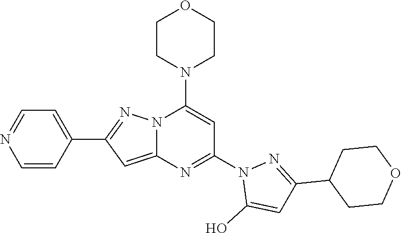 [SMILES: Oc1cc(C2CCOCC2)nn1-c1cc(N2CCOCC2)n2nc(-c3ccncc3)cc2n1] | 0.253 | 9.105 |
| 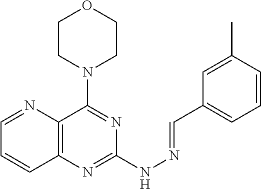 [SMILES: Cc1cccc(/C=N/Nc2nc(N3CCOCC3)c3ncccc3n2)c1] | 0.018 | 8.214 |
| 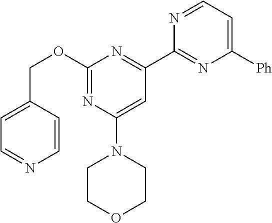 [SMILES: c1ccc(-c2ccnc(-c3cc(N4CCOCC4)nc(OCc4ccncc4)n3)n2)cc1] | 0.032 | 1.447 |
| 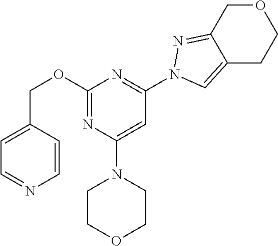 [SMILES: c1cc(COc2nc(N3CCOCC3)cc(-n3cc4c(n3)COCC4)n2)ccn1] | 1.343 | >15 |
| 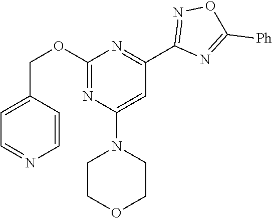 [SMILES: c1ccc(-c2nc(-c3cc(N4CCOCC4)nc(OCc4ccncc4)n3)no2)cc1] | >10 | >15 |
| 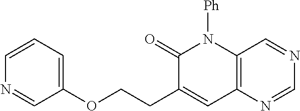 [SMILES: O=c1c(CCOc2cccnc2)cc2ncncc2n1-c1ccccc1] | >10 | >15 |
| 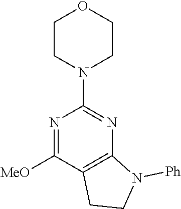 [SMILES: COc1nc(N2CCOCC2)nc2c1CCN2c1ccccc1] | 0.085 | 4.273 |
| 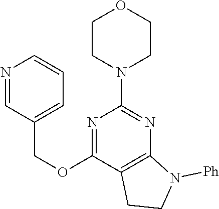 [SMILES: c1ccc(N2CCc3c(OCc4cccnc4)nc(N4CCOCC4)nc32)cc1] | 0.042 | 2.685 |
| 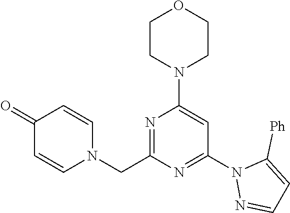 [SMILES: O=c1ccn(Cc2nc(N3CCOCC3)cc(-n3nccc3-c3ccccc3)n2)cc1] | >10 | >15 |
| 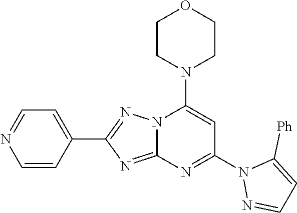 [SMILES: c1ccc(-c2ccnn2-c2cc(N3CCOCC3)n3nc(-c4ccncc4)nc3n2)cc1] | 0.767 | >15 |
| 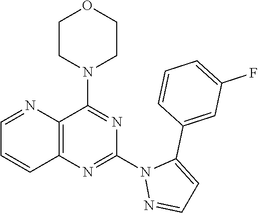 [SMILES: Fc1cccc(-c2ccnn2-c2nc(N3CCOCC3)c3ncccc3n2)c1] | >10 | 5.754 |

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A compound of formula (I): 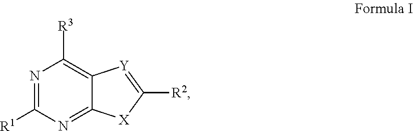
or a pharmaceutically acceptable salt thereof,
wherein
X is NR^{A}, S, or O;
Y is CR A or N;
R¹ is optionally substituted C₁₋₉ heteroaryl comprising a 5-membered ring having a nitrogen atom at position 2 relative to the bond to the core, optionally substituted pyrimidin-6-yl, or optionally substituted benzodioxanyl;
R² is optionally substituted C₆₋₁₀ aryl, optionally substituted C₁₋₉ heterocyclyl, or optionally C₁₋₉ substituted heteroaryl; and
R³ is 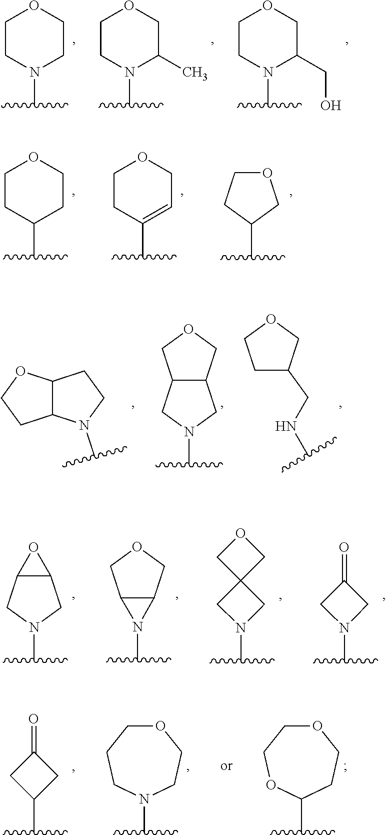
and
each R^{A} is independently H, optionally substituted C₁₋₆ alkyl, optionally substituted C₆₋₁₀ aryl, optionally substituted C₁₋₉ heteroaryl.

2. The compound of claim 1, wherein X is NR^{A}.

3. The compound of claim 1, wherein Y is N.

4. The compound of claim 1, wherein R³ is

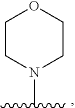 [SMILES: *N1CCOCC1]

5. The compound of claim 1, wherein the compound is of formula Ia: 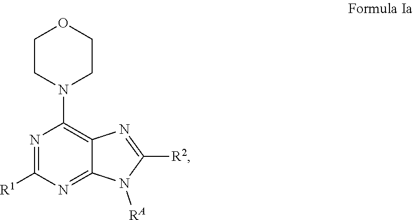
or a pharmaceutically acceptable salt thereof.

6. The compound of claim 1, wherein R^{A} is optionally substituted C₁₋₆ alkyl.

7. The compound of claim 1, wherein R^{A} is H.

8. The compound of claim 1, wherein the compound is of formula Ib: 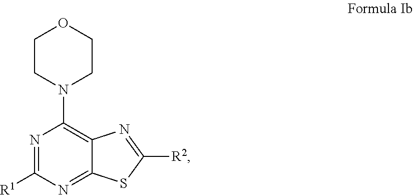
or a pharmaceutically acceptable salt thereof.

9. The compound of claim 1, wherein the compound is of formula Ic: 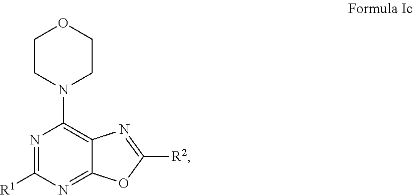
or a pharmaceutically acceptable salt thereof.

10. The compound of claim 1, wherein the compound is of formula Id: 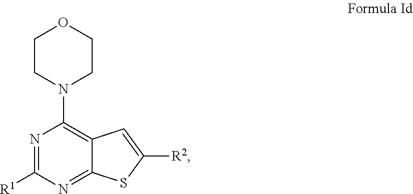
or a pharmaceutically acceptable salt thereof.

11. (canceled)

12. The compound of claim 1, wherein R¹ is optionally substituted pyrazol-1-yl.

13. (canceled)

14. (canceled)

15. The compound of claim 1, wherein R¹ is optionally substituted pyrazol-3-yl.

16. (canceled)

17. (canceled)

18. The compound of claim 1, wherein R¹ is optionally substituted pyrimidin-6-yl.

19. The compound of claim 1, wherein R² is optionally substituted C₁₋₉ heteroaryl.

20. The compound of claim 19, wherein R² is optionally substituted pyridyl.

21. The compound of claim 1, wherein R² is optionally substituted tetrahydropyranyl, optionally substituted dihydropyranyl, optionally substituted piperidinyl, or optionally substituted azetidinyl.

22. (canceled)

23. A compound of the following structure: 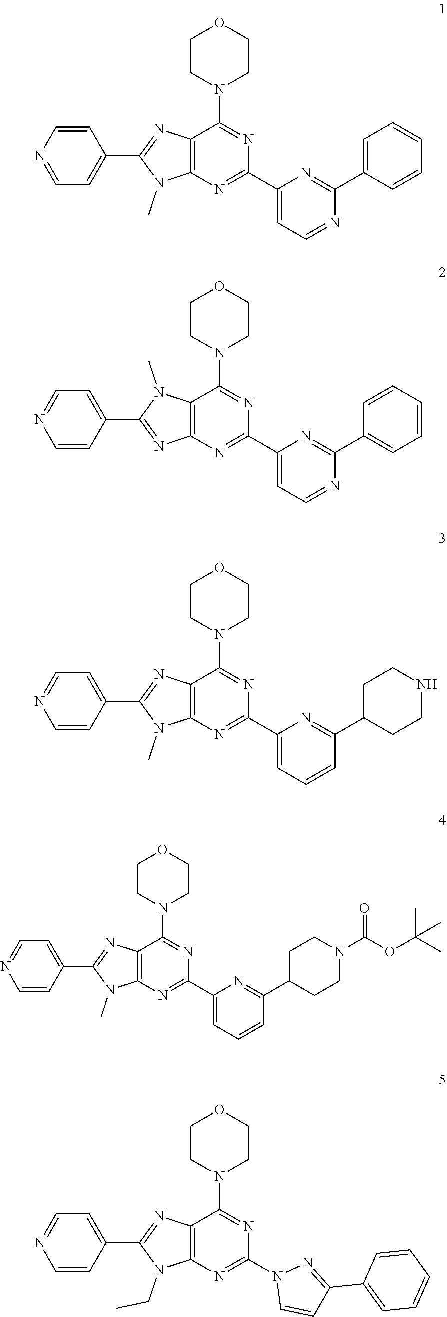 [SMILES: Cn1c(-c2ccncc2)nc2c(N3CCOCC3)nc(-c3ccnc(-c4ccccc4)n3)nc21 ; Cn1c(-c2ccncc2)nc2nc(-c3ccnc(-c4ccccc4)n3)nc(N3CCOCC3)c21 ; Cn1c(-c2ccncc2)nc2c(N3CCOCC3)nc(-c3cccc(C4CCNCC4)n3)nc21 ; Cn1c(-c2ccncc2)nc2c(N3CCOCC3)nc(-c3cccc(C4CCN(C(=O)OC(C)(C)C)CC4)n3)nc21 ; CCn1c(-c2ccncc2)nc2c(N3CCOCC3)nc(-n3ccc(-c4ccccc4)n3)nc21] 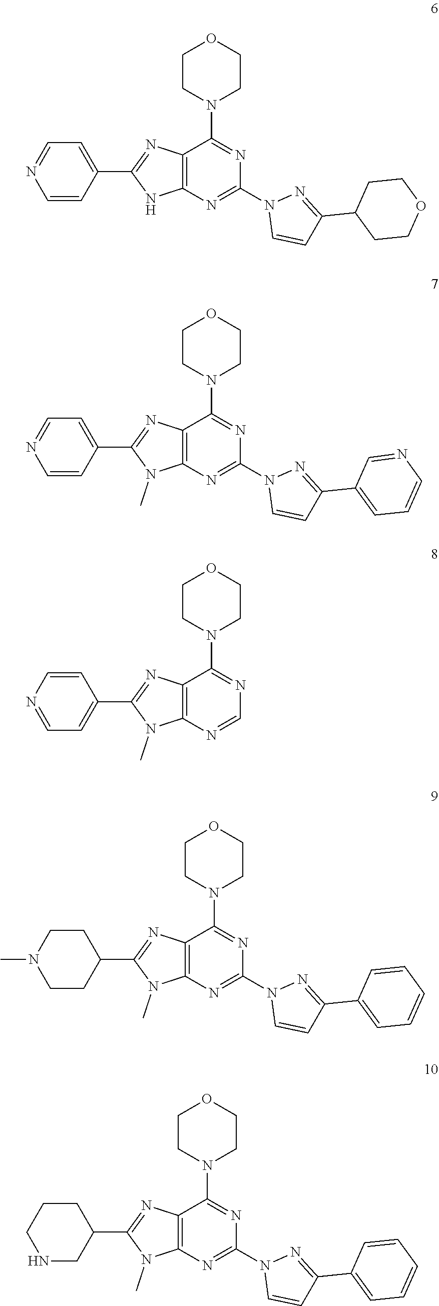 [SMILES: c1cc(-c2nc3c(N4CCOCC4)nc(-n4ccc(C5CCOCC5)n4)nc3[nH]2)ccn1 ; Cn1c(-c2ccncc2)nc2c(N3CCOCC3)nc(-n3ccc(-c4cccnc4)n3)nc21 ; Cn1c(-c2ccncc2)nc2c(N3CCOCC3)ncnc21 ; CN1CCC(c2nc3c(N4CCOCC4)nc(-n4ccc(-c5ccccc5)n4)nc3n2C)CC1 ; Cn1c(C2CCCNC2)nc2c(N3CCOCC3)nc(-n3ccc(-c4ccccc4)n3)nc21] 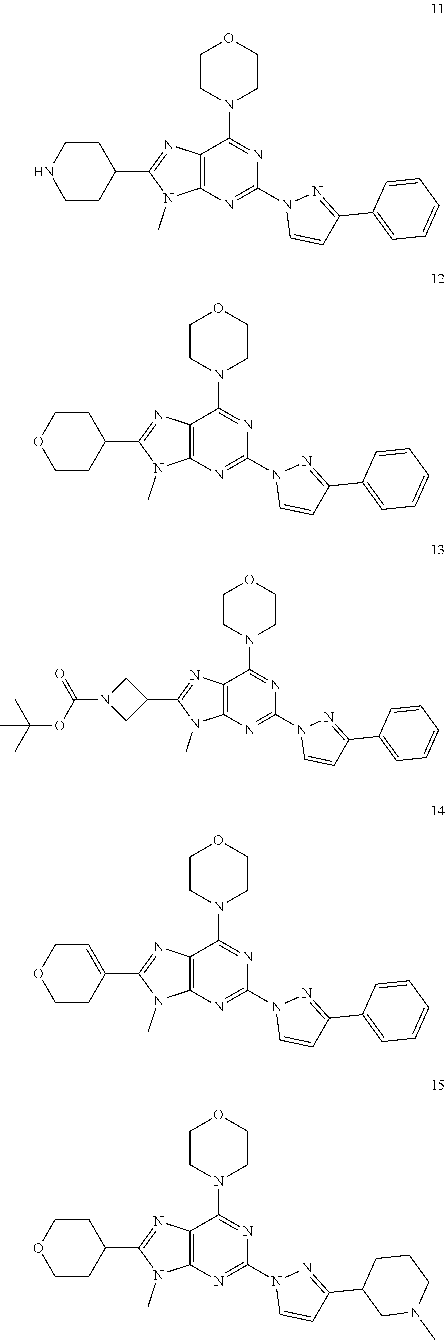 [SMILES: Cn1c(C2CCNCC2)nc2c(N3CCOCC3)nc(-n3ccc(-c4ccccc4)n3)nc21 ; Cn1c(C2CCOCC2)nc2c(N3CCOCC3)nc(-n3ccc(-c4ccccc4)n3)nc21 ; Cn1c(C2CN(C(=O)OC(C)(C)C)C2)nc2c(N3CCOCC3)nc(-n3ccc(-c4ccccc4)n3)nc21 ; Cn1c(C2=CCOCC2)nc2c(N3CCOCC3)nc(-n3ccc(-c4ccccc4)n3)nc21 ; CN1CCCC(c2ccn(-c3nc(N4CCOCC4)c4nc(C5CCOCC5)n(C)c4n3)n2)C1] 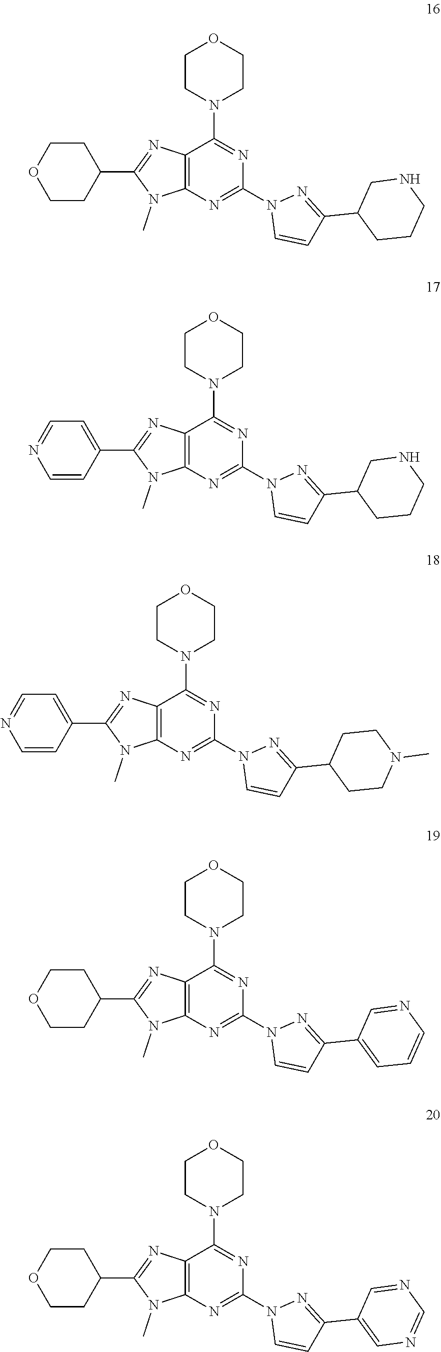 [SMILES: Cn1c(C2CCOCC2)nc2c(N3CCOCC3)nc(-n3ccc(C4CCCNC4)n3)nc21 ; Cn1c(-c2ccncc2)nc2c(N3CCOCC3)nc(-n3ccc(C4CCCNC4)n3)nc21 ; CN1CCC(c2ccn(-c3nc(N4CCOCC4)c4nc(-c5ccncc5)n(C)c4n3)n2)CC1 ; Cn1c(C2CCOCC2)nc2c(N3CCOCC3)nc(-n3ccc(-c4cccnc4)n3)nc21 ; Cn1c(C2CCOCC2)nc2c(N3CCOCC3)nc(-n3ccc(-c4cncnc4)n3)nc21] 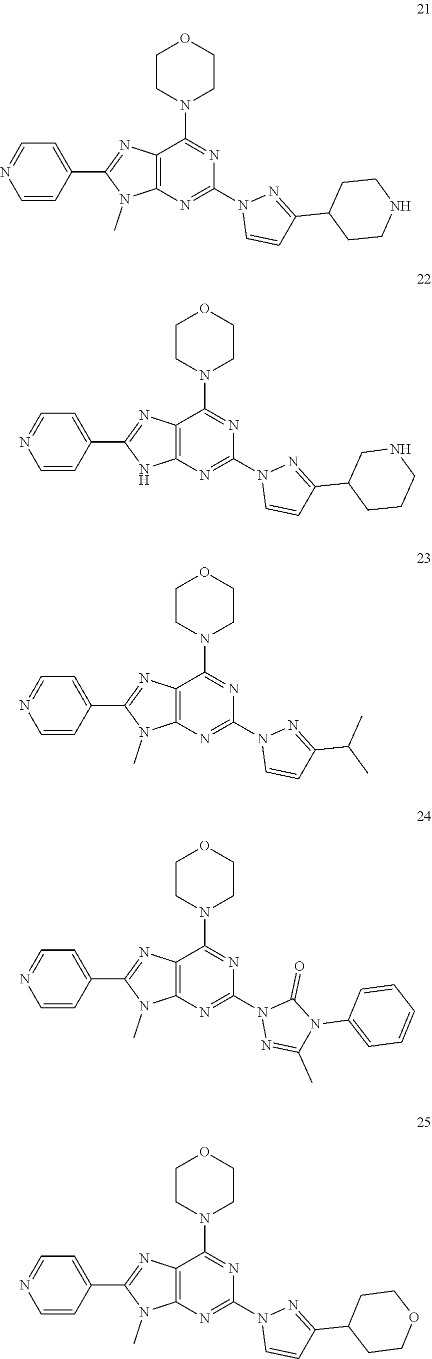 [SMILES: Cn1c(-c2ccncc2)nc2c(N3CCOCC3)nc(-n3ccc(C4CCNCC4)n3)nc21 ; c1cc(-c2nc3c(N4CCOCC4)nc(-n4ccc(C5CCCNC5)n4)nc3[nH]2)ccn1 ; CC(C)c1ccn(-c2nc(N3CCOCC3)c3nc(-c4ccncc4)n(C)c3n2)n1 ; Cc1nn(-c2nc(N3CCOCC3)c3nc(-c4ccncc4)n(C)c3n2)c(=O)n1-c1ccccc1 ; Cn1c(-c2ccncc2)nc2c(N3CCOCC3)nc(-n3ccc(C4CCOCC4)n3)nc21] 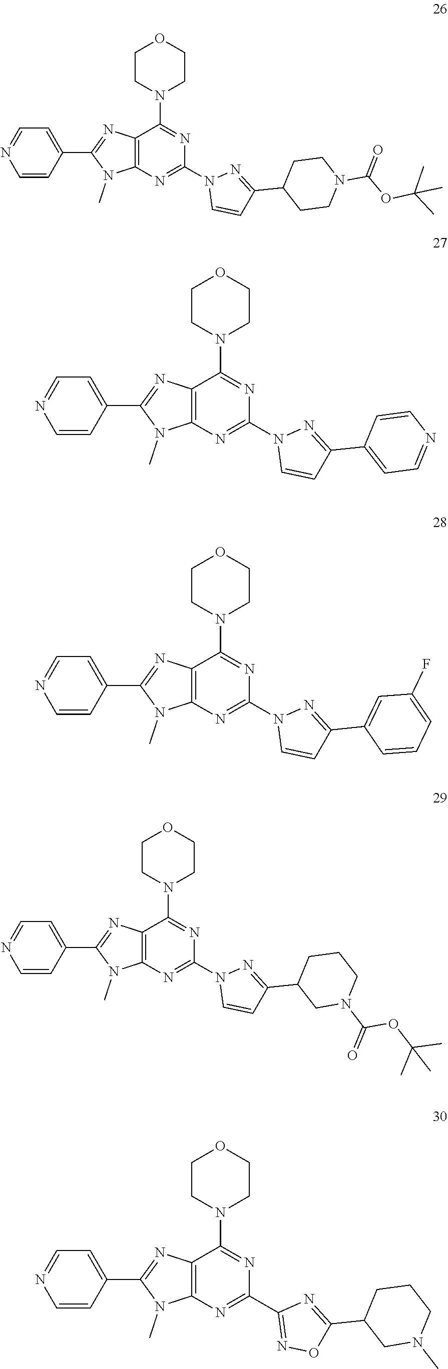 [SMILES: Cn1c(-c2ccncc2)nc2c(N3CCOCC3)nc(-n3ccc(C4CCN(C(=O)OC(C)(C)C)CC4)n3)nc21 ; Cn1c(-c2ccncc2)nc2c(N3CCOCC3)nc(-n3ccc(-c4ccncc4)n3)nc21 ; Cn1c(-c2ccncc2)nc2c(N3CCOCC3)nc(-n3ccc(-c4cccc(F)c4)n3)nc21 ; Cn1c(-c2ccncc2)nc2c(N3CCOCC3)nc(-n3ccc(C4CCCN(C(=O)OC(C)(C)C)C4)n3)nc21 ; CN1CCCC(c2nc(-c3nc(N4CCOCC4)c4nc(-c5ccncc5)n(C)c4n3)no2)C1] 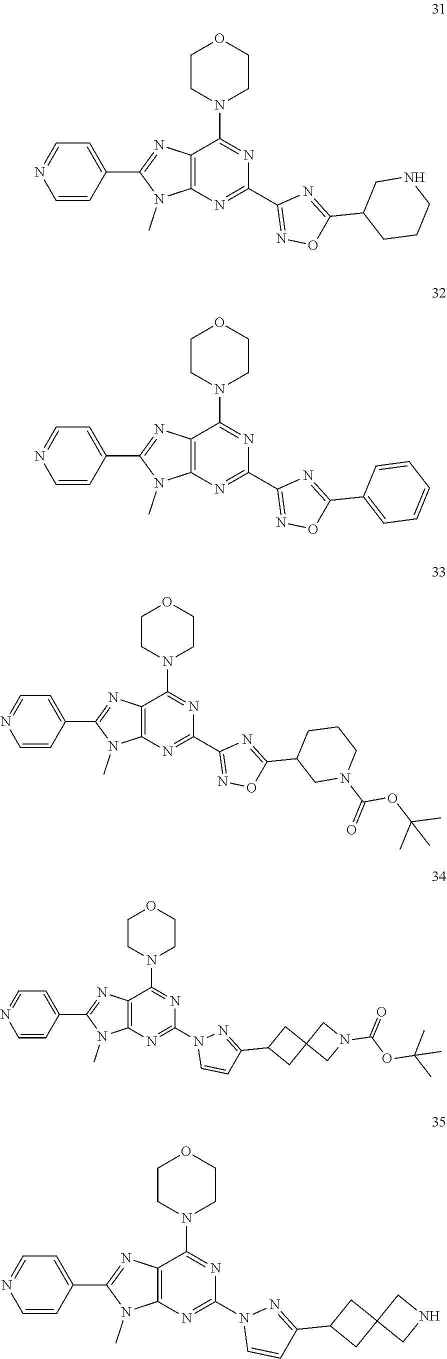 [SMILES: Cn1c(-c2ccncc2)nc2c(N3CCOCC3)nc(-c3noc(C4CCCNC4)n3)nc21 ; Cn1c(-c2ccncc2)nc2c(N3CCOCC3)nc(-c3noc(-c4ccccc4)n3)nc21 ; Cn1c(-c2ccncc2)nc2c(N3CCOCC3)nc(-c3noc(C4CCCN(C(=O)OC(C)(C)C)C4)n3)nc21 ; Cn1c(-c2ccncc2)nc2c(N3CCOCC3)nc(-n3ccc(C4CC5(C4)CN(C(=O)OC(C)(C)C)C5)n3)nc21 ; Cn1c(-c2ccncc2)nc2c(N3CCOCC3)nc(-n3ccc(C4CC5(CNC5)C4)n3)nc21] 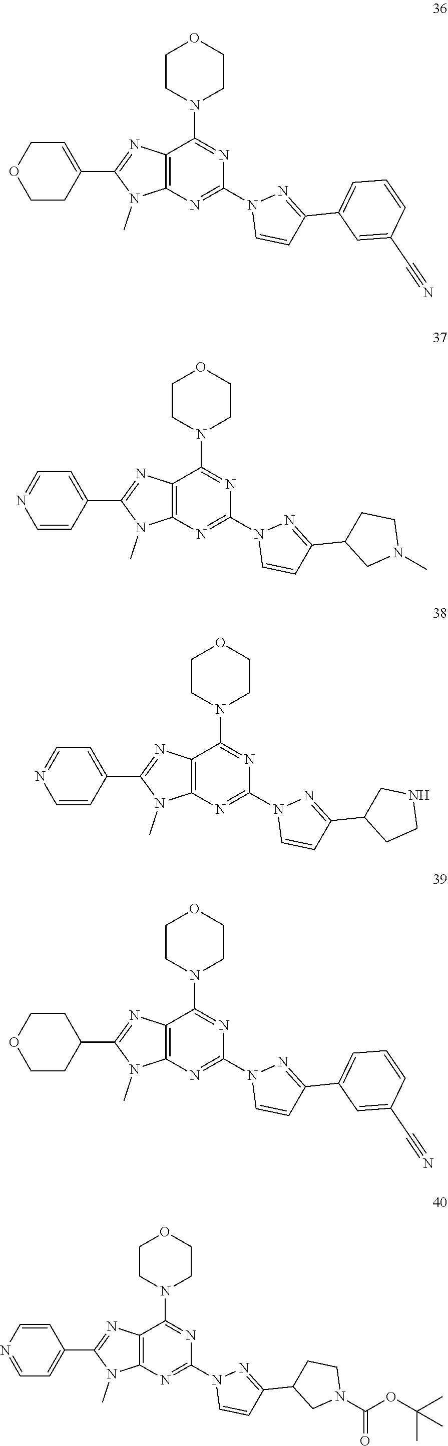 [SMILES: Cn1c(C2=CCOCC2)nc2c(N3CCOCC3)nc(-n3ccc(-c4cccc(C#N)c4)n3)nc21 ; CN1CCC(c2ccn(-c3nc(N4CCOCC4)c4nc(-c5ccncc5)n(C)c4n3)n2)C1 ; Cn1c(-c2ccncc2)nc2c(N3CCOCC3)nc(-n3ccc(C4CCNC4)n3)nc21 ; Cn1c(C2CCOCC2)nc2c(N3CCOCC3)nc(-n3ccc(-c4cccc(C#N)c4)n3)nc21 ; Cn1c(-c2ccncc2)nc2c(N3CCOCC3)nc(-n3ccc(C4CCN(C(=O)OC(C)(C)C)C4)n3)nc21] 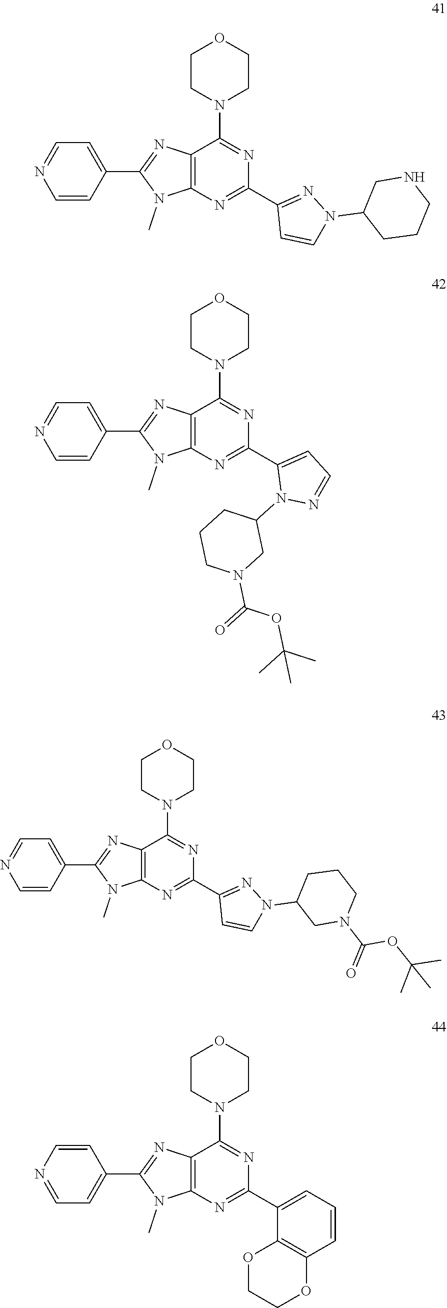 [SMILES: Cn1c(-c2ccncc2)nc2c(N3CCOCC3)nc(-c3ccn(C4CCCNC4)n3)nc21 ; Cn1c(-c2ccncc2)nc2c(N3CCOCC3)nc(-c3ccnn3C3CCCN(C(=O)OC(C)(C)C)C3)nc21 ; Cn1c(-c2ccncc2)nc2c(N3CCOCC3)nc(-c3ccn(C4CCCN(C(=O)OC(C)(C)C)C4)n3)nc21 ; Cn1c(-c2ccncc2)nc2c(N3CCOCC3)nc(-c3cccc4c3OCCO4)nc21] 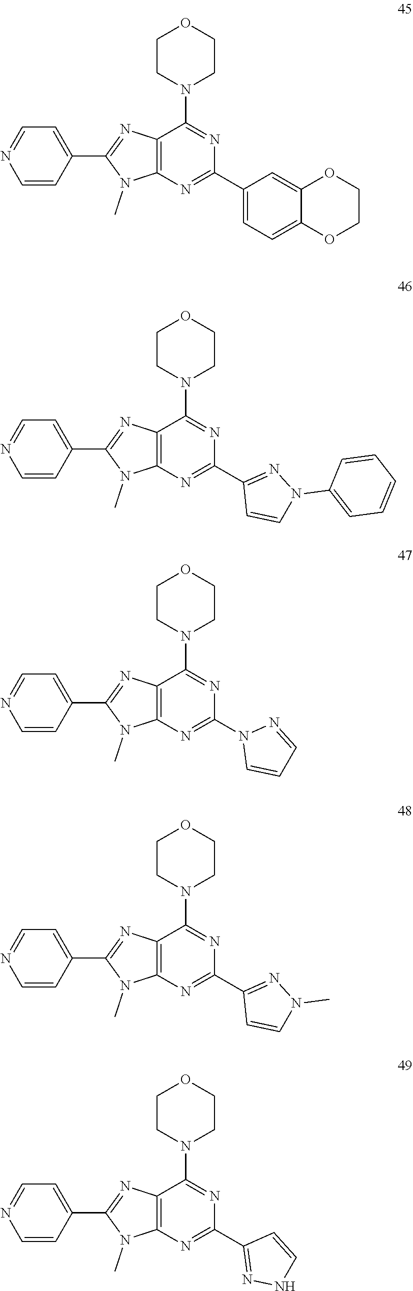 [SMILES: Cn1c(-c2ccncc2)nc2c(N3CCOCC3)nc(-c3ccc4c(c3)OCCO4)nc21 ; Cn1c(-c2ccncc2)nc2c(N3CCOCC3)nc(-c3ccn(-c4ccccc4)n3)nc21 ; Cn1c(-c2ccncc2)nc2c(N3CCOCC3)nc(-n3cccn3)nc21 ; Cn1ccc(-c2nc(N3CCOCC3)c3nc(-c4ccncc4)n(C)c3n2)n1 ; Cn1c(-c2ccncc2)nc2c(N3CCOCC3)nc(-c3cc[nH]n3)nc21] 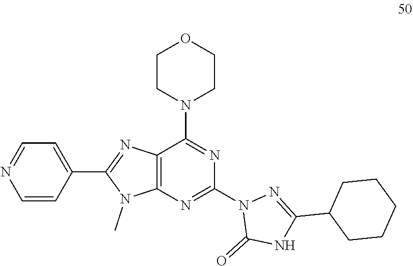 [SMILES: Cn1c(-c2ccncc2)nc2c(N3CCOCC3)nc(-n3nc(C4CCCCC4)[nH]c3=O)nc21]
or a pharmaceutically acceptable salt thereof.

24. A compound of the following structure: 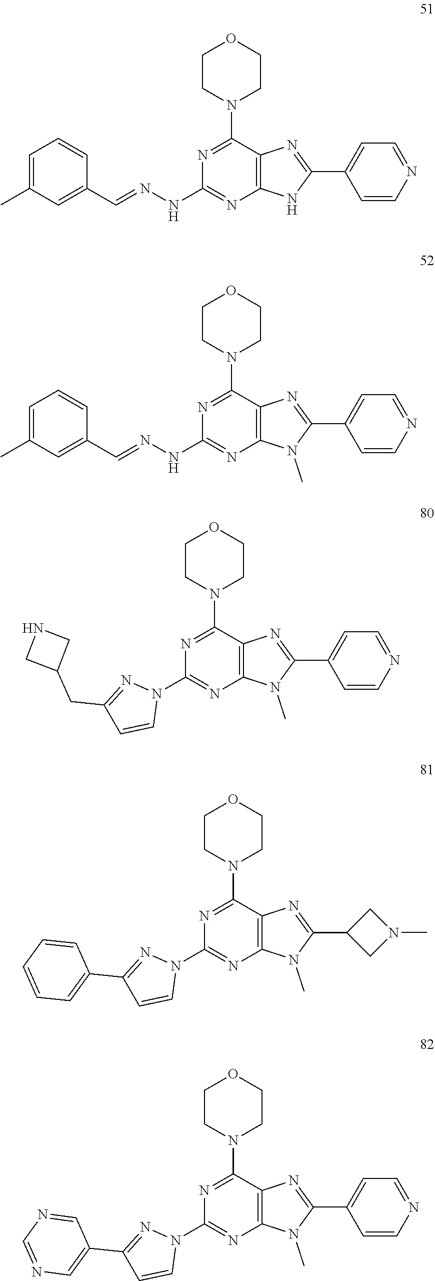 [SMILES: Cc1cccc(/C=N/Nc2nc(N3CCOCC3)c3nc(-c4ccncc4)[nH]c3n2)c1 ; Cc1cccc(/C=N/Nc2nc(N3CCOCC3)c3nc(-c4ccncc4)n(C)c3n2)c1 ; Cn1c(-c2ccncc2)nc2c(N3CCOCC3)nc(-n3ccc(CC4CNC4)n3)nc21 ; CN1CC(c2nc3c(N4CCOCC4)nc(-n4ccc(-c5ccccc5)n4)nc3n2C)C1 ; Cn1c(-c2ccncc2)nc2c(N3CCOCC3)nc(-n3ccc(-c4cncnc4)n3)nc21] 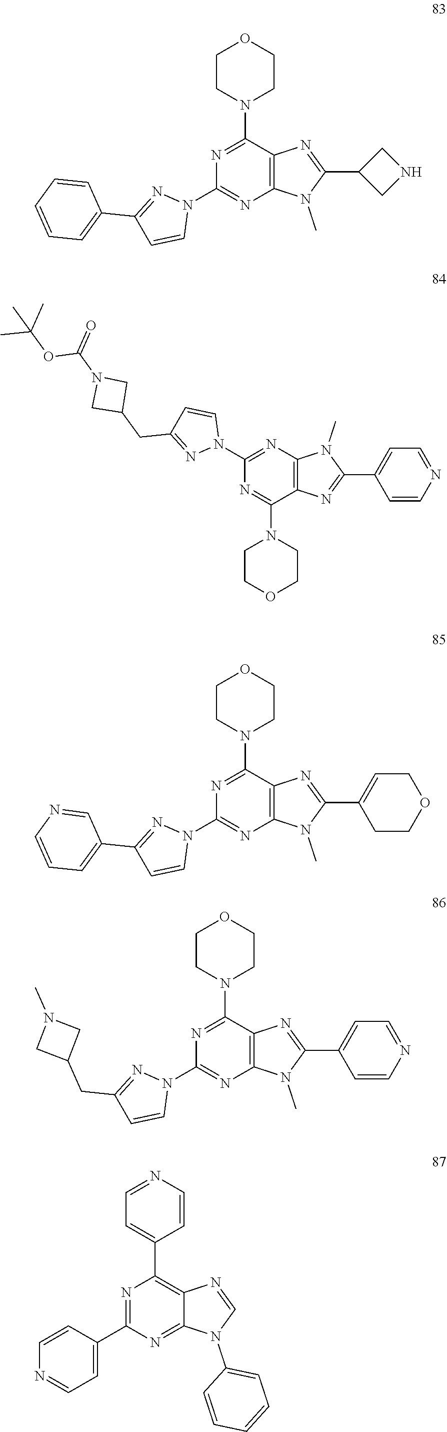 [SMILES: Cn1c(C2CNC2)nc2c(N3CCOCC3)nc(-n3ccc(-c4ccccc4)n3)nc21 ; Cn1c(-c2ccncc2)nc2c(N3CCOCC3)nc(-n3ccc(CC4CN(C(=O)OC(C)(C)C)C4)n3)nc21 ; Cn1c(C2=CCOCC2)nc2c(N3CCOCC3)nc(-n3ccc(-c4cccnc4)n3)nc21 ; CN1CC(Cc2ccn(-c3nc(N4CCOCC4)c4nc(-c5ccncc5)n(C)c4n3)n2)C1 ; c1ccc(-n2cnc3c(-c4ccncc4)nc(-c4ccncc4)nc32)cc1] 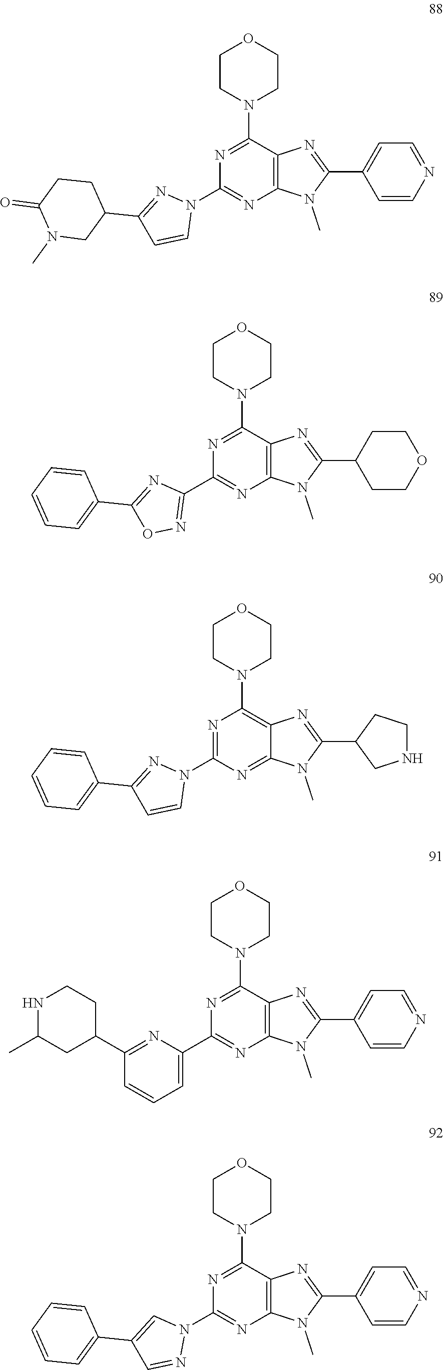 [SMILES: CN1CC(c2ccn(-c3nc(N4CCOCC4)c4nc(-c5ccncc5)n(C)c4n3)n2)CCC1=O ; Cn1c(C2CCOCC2)nc2c(N3CCOCC3)nc(-c3noc(-c4ccccc4)n3)nc21 ; Cn1c(C2CCNC2)nc2c(N3CCOCC3)nc(-n3ccc(-c4ccccc4)n3)nc21 ; CC1CC(c2cccc(-c3nc(N4CCOCC4)c4nc(-c5ccncc5)n(C)c4n3)n2)CCN1 ; Cn1c(-c2ccncc2)nc2c(N3CCOCC3)nc(-n3cc(-c4ccccc4)cn3)nc21] 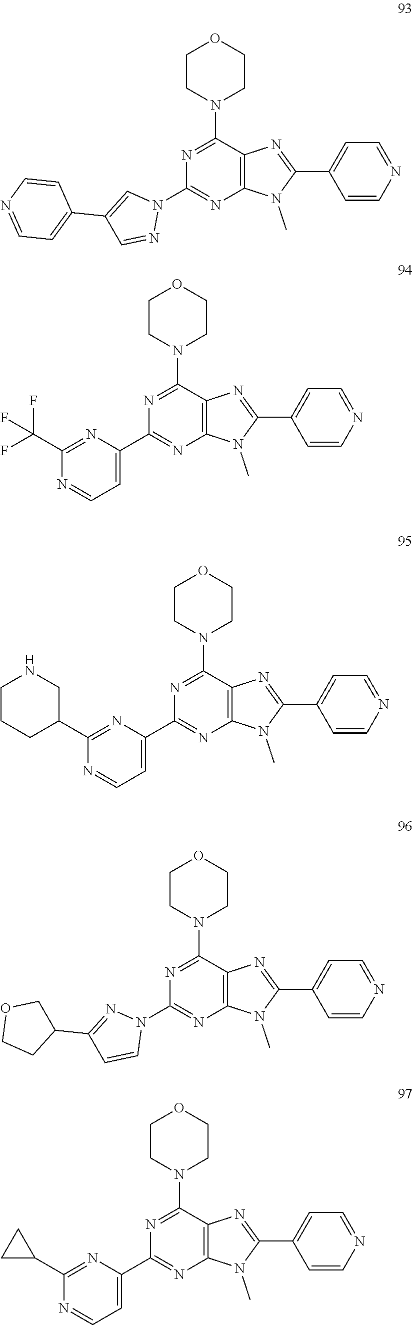 [SMILES: Cn1c(-c2ccncc2)nc2c(N3CCOCC3)nc(-n3cc(-c4ccncc4)cn3)nc21 ; Cn1c(-c2ccncc2)nc2c(N3CCOCC3)nc(-c3ccnc(C(F)(F)F)n3)nc21 ; Cn1c(-c2ccncc2)nc2c(N3CCOCC3)nc(-c3ccnc(C4CCCNC4)n3)nc21 ; Cn1c(-c2ccncc2)nc2c(N3CCOCC3)nc(-n3ccc(C4CCOC4)n3)nc21 ; Cn1c(-c2ccncc2)nc2c(N3CCOCC3)nc(-c3ccnc(C4CC4)n3)nc21] 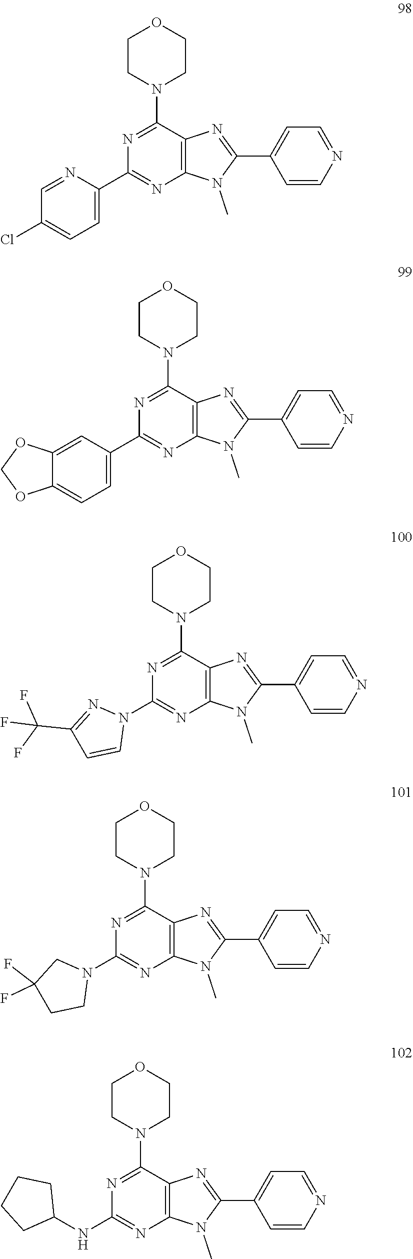 [SMILES: Cn1c(-c2ccncc2)nc2c(N3CCOCC3)nc(-c3ccc(Cl)cn3)nc21 ; Cn1c(-c2ccncc2)nc2c(N3CCOCC3)nc(-c3ccc4c(c3)OCO4)nc21 ; Cn1c(-c2ccncc2)nc2c(N3CCOCC3)nc(-n3ccc(C(F)(F)F)n3)nc21 ; Cn1c(-c2ccncc2)nc2c(N3CCOCC3)nc(N3CCC(F)(F)C3)nc21 ; Cn1c(-c2ccncc2)nc2c(N3CCOCC3)nc(NC3CCCC3)nc21] 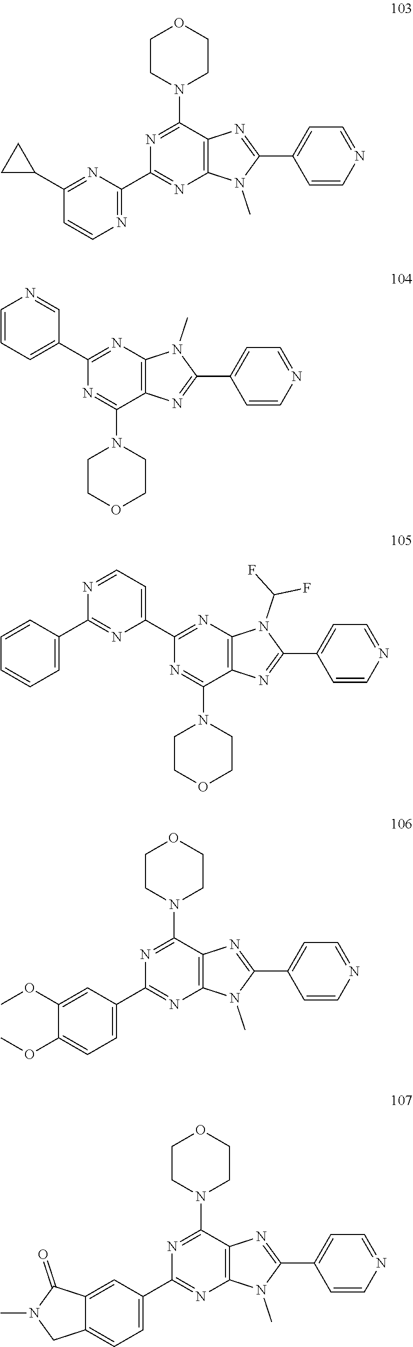 [SMILES: Cn1c(-c2ccncc2)nc2c(N3CCOCC3)nc(-c3nccc(C4CC4)n3)nc21 ; Cn1c(-c2ccncc2)nc2c(N3CCOCC3)nc(-c3cccnc3)nc21 ; FC(F)n1c(-c2ccncc2)nc2c(N3CCOCC3)nc(-c3ccnc(-c4ccccc4)n3)nc21 ; COc1ccc(-c2nc(N3CCOCC3)c3nc(-c4ccncc4)n(C)c3n2)cc1OC ; CN1Cc2ccc(-c3nc(N4CCOCC4)c4nc(-c5ccncc5)n(C)c4n3)cc2C1=O] 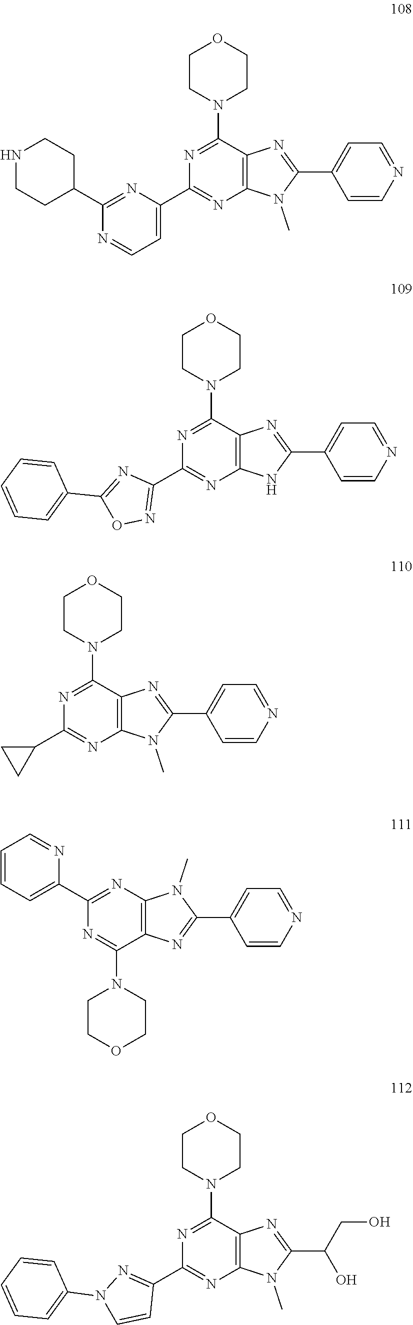 [SMILES: Cn1c(-c2ccncc2)nc2c(N3CCOCC3)nc(-c3ccnc(C4CCNCC4)n3)nc21 ; c1ccc(-c2nc(-c3nc(N4CCOCC4)c4nc(-c5ccncc5)[nH]c4n3)no2)cc1 ; Cn1c(-c2ccncc2)nc2c(N3CCOCC3)nc(C3CC3)nc21 ; Cn1c(-c2ccncc2)nc2c(N3CCOCC3)nc(-c3ccccn3)nc21 ; Cn1c(C(O)CO)nc2c(N3CCOCC3)nc(-c3ccn(-c4ccccc4)n3)nc21] 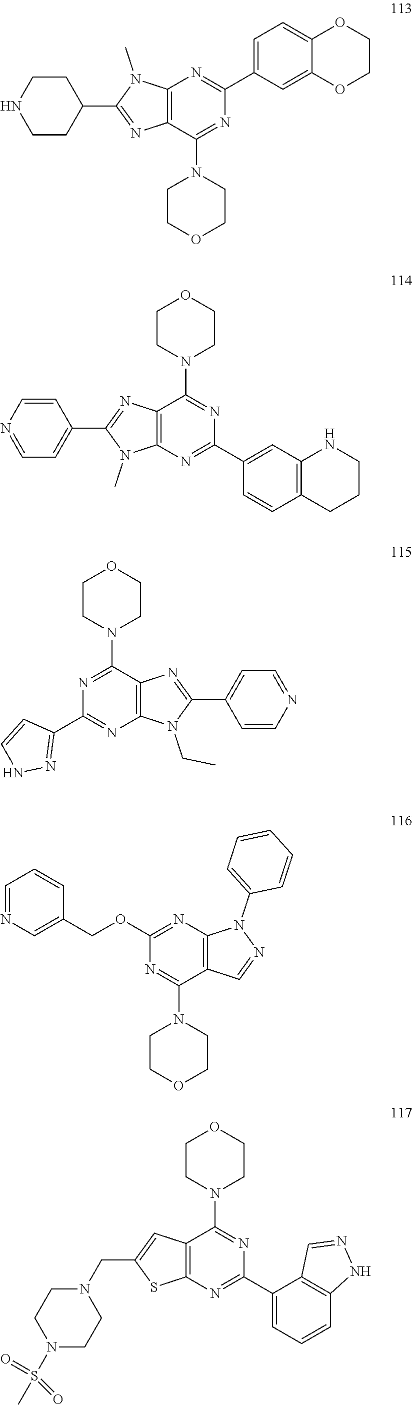 [SMILES: Cn1c(C2CCNCC2)nc2c(N3CCOCC3)nc(-c3ccc4c(c3)OCCO4)nc21 ; Cn1c(-c2ccncc2)nc2c(N3CCOCC3)nc(-c3ccc4c(c3)NCCC4)nc21 ; CCn1c(-c2ccncc2)nc2c(N3CCOCC3)nc(-c3cc[nH]n3)nc21 ; c1ccc(-n2ncc3c(N4CCOCC4)nc(OCc4cccnc4)nc32)cc1 ; CS(=O)(=O)N1CCN(Cc2cc3c(N4CCOCC4)nc(-c4cccc5[nH]ncc45)nc3s2)CC1] 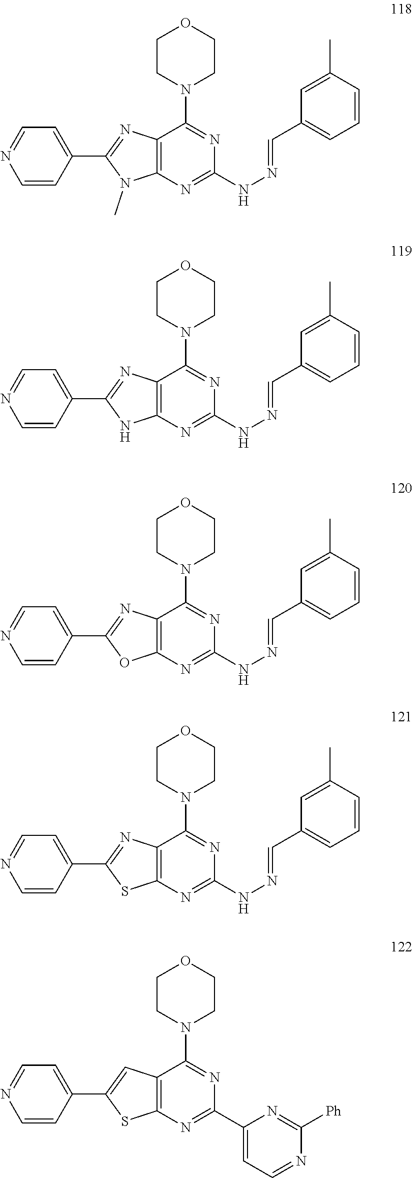 [SMILES: Cc1cccc(/C=N/Nc2nc(N3CCOCC3)c3nc(-c4ccncc4)n(C)c3n2)c1 ; Cc1cccc(/C=N/Nc2nc(N3CCOCC3)c3nc(-c4ccncc4)[nH]c3n2)c1 ; Cc1cccc(/C=N/Nc2nc(N3CCOCC3)c3nc(-c4ccncc4)oc3n2)c1 ; Cc1cccc(/C=N/Nc2nc(N3CCOCC3)c3nc(-c4ccncc4)sc3n2)c1 ; c1ccc(-c2nccc(-c3nc(N4CCOCC4)c4cc(-c5ccncc5)sc4n3)n2)cc1] 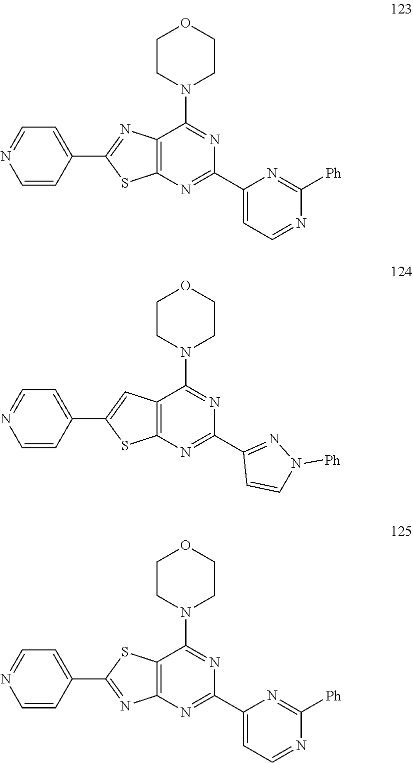 [SMILES: c1ccc(-c2nccc(-c3nc(N4CCOCC4)c4nc(-c5ccncc5)sc4n3)n2)cc1 ; c1ccc(-n2ccc(-c3nc(N4CCOCC4)c4cc(-c5ccncc5)sc4n3)n2)cc1 ; c1ccc(-c2nccc(-c3nc(N4CCOCC4)c4sc(-c5ccncc5)nc4n3)n2)cc1]
or a pharmaceutically acceptable salt thereof.

25. A pharmaceutical composition comprising the compound of claim 1, or a pharmaceutically acceptable salt thereof, and a pharmaceutically acceptable excipient.

26. A method of treating a neurological disorder in a subject in need thereof, the method comprising administering to the subject an effective amount of the compound of claim 1, or a pharmaceutically acceptable salt thereof.

27.-58. (canceled)
